(19) United States
(12) Patent Application Publication
(10) Pub. No.: US 2010/0112568 A1
(43) Pub. Date: May 6, 2010

(54) METHODS AND KITS FOR DIAGNOSIS OF MULTIPLE SCLEROSIS IN PROBABLE MULTIPLE SCLEROSIS SUBJECTS

(75) Inventors: Anat Achiron, Tel-Aviv (IL); Michael Gurevich, Rechovot (IL)

Correspondence Address: MARTIN D. MOYNIHAN d/b/a PRTSI, INC., P.O. BOX 16446, ARLINGTON, VA, 22215, US

(73) Assignee: TEL HASHOMER MEDICAL RESEARCH INFRASTRUCTURE AND S, Ramat-Gan (IL)

(21) Appl. No.: 12/448,612

(22) PCT Filed: Dec. 27, 2007
(86) PCT No.: PCT/IL2007/001616

§ 371 Date: Jan. 6, 2010

Related U.S. Application Data
(60) Provisional application No. 60/877,682, filed on Dec. 29, 2006.

(51) Int. Cl. C12Q 1/68

(52) U.S. Cl. 435/6

(57) ABSTRACT

Provided are methods and kits for determining the probability of a subject diagnosed with probable multiple sclerosis to develop definite diagnosis of multiple sclerosis by determining the expression level of polynucleotides which are differentially expressed between subjects diagnosed with probable multiple sclerosis and which further develop definite multiple sclerosis and unaffected subjects. Also provided are methods and kits for selecting a treatment regimen of a subject diagnosed with probable multiple sclerosis.

### FIELD AND BACKGROUND OF THE INVENTION

The present invention, in some embodiments thereof, relates to genetic markers which are differentially expressed between subjects diagnosed with probable multiple sclerosis (MS) which further develop the definite diagnosis of MS and control subjects, more particularly, but not exclusively, to methods and kits using same for determining the probability of a subject diagnosed with probable multiple sclerosis to develop a definite diagnosis of multiple sclerosis and for treating subjects diagnosed with probable multiple sclerosis.

Multiple sclerosis (MS) is the most common central nervous system (CNS) disease affecting young adults (disease onset between 20 to 40 years of age), and the third leading cause for disability after trauma and rheumatic diseases. Disease prevalence in USA is 120/100,000, (250,000 to 350,000 cases), and in Israel about 30/100,000. MS is a multifactorial disease that develops in genetically predisposed subjects exposed to yet undefined environmental factors and which results in irreversible neurological disability.

The diagnosis of MS is defined primary by clinical terms and relies on a combination of history, neurological examination and ancillary laboratory and neuro-imaging studies. Typically, at onset of MS, an otherwise healthy person presents with the acute or sub-acute neurological symptomatology (attack). The symptoms usually remain for several days to few weeks, and then partially or completely resolve. The neurological symptoms are accompanied by demyelinating lesions on brain MRI. Thus, the laboratory diagnosis of probable MS is based on: 1) Cerebro-spinal fluid (CSF) evaluation of IgG synthesis, oligoclonal bands; and 2) MRI of the brain and spinal cord. After a period of remission, a second attack will occur. During this period between the first and second attacks, the patient is diagnosed as probable MS. Only when the second attack occurs, the diagnosis of clinically definite MS is established.

In about 85% of the patients with definite diagnosis of MS, the disease course is relapsing-remitting definite MS (RRMS), which is characterized by attacks during which new neurological symptoms and signs appear, or existing neurological symptoms and signs worsen. Usually an attack develops within a period of several days, lasts for 6-8 weeks, and then gradually resolves. During an acute attack, scattered inflammatory and demyelinating CNS lesions produce varying combinations of motor, sensory, coordination, visual, and cognitive impairments, as well as symptoms of fatigue and urinary tract dysfunction. The outcome of an attack is unpredictable in terms of neurological squeal, but it is well established that with each attack, the probability of complete clinical remission decreases, and neurological disability and handicap are liable to develop. In about 15% of patients the disease has a primary progressive course, characterized by gradual onset of neurological symptoms that progress over time. In a subset of patients (about 40%), the disease has a secondary progressive course, i.e., it is first characterized by relapses and remission and then gradually progresses (See FIGS. 4a-c). The only course of MS in which treatment was effectively established is RRMS. Various immunomodulatory drugs have been shown to reduce the number and severity of acute attacks, and thereby to decrease the accumulation of neurological disability.

The main pathologic findings in MS are the presence of infiltrating mononuclear cells predominantly T lymphocytes and macrophages that surpass the blood brain barrier and induce an active inflammation within the brain and spinal cord, attacking the myelin and resulting in gliotic scars and axonal loss. These inflammatory (acute and chronic) processes can be visualized by brain and spinal cord magnetic resonance imaging (MRI) as hyperintense T2 or hypointense T1 lesions. Thus, MRI examination can serve for the diagnosis of the disease and as a surrogate marker to follow disease activity by measuring lesion load within the brain.

The etiology of MS is still unknown. The pathogenesis of MS involves autoimmune mechanisms associated with autoreactive T cells against myelin antigens. It is well established that not one dominant gene determines genetic susceptibility to develop MS, but rather many genes, each with different influence, are involved. The initial pathogenic process that triggers the disease might be caused by one group of genes, while other groups are probably involved in disease activity and progression (5, 6).

In a previous epidemiological study the present inventors have shown that 57.6% of patients with probable MS experience a second attack within one year from onset, and thus convert to definite MS (7). In other studies, the progression to clinically definite MS in patients with an abnormal brain MRI was 49% and 65% in the first 5 years, 41% and 68% within 2 years, and 24% and 45% within 1 year (8, 9, respectively). Prediction of disease progression rate is especially important during the initial stage, when patients first present with neurological symptomatology and are defined to suffer from probable MS. At this early stage the immunological process of epitope spreading which is associated with exposure of the immune system to myelin antigens is still limited and significant disability has not yet developed.

The potential application of DNA microarray technology for understanding neurological disorders was discussed in a recent review (12). In MS, microarray analysis of brain lesions and brains of mice with experimental allergic encephalomyelitis (EAE)—the experimental animal model of MS —identified genes that contribute to lesion pathology (13). Similarly, different expression of transcribed genes encoding inflammatory cytokines was demonstrated in acute inflammatory brain lesions compared with ‘silent’ lesions without inflammation, using a large-scale gene microarray analysis (14).

In the peripheral blood of MS patients, simultaneous inhibitory and stimulatory effects of inflammatory T cells and macrophages reflect their potential role within the ongoing autoimmune response was reported. Analysis of the expression pattern in peripheral blood mononuclear cells (PBMC) obtained from MS patients during a stable clinical remission revealed 34 genes out of more than 4000 tested that were significantly different from controls (15). In a previous study by the present inventors (16) PBMC gene expression pattern of 26 RRMS patients and 18 healthy subjects demonstrated significantly different pattern of 1109 genes between patients and healthy subjects. This signature contains genes that implicate the underlying processes involved in MS pathogenesis including T-cell activation and expansion, inflammation and apoptosis. To determine disease stage related gene expression signatures MS patients were evaluated during an acute relapse and in remission (16; PCT Pub. No. WO03081201A2, EP1532268A2, AU3214604AH, US20060003327A1, to the present inventors). This analysis demonstrated 721 differentiating genes including genes that play a regulatory role in epitope spreading and in macrophage recruitment to the inflammatory injury. Apoptotic-related genes such as cyclin G1 (CCG1)—the mediator of p53-dependent apoptosis and the caspases 2, 8 and 10 were significantly down-expressed.

### SUMMARY OF THE INVENTION

According to an aspect of some embodiments of the present invention there is provided a method of determining a probability of a subject diagnosed with probable multiple sclerosis to develop definite multiple sclerosis, comprising determining in a cell of the subject a level of expression of at least one polynucleotide sequence selected from the group consisting of SEQ ID NOs:4, 16, 5, 56, 20, 3, 1, 10, 57, 24, 14, 49, 13, 37, 6, 47, 50, 21, 46, 8, 26, 2, 15, 51, 44, 19, 17, 25, 33, 48, 36, 34, 12, 29, 23, 11, 45, 53, 41, 40, 31, 58, 27, 43, 35, 30, 52, 55, 7, 9, 42, 28, 54, 32, 22, 18, 38, and 39, wherein an alteration above a predetermined threshold in the level of expression of the at least one polynucleotide sequence in the cell of the subject relative to a level of expression of the at least one polynucleotide sequence in a reference cell is indicative of the probability of the subject diagnosed with probable multiple sclerosis to develop definite multiple sclerosis.

According to an aspect of some embodiments of the present invention there is provided a method of treating a subject diagnosed with probable multiple sclerosis, comprising: (a) determining in a cell of the subject a level of expression of at least one polynucleotide sequence selected from the group consisting of SEQ ID NOs:4, 16, 5, 56, 20, 3, 1, 10, 57, 24, 14, 49, 13, 37, 6, 47, 50, 21, 46, 8, 26, 2, 15, 51, 44, 19, 17, 25, 33, 48, 36, 34, 12, 29, 23, 11, 45, 53, 41, 40, 31, 58, 27, 43, 35, 30, 52, 55, 7, 9, 42, 28, 54, 32, 22, 18, 38, and 39, wherein an alteration above a predetermined threshold in the level of expression of the at least one polynucleotide sequence in the cell of the subject relative to a level of expression of the at least one polynucleotide sequence in a reference cell is indicative of a probability of the subject diagnosed with probable multiple sclerosis to develop definite multiple sclerosis, and; (b) selecting a treatment regimen based on the probability; thereby treating the subject diagnosed with probable multiple sclerosis.

According to an aspect of some embodiments of the present invention there is provided a kit for determining a probability of a subject diagnosed with probable multiple sclerosis to develop definite multiple sclerosis, comprising no more than 500 isolated nucleic acid sequences, wherein each of the isolated nucleic acid sequences is capable of specifically recognizing at least one polynucleotide sequence selected from the group consisting of SEQ ID NOs:4, 16, 5, 56, 20, 3, 1, 10, 57, 24, 14, 49, 13, 37, 6, 47, 50, 21, 46, 8, 26, 2, 15, 51, 44, 19, 17, 25, 33, 48, 36, 34, 12, 29, 23, 11, 45, 53, 41, 40, 31, 58, 27, 43, 35, 30, 52, 55, 7, 9, 42, 28, 54, 32, 22, 18, 38, and 39.

According to an aspect of some embodiments of the present invention there is provided a probeset comprising a plurality of oligonucleotides and no more than 500 oligonucleotides wherein each of the plurality of oligonucleotides is capable of specifically recognizing at least one polynucleotide sequence selected from the group consisting of SEQ ID NOs:4, 16, 5, 56, 20, 3, 1, 10, 57, 24, 14, 49, 13, 37, 6, 47, 50, 21, 46, 8, 26, 2, 15, 51, 44, 19, 17, 25, 33, 48, 36, 34, 12, 29, 23, 11, 45, 53, 41, 40, 31, 58, 27, 43, 35, 30, 52, 55, 7, 9, 42, 28, 54, 32, 22, 18, 38, and 39.

According to some embodiments of the invention, the kit further comprising a reference cell.

According to some embodiments of the invention, each of the isolated nucleic acid sequences or the plurality of oligonucleotides is bound to a solid support.

According to some embodiments of the invention, the plurality of oligonucleotides are bound to the solid support in an addressable location.

According to some embodiments of the invention, the reference cell is of an unaffected subject.

According to some embodiments of the invention, the alteration is upregulation of the expression level of the at least one polynucleotide sequence in the cell of the subject relative to the reference cell, whereas the at least one polynucleotide sequence is selected from the group consisting of SEQ ID NOs:32-58.

According to some embodiments of the invention, the probability of the subject diagnosed with probable multiple sclerosis to develop definite multiple sclerosis is higher than about 75%.

According to some embodiments of the invention, the alteration is downregulation of the expression level of the at least one polynucleotide sequence in the cell of the subject relative to the reference cell, whereas the at least one polynucleotide sequence is selected from the group consisting of SEQ ID NOs:1-31.

According to some embodiments of the invention, the probability of the subject diagnosed with probable multiple sclerosis to develop definite multiple sclerosis is higher than about 75%.

According to some embodiments of the invention, detecting the level of expression is effected using an RNA detection method.

According to some embodiments of the invention, the kit further comprising at least one reagent suitable for detecting hybridization of the isolated nucleic acid sequences and at least one RNA transcript corresponding to the at least one polynucleotide sequence selected from the group consisting of SEQ ID NOs:4, 16, 5, 56, 20, 3, 1, 10, 57, 24, 14, 49, 13, 37, 6, 47, 50, 21, 46, 8, 26, 2, 15, 51, 44, 19, 17, 25, 33, 48, 36, 34, 12, 29, 23, 11, 45, 53, 41, 40, 31, 58, 27, 43, 35, 30, 52, 55, 7, 9, 42, 28, 54, 32, 22, 18, 38, and 39.

According to some embodiments of the invention, the kit further comprising packaging materials packaging the at least one reagent and instructions for use in determining the probability of the subject diagnosed with probable multiple sclerosis to develop definite multiple sclerosis.

According to some embodiments of the invention, the at least one polynucleotide sequence is as set forth by the polynucleotide sequences of SEQ ID NOs:4, 16, 5, 56, 20, 3, 1, 10, 57, 24, 14, 49, 13, 37, 6, 47, 50, 21, 46, 8, 26, 2, 15, 51, 44, 19, 17, 25, 33, 48, 36, 34, 12, 29, 23, 11, 45, 53, 41, 40, 31, 58, 27, 43, 35, 30, 52, 55, 7, 9, 42, 28, 54, 32, 22, 18, 38, and 39.

According to some embodiments of the invention, the cell of the subject is a blood cell.

According to some embodiments of the invention, wherein said detecting said level of expression is effected using a protein detection method.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1a-b depicts the most informative genes differentially expressed between probable MS and healthy subjects. FIG. 1a—Infogramm of 554 most informative genes (listed in Table 1) discriminated between probable MS patients (N=28) and healthy subjects (N=10); Each row represents a gene and each column represents a patient's sample. FIG. 1b—PCA analysis demonstrating only two classification errors (marked by circles; 5%) using the 554 most informative genes. Blue dots—healthy subjects, red dots—probable MS patients;

FIGS. 2a-b depicts the most informative genes differentially expressed between probable MS patients that converted to definite MS during a 2-year follow-up period and healthy subjects. FIG. 2a—Infogramm of the 1517 most informative genes (listed in Table 2) that discriminated between probable MS patients (N=12) that converted to definite MS during a 2-year follow-up period and healthy subjects (N=11); Each row represents a gene and each column represents a patient's sample. All genes passed FDR criteria at p<0.03 and 8 genes passed Bonfferoni correction at p<3.2×10⁻⁵. FIG. 2b—PCA applied to the 1517 most informative genes resulted in two clusters, healthy subjects (blue dots) and probable MS patients (red dots) with no classification errors (0 classification errors).

FIG. 3 is a Van-diagram demonstrating intersecting genes between probable which further developed a definite diagnosis of MS (the 1517 genes listed in Table 2 which differentiate between probable patients that developed to definite MS during 2 years follow up and healthy controls) and definite (the 722 genes listed in Table 4 which differentiate between MS patients with a definite diagnosis of MS, i.e., after at least the second neurological attack and healthy controls) PBMC gene expression signatures. Note that the 58 intersecting genes (listed in Table 5) share the same expression pattern, i.e., either upregulation or downregulation relative to control subjects in both subjects diagnosed with probable MS which further developed definite MS within a 2-year period (i.e., probable MS subjects who develop clinical symptoms/MRI pattern which fit the diagnosis of a definite MS) and the subjects diagnosed with definite MS (i.e., an expression pattern determined in subjects with a definite diagnosis of MS);

FIGS. 4a-c depict the various multiple sclerosis subtypes. FIG. 4a—a flow chart of the MS clinical subtypes. A subject diagnosed with probable MS can develop a diagnosis of definite MS (in about 85% of the cases) during 5 years follow up period or remain diagnosed as probable MS (in 15% of the cases). Of the subjects diagnosed with definite MS, 85% exhibit a disease course of relapsing-remitting MS (RRMS) and about 15% exhibit a primary progressive course of disease. Of the patients developed RRMS, 40% will develop a secondary progressive MS course. FIG. 4b schematically illustrates the disease courses of RRMS or secondary progressive MS. FIG. 4c schematically illustrates the disease course of primary progressive MS with or without attacks/remission periods.

### DESCRIPTION OF SPECIFIC EMBODIMENTS OF THE INVENTION

The present invention, in some embodiments thereof, relates to genetic markers which are differentially expressed between probable multiple sclerosis (MS) subjects that further converted to the definite diagnosis of MS and healthy controls. More particularly, but not exclusively, such differentially expressed markers can be used to determine the probability of a subject diagnosed with probable MS to develop a definite diagnosis of MS. In addition, the present invention, in some embodiments thereof, can be used to select a treatment regimen for subjects diagnosed with probable MS based on the expression pattern of such genetic markers.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

While reducing the present invention to practice, the present inventors have uncovered genetic markers which are predictive to the definite diagnosis of MS in subjects diagnosed with probable MS, i.e., following the first neurological attack.

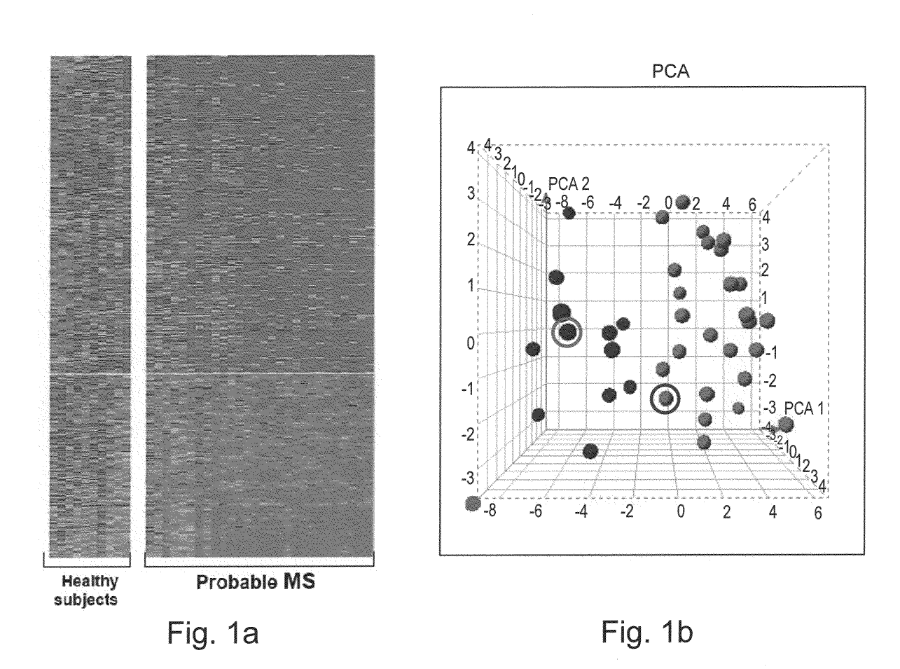

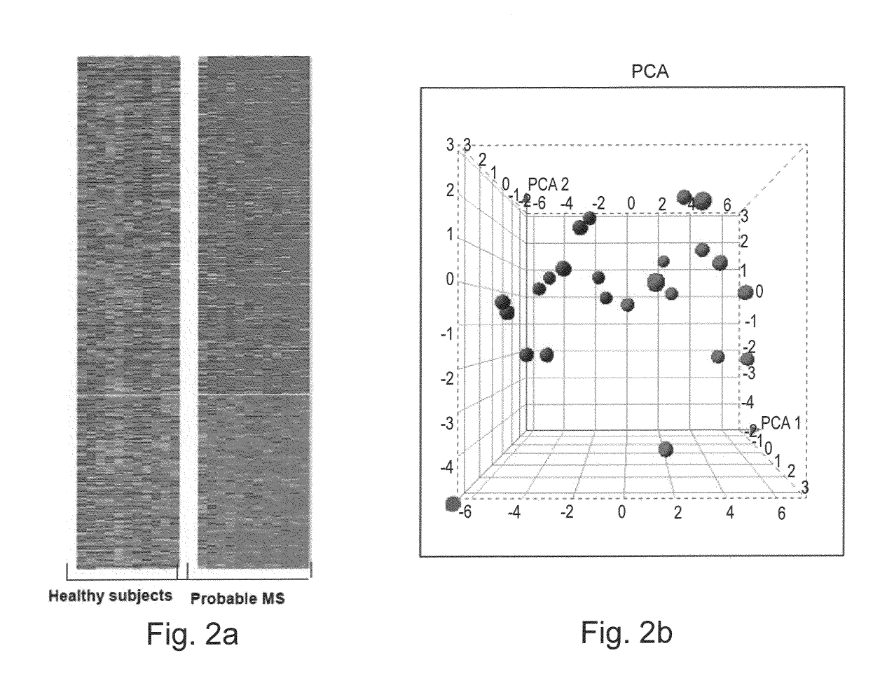

As is shown in FIGS. 1a-b, Table 1 and is described in Example 1 of the Examples section which follows, the present inventors have uncovered 554 genes which are differentially expressed in PBMC between subjects with probable MS and healthy controls. Following a 2-years follow up, the subjects diagnosed with probable MS were divided to those who eventually developed a diagnosis of definite MS (convertors to definite MS) or sustained the diagnosis of probable MS (non-convertors to definite MS). Analysis of the gene expression pattern of probable MS subjects which further converted to definite MS revealed 1517 genes which are differentially expressed as compared to healthy controls (Table 2, FIGS. 2a-b and Example 2 of the Examples section which follows). In addition, analysis of the gene expression pattern of probable MS subjects which did not convert to definite MS within a period of 2 years revealed 503 genes which are differentially expressed as compared to healthy controls (Table 3, Example 2 of the Examples section which follows). Moreover, comparison of the gene expression pattern of subjects with a definite diagnosis of MS to that of healthy controls revealed 722 genes which are differentially expressed (Table 4, Example 2 of the Examples section which follows). Furthermore, comparison of the differentiating genes between probable MS subjects which further converted to definite MS to that of subjects with the definite diagnosis of MS revealed that the expression pattern of 58 genes is common between the two groups of samples (Table 5, Example 3 of the Examples section which follows). In addition, application of the SVM software based on RBF kernel on a randomly assigned training set of 80% of the 40 probable MS patients revealed optimal sets of genes and their prediction power (average error in test set) of the probability of a subject diagnosed with probable MS to develop the definite diagnosis of MS (Table 6, Example 3 of the Examples section which follows). These results suggest that the expression pattern of each of the 58 genes and/or a combination of several or all of the 58 genes has a predictive value in determining the probability of a subject diagnosed with probable MS to develop the definite diagnosis of MS.

Thus, according to one aspect of the present invention there is provided a method of determining a probability of a subject diagnosed with probable multiple sclerosis to develop definite multiple sclerosis. The method is effected by determining in a cell of the subject a level of expression of at least one polynucleotide sequence selected from the group consisting of SEQ ID NOs:4, 16, 5, 56, 20, 3, 1, 10, 57, 24, 14, 49, 13, 37, 6, 47, 50, 21, 46, 8, 26, 2, 15, 51, 44, 19, 17, 25, 33, 48, 36, 34, 12, 29, 23, 11, 45, 53, 41, 40, 31, 58, 27, 43, 35, 30, 52, 55, 7, 9, 42, 28, 54, 32, 22, 18, 38, and 39, wherein an alteration above a predetermined threshold in the level of expression of the at least one polynucleotide sequence in the cell of the subject relative to a level of expression of the at least one polynucleotide sequence in a reference cell is indicative of the probability of the subject diagnosed with probable multiple sclerosis to develop definite multiple sclerosis.

As used herein, the phrase “a subject diagnosed with probable multiple sclerosis” refers to a mammal, preferably a human being, who is diagnosed with probable multiple sclerosis, e.g., a subject who experienced one neurological attack affecting the CNS and accompanied by demyelinating lesions on brain magnetic resonance imaging (MRI). The neurological attack can involve acute or sub-acute neurological symptomatology (attack) manifested by various clinical presentations like unilateral loss of vision, vertigo, ataxia, incoordination, gait difficulties, sensory impairment characterized by paresthesia, dysesthesia, sensory loss, urinary disturbances until incontinence, diplopia, dysarthria, various degrees of motor weakness until paralysis, cognitive decline either as a monosymptomatic or in combination. The symptoms usually remain for several days to few weeks, and then partially or completely resolve.

The diagnosis of probable MS can also include laboratory tests involving evaluation of IgG synthesis and oligoclonal bands (immunoglobulins found in 85-95% of subjects diagnosed with definite MS) in the cerebrospinal fluid (CSF, obtained by e.g., lumbar puncture) which provide evidence of chronic inflammation of the central nervous system. Combined with MRI of the brain and spinal cord and clinical data, the presence of oligoclonal bands can help make a definite diagnosis of MS.

As used herein, the phrase “determining a probability” refers to the likelihood of a subject diagnosed with probable MS to develop the definite diagnosis of MS within a certain time period. According to an embodiment of the invention, such probability can be high, e.g., more than 51%, at least 60%, at least 70%, at least 80%, at least 85%, at least 87%, at least 90%, at least 95%, at least 99%, e.g., 100%, that a subject diagnosed with probable MS will develop the definite diagnosis of MS. It will be appreciated that the time period during which the subject diagnosed with probable MS will convert to the definite diagnosis of MS can be within 1 year since onset of probable MS, within 2-3 years, within 3-5 years, or more.

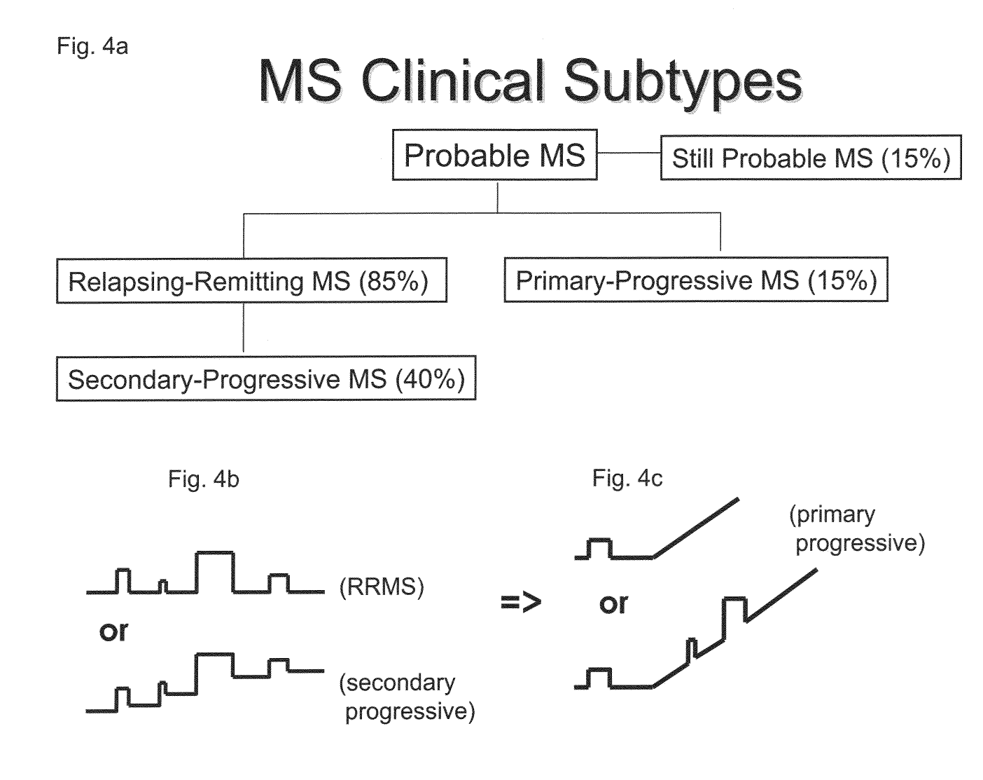

As used herein the phrase “develop definite multiple sclerosis” refers to a subject who is diagnosed with probable MS and which experiences at least a second neurological attack affecting the CNS and accompanied by demyelinating lesions on brain magnetic resonance imaging (MRI), wherein the neurological attacks are associated with the appearance of new neurological symptoms and signs or the worsening of existing neurological symptoms and signs. It will be appreciated that the disease course of patients diagnosed with definite MS can be a relapsing-remitting multiple sclerosis (RRMS) (occurring in 85% of the patients), a primary progressive multiple sclerosis (occurring in 15% of the patients) or a secondary progressive multiple sclerosis (occurring in 40% of the RRMS patients; see FIG. 4).

As mentioned, the method according to this aspect of the present invention is effected by determining in a cell of the subject a level of expression of at least one polynucleotide sequence selected from the group consisting of SEQ ID NOs:4, 16, 5, 56, 20, 3, 1, 10, 57, 24, 14, 49, 13, 37, 6, 47, 50, 21, 46, 8, 26, 2, 15, 51, 44, 19, 17, 25, 33, 48, 36, 34, 12, 29, 23, 11, 45, 53, 41, 40, 31, 58, 27, 43, 35, 30, 52, 55, 7, 9, 42, 28, 54, 32, 22, 18, 38, and 39.

According to an embodiment of the invention, the method is effected by determining in a cell of the subject a level of expression of at least two, at least three, at least four, at least five, at least six, at least seven, at least eight, at least nine, at least 10 polynucleotide sequences, at least 20, at least 30, at least 40, at least 50 polynucleotide sequences, e.g., 58 polynucleotide sequences selected from the group consisting of SEQ ID NOs:4, 16, 5, 56, 20, 3, 1, 10, 57, 24, 14, 49, 13, 37, 6, 47, 50, 21, 46, 8, 26, 2, 15, 51, 44, 19, 17, 25, 33, 48, 36, 34, 12, 29, 23, 11, 45, 53, 41, 40, 31, 58, 27, 43, 35, 30, 52, 55, 7, 9, 42, 28, 54, 32, 22, 18, 38, and 39, wherein an alteration above a predetermined threshold in the level of expression of each of the polynucleotide sequences in the cell of the subject relative to a level of expression of the same polynucleotide sequences in a reference cell is indicative of the probability of the subject diagnosed with probable multiple sclerosis to develop definite multiple sclerosis.

As mentioned above, shown in Table 6 and described in Example 3 of the Examples section which follows, the prediction power of the selected polynucleotides set forth by SEQ ID NOs:1-58 in determining the probability of a subject diagnosed with probable MS to develop definite MS within 2 years was computed using the SVM based on RBF kernel when applied on a set of 40 probable MS subjects, randomly divided to 80% as training set and 20% as test set. The polynucleotide sequence exhibiting the best prediction power as a single gene, which can be used to determine the probability of a subject diagnosed with probable MS to develop definite multiple sclerosis is set forth in SEQ ID NO:4 (average error: 0.216; prediction accuracy of 78.4%).

As is further shown in Table 6 (Example 3) several groups of genes can predict the probability of a subject diagnosed with probable MS to develop a definite multiple sclerosis within 2 years with about 87% accuracy (average error of about 0.13).

According to an embodiment of the invention, the polynucleotide sequences which expression level are determined in the cell of the subject diagnosed with probable MS are those depicted in any of the following groups of row numbers of Table 6 in Example 3 of the Examples section which follows: rows 1-33; rows 1-34; rows 1-35; rows 1-40; rows 1-44; and rows 1-45.

As is further shown in Table 6 (Example 3) several groups of genes can predict the probability of a subject diagnosed with probable MS to develop a definite multiple sclerosis within 2 years with about 84-86% accuracy (average error of about 0.14-0.16).

According to an embodiment of the invention, the polynucleotide sequences which expression level are determined in the cell of the subject diagnosed with probable MS are those depicted in any of the following groups of row numbers of Table 6 in Example 3 of the Examples section which follows: rows 1-6; rows 1-14; rows 1-15; rows 1-16; rows 1-17; rows 1-18; rows 1-19; rows 1-29; rows 1-31; rows 1-32; rows 1-36; rows 1-37; rows 1-38; rows 1-39; rows 1-40; rows 1-41; rows 1-42; rows 1-43; rows 1-46; rows 1-47; rows 1-48; rows 1-49; rows 1-50; and rows 1-52.

As is further shown in Table 6 (Example 3) several groups of genes can predict the probability of a subject diagnosed with probable MS to develop a definite multiple sclerosis diagnosis with about 80-83% accuracy (average error of about 0.17-0.20).

According to an embodiment of the invention, the polynucleotide sequences which expression level are determined in the cell of the subject diagnosed with probable MS are those depicted in any of the following groups of row numbers of Table 6 in Example 3 of the Examples section which follows: rows 1-7; rows 1-8; rows 1-9; rows 1-10; rows 1-12; rows 1-13; rows 1-20; rows 1-21; rows 1-22; rows 1-23; rows 1-24; rows 1-25; rows 1-26; rows 1-27; rows 1-28; rows 1-30; rows 1-51; rows 1-53; rows 1-54; rows 1-55; rows 1-56; rows 1-57; and rows 1-58.

As is further shown in Table 6 (Example 3) several groups of genes can predict the probability of a subject diagnosed with probable MS to develop a definite multiple sclerosis diagnosis with about 75-79% accuracy (average error of about 0.21-0.25).

According to an embodiment of the invention, the polynucleotide sequences which expression level are determined in the cell of the subject diagnosed with probable MS are those depicted in any of the following groups of row numbers of Table 6 in Example 3 of the Examples section which follows: rows 1-2; rows 1-3; rows 1-4; rows 1-5; and rows 1-11.

As used herein, the phrase “level of expression” refers to the degree of gene expression and/or gene product activity in a specific cell. For example, up-regulation or down-regulation of various genes can affect the level of the gene product (i.e., RNA and/or protein) in a specific cell.

As used herein the phrase “a cell of the subject” refers to any cell content and/or cell secreted content which contains RNA and/or proteins of the subject.

Examples include a blood cell, a bone marrow cell, a cell obtained from any tissue biopsy (e.g., CSF, brain biopsy), body fluids such as plasma, serum, saliva, spinal fluid, lymph fluid, the external sections of the skin, respiratory, intestinal, and genitourinary tracts, tears, saliva, sputum and milk. According to an embodiment of the invention, the cell is a blood cell (e.g., white blood cells, macrophages, B- and T-lymphocytes, monocytes, neutrophiles, eosinophiles, and basophiles) which can be obtained using a syringe needle from a vein of the subject. It will be appreciated that PBMC is the most accessible tissue and could be useful as a minimally invasive approach for gene expression differential diagnosis. It should be noted that a “cell of the subject” may also optionally comprise a cell that has not been physically removed from the subject (e.g., in vivo detection).

According to an embodiment of the invention, the white blood cell comprises peripheral blood mononuclear cells (PBMC). The phrase, “peripheral blood mononuclear cells (PBMCs)” as used herein, refers to a mixture of monocytes and lymphocytes. Several methods for isolating white blood cells are known in the art. For example, PBMCs can be isolated from whole blood samples using density gradient centrifugation procedures. Typically, anticoagulated whole blood is layered over the separating medium. At the end of the centrifugation step, the following layers are visually observed from top to bottom: plasma/platelets, PBMCs, separating medium and erythrocytes/granulocytes. The PBMC layer is then removed and washed to remove contaminants (e.g., red blood cells) prior to determining the expression level of the polynucleotide(s) therein.

It will be appreciated that the cell of the subject can be obtained at any time, e.g., immediately after an attack or at any time during remission.

According to preferred embodiments of the present invention, detecting the level of expression of the polynucleotide sequences of the present invention is effected using RNA or protein molecules which are extracted from the cell of the subject.

Methods of extracting RNA or protein molecules from cells of a subject are well known in the art.

Once obtained, the RNA or protein molecules can be characterized for the expression and/or activity level of various RNA and/or protein molecules using methods known in the arts.

Non-limiting examples of methods of detecting RNA molecules in a cell sample include Northern blot analysis, RT-PCR, RNA in situ hybridization (using e.g., DNA or RNA probes to hybridize RNA molecules present in the cells or tissue sections), in situ RT-PCR (e.g., as described in Nuovo G J, et al. Am J Surg Pathol. 1993, 17: 683-90; Komminoth P, et al. Pathol Res Pract. 1994, 190: 1017-25), and oligonucleotide microarray (e.g., by hybridization of polynucleotide sequences derived from a sample to oligonucleotides attached to a solid surface [e.g., a glass wafer) with addressable location, such as Affymetrix microarray (Affymetrix®, Santa Clara, Calif.)].

Non-limiting examples of methods of detecting the level and/or activity of specific protein molecules in a cell sample include Enzyme linked immunosorbent assay (ELISA), Western blot analysis, radio-immunoassay (RIA), Fluorescence activated cell sorting (FACS), immunohistochemical analysis, in situ activity assay (using e.g., a chromogenic substrate applied on the cells containing an active enzyme), in vitro activity assays (in which the activity of a particular enzyme is measured in a protein mixture extracted from the cells).

For example, in case the detection of the expression level of a secreted protein is desired, ELISA assay may be performed on a sample of fluid obtained from the subject (e.g., serum), which contains cell-secreted content.

As used herein the phrase “reference cell” refers to any cell as described hereinabove of an unaffected subject (i.e., a subject devoid of any neurological attack resembling MS or probable MS) such as a healthy subject, which can be an age and/or gender-matched unaffected subject (e.g., a healthy subject from the same age and/or gender as of the subject diagnosed with probable MS). Such a reference cell can be a blood cell, a bone marrow cell, a cell obtained from any tissue biopsy (e.g., CSF), body fluids such as plasma, serum, saliva, spinal fluid, lymph fluid, the external sections of the skin, respiratory, intestinal, and genitourinary tracts, tears, saliva, sputum and milk. It will be appreciated that the level of expression of the above referenced polynucleotides/polypeptides may be obtained from scientific literature.

Since as is shown in Table 5 and is described in Example 2 of the Examples section which follows, 27 polynucleotide sequences displayed elevated expression in the subjects diagnosed with probable MS which further developed the definite diagnosis of MS relative to healthy subjects, in order to determine the probability of a subject diagnosed with probable MS to develop definite MS, the level of expression of at least one polynucleotide sequence selected from the group consisting of SEQ ID NOs:32-58 is determined and compared to the level of expression of the same polynucleotide sequences in a reference cell derived from an unaffected subject, wherein an upregulation (increase) in the expression level of the at least one polynucleotide sequence above a predetermined threshold relative to the reference cell is indicative of a high probability (e.g., higher than about 75%, about 80%, about 85%, about 87%) of the subject diagnosed with probable MS to develop definite MS (e.g., to convert to definite MS within a period of about 2 years). On the other hand, downregulation or no significant change in the level of expression, of the same at least one polynucleotide sequence relative to the reference cell is indicative of low probability (e.g., less than about 75%, e.g., less than 50%, e.g., less than 30%) of the subject diagnosed with probable MS to develop definite MS (e.g., to convert to definite MS within a period of about 2 years). It will be appreciated that such a subject can eventually develop definite MS following a longer period of time, e.g. more than 2 years, e.g., 10-20 years.

Additionally or alternatively, since as is further shown in Table 2 and is described in Example 2 of the Examples section which follows, the level of expression of 31 polynucleotide sequences was downregulated in subjects diagnosed with probable MS relative to the healthy control subjects, in order to determine the probability of a subject diagnosed with probable MS to develop definite MS, the level of expression of at least one polynucleotide sequence selected from the group consisting of SEQ ID NOs:1-31 is determined and compared to the level of expression of the same polynucleotide sequences in a reference cell derived from an unaffected subject, wherein downregulation (decrease) in the expression level of the at least one polynucleotide sequence above a predetermined threshold relative to the reference cell is indicative of high probability (e.g., higher than about 75%, about 80%, about 85%, about 87%) of the subject diagnosed with probable MS to develop definite MS (e.g., to convert to definite MS within a period of about 2 years). On the other hand, upregulation or no significant change in the level of expression of the same at least one polynucleotide sequence relative to the reference cell is indicative of low probability (e.g., lower than 75%, e.g., less than 50%, e.g., less than 30%) of the subject diagnosed with probable MS to develop definite MS (e.g., to convert to definite MS within a period of about 2 years). It will be appreciated that such a subject can eventually develop definite MS following a longer period of time, e.g., more than 2 years.

As used herein the phrase “an alteration above a predetermined threshold” refers to the increase or decrease (i.e., degree of upregulation or downregulation, respectively) which is higher than a predetermined threshold such as at least twice, at least three times, at least four times, at least five times, at least six times, at least seven times, at least eight times, at least nine times, at least 10 times, at least 20 times, at least 50 times, at least 100 times, at least 500 times relative to the reference cell.

For example, as is shown in Table 5, while the level of expression of the polynucleotide sequences set forth by SEQ ID NOs:1-16, is at least twice lower in subjects diagnosed with probable MS which further developed definite MS as compared to unaffected subjects, the level of expression of the polynucleotide sequences set forth by SEQ ID NOs:17-26, the polynucleotide sequences set forth by SEQ ID NOs:27-29, or the polynucleotides set forth by SEQ ID NOs:30-31 is at least 5, 10, or 50 times, respectively, lower in cells of subjects diagnosed with probable MS which further developed definite MS as compared to unaffected, healthy subjects.

In addition, as is further shown in Table 2, while the level of expression of the polynucleotide sequences set forth by SEQ ID NOs:32-46, is at least twice higher in subjects diagnosed with probable MS which further developed definite MS as compared to unaffected, healthy subjects, the level of expression of the polynucleotide sequences set forth by SEQ ID NOs:47-52, the polynucleotides set forth by SEQ ID NOs:53-56, or the polynucleotide set forth by SEQ ID NOs:57-58 is at least 5, 10, or 50 times, respectively, higher in cells of subjects diagnosed with probable MS which further developed definite MS as compared to unaffected, healthy subjects.

It will be appreciated that higher fold change in the expression level of the at least one polynucleotide in the cell of the subject relative the reference cell, and/or alteration in the level of expression of the polynucleotides which exhibit high fold change in Table 5 of Example 2 (e.g., SEQ ID NOs:17-26 and/or 47-52, SEQ ID NOs:27-29 and/or 53-56, 30-31 and/or 57-58), and/or alteration above the predetermined threshold in a significant number of polynucleotides from the polynucleotides set forth by SEQ ID NOs:4, 16, 5, 56, 20, 3, 1, 10, 57, 24, 14, 49, 13, 37, 6, 47, 50, 21, 46, 8, 26, 2, 15, 51, 44, 19, 17, 25, 33, 48, 36, 34, 12, 29, 23, 11, 45, 53, 41, 40, 31, 58, 27, 43, 35, 30, 52, 55, 7, 9, 42, 28, 54, 32, 22, 18, 38, and 39 (e.g., at least 30, at least 40, at least 45, at least 50, or 58) will indicate high probability that the subject diagnosed with probable MS will develop definite MS within a short period of time (during 2 years).

Thus, the method of determining the probability of a subject diagnosed with probable MS to develop definite MS according to the invention enables the classification of probable MS patients to those that will develop definite MS within a predetermined time (e.g., about 2 years, fast convertors) and to those who will sustain the diagnosis of probable MS and will either not convert to definite MS or will convert to definite MS following an extended period of time (e.g., more than 2 years, e.g., at least 10 years).

Thus, the teachings of the present invention can be used to improve the diagnosis of definite MS following the first neurological attack, without needing to rely on the appearance of the second neurological attack.

It will be appreciated that determining the probability of a subject diagnosed with probable MS to develop definite MS can be used to select the treatment regimen of the subject and thereby to treat the subject diagnosed with probable MS.

Thus, according to an aspect of some embodiments of the present invention there is provided a method of treating a subject diagnosed with probable multiple sclerosis. The method is effected by: (a) determining in a cell of the subject a level of expression of at least one polynucleotide sequence selected from the group consisting of SEQ ID NOs:4, 16, 5, 56, 20, 3, 1, 10, 57, 24, 14, 49, 13, 37, 6, 47, 50, 21, 46, 8, 26, 2, 15, 51, 44, 19, 17, 25, 33, 48, 36, 34, 12, 29, 23, 11, 45, 53, 11, 40, 31, 58, 27, 43, 35, 30, 52, 55, 7, 9, 42, 28, 54, 32, 22, 18, 38, and 39, wherein an alteration above a predetermined threshold in the level of expression of the at least one polynucleotide sequence in the cell of the subject relative to a level of expression of the at least one polynucleotide sequence in a reference cell is indicative of a probability of the subject diagnosed with probable multiple sclerosis to develop definite multiple sclerosis, and (b) selecting a treatment regimen based on the probability, thereby treating the subject diagnosed with probable multiple sclerosis.

As used herein the phrase “treating” refers to inhibiting or arresting the development of a pathology [multiple sclerosis, e.g., RRMS or progressive (e.g., primary or secondary) MS] and/or causing the reduction, remission, or regression of a pathology and/or optimally curing the pathology. Those of skill in the art will understand that various methodologies and assays can be used to assess the development of a pathology, and similarly, various methodologies and assays may be used to assess the reduction, remission or regression of the pathology.

According to an embodiment of the invention, when the probability as determined according to the method of the invention of the subject diagnosed with probable MS to develop definite MS is high [e.g., at least 89.9% that the subject will develop definite MS within 2 years], the treatment regimen selected for treating such a subject comprises preventive medications which will prevent the reaction leading to neurological disability. It will be appreciated that the currently available medications for treating definite MS are not allowed for treating subjects diagnosed with probable MS. Thus, teachings of the invention can be used to prevent the neurological deterioration of subjects diagnosed with probable MS.

Thus, by determining the probability of the subject diagnosed with probable MS to develop definite MS, the subject can be treated early, prior to experiencing the second neurological attack, with suitable therapeutics that can prevent deterioration of clinical symptoms and can increase the chances of achieving cure and remission of symptoms in the affected subjects.

It will be appreciated that classification of subjects diagnosed with probable MS to those that will convert fast to definite MS and to those that will sustain the diagnosis of probable MS can be also used in order to assess the efficacy of a treatment regimen on probable MS patients which are likely to develop definite MS. Thus, by treating subjects with probable MS and high probability to develop definite MS (as determined by the method of the invention) with candidate preventive and/or therapeutic drugs and monitoring the subjects' health in terms of MS progression (e.g., EDSS evaluation and number of relapses), the efficacy of the drugs can be assessed.

The teachings of the invention are of utmost importance and have relevant medical, economical and social aspects. While the MS disease prevalence in USA is at the range of 250.000 to 350.000 cases, the annual cost of MS in USA is anticipated to be 34,000 $ per patient, leading to 2.2 million $ total lifetime cost per patient or 6.8 billion $ yearly, in a conservative estimate of the national annual cost. The possibility to early identify the patients which will develop definite MS among the patients with the diagnosis of probable MS is of utmost importance, as it would be possible to start preventive treatment early and delay accumulation of irreversible neurological disability, inhibition/suppression of disease progression as well as reduce annual cost of disease.

It will be appreciated that the reagents utilized by any of the methods of the present invention which are described hereinabove can form a part of a diagnostic kit/article of manufacture.

The kit of the invention comprises at least one and no more than 500 isolated nucleic acid sequences, e.g., at least 2 and no more than 500 isolated nucleic acid sequences, e.g., at least 4 and no more than 400 isolated nucleic acid sequences, e.g., at least 6 and no more than 300 isolated nucleic acid sequences, e.g., at least 8 and no more than 200 isolated nucleic acid sequences, e.g., at least 2 and no more than 100 isolated nucleic acid sequences, e.g., at least 2 and no more than 58 isolated nucleic acid sequences, wherein each of the at least one and no more than 500 isolated nucleic acid sequences is capable of specifically recognizing at least one specific polynucleotide sequence selected from the group consisting of SEQ ID NOs:4, 16, 5, 56, 20, 3, 1, 10, 57, 24, 14, 49, 13, 37, 6, 47, 50, 21, 46, 8, 26, 2, 15, 51, 44, 19, 17, 25, 33, 48, 36, 34, 12, 29, 23, 11, 45, 53, 41, 40, 31, 58, 27, 43, 35, 30, 52, 55, 7, 9, 42, 28, 54, 32, 22, 18, 38, and 39.

The isolated nucleic acid sequences included in the kit of the present invention can be single-stranded or double-stranded, naturally occurring or synthetic nucleic acid sequences such as oligonucleotides, RNA molecules, genomic DNA molecules, cDNA molecules and/or cRNA molecules. The isolated nucleic acid sequences of the kit can be composed of naturally occurring bases, sugars, and covalent internucleoside linkages (e.g., backbone), as well as non-naturally occurring portions, which function similarly to respective naturally occurring portions.

Synthesis of the isolated nucleic acid sequences of the kit can be performed using enzymatic synthesis or solid-phase synthesis. Equipment and reagents for executing solid-phase synthesis are commercially available from, for example, Applied Biosystems. Any other means for such synthesis may also be employed; the actual synthesis of the oligonucleotides is well within the capabilities of one skilled in the art and can be accomplished via established methodologies as detailed in, for example: Sambrook, J. and Russell, D. W. (2001), “Molecular Cloning: A Laboratory Manual”; Ausubel, R. M. et al., eds. (1994, 1989), “Current Protocols in Molecular Biology,” Volumes I-III, John Wiley & Sons, Baltimore, Md.; Perbal, B. (1988), “A Practical Guide to Molecular Cloning,” John Wiley & Sons, New York; and Gait, M. J., ed. (1984), “Oligonucleotide Synthesis”; utilizing solid-phase chemistry, e.g. cyanoethyl phosphoramidite followed by deprotection, desalting, and purification by, for example, an automated trityl-on method or HPLC.

According to an embodiment of the invention, each of the isolated nucleic acid sequences included in the kit of present invention comprises at least 10 and no more than 50 nucleic acids, e.g., at least 15 and no more than 45, e.g., between 15-40, e.g., between 20-35, e.g., between 20-30, e.g., between 20-25 nucleic acids.

According to an embodiment of the invention the kit includes at least one reagent as described hereinabove which is suitable for recognizing the at least one specific polynucleotide sequence selected from the group consisting of SEQ ID NOs:4, 16, 5, 56, 20, 3, 1, 10, 57, 24, 14, 49, 13, 37, 6, 47, 50, 21, 46, 8, 26, 2, 15, 51, 44, 19, 17, 25, 33, 48, 36, 34, 12, 29, 23, 11, 45, 53, 41, 40, 31, 58, 27, 43, 35, 30, 52, 55, 7, 9, 42, 28, 54, 32, 22, 18, 38, and 39. Examples include reagents suitable for hybridization or annealing of a specific polynucleotide of the kit to a specific target polynucleotide sequence (e.g., RNA transcript derived from the cell of the subject or a cDNA derived therefrom) such as formamide, sodium chloride, and sodium citrate), reagents which can be used to labeled polynucleotides (e.g., radiolabeled nucleotides, biotinylated nucleotides, digoxigenin-conjugated nucleotides, fluorescent-conjugated nucleotides) as well as reagents suitable for detecting the labeled polynucleotides (e.g., antibodies conjugated to fluorescent dyes, antibodies conjugated to enzymes, radiolabeled antibodies and the like).

Additionally or alternatively, the kit of the present invention comprises at least one reagent suitable for detecting the expression level and/or activity of at least one polypeptide encoded by at least one polynucleotides selected from the group consisting of SEQ ID NOs:4, 16, 5, 56, 20, 3, 1, 10, 57, 24, 14, 49, 13, 37, 6, 47, 50, 21, 46, 8, 26, 2, 15, 51, 44, 19, 17, 25, 33, 48, 36, 34, 12, 29, 23, 11, 45, 53, 41, 40, 31, 58, 27, 43, 35, 30, 52, 55, 7, 9, 42, 28, 54, 32, 22, 18, 38, and 39. Such a reagent can be, for example, an antibody capable of specifically binding to at least one epitope of the polypeptide. Additionally or alternatively, the reagent included in the kit can be a specific substrate capable of binding to an active site of the polypeptide. In addition, the kit may also include reagents such as fluorescent conjugates, secondary antibodies and the like which are suitable for detecting the binding of a specific antibody and/or a specific substrate to the polypeptide.

According to an embodiment of the invention the kit includes a reference cell which comprises a cell of an unaffected subject as described hereinabove.

According to an embodiment of the invention, the kit of the invention includes packaging material packaging the at least one reagent and a notification in or on the packaging material. Such a notification identifies the kit for use in determining the probability of a subject diagnosed with probable MS to develop definite MS and selecting a treatment regimen of a subject and thereby treating the subject diagnosed with probable MS. The kit may also include instructions for use in determining the probability of a subject diagnosed with probable MS to develop definite MS and selecting a treatment regimen of a subject and thereby treating the subject diagnosed with probable MS. The kit may also include appropriate buffers and preservatives for improving the shelf-life of the kit.

It will be appreciated that the isolated nucleic acid sequences described hereinabove (e.g., oligonucleotides) can form a part of a probeset. The probeset comprises a plurality of oligonucleotides and no more than 500 oligonucleotides wherein each of the plurality of oligonucleotides is capable of specifically recognizing at least one polynucleotide sequence selected from the group consisting of SEQ ID NOs:4, 16, 5, 56, 20, 3, 1, 10, 57, 24, 14, 49, 13, 37, 6, 47, 50, 21, 46, 8, 26, 2, 15, 51, 44, 19, 17, 25, 33, 48, 36, 34, 12, 29, 23, 11, 45, 53, 41, 40, 31, 58, 27, 43, 35, 30, 52, 55, 7, 9, 42, 28, 54, 32, 22, 18, 38, and 39.

It will be appreciated that the isolated nucleic acid sequences included in the kit or the probeset of the present invention can be bound to a solid support e.g., a glass wafer in a specific order, i.e., in the form of a microarray. Alternatively, isolated nucleic acid sequences can be synthesized directly on the solid support using well known prior art approaches (Seo T S, et al., 2004, Proc. Natl. Acad. Sci. USA, 101: 5488-93.). In any case, the isolated nucleic acid sequences are attached to the support in a location specific manner such that each specific isolated nucleic acid sequence has a specific address on the support (i.e., an addressable location) which denotes the identity (i.e., the sequence) of that specific isolated nucleic acid sequence.

According to preferred embodiments of the present invention the microarray comprises no more than 500 isolated nucleic acid sequences, wherein each of the isolated nucleic acid sequences is capable of specifically recognizing at least one specific polynucleotide sequence selected from the group consisting of SEQ ID NOs: 4, 16, 5, 56, 20, 3, 1, 10, 57, 24, 14, 49, 13, 37, 6, 47, 50, 21, 46, 8, 26, 2, 15, 51, 44, 19, 17, 25, 33, 48, 36, 34, 12, 29, 23, 11, 45, 53, 41, 40, 31, 58, 27, 43, 35, 30, 52, 55, 7, 9, 42, 28, 54, 32, 22, 18, 38, and 39.

As used herein the term “about” refers to ±10%.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions; illustrate the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### General Materials and Experimental Methods

Study design and study subjects—40 patients with probable MS of up to 3 months duration according to Poser's criteria (1983, CPMS C3: 1 attack, at least 1 clinical manifestation in addition to positive brain MRI signifying paraclinical evidence) were included in the study. Positive brain MM was defined according to Fazekas's criteria (1999) by at least 4 focal lesions involving the white matter or 3 lesions if one is periventricular; ≧3 mm in diameter, each. For the evaluation of probable MS related transcription fingerprints the large-scale gene expression profile of patients was compared with the data of 10 sex- and age-matched healthy subjects. Verification of probable PBMC gene expression signature was performed on Signed written informed consent was obtained from all participants.

Clinical neurological assessment—Neurological examination and assessment of disability was performed by the Expanded Disability Status Scale (EDSS) score (17) performed at screening, baseline visit, and at 24, 36 and 48 weeks of follow-up visits. The occurrence of a second acute attack, the time to second attack (progression to definite MS) and the change in neurological disability assessed by the EDSS were recorded in each patient. Second attack was defined as the onset of new neurological symptoms or worsening of previous ones occurring at least 30 days after the first attack, lasting for at least 48 hours and involving an objective increase by at least 0.5 point in the EDSS.

MRI examination—Brain MRI was performed using a 3.0 Tesla Imager (GE): Axial dual spin-echo (PD and T2 sequences) and T1-weighted images before and after intravenous administration of Gd-DTPA. Brain lesion load was quantified using the MSAnalyze software (18). This automated technique is based on several mathematical algorithms (e.g., Bayesian classification, near-neighborhood) leading to brain tissue segmentation enabling precise 3-dimensional lesions' identification and volumetric quantification.

PBMC preparation—Blood sample (20 ml) was drawn from all study subjects. No corticosteroid treatment was given for at least 4 weeks prior to blood drawing. PBMC were separated on Ficoll hypaque, washed with PBS and the pellet frozen in liquid nitrogen.

RNA extraction—Frozen PBMC were homogenized in ice cold trizol and total RNA extracted and used as a template for double stranded cDNA synthesis (Affymetrix, Santa Clara, Calif.). RNA quantity was determined by optical density measurements at 260 nm and its quality by running the RNA on a formamide-formaldehyde denaturing gel.

Preparation of labeled cRNA—Double stranded cDNA was performed using a cDNA synthesis kit (Life Technologies Superscript cDNA Synthesis System) with an oligo (dT) primer containing a T7 RNA polymerase promoter site added to the 3′. The cDNA was used as a template for in vitro transcription with biotin labeled nucleotides (Enzo Diagnostics). Labeled cRNA was used for hybridization.

Hybridization of microarrays—Each Genechip array (U133A) was hybridized with 10 μg/200 μl hybridization mix, stained with streptavidin phycoerythrin (Molecular Probes), hybridized with biotin labeled anti-streptavidin phycoerythrin antibody, re-stained with streptavidin phycoerythrin and scanned (Hewlett Packard, GeneArray-™ scanner G2500A).

Data analysis—Data were analyzed using ScoreGene software. To correct for multiple testing the False Discovery Rate (FDR) method and the stringent Bonferroni correction were applied. Overabundance analysis was performed to examine the observed results in comparison to expected results. To assess and validate the predictive power of the gene expression signature, the following methods were applied: Leave-One-Out-Cross-Validation (LOOCV) (21; Ben-Dor A et al., 2000) Principal Component Analysis (PCA), and Support vector machine (SVM) [(http://ro.utia.cz/fs/fs_algorithms.html), (19, 20). The study involves various comparisons between subjects of the data set. Due to the reasons of controls compatibility, the number of controls changes from one comparison to another.

Computation of the average error in determining which probable multiple sclerosis (MS) subjects exhibit high probability (predisposition) to develop the definite diagnosis of MS—For each of the 58 differentiating genes (SEQ ID NOs:1-58) the sample of 40 probable MS patients was randomly divided into 80% as a “training set” and 20% as a “test set”. The SVM used RBF (radial basic function) kernel to build a model based on the “training set”, which was further tested on the “test set” while saving the error rate. This procedure was repeated 25 times for each gene and the average error for each gene was calculated. Genes with the lowest average error were selected. Then, for each selected gene, the remaining genes were added one after the other, by selecting the next gene such that the average error after 25 repeats of the group of genes including the new gene has the lowest average error as compared to the addition of another gene. This process was repeated 57 times for each additional genes added to the previous group of genes. The results are shown in Table 6 and described in Example 3 hereinbelow.

### Example 1

### Identification of Transcripts Differentiating Between Probable MS and Healthy Subjects

Experimental and Statistical Results

Analysis of large scale gene expression pattern—Analysis of large-scale gene expression patterns of PBMC samples obtained from 28 patients with probable MS (mean±SE, age 36.0±1.9 years, EDSS 1.5±0.2) and 10 healthy matched controls was performed. Gene expression patterns of PBMC in probable MS patients were significantly different from healthy subject. Table 1, hereinbelow, depicts 554 genes that passed the 95% confidence level in all 3 statistical scores (TNoM, Info, T-test); 352 genes were over-expressed and 202 under-expressed. These genes were defined as the most informative (FIG. 1a). PCA performed on the 554 most informative genes divided all samples into two separated clusters that represented probable MS patients and healthy subjects with only 2 (5%) classification errors (FIG. 1b).

TABLE 1
Differentially expressed markers between probable
multiple sclerosis subjects and healthy controls

| | GenBank | | |
|---|---|---|---|
| Probeset | Acc. No. | Dir | Gene Symbol |
| 1494_f_at | M33318 | −1 | CYP2A6 |
| 200797_s_at | AI275690 | −1 | MCL1 |
| 200798_x_at | NM_021960 | −1 | MCL1 |
| 201041_s_at | NM_004417 | −1 | DUSP1 |
| 201044_x_at | AA530892 | −1 | DUSP1 |
| 201109_s_at | AV726673 | −1 | THBS1 |
| 201110_s_at | NM_003246 | −1 | THBS1 |
| 201235_s_at | BG339064 | −1 | BTG2 |
| 201236_s_at | BG339064 | −1 | BTG2 |
| 201464_x_at | BC002646 | −1 | JUN |
| 201473_at | NM_002229 | −1 | JUNB |
| 201489_at | BC005020 | −1 | PPIF |
| 201490_s_at | NM_005729 | −1 | PPIF |
| 201502_s_at | NM_020529 | −1 | NFKBIA |
| 201531_at | NM_003407 | −1 | ZFP36 |
| 201631_s_at | NM_003897 | −1 | IER3 |
| 201647_s_at | NM_005506 | −1 | SCARB2 |
| 201668_x_at | AW163148 | −1 | MARCKS |
| 201669_s_at | NM_002356 | −1 | MARCKS |
| 201670_s_at | M68956 | −1 | MARCKS |
| 201693_s_at | NM_001964 | −1 | EGR1 |
| 201694_s_at | NM_001964 | −1 | EGR1 |
| 202672_s_at | NM_001674 | −1 | ATF3 |
| 204224_s_at | NM_000161 | −1 | GCH1 |
| 201884_at | NM_004363 | −1 | CEACAM5 |
| 202014_at | NM_014330 | −1 | PPP1R15A |
| 202068_s_at | NM_000527 | −1 | LDLR |
| 202081_at | NM_004907 | −1 | IER2 |
| 202286_s_at | J04152 | −1 | TACSTD2 |
| 202340_x_at | NM_002135 | −1 | NR4A1 |
| 202570_s_at | NM_014902 | −1 | DLGAP4 |
| 202637_s_at | AI608725 | −1 | ICAM1 |
| 202638_s_at | NM_000201 | −1 | ICAM1 |
| 202643_s_at | AI738896 | −1 | TNFAIP3 |
| 202644_s_at | NM_006290 | −1 | TNFAIP3 |
| 202859_x_at | NM_000584 | −1 | IL8 |
| 202895_s_at | D86043 | −1 | PTPNS1 |
| 202924_s_at | AF006005 | −1 | PLAGL2 |
| 202925_s_at | NM_002657 | −1 | PLAGL2 |
| 203317_at | NM_012455 | −1 | PSD4 |
| 203394_s_at | BE973687 | −1 | HES1 |
| 203470_s_at | AI433595 | −1 | PLEK |
| 203471_s_at | NM_002664 | −1 | PLEK |
| 203672_x_at | U12387 | −1 | TPMT |
| 203927_at | NM_004556 | −1 | NFKBIE |
| 204103_at | NM_002984 | −1 | CCL4 |
| 204393_s_at | NM_001099 | −1 | ACPP |
| 204440_at | NM_004233 | −1 | CD83 |
| 204469_at | NM_002851 | −1 | PTPRZ1 |
| 204470_at | NM_001511 | −1 | CXCL1 |
| 204533_at | NM_001565 | −1 | CXCL10 |
| 204748_at | NM_000963 | −1 | PTGS2 |
| 204794_at | NM_004418 | −1 | DUSP2 |
| 204897_at | AA897516 | −1 | PTGER4 |
| 204907_s_at | AI829875 | −1 | BCL3 |
| 204970_s_at | NM_002359 | −1 | MAFG |
| 205045_at | NM_007202 | −1 | AKAP10 |
| 205067_at | NM_000576 | −1 | IL1B |
| 205114_s_at | NM_002983 | −1 | CCL3, CCL3L1 |
| 205115_s_at | NM_016196 | −1 | RBM19 |
| 205207_at | NM_000600 | −1 | IL6 |
| 205220_at | NM_006018 | −1 | GPR109B |
| 205318_at | NM_004984 | −1 | KIF5A |
| 205558_at | NM_004620 | −1 | TRAF6 |
| 205643_s_at | NM_004576 | −1 | PPP2R2B |
| 205681_at | NM_004049 | −1 | BCL2A1 |
| 205767_at | NM_001432 | −1 | EREG |
| 206025_s_at | AW188198 | −1 | TNFAIP6 |
| 206087_x_at | NM_000410 | −1 | HFE |
| 206157_at | NM_002852 | −1 | PTX3 |
| 206207_at | NM_001828 | −1 | CLC |
| 206256_at | NM_001308 | −1 | CPN1 |
| 206295_at | NM_001562 | −1 | IL18 |
| 206704_at | NM_000084 | −1 | CLCN5 |
| 206765_at | AF153820 | −1 | KCNJ2 |
| 206880_at | NM_005446 | −1 | P2RXL1 |
| 207075_at | NM_004895 | −1 | CIAS1 |
| 207113_s_at | NM_000594 | −1 | TNF |
| 207287_at | NM_025026 | −1 | FLJ14107 |
| 207490_at | NM_025019 | −1 | TUBA4 |
| 207535_s_at | NM_002502 | −1 | NFKB2 |
| 207574_s_at | NM_015675 | −1 | GADD45B |
| 207850_at | NM_002090 | −1 | CXCL3 |
| 207901_at | NM_002187 | −1 | IL12B |
| 207984_s_at | NM_005374 | −1 | MPP2 |
| 208025_s_at | NM_003483 | −1 | HMGA2 |
| 208039_at | NM_003048 | −1 | NM_003048 |
| 208120_x_at | NM_031221 | −1 | — |
| 208455_at | NM_002855 | −1 | PVRL1 |
| 208695_s_at | BC001019 | −1 | RPL39 |
| 208886_at | BC000145 | −1 | H1F0 |
| 208961_s_at | AB017493 | −1 | COPEB |
| 209034_at | AF279899 | −1 | PNRC1 |
| 209039_x_at | AF001434 | −1 | EHD1 |
| 209124_at | U70451 | −1 | MYD88 |
| 209304_x_at | AF087853 | −1 | GADD45B |
| 209305_s_at | AF078077 | −1 | GADD45B |
| 209545_s_at | AF027706 | −1 | RIPK2 |
| 209738_x_at | M31125 | −1 | M31125 |
| 209774_x_at | M57731 | −1 | CXCL2 |
| 209874_x_at | AK023066 | −1 | CNNM2 |
| 209939_x_at | AF005775 | −1 | CFLAR |
| 210001_s_at | AB005043 | −1 | SOCS1 |
| 210118_s_at | M15329 | −1 | IL1A |
| 210175_at | BC000853 | −1 | C2orf3 |
| 210254_at | L35848 | −1 | MS4A3 |
| 210275_s_at | AF062347 | −1 | ZA20D2 |
| 210414_at | AF169675 | −1 | FLRT1 |
| 210592_s_at | M55580 | −1 | SAT |
| 210651_s_at | L41939 | −1 | EPHB2 |
| 211307_s_at | U43677 | −1 | FCAR |
| 211332_x_at | AF144241 | −1 | HFE |
| 211403_x_at | AF167079 | −1 | VCX-(C,2,3); |
| | | | VCY |
| 211434_s_at | AF015524 | −1 | CCRL2 |
| 211506_s_at | AF043337 | −1 | IL8 |
| 211578_s_at | M60725 | −1 | RPS6KB1 |
| 211610_at | U51869 | −1 | COPEB |
| 213575_at | AW978896 | −1 | TRA2A |
| 215462_at | AI978990 | −1 | LOC149478 |
| 211863_x_at | AF079408 | −1 | HFE |
| 211924_s_at | AY029180 | −1 | PLAUR |
| 211973_at | AW341200 | −1 | NUDT3 |
| 212099_at | AI263909 | −1 | RHOB |
| 212291_at | AI393355 | −1 | HIPK1 |
| 212602_at | AI806395 | −1 | WDFY3 |
| 212657_s_at | AW083357 | −1 | IL1RN |
| 212659_s_at | AW083357 | −1 | IL1RN |
| 213002_at | AA770596 | −1 | AA770596 |
| 213038_at | AL031602 | −1 | IBRDC3 |
| 213146_at | AA521267 | −1 | JMJD3 |
| 213281_at | BE327172 | −1 | BE327172 |
| 213593_s_at | AW978896 | −1 | TRA2A |
| 213632_at | M94065 | −1 | DHODH |
| 213675_at | W61005 | −1 | FLJ25106 |
| 213676_at | AL038824 | −1 | AW125688 |
| 213988_s_at | BE971383 | −1 | SAT |
| 214211_at | AA083483 | −1 | FTH1 |
| 214349_at | AV764378 | −1 | ORF2 |
| 214421_x_at | AV652420 | −1 | CYP2C9 |
| 214637_at | BG437034 | −1 | OSM |
| 214657_s_at | AU134977 | −1 | TncRNA |
| 215006_at | AK023816 | −1 | EZH2 |
| 215078_at | AL050388 | −1 | SOD2 |
| 215189_at | X99142 | −1 | KRTHB6 |
| 215223_s_at | W46388 | −1 | SOD2 |
| 215308_at | AF052148 | −1 | G22P1 |
| 215485_s_at | AA284705 | −1 | ICAM1 |
| 215577_at | AU146791 | −1 | UBE2E1 |
| 215758_x_at | AC007204 | −1 | ZNF505 |
| 215775_at | BF084105 | −1 | BF084105 |
| 215899_at | AK022331 | −1 | AK022331 |
| 215987_at | AV654984 | −1 | AV654984 |
| 216015_s_at | AK027194 | −1 | CIAS1 |
| 216016_at | AK027194 | −1 | CIAS1 |
| 216084_at | AL080137 | −1 | LOC389715 |
| 216114_at | AL049430 | −1 | NCKIPSD |
| 216153_x_at | AK022897 | −1 | RECK |
| 216243_s_at | BE563442 | −1 | IL1RN |
| 216336_x_at | AL031602 | −1 | AL031602 |
| 216366_x_at | AF047245 | −1 | AF047245 |
| 216438_s_at | AL133228 | −1 | TMSB4X, |
| | | | TMSL3 |
| 216678_at | AK000773 | −1 | AK000773 |
| 216774_at | AK025325 | −1 | AK025325 |
| 216973_s_at | S49765 | −1 | HOXB7 |
| 216997_x_at | AL358975 | −1 | TLE4 |
| 217362_x_at | AF005487 | −1 | HLA-DRB6 |
| 217415_at | M21610 | −1 | M21610 |
| 217489_s_at | S72848 | −1 | IL6R |
| 217741_s_at | NM_006007 | −1 | ZA20D2 |
| 217996_at | NM_007350 | −1 | PHLDA1 |
| 218177_at | NM_020412 | −1 | CHMP1.5 |
| 218198_at | NM_018180 | −1 | DHX32 |
| 218611_at | NM_016545 | −1 | IER5 |
| 218810_at | NM_025079 | −1 | FLJ23231 |
| 219312_s_at | NM_023929 | −1 | ZBTB10 |
| 219358_s_at | NM_018404 | −1 | CENTA2 |
| 219397_at | NM_025147 | −1 | FLJ13448 |
| 219450_at | NM_018302 | −1 | FLJ11017 |
| 219617_at | NM_024766 | −1 | FLJ23451 |
| 219901_at | NM_018351 | −1 | FGD6 |
| 219935_at | NM_007038 | −1 | ADAMTS5 |
| 220054_at | NM_016584 | −1 | IL23A |
| 220091_at | NM_017585 | −1 | SLC2A6 |
| 220215_at | NM_024804 | −1 | FLJ12606 |
| 220712_at | NM_024984 | −1 | NM_024984 |
| 220737_at | AF184965 | −1 | AF184965 |
| 220740_s_at | NM_005135 | −1 | SLC12A6 |
| 220776_at | NM_013348 | −1 | KCNJ14 |
| 220924_s_at | NM_018976 | −1 | SLC38A2 |
| 221236_s_at | NM_030795 | −1 | STMN4 |
| 221323_at | NM_025218 | −1 | ULBP1 |
| 221345_at | NM_005306 | −1 | GPR43 |
| 221477_s_at | BC001980 | −1 | gasdermin |
| 222136_x_at | AK022905 | −1 | ZNF43 |
| 222303_at | AV700891 | −1 | AV700891 |
| 222326_at | AW973834 | −1 | AW973834 |
| 222329_x_at | AW974816 | −1 | AW974816 |
| 36564_at | W27419 | −1 | IBRDC3 |
| 37028_at | U83981 | −1 | PPP1R15A |
| 39402_at | M15330 | −1 | IL1B |
| 41386_i_at | AB002344 | −1 | JMJD3 |
| 41387_r_at | AB002344 | −1 | JMJD3 |
| 200023_s_at | AI001896 | 1 | EIF3S5 |
| 200028_s_at | NM_020151 | 1 | STARD7 |
| 200030_s_at | NM_002635 | 1 | SLC25A3 |
| 200083_at | AA621731 | 1 | USP22 |
| 200098_s_at | T33068 | 1 | ANAPC5 |
| 200609_s_at | NM_017491 | 1 | WDR1 |
| 200625_s_at | NM_006367 | 1 | CAP1 |
| 200688_at | D13642 | 1 | SF3B3 |
| 200816_s_at | NM_000430 | 1 | PAFAH1B1 |
| 200830_at | NM_002808 | 1 | PSMD2 |
| 200840_at | NM_005548 | 1 | KARS |
| 200870_at | NM_007178 | 1 | STRAP |
| 200899_s_at | AK002091 | 1 | MGEA5 |
| 200910_at | NM_005998 | 1 | CCT3 |
| 200932_s_at | NM_006400 | 1 | DCTN2 |
| 200961_at | NM_012248 | 1 | SEPHS2 |
| 201010_s_at | NM_006472 | 1 | TXNIP |
| 201027_s_at | NM_015904 | 1 | EIF5B |
| 201036_s_at | NM_005327 | 1 | HADHSC |
| 201106_at | NM_002085 | 1 | GPX4 |
| 201174_s_at | NM_018975 | 1 | TERF2IP |
| 201229_s_at | BC000422 | 1 | ARIH2 |
| 201405_s_at | NM_006833 | 1 | COPS6 |
| 201540_at | NM_001449 | 1 | FHL1 |
| 201541_s_at | NM_006349 | 1 | ZNHIT1 |
| 201569_s_at | NM_015380 | 1 | CGI-51 |
| 201612_at | NM_000696 | 1 | ALDH9A1 |
| 201672_s_at | NM_005151 | 1 | USP14 |
| 201688_s_at | BG389015 | 1 | TPD52 |
| 201689_s_at | NM_005079 | 1 | TPD52 |
| 202042_at | NM_002109 | 1 | HARS |
| 202658_at | NM_003846 | 1 | PEX11B |
| 202836_s_at | BC001046 | 1 | TXNL4A |
| 204220_at | NM_004877 | 1 | GMFG |
| 201805_at | NM_002733 | 1 | PRKAG1 |
| 201863_at | NM_014077 | 1 | FAM32A |
| 201959_s_at | AA488899 | 1 | MYCBP2 |
| 201964_at | N64643 | 1 | N64643 |
| 202009_at | NM_007284 | 1 | PTK9L |
| 202020_s_at | NM_006055 | 1 | LANCL1 |
| 202057_at | AW051311 | 1 | KPNA1 |
| 202116_at | NM_006268 | 1 | DPF2 |
| 202127_at | AB011108 | 1 | PRPF4B |
| 202167_s_at | NM_022362 | 1 | MMS19L |
| 202220_at | NM_014949 | 1 | KIAA0907 |
| 202244_at | NM_002796 | 1 | PSMB4 |
| 202259_s_at | NM_014887 | 1 | PFAAP5 |
| 202265_at | NM_005180 | 1 | BMI1 |
| 202271_at | AB007952 | 1 | AB007952 |
| 202296_s_at | NM_007033 | 1 | RER1 |
| 202306_at | NM_002696 | 1 | POLR2G |
| 202342_s_at | NM_015271 | 1 | TRIM2 |
| 202353_s_at | NM_002816 | 1 | PSMD12 |
| 202365_at | BC004815 | 1 | MGC5139 |
| 202439_s_at | NM_000202 | 1 | IDS |
| 202475_at | NM_006326 | 1 | NIFIE14 |
| 202529_at | NM_002766 | 1 | PRPSAP1 |
| 202560_s_at | NM_015607 | 1 | DKFZP547E1010 |
| 202564_x_at | NM_001667 | 1 | ARL2 |
| 202568_s_at | AI745639 | 1 | MARK3 |
| 202759_s_at | BE879367 | 1 | PALM2-AKAP2 |
| 202761_s_at | NM_015180 | 1 | SYNE2 |
| 202792_s_at | NM_014678 | 1 | KIAA0685 |
| 202811_at | NM_006463 | 1 | STAMBP |
| 202892_at | NM_004661 | 1 | CDC23 |
| 202960_s_at | NM_000255 | 1 | MUT |
| 202983_at | AI760760 | 1 | SMARCA3 |
| 203117_s_at | NM_014871 | 1 | USP52 |
| 203194_s_at | NM_005387 | 1 | NUP98 |
| 203221_at | NM_005077 | 1 | TLE1 |
| 203264_s_at | NM_015185 | 1 | ARHGEF9 |
| 203266_s_at | NM_003010 | 1 | MAP2K4 |
| 203301_s_at | NM_021145 | 1 | DMTF1 |
| 203378_at | AB020631 | 1 | PCF11 |
| 203445_s_at | NM_005730 | 1 | CTDSP2 |
| 203482_at | AL133215 | 1 | C10orf6 |
| 203487_s_at | NM_015396 | 1 | ARMC8 |
| 203517_at | NM_006554 | 1 | MTX2 |
| 203583_at | NM_014044 | 1 | UNC50 |
| 203683_s_at | NM_003377 | 1 | VEGFB |
| 203710_at | NM_002222 | 1 | ITPR1 |
| 203721_s_at | NM_016001 | 1 | CGI-48 |
| 203738_at | AI421192 | 1 | FLJ11193 |
| 203775_at | NM_014251 | 1 | SLC25A13 |
| 203804_s_at | NM_006107 | 1 | CROP |
| 203865_s_at | NM_015833 | 1 | ADARB1 |
| 203981_s_at | AL574660 | 1 | ABCD4 |
| 204000_at | NM_016194 | 1 | GNB5 |
| 204004_at | AI336206 | 1 | PAWR |
| 204060_s_at | NM_005044 | 1 | PRKX, PRKY |
| 204139_x_at | NM_003422 | 1 | ZNF42 |
| 204153_s_at | AI738965 | 1 | MFNG |
| 204178_s_at | NM_006328 | 1 | RBM14 |
| 204185_x_at | NM_005038 | 1 | PPID |
| 204202_at | NM_017604 | 1 | IQCE |
| 204279_at | NM_002800 | 1 | PSMB9 |
| 204291_at | NM_014803 | 1 | ZNF518 |
| 204327_s_at | NM_003455 | 1 | ZNF202 |
| 204396_s_at | NM_005308 | 1 | GRK5 |
| 204410_at | NM_004681 | 1 | EIF1AY |
| 204510_at | NM_003503 | 1 | CDC7 |
| 204528_s_at | NM_004537 | 1 | NAP1L1 |
| 204581_at | NM_001771 | 1 | CD22 |
| 204593_s_at | AA046752 | 1 | FLJ20232 |
| 204618_s_at | NM_005254 | 1 | GABPB2 |
| 204640_s_at | NM_003563 | 1 | SPOP |
| 204772_s_at | NM_007344 | 1 | TTF1 |
| 204848_x_at | NM_000559 | 1 | HBG1, HBG2 |
| 204873_at | NM_000466 | 1 | PEX1 |
| 204950_at | NM_014959 | 1 | CARD8 |
| 205034_at | NM_004702 | 1 | CCNE2 |
| 205049_s_at | NM_001783 | 1 | CD79A |
| 208985_s_at | BC002719 | 1 | EIF3S1 |
| 209685_s_at | M13975 | 1 | PRKCB1 |
| 205296_at | AL365505 | 1 | RBL1 |
| 205297_s_at | NM_000626 | 1 | CD79B |
| 205353_s_at | NM_002567 | 1 | PBP |
| 205612_at | NM_007351 | 1 | MMRN1 |
| 205671_s_at | NM_002120 | 1 | HLA-DOB |
| 205788_s_at | NM_014827 | 1 | NM_014827 |
| 205790_at | NM_003726 | 1 | SCAP1 |
| 205933_at | NM_015559 | 1 | SETBP1 |
| 206272_at | NM_006542 | 1 | SPHAR |
| 206493_at | NM_000419 | 1 | ITGA2B |
| 206494_s_at | NM_000419 | 1 | ITGA2B |
| 206652_at | NM_016384 | 1 | DKFZp779H233 |
| 206854_s_at | NM_003188 | 1 | MAP3K7 |
| 207081_s_at | NM_002650 | 1 | PIK4CA |
| 207170_s_at | NM_015416 | 1 | LETMD1 |
| 207180_s_at | NM_006410 | 1 | HTATIP2 |
| 207314_x_at | NM_006737 | 1 | KIR3DL2 |
| 207405_s_at | NM_002873 | 1 | RAD17 |
| 207522_s_at | NM_005173 | 1 | ATP2A3 |
| 207655_s_at | NM_013314 | 1 | BLNK |
| 207812_s_at | NM_015530 | 1 | GORASP2 |
| 207830_s_at | NM_002713 | 1 | PPP1R8 |
| 207842_s_at | NM_007359 | 1 | CASC3 |
| 208073_x_at | NM_003316 | 1 | TTC3 |
| 208184_s_at | NM_003274 | 1 | TMEM1 |
| 208622_s_at | AA670344 | 1 | VIL2 |
| 208640_at | BG292367 | 1 | RAC1 |
| 208651_x_at | BG327863 | 1 | CD24 |
| 208722_s_at | BC001081 | 1 | ANAPC5 |
| 208752_x_at | AI888672 | 1 | NAP1L1 |
| 208758_at | D89976 | 1 | ATIC |
| 208792_s_at | M25915 | 1 | CLU |
| 208809_s_at | AL136632 | 1 | C6orf62 |
| 208837_at | BC000027 | 1 | C15orf22 |
| 209007_s_at | AF247168 | 1 | NPD014 |
| 209058_at | AB002282 | 1 | EDF1 |
| 209075_s_at | AY009128 | 1 | ISCU |
| 209139_s_at | AF083033 | 1 | PRKRA |
| 209153_s_at | M31523 | 1 | TCF3 |
| 209215_at | L11669 | 1 | TETRAN |
| 209234_at | BF939474 | 1 | KIF1B |
| 209273_s_at | BG387555 | 1 | HBLD2 |
| 209284_s_at | AI922509 | 1 | RAP140 |
| 209340_at | S73498 | 1 | UAP1 |
| 209431_s_at | AF254083 | 1 | ZNF278 |
| 209447_at | AF043290 | 1 | SYNE1 |
| 209449_at | AF196468 | 1 | LSM2 |
| 209452_s_at | AF035824 | 1 | VTI1B |
| 209482_at | BC001430 | 1 | POP7 |
| 209486_at | BC004546 | 1 | SAS10 |
| 209503_s_at | AF035309 | 1 | PSMC5 |
| 209549_s_at | BC001121 | 1 | DGUOK |
| 209572_s_at | AF080227 | 1 | EED |
| 209625_at | BC004100 | 1 | PIGH |
| 209630_s_at | U87460 | 1 | FBXW2 |
| 209659_s_at | AF164598 | 1 | CDC16 |
| 209729_at | BC001782 | 1 | GAS2L1 |
| 209771_x_at | AA761181 | 1 | CD24 |
| 209778_at | AF007217 | 1 | TRIP11 |
| 209903_s_at | U49844 | 1 | ATR |
| 209917_s_at | BC002709 | 1 | TP53AP1 |
| 210046_s_at | U52144 | 1 | IDH2 |
| 210356_x_at | BC002807 | 1 | MS4A1 |
| 210378_s_at | BC004118 | 1 | SSNA1 |
| 210627_s_at | BC002804 | 1 | GCS1 |
| 210690_at | U96845 | 1 | KLRC4 |
| 210715_s_at | AF027205 | 1 | SPINT2 |
| 210719_s_at | BC002552 | 1 | HMG20B |
| 210886_x_at | AB007457 | 1 | TP53AP1 |
| 210927_x_at | BC004239 | 1 | JTB |
| 211036_x_at | BC006301 | 1 | ANAPC5 |
| 211479_s_at | M81778 | 1 | HTR2C |
| 211881_x_at | AB014341 | 1 | IGLJ3 |
| 213351_s_at | AB018322 | 1 | KIAA0779 |
| 213364_s_at | AI052536 | 1 | SNX1 |
| 211784_s_at | BC006181 | 1 | SFRS1 |
| 211945_s_at | BG500301 | 1 | ITGB1 |
| 211954_s_at | BC000947 | 1 | RANBP5 |
| 211979_at | AB046844 | 1 | GPR107 |
| 212040_at | BG249599 | 1 | TGOLN2 |
| 212068_s_at | AB011087 | 1 | KIAA0515 |
| 212114_at | BE967207 | 1 | microtubule |
| | | | protein |
| 212121_at | BE962354 | 1 | C10orf61 |
| 212140_at | AB014548 | 1 | SCC-112 |
| 212201_at | AW274877 | 1 | KIAA0692 |
| 212244_at | AL050091 | 1 | GRINL1A |
| 212247_at | AW008531 | 1 | NUP205 |
| 212321_at | BE999972 | 1 | BE999972 |
| 212326_at | AB007922 | 1 | VPS13D |
| 212361_s_at | AA805753 | 1 | ATP2A2 |
| 212548_s_at | BF515124 | 1 | KIAA0826 |
| 212559_at | AU148827 | 1 | AU148827 |
| 212566_at | AL523310 | 1 | MAP4 |
| 212583_at | AB011132 | 1 | AB011132 |
| 212611_at | AV728526 | 1 | DTX4 |
| 212632_at | N32035 | 1 | STX7 |
| 212655_at | AB011151 | 1 | ZCCHC14 |
| 212667_at | AL575922 | 1 | SPARC |
| 212690_at | AB018268 | 1 | DDHD2 |
| 212714_at | AL050205 | 1 | LOC113251 |
| 212750_at | AB020630 | 1 | PPP1R16B |
| 212760_at | AB002347 | 1 | C6orf133 |
| 212813_at | AA149644 | 1 | JAM3 |
| 212855_at | D87466 | 1 | KIAA0276 |
| 212955_s_at | AL037557 | 1 | POLR2I |
| 213061_s_at | AA643304 | 1 | NTAN1 |
| 213065_at | AB011118 | 1 | MGC23401 |
| 213088_s_at | BE551340 | 1 | DNAJC9 |
| 213090_s_at | AI744029 | 1 | TAF4 |
| 213106_at | AI769688 | 1 | ATP8A1 |
| 213165_at | AI041204 | 1 | CAP350 |
| 213213_at | AL035669 | 1 | DATF1 |
| 213267_at | AL162056 | 1 | KIAA1117 |
| 213278_at | AW014788 | 1 | MTMR9 |
| 213375_s_at | N80918 | 1 | CG018 |
| 213408_s_at | AK024034 | 1 | PIK4CA |
| 213410_at | AL050102 | 1 | C10orf137 |
| 213460_x_at | N29665 | 1 | WBSCR20C |
| 213483_at | AK025679 | 1 | KIAA0073 |
| 213515_x_at | AI133353 | 1 | HBG2 |
| 213528_at | AL035369 | 1 | MGC9084 |
| 213626_at | AL049442 | 1 | CBR4 |
| 213634_s_at | AL031588 | 1 | TRMT1 |
| 213653_at | AW069290 | 1 | METTL3 |
| 213659_at | AA209420 | 1 | ZNF75 |
| 213672_at | AA621558 | 1 | MARS |
| 213838_at | AA191426 | 1 | AA191426 |
| 214130_s_at | AI821791 | 1 | PDE4DIP |
| 214669_x_at | BG485135 | 1 | BG485135 |
| 214749_s_at | AK000818 | 1 | ARMCX6 |
| 214948_s_at | AL050136 | 1 | TMF1 |
| 215158_s_at | AK022531 | 1 | DEDD |
| 215227_x_at | BG035989 | 1 | ACP1 |
| 215318_at | AL049782 | 1 | CG012 |
| 215343_at | AF070587 | 1 | 0610010D24Rik |
| 215648_at | AU144324 | 1 | KIAA1068 |
| 215925_s_at | AF283777 | 1 | CD72 |
| 216237_s_at | AA807529 | 1 | MCM5 |
| 216261_at | AI151479 | 1 | ITGB3 |
| 216338_s_at | AK021433 | 1 | C6orf109 |
| 218100_s_at | NM_018010 | 1 | ESRRBL1 |
| 219802_at | NM_024854 | 1 | FLJ22028 |
| 216379_x_at | AK000168 | 1 | CD24 |
| 216907_x_at | X93596 | 1 | KIR3DL2 |
| 216956_s_at | AF098114 | 1 | ITGA2B |
| 217043_s_at | U95822 | 1 | MFN1 |
| 217418_x_at | X12530 | 1 | MS4A1 |
| 217645_at | AW088547 | 1 | AW088547 |
| 217730_at | NM_022152 | 1 | PP1201 |
| 217772_s_at | NM_014342 | 1 | MTCH2 |
| 217792_at | NM_014426 | 1 | SNX5 |
| 217815_at | NM_007192 | 1 | SUPT16H |
| 217896_s_at | NM_024946 | 1 | NIP30 |
| 217906_at | NM_014315 | 1 | KLHDC2 |
| 217908_s_at | NM_018442 | 1 | IQWD1 |
| 217938_s_at | AI743396 | 1 | KCMF1 |
| 217978_s_at | NM_017582 | 1 | UBE2Q |
| 217979_at | NM_014399 | 1 | TM4SF13 |
| 217986_s_at | AA102574 | 1 | BAZ1A |
| 218004_at | NM_018045 | 1 | FLJ10276 |
| 218082_s_at | NM_014517 | 1 | UBP1 |
| 218124_at | NM_017750 | 1 | FLJ20296 |
| 218132_s_at | NM_024075 | 1 | LENG5 |
| 218179_s_at | NM_021942 | 1 | FLJ12716 |
| 218212_s_at | NM_004531 | 1 | MOCS2 |
| 218220_at | NM_021640 | 1 | C12orf10 |
| 218237_s_at | NM_030674 | 1 | SLC38A1 |
| 218250_s_at | NM_013354 | 1 | CNOT7 |
| 218301_at | NM_018226 | 1 | RNPEPL1 |
| 218328_at | NM_016035 | 1 | COQ4 |
| 218339_at | NM_014180 | 1 | MRPL22 |
| 218361_at | NM_018178 | 1 | GPP34R |
| 218371_s_at | AA969958 | 1 | PSPC1 |
| 218432_at | NM_012175 | 1 | FBXO3 |
| 218440_at | NM_020166 | 1 | MCCC1 |
| 218456_at | NM_023925 | 1 | C1QDC1 |
| 218462_at | NM_025065 | 1 | RPF1 |
| 218504_at | NM_016044 | 1 | FAHD2A |
| 218533_s_at | NM_017859 | 1 | UCKL1 |
| 218575_at | NM_022662 | 1 | ANAPC1 |
| 218607_s_at | NM_018115 | 1 | SDAD1 |
| 218608_at | NM_022089 | 1 | HSA9947 |
| 218626_at | NM_019843 | 1 | EIF4ENIF1 |
| 218639_s_at | NM_025112 | 1 | MGC11349 |
| 218654_s_at | NM_016071 | 1 | MRPS33 |
| 218667_at | NM_022368 | 1 | PJA1 |
| 218679_s_at | NM_016208 | 1 | VPS28 |
| 218771_at | NM_018216 | 1 | PANK4 |
| 219067_s_at | NM_017615 | 1 | C10orf86 |
| 219126_at | NM_018288 | 1 | PHF10 |
| 219151_s_at | NM_007081 | 1 | RABL2A, |
| | | | RABL2B |
| 219180_s_at | AI817074 | 1 | PEX26 |
| 219185_at | NM_012241 | 1 | SIRT5 |
| 219283_at | NM_014158 | 1 | C1GALT2 |
| 219292_at | NM_018105 | 1 | THAP1 |
| 219317_at | NM_007195 | 1 | POLI |
| 219470_x_at | NM_019084 | 1 | CCNJ |
| 219581_at | NM_025265 | 1 | SEN2L |
| 219627_at | NM_024910 | 1 | FLJ12700 |
| 219822_at | NM_004294 | 1 | MTRF1 |
| 219880_at | NM_022907 | 1 | FLJ23053 |
| 219979_s_at | NM_016401 | 1 | HSPC138 |
| 220044_x_at | NM_016424 | 1 | CROP |
| 220059_at | NM_012108 | 1 | BRDG1 |
| 220169_at | NM_024943 | 1 | FLJ23235 |
| 220216_at | NM_019607 | 1 | FLJ11267 |
| 220261_s_at | NM_018106 | 1 | ZDHHC4 |
| 220387_s_at | NM_007071 | 1 | HHLA3 |
| 220418_at | NM_018961 | 1 | UBASH3A |
| 220936_s_at | NM_018267 | 1 | H2AFJ |
| 221208_s_at | NM_024631 | 1 | FLJ23342 |
| 221253_s_at | NM_030810 | 1 | TXNDC5 |
| 221476_s_at | AF279903 | 1 | RPL15 |
| 221515_s_at | BC001214 | 1 | LCMT1 |
| 221516_s_at | BC002587 | 1 | FLJ20232 |
| 221548_s_at | AY024365 | 1 | ILKAP |
| 221586_s_at | U15642 | 1 | E2F5 |
| 221736_at | BG236163 | 1 | KIAA1219 |
| 221791_s_at | BG167522 | 1 | HSPC016 |
| 221811_at | BF033007 | 1 | PERLD1 |
| 221932_s_at | AA133341 | 1 | C14orf87 |
| 222111_at | AU145293 | 1 | KIAA1164 |
| 222130_s_at | AK024635 | 1 | FTSJ2 |
| 222155_s_at | AK021918 | 1 | GPR172A |
| 222233_s_at | AK022922 | 1 | DCLRE1C |
| 222273_at | AI419423 | 1 | AI419423 |
| 266_s_at | L33930 | 1 | CD24 |
| 32259_at | AB002386 | 1 | EZH1 |
| 33307_at | AL022316 | 1 | CGI-96 |
| 37965_at | AA181053 | 1 | PARVB |
| 37966_at | AA187563 | 1 | PARVB |
| 38043_at | X55448 | 1 | FAM3A |
| 45828_at | AI768100 | 1 | FLJ10241 |
| 48825_at | AA887083 | 1 | ING4 |
| 50374_at | AA150503 | 1 | LOC339229 |
| 52164_at | AA065185 | 1 | C11orf24 |
| 64432_at | W05463 | 1 | FLJ39616 |
| 65472_at | AI161338 | 1 | AI161338 |

Table 1: Provided are genetic markers which are differentially expressed between subjects diagnosed with probable multiple sclerosis and healthy controls (the Probeset ID of the Affymetrix Gene Chip), along with the corresponding GenBank accession number (GenBank Acc. No.), the gene symbol and the direction (Dir) of change in gene expression (“1”- upregulation; “−1” - downregulation). Note that the p values of the TNOM, Info and t-Test statistical tests all passed the 95% confidence level.

Independent verification by support vector machine (SVM)—Verification of the probable PBMC gene expression signature (554 genes) was performed on an independent group of 15 subjects (12 patients, 3 controls) by SVM analysis and resulted in high classification rate of 80%. These findings suggest that the identified gene expression signature in probable MS is reliable and not related to spurious difference due to multiple testing.

### Example 2

### Gene Expression Analysis of Subjects Whose Diagnosis Converted to Definite MS or Sustained Probable MS

Experimental Results

Conversion to definite MS—2-year results—During the follow up period of 2 year, 30% of patients (12/40) experienced a second attack and progressed to definite MS disease (defined as early convectors to definite MS). Comparison of the gene expression pattern of only these probable patients who further experienced a second attack and therefore defined as definite MS (12 patients) (using the blood cell samples obtained when the subjects were defined as probable multiple sclerosis, i.e., after the first neurological attack) to matched control group (11 subjects) identified 1517 most informative genes (Table 2, hereinbelow and FIG. 2a), all passed FDR at p<0.03 and 8 genes—Bonfferoni correction at p<3.2×10⁻⁵. PCA performed on these 1517 most informative genes demonstrated correct classification rate with no errors (FIG. 2b).

TABLE 2
Differentially expressed markers between probable multiple sclerosis
subjects which further developed definite multiple sclerosis and
healthy controls

| | GenBank | | |
|---|---|---|---|
| Probeset | Acc. No. | Dir | Gene Symbol |
| 214376_at | AI263044 | −1 | AI263044 |
| 217446_x_at | AL080160 | −1 | AL080160 |
| 215185_at | AK024177 | −1 | AK024177 |
| 217449_at | AL137284 | −1 | DKFZp434D1516 |
| 221877_at | BF508835 | −1 | FKSG27 |
| 222135_at | AK022663 | −1 | KIAA1956 |
| 217381_s_at | X69383 | −1 | T-cell receptor |
| | | | gamma |
| 221477_s_at | BC001980 | −1 | NP_079012.2 |
| 215995_x_at | AU147598 | −1 | LOC283585 |
| 214826_at | U79276 | −1 | 2′-PDE |
| 44702_at | R77097 | −1 | 7h3 |
| 201269_s_at | AB028991 | −1 | AB028991 |
| 214979_at | AK000791 | −1 | ABCC3 |
| 205566_at | NM_007011 | −1 | ABHD2 |
| 207268_x_at | NM_005759 | −1 | ABI2 |
| 217024_x_at | AC004832 | −1 | AC004832 |
| 206776_x_at | NM_001612 | −1 | ACRV1 |
| 207973_x_at | NM_020110 | −1 | ACRV1 |
| 207990_x_at | NM_020114 | −1 | ACRV1 |
| 207991_x_at | NM_020113 | −1 | ACRV1 |
| 211489_at | D32201 | −1 | ADRA1A |
| 38447_at | U08438 | −1 | ADRBK1 |
| 201205_at | AF006751 | −1 | AF006751 |
| 216366_x_at | AF047245 | −1 | AF047245 |
| 211189_x_at | AF054816 | −1 | AF054816 |
| 215837_x_at | AF113018 | −1 | AF113018 |
| 208849_at | AF118091 | −1 | AF118091 |
| 219558_at | NM_024524 | −1 | AFURS1 |
| 205357_s_at | NM_000685 | −1 | AGTR1 |
| 207464_at | NM_014121 | −1 | AHCYL1 |
| 214139_at | AI051476 | −1 | AI051476 |
| 221960_s_at | AI189609 | −1 | AI189609 |
| 214379_at | AI954458 | −1 | AI954458 |
| 216678_at | AK000773 | −1 | AK000773 |
| 216682_s_at | AK021457 | −1 | AK021457 |
| 216051_x_at | AK022045 | −1 | AK022045 |
| 217335_at | AK023539 | −1 | AK023539 |
| 216739_at | AK024527 | −1 | AK024527 |
| 216746_at | AK024606 | −1 | AK024606 |
| 216437_at | AK024949 | −1 | AK024949 |
| 215405_at | AK025072 | −1 | AK025072 |
| 216774_at | AK025325 | −1 | AK025325 |
| 217218_at | AK027005 | −1 | AK027005 |
| 210517_s_at | AB003476 | −1 | AKAP12 |
| 205359_at | NM_004274 | −1 | AKAP6 |
| 211172_x_at | AF161075 | −1 | AKAP7 |
| 204151_x_at | NM_001353 | −1 | AKR1C1 |
| 216336_x_at | AL031602 | −1 | AL031602 |
| 41856_at | AL049370 | −1 | AL049370 |
| 216499_at | AL137590 | −1 | AL137590 |
| 216828_at | AL441988 | −1 | AL441988 |
| 210544_s_at | BC002430 | −1 | ALDH3A2 |
| 214423_x_at | AV652403 | −1 | ALDOB |
| 207755_at | NM_025017 | −1 | AMOTL2 |
| 213001_at | AF007150 | −1 | ANGPTL2 |
| 218418_s_at | NM_015493 | −1 | ANKRD25 |
| 203074_at | NM_001630 | −1 | ANXA8 |
| 211553_x_at | AF149794 | −1 | APAF1 |
| 206350_at | NM_001639 | −1 | APCS |
| 202914_s_at | NM_014784 | −1 | ARHGEF11 |
| 207919_at | NM_004314 | −1 | ART1 |
| 202672_s_at | NM_001674 | −1 | ATF3 |
| 216070_at | AL049331 | −1 | ATP1B1 |
| 214132_at | BG232034 | −1 | ATP5C1 |
| 204624_at | NM_000053 | −1 | ATP7B |
| 222303_at | AV700891 | −1 | AV700891 |
| 206251_s_at | NM_000706 | −1 | AVPR1A |
| 212999_x_at | AW276186 | −1 | AW276186 |
| 222363_at | AW979018 | −1 | AW979018 |
| 219326_s_at | AF288208 | −1 | B3GNT1 |
| 209413_at | BC002431 | −1 | B4GALT2 |
| 91920_at | AI205180 | −1 | BCAN |
| 219444_at | NM_021946 | −1 | BCORL1 |
| 213281_at | BE327172 | −1 | BE327172 |
| 221917_s_at | BF058465 | −1 | BF058465 |
| 213561_at | BF062335 | −1 | BF062335 |
| 215775_at | BF084105 | −1 | BF084105 |
| 212002_at | BG171020 | −1 | BG171020 |
| 220580_at | NM_025044 | −1 | BICC1 |
| 218955_at | NM_018310 | −1 | BRF2 |
| 201235_s_at | BG339064 | −1 | BTG2 |
| 215072_x_at | AK025270 | −1 | C10orf137 |
| 43934_at | AA479495 | −1 | C11ORF4 |
| 219720_s_at | NM_017972 | −1 | C14orf118 |
| 215219_at | AK025095 | −1 | C21orf5 |
| 48030_i_at | H93077 | −1 | C5orf4 |
| 220231_at | NM_006658 | −1 | C7orf16 |
| 61874_at | AL042496 | −1 | C9orf7 |
| 205949_at | M33987 | −1 | CA1 |
| 205199_at | NM_001216 | −1 | CA9 |
| 214845_s_at | AF257659 | −1 | CALU |
| 34846_at | AF112472 | −1 | CAMK2B |
| 210787_s_at | AF140507 | −1 | CAMKK2 |
| 205114_s_at | NM_002983 | −1 | CCL3, CCL3L1, |
| | | | MGC12815 |
| 202705_at | NM_004701 | −1 | CCNB2 |
| 205098_at | AI421071 | −1 | CCR1 |
| 208304_at | NM_001837 | −1 | CCR3 |
| 207445_s_at | AF145439 | −1 | CCR9 |
| 210325_at | M28825 | −1 | CD1A |
| 207176_s_at | NM_005191 | −1 | CD80 |
| 211190_x_at | AF054817 | −1 | CD84 |
| 209287_s_at | AI754416 | −1 | CDC42EP3 |
| 209057_x_at | AB007892 | −1 | CDC5L |
| 220115_s_at | NM_006727 | −1 | CDH10 |
| 211804_s_at | AB012305 | −1 | CDK2 |
| 211883_x_at | M76742 | −1 | CEACAM1 |
| 40020_at | AB011536 | −1 | CELSR3 |
| 220885_s_at | NM_018451 | −1 | CENPJ |
| 202938_x_at | NM_015703 | −1 | CGI-96, |
| | | | dJ222E13.2 |
| 207486_x_at | NM_004067 | −1 | CHN2 |
| 206635_at | NM_000748 | −1 | CHRNB2 |
| 215916_at | AL157418 | −1 | CHRNE |
| 207075_at | NM_004895 | −1 | CIAS1 |
| 216016_at | AK027194 | −1 | CIAS1 |
| 206818_s_at | NM_017649 | −1 | CNNM2 |
| 209874_x_at | AK023066 | −1 | CNNM2 |
| 206586_at | NM_001841 | −1 | CNR2 |
| 211980_at | AI922605 | −1 | COL4A1 |
| 216898_s_at | U02520 | −1 | COL4A3 |
| 52651_at | AI806793 | −1 | COL8A2 |
| 205624_at | NM_001870 | −1 | CPA3 |
| 222301_at | BF530257 | −1 | CROC4 |
| 202468_s_at | NM_003798 | −1 | CTNNAL1 |
| 219080_s_at | NM_019857 | −1 | CTPS2 |
| 206297_at | NM_007272 | −1 | CTRC |
| 205927_s_at | NM_001910 | −1 | CTSE |
| 206775_at | NM_001081 | −1 | CUBN |
| 204470_at | NM_001511 | −1 | CXCL1 |
| 209774_x_at | M57731 | −1 | CXCL2 |
| 207850_at | NM_002090 | −1 | CXCL3 |
| 206336_at | NM_002993 | −1 | CXCL6 |
| 205088_at | NM_005491 | −1 | CXorf6 |
| 214610_at | AV702430 | −1 | CYP11B1 |
| 217558_at | BE971373 | −1 | CYP2C9 |
| 213873_at | D29810 | −1 | D29810 |
| 217025_s_at | AL110225 | −1 | DBN1 |
| 219490_s_at | NM_022836 | −1 | DCLRE1B |
| 205338_s_at | NM_001922 | −1 | DCT |
| 213632_at | M94065 | −1 | DHODH |
| 219799_s_at | NM_005771 | −1 | DHRS9 |
| 204494_s_at | AW516789 | −1 | DKFZP434H132 |
| 214699_x_at | AK024279 | −1 | DKFZP434J154 |
| 219872_at | NM_016613 | −1 | DKFZp434L142 |
| 78495_at | R61320 | −1 | DKFZp762P2111 |
| 208216_at | NM_001934 | −1 | DLX4 |
| 33768_at | L19267 | −1 | DMWD |
| 202866_at | BG283782 | −1 | DNAJB12 |
| 219746_at | NM_012074 | −1 | DPF3 |
| 206590_x_at | NM_016574 | −1 | DRD2 |
| 216938_x_at | S69899 | −1 | DRD2 |
| 211541_s_at | U52373 | −1 | DYRK1A |
| 218660_at | NM_003494 | −1 | DYSF |
| 203692_s_at | AI640363 | −1 | E2F3 |
| 208112_x_at | NM_006795 | −1 | EHD1 |
| 209038_s_at | AL579035 | −1 | EHD1 |
| 209039_x_at | AF001434 | −1 | EHD1 |
| 210376_x_at | M25269 | −1 | ELK1 |
| 205994_at | NM_001973 | −1 | ELK4 |
| 396_f_at | X97671 | −1 | EPOR |
| 206794_at | NM_005235 | −1 | ERBB4 |
| 205225_at | NM_000125 | −1 | ESR1 |
| 206501_x_at | NM_004956 | −1 | ETV1 |
| 215006_at | AK023816 | −1 | EZH2 |
| 205189_s_at | NM_000136 | −1 | FANCC |
| 204819_at | NM_004463 | −1 | FGD1 |
| 219901_at | NM_018351 | −1 | FGD6 |
| 208417_at | NM_020996 | −1 | FGF6 |
| 219389_at | NM_017982 | −1 | FLJ10052 |
| 219501_at | NM_017993 | −1 | FLJ10094 |
| 218815_s_at | NM_018022 | −1 | FLJ10199 |
| 218814_s_at | NM_018252 | −1 | FLJ10874 |
| 219450_at | NM_018302 | −1 | FLJ11017 |
| 220465_at | NM_024988 | −1 | FLJ12355 |
| 220578_at | NM_025008 | −1 | FLJ13544 |
| 220149_at | NM_024861 | −1 | FLJ22671 |
| 221224_s_at | NM_024819 | −1 | FLJ22955 |
| 215062_at | AL390143 | −1 | FMNL2 |
| 206263_at | NM_002022 | −1 | FMO4 |
| 202768_at | NM_006732 | −1 | FOSB |
| 219889_at | NM_005479 | −1 | FRAT1 |
| 211628_x_at | J04755 | −1 | FTHP1 |
| 215744_at | AW514140 | −1 | FUS |
| 209893_s_at | M58596 | −1 | FUT4 |
| 202488_s_at | NM_005971 | −1 | FXYD3 |
| 204452_s_at | AF072872 | −1 | FZD1 |
| 213524_s_at | NM_015714 | −1 | G0S2 |
| 214772_at | H08993 | −1 | G2 |
| 208217_at | NM_002043 | −1 | GABRR2 |
| 207574_s_at | NM_015675 | −1 | GADD45B |
| 209305_s_at | AF078077 | −1 | GADD45B |
| 207357_s_at | NM_017540 | −1 | GALNT10 |
| 220929_at | NM_017417 | −1 | GALNT8 |
| 207954_at | NM_002050 | −1 | GATA2 |
| 209710_at | AL563460 | −1 | GATA2 |
| 210358_x_at | BC002557 | −1 | GATA2 |
| 221314_at | NM_005260 | −1 | GDF9 |
| 205100_at | NM_005110 | −1 | GFPT2 |
| 211815_s_at | AF219138 | −1 | GGA3 |
| 207131_x_at | NM_013430 | −1 | GGT1 |
| 208284_x_at | NM_013421 | −1 | GGT1 |
| 209919_x_at | L20490 | −1 | GGT1 |
| 211417_x_at | L20493 | −1 | GGT1 |
| 215977_x_at | X68285 | −1 | GK |
| 207034_s_at | NM_030379 | −1 | GLI2 |
| 204762_s_at | NM_020988 | −1 | GNAO1 |
| 209220_at | L47125 | −1 | GPC3 |
| 206264_at | L11702 | −1 | GPLD1 |
| 209168_at | AW148844 | −1 | GPM6B |
| 211977_at | AK024651 | −1 | GPR107 |
| 205220_at | NM_006018 | −1 | GPR109B |
| 221394_at | NM_014626 | −1 | GPR58 |
| 214655_at | U18549 | −1 | GPR6 |
| 220481_at | NM_006794 | −1 | GPR75 |
| 205276_s_at | U87964 | −1 | GTPBP1 |
| 44783_s_at | R61374 | −1 | HEY1 |
| 210864_x_at | AF144240 | −1 | HFE |
| 211328_x_at | AF144244 | −1 | HFE |
| 211329_x_at | AF115264 | −1 | HFE |
| 211332_x_at | AF144241 | −1 | HFE |
| 211863_x_at | AF079408 | −1 | HFE |
| 205425_at | NM_005338 | −1 | HIP1 |
| 205426_s_at | NM_005338 | −1 | HIP1 |
| 209398_at | BC002649 | −1 | HIST1H1C |
| 214290_s_at | AI313324 | −1 | HIST2H2AA |
| 218280_x_at | BC001629 | −1 | HIST2H2AA |
| 208812_x_at | BC004489 | −1 | HLA-C, HLA-B |
| 211911_x_at | L07950 | −1 | HLA-C, HLA-B |
| 211654_x_at | M17565 | −1 | HLA-DQB1 |
| 211528_x_at | M90685 | −1 | HLA-G |
| 211529_x_at | M90684 | −1 | HLA-G |
| 213844_at | NM_019102 | −1 | HOXA5 |
| 205601_s_at | NM_002147 | −1 | HOXB5 |
| 216973_s_at | S49765 | −1 | HOXB7 |
| 204221_x_at | U16307 | −1 | HRB2 |
| 214085_x_at | AI912583 | −1 | HRB2 |
| 209192_x_at | BC000166 | −1 | HTATIP |
| 207404_s_at | NM_000865 | −1 | HTR1E |
| 36564_at | W27419 | −1 | IBRDC3 |
| 202637_s_at | AI608725 | −1 | ICAM1 |
| 202638_s_at | NM_000201 | −1 | ICAM1 |
| 215485_s_at | AA284705 | −1 | ICAM1 |
| 203328_x_at | NM_004969 | −1 | IDE |
| 202081_at | NM_004907 | −1 | IER2 |
| 201631_s_at | NM_003897 | −1 | IER3 |
| 208261_x_at | NM_002171 | −1 | IFNA10 |
| 214569_at | NM_002169 | −1 | IFNA5 |
| 208548_at | NM_021002 | −1 | IFNA6 |
| 208441_at | NM_015883 | −1 | IGF1R |
| 202421_at | AB007935 | −1 | IGSF3 |
| 209032_s_at | AF132811 | −1 | IGSF4 |
| 207901_at | NM_002187 | −1 | IL12B |
| 64440_at | AI560217 | −1 | IL17RC |
| 206295_at | NM_001562 | −1 | IL18 |
| 205067_at | NM_000576 | −1 | IL1B |
| 39402_at | M15330 | −1 | IL1B |
| 212659_s_at | AW083357 | −1 | IL1RN |
| 216243_s_at | BE563442 | −1 | IL1RN |
| 220056_at | NM_021258 | −1 | IL22RA1 |
| 202859_x_at | NM_000584 | −1 | IL8 |
| 211506_s_at | AF043337 | −1 | IL8 |
| 202531_at | NM_002198 | −1 | IRF1 |
| 206766_at | AF112345 | −1 | ITGA10 |
| 37201_at | D38535 | −1 | ITIH4 |
| 203682_s_at | NM_002225 | −1 | IVD |
| 209098_s_at | U73936 | −1 | JAG1 |
| 201465_s_at | BC002646 | −1 | JUN |
| 211806_s_at | D87291 | −1 | KCNJ15 |
| 58916_at | AI672101 | −1 | KCTD14 |
| 211028_s_at | BC006233 | −1 | KHK |
| 216251_s_at | BF965437 | −1 | KIAA0153 |
| 210954_s_at | AF201292 | −1 | KIAA0669 |
| 31826_at | AB014574 | −1 | KIAA0674 |
| 212054_x_at | AK026096 | −1 | KIAA0676 |
| 213358_at | AB018345 | −1 | KIAA0802 |
| 207705_s_at | NM_025176 | −1 | KIAA0980 |
| 213478_at | AB028949 | −1 | KIAA1026 |
| 216294_s_at | AL137254 | −1 | KIAA1109 |
| 221078_s_at | NM_018084 | −1 | KIAA1212 |
| 222367_at | AI921841 | −1 | KIAA1971, |
| | | | LOC339005 |
| 205318_at | NM_004984 | −1 | KIF5A |
| 207908_at | NM_000423 | −1 | KRT2A |
| 215189_at | X99142 | −1 | KRTHB6 |
| 221717_at | L25664 | −1 | L25664 |
| 219813_at | NM_004690 | −1 | LATS1 |
| 204012_s_at | AL529189 | −1 | LCMT2 |
| 215462_at | AI978990 | −1 | LOC149478 |
| 51774_s_at | AW014299 | −1 | LOC222070 |
| 58900_at | AW025284 | −1 | LOC222070 |
| 217520_x_at | BG396614 | −1 | LOC283683 |
| 213751_at | AW873594 | −1 | LOC284352 |
| 214838_at | AL035297 | −1 | LOC375035 |
| 219071_x_at | NM_016458 | −1 | LOC51236 |
| 221629_x_at | AF151022 | −1 | LOC51236 |
| 220244_at | NM_013343 | −1 | LOH3CR2A |
| 34697_at | AF074264 | −1 | LRP6 |
| 219886_at | NM_024548 | −1 | LRRIQ2 |
| 204682_at | NM_000428 | −1 | LTBP2 |
| 202018_s_at | NM_002343 | −1 | LTF |
| 209480_at | M16276 | −1 | M16276 |
| 209738_x_at | M31125 | −1 | M31125 |
| 211241_at | M62895 | −1 | M62895 |
| 217920_at | BE543064 | −1 | MAN1A2 |
| 209951_s_at | AF006689 | −1 | MAP2K7 |
| 214969_at | AF251442 | −1 | MAP3K9 |
| 204813_at | U34819 | −1 | MAPK10 |
| 201668_x_at | AW163148 | −1 | MARCKS |
| 202485_s_at | NM_003927 | −1 | MBD2 |
| 200797_s_at | AI275690 | −1 | MCL1 |
| 202618_s_at | L37298 | −1 | MECP2 |
| 206028_s_at | NM_006343 | −1 | MERTK |
| 211913_s_at | L08961 | −1 | MERTK |
| 211599_x_at | U19348 | −1 | MET |
| 209758_s_at | U37283 | −1 | MFAP5 |
| 211717_at | BC005853 | −1 | MGC15396 |
| 212340_at | BE673723 | −1 | MGC21416 |
| 217548_at | AA491625 | −1 | MGC61550 |
| 205905_s_at | NM_000247 | −1 | MICA, MICB |
| 208384_s_at | NM_012216 | −1 | MID2 |
| 212021_s_at | AU132185 | −1 | MKI67 |
| 218211_s_at | NM_024101 | −1 | MLPH |
| 160020_at | Z48481 | −1 | MMP14 |
| 203936_s_at | NM_004994 | −1 | MMP9 |
| 203949_at | NM_000250 | −1 | MPO |
| 206186_at | NM_001932 | −1 | MPP3 |
| 210594_x_at | AF239756 | −1 | MPZL1 |
| 210223_s_at | AF031469 | −1 | MR1 |
| 210224_at | AF031469 | −1 | MR1 |
| 210242_x_at | AF249277 | −1 | MTHFS |
| 212093_s_at | AI695017 | −1 | MTUS1 |
| 207847_s_at | NM_002456 | −1 | MUC1 |
| 212365_at | BF215996 | −1 | MYO1B |
| 33197_at | U39226 | −1 | MYO7A |
| 210048_at | BC001889 | −1 | NAPG |
| 218742_at | NM_022493 | −1 | NARFL |
| 217045_x_at | AL136967 | −1 | NCR2 |
| 202150_s_at | U64317 | −1 | NEDD9 |
| 203927_at | NM_004556 | −1 | NFKBIE |
| 207986_x_at | NM_001915 | −1 | NM_001915 |
| 208039_at | NM_003048 | −1 | NM_003048 |
| 208180_s_at | NM_003543 | −1 | NM_003543 |
| 220814_at | NM_017964 | −1 | NM_017964 |
| 221105_at | NM_018395 | −1 | NM_018395 |
| 220872_at | NM_018547 | −1 | NM_018547 |
| 208540_x_at | NM_021039 | −1 | NM_021039 |
| 219680_at | NM_024618 | −1 | NOD9 |
| 39548_at | U77970 | −1 | NPAS2 |
| 210730_s_at | U36269 | −1 | NPY2R |
| 202340_x_at | NM_002135 | −1 | NR4A1 |
| 204105_s_at | NM_005010 | −1 | NRCAM |
| 216959_x_at | U55258 | −1 | NRCAM |
| 210022_at | BC004952 | −1 | NSPC1 |
| 217377_x_at | AF041811 | −1 | NTRK3 |
| 206553_at | NM_002535 | −1 | OAS2 |
| 205660_at | NM_003733 | −1 | OASL |
| 214485_at | NM_024410 | −1 | ODF1 |
| 219105_x_at | NM_014321 | −1 | ORC6L |
| 205729_at | NM_003999 | −1 | OSMR |
| 205815_at | NM_002580 | −1 | PAP |
| 213332_at | AL031290 | −1 | PAPPA2 |
| 204752_x_at | NM_005484 | −1 | PARP2 |
| 215773_x_at | AJ236912 | −1 | PARP2 |
| 215418_at | AK022316 | −1 | PARVA |
| 121_at | X69699 | −1 | PAX8 |
| 205656_at | NM_014459 | −1 | PCDH17 |
| 211066_x_at | BC006439 | −1 | PDGHGA1-12 |
| | | | PDGHGB1-7 |
| 206388_at | U36798 | −1 | PDE3A |
| 216061_x_at | AK022920 | −1 | PDGFB |
| 220236_at | NM_017990 | −1 | PDPR |
| 220944_at | NM_020393 | −1 | PGLYRP4 |
| 215622_x_at | AL137671 | −1 | PHF7 |
| 209346_s_at | AL561930 | −1 | PI4KII |
| 215832_x_at | AV722190 | −1 | PICALM |
| 210256_s_at | U78576 | −1 | PIP5K1A |
| 204267_x_at | NM_004203 | −1 | PKMYT1 |
| 202924_s_at | AF006005 | −1 | PLAGL2 |
| 202925_s_at | NM_002657 | −1 | PLAGL2 |
| 203471_s_at | NM_002664 | −1 | PLEK |
| 39854_r_at | AF055000 | −1 | PNPLA2 |
| 206654_s_at | NM_006467 | −1 | POLR3G |
| 210809_s_at | D13665 | −1 | POSTN |
| 207725_at | NM_004575 | −1 | POU4F2 |
| 216330_s_at | L14482 | −1 | POU6F1 |
| 212226_s_at | AA628586 | −1 | PPAP2B |
| 210235_s_at | U22815 | −1 | PPFIA1 |
| 201489_at | BC005020 | −1 | PPIF |
| 201490_s_at | NM_005729 | −1 | PPIF |
| 202014_at | NM_014330 | −1 | PPP1R15A |
| 37028_at | U83981 | −1 | PPP1R15A |
| 221088_s_at | NM_017650 | −1 | PPP1R9A |
| 204506_at | AL544951 | −1 | PPP3R1 |
| 210499_s_at | AB041834 | −1 | PQBP1 |
| 209766_at | AF118073 | −1 | PRDX3 |
| 219732_at | NM_017753 | −1 | PRG-3 |
| 217269_s_at | AP001672 | −1 | PRSS7 |
| 210195_s_at | M34715 | −1 | PSG1 |
| 204897_at | AA897516 | −1 | PTGER4 |
| 204748_at | NM_000963 | −1 | PTGS2 |
| 202895_s_at | D86043 | −1 | PTPNS1 |
| 206157_at | NM_002852 | −1 | PTX3 |
| 202148_s_at | NM_006907 | −1 | PYCR1 |
| 211471_s_at | AF133588 | −1 | RAB36 |
| 37793_r_at | AF034956 | −1 | RAD51L3 |
| 204916_at | NM_005855 | −1 | RAMP1 |
| 217020_at | X04014 | −1 | RARB |
| 215688_at | AL359931 | −1 | RASGRF1 |
| 205115_s_at | NM_016196 | −1 | RBM19 |
| 215761_at | AK000156 | −1 | RC3 |
| 216153_x_at | AK022897 | −1 | RECK |
| 205879_x_at | BC004257 | −1 | RET |
| 207936_x_at | NM_006604 | −1 | RFPL3 |
| 209637_s_at | AF030111 | −1 | RGS12 |
| 209545_s_at | AF027706 | −1 | RIPK2 |
| 221287_at | NM_021133 | −1 | RNASEL |
| 208270_s_at | NM_020216 | −1 | RNPEP |
| 218441_s_at | NM_015540 | −1 | RPAP1 |
| 213223_at | AK025866 | −1 | RPL28 |
| 211180_x_at | D89788 | −1 | RUNX1 |
| 205485_at | NM_000540 | −1 | RYR1 |
| 217033_x_at | S76475 | −1 | S76475 |
| 210593_at | M55580 | −1 | SAT |
| 217331_at | U63542 | −1 | SCC-112 |
| 59705_at | AA911739 | −1 | SCLY |
| 207413_s_at | NM_000335 | −1 | SCN5A |
| 203788_s_at | AI962897 | −1 | SEMA3C |
| 35666_at | U38276 | −1 | SEMA3F |
| 218122_s_at | NM_021627 | −1 | SENP2 |
| 209722_s_at | BC002538 | −1 | SERPINB9 |
| 214197_s_at | AI762193 | −1 | SETDB1 |
| 33323_r_at | X57348 | −1 | SFN |
| 204051_s_at | NM_003014 | −1 | SFRP4 |
| 201742_x_at | M69040 | −1 | SFRS1 |
| 37004_at | J02761 | −1 | SFTPB |
| 201739_at | NM_005627 | −1 | SGK |
| 211211_x_at | AF100542 | −1 | SH2D1A |
| 210796_x_at | D86359 | −1 | SIGLEC6 |
| 207250_at | NM_007374 | −1 | SIX6 |
| 207095_at | NM_000452 | −1 | SLC10A2 |
| 217473_x_at | AF229163 | −1 | SLC11A1 |
| 203125_x_at | AF046997 | −1 | SLC11A2 |
| 202236_s_at | NM_003051 | −1 | SLC16A1 |
| 204230_s_at | NM_020309 | −1 | SLC17A7 |
| 208389_s_at | NM_004171 | −1 | SLC1A2 |
| 207408_at | NM_004803 | −1 | SLC22A14 |
| 219344_at | NM_018344 | −1 | SLC29A3 |
| 216236_s_at | AL110298 | −1 | SLC2A3 |
| 219991_at | NM_020041 | −1 | SLC2A9 |
| 210739_x_at | AF069510 | −1 | SLC4A4 |
| 211123_at | D87920 | −1 | SLC5A5 |
| 205920_at | NM_003043 | −1 | SLC6A6 |
| 216092_s_at | AL365347 | −1 | SLC7A8 |
| 207499_x_at | NM_017979 | −1 | SMAP-1 |
| 214708_at | BG484314 | −1 | SNTB1 |
| 215366_at | AL353943 | −1 | SNX13 |
| 205482_x_at | NM_013306 | −1 | SNX15 |
| 206663_at | NM_003112 | −1 | SP4 |
| 205406_s_at | NM_017425 | −1 | SPA17 |
| 206239_s_at | NM_003122 | −1 | SPINK1 |
| 216981_x_at | X60502 | −1 | SPN |
| 219677_at | NM_025106 | −1 | SSB1 |
| 203019_x_at | NM_014021 | −1 | SSX2IP |
| 214441_at | NM_005819 | −1 | STX6 |
| 204287_at | NM_004711 | −1 | SYNGR1 |
| 203999_at | AV731490 | −1 | SYT1 |
| 212679_at | AK026529 | −1 | TBL2 |
| 206838_at | NM_005149 | −1 | TBX19 |
| 211590_x_at | U11271 | −1 | TBXA2R |
| 205513_at | NM_001062 | −1 | TCN1 |
| 221473_x_at | U49188 | −1 | TDE1 |
| 215902_at | AF009267 | −1 | TEB4 |
| 204653_at | BF343007 | −1 | TFAP2A |
| 205016_at | NM_003236 | −1 | TGFA |
| 203834_s_at | NM_006464 | −1 | TGOLN2 |
| 204064_at | NM_005131 | −1 | THOC1 |
| 203167_at | NM_003255 | −1 | TIMP2 |
| 210176_at | AL050262 | −1 | TLR1 |
| 206271_at | NM_003265 | −1 | TLR3 |
| 214657_s_at | AU134977 | −1 | TncRNA |
| 207113_s_at | NM_000594 | −1 | TNF |
| 206025_s_at | AW188198 | −1 | TNFAIP6 |
| 209294_x_at | BC001281 | −1 | TNFRSF10B |
| 210405_x_at | AF153687 | −1 | TNFRSF10B |
| 206729_at | NM_001243 | −1 | TNFRSF8 |
| 211333_s_at | AF288573 | −1 | TNFSF6 |
| 206990_at | NM_003285 | −1 | TNR |
| 202807_s_at | NM_005488 | −1 | TOM1 |
| 204946_s_at | NM_004618 | −1 | TOP3A |
| 215781_s_at | D87012 | −1 | TOP3B |
| 211943_x_at | AL565449 | −1 | TPT1 |
| 36742_at | U34249 | −1 | TRIM15 |
| 206911_at | NM_005082 | −1 | TRIM25 |
| 208178_x_at | NM_007118 | −1 | TRIO |
| 208349_at | NM_007332 | −1 | TRPA1 |
| 214205_x_at | AK022131 | −1 | TXNL2 |
| 210803_at | AF201385 | −1 | TXNRD2 |
| 215511_at | U19345 | −1 | U19345 |
| 209947_at | BC003170 | −1 | UBAP2L |
| 220422_at | NM_017481 | −1 | UBQLN3 |
| 221323_at | NM_025218 | −1 | ULBP1 |
| 205536_at | NM_003371 | −1 | VAV2 |
| 210513_s_at | AF091352 | −1 | VEGF |
| 209822_s_at | L22431 | −1 | VLDLR |
| 204787_at | NM_007268 | −1 | VSIG4 |
| 38964_r_at | U12707 | −1 | WAS |
| 221927_s_at | AI923458 | −1 | WBSCR21 |
| 71933_at | AI218134 | −1 | WNT6 |
| 210200_at | BC000108 | −1 | WWP2 |
| 206365_at | NM_002995 | −1 | XCL1 |
| 216809_at | Z22780 | −1 | Z22780 |
| 201531_at | NM_003407 | −1 | ZFP36 |
| 212892_at | AW130128 | −1 | ZNF282 |
| 216692_at | AL137428 | −1 | ZNF337 |
| 219089_s_at | NM_024327 | −1 | ZNF576 |
| 215376_at | AU147830 | 1 | MAMMA1001818 |
| 212829_at | BE878277 | 1 | OVARC1000964 |
| 213686_at | AI186145 | 1 | AI186145 |
| 215287_at | AA975427 | 1 | ELISC-1 |
| 213750_at | AA928506 | 1 | YH77E09 |
| 217529_at | BE547674 | 1 | LOC402578 |
| 215343_at | AF070587 | 1 | 0610010D24Rik |
| 212114_at | BE967207 | 1 | microtubule |
| | | | proteins |
| 212413_at | D50918 | 1 | 6-Sep |
| 212414_s_at | D50918 | 1 | 6-Sep |
| 213970_at | AA744682 | 1 | AA744682 |
| 209307_at | AB014540 | 1 | AB014540 |
| 207819_s_at | NM_000443 | 1 | ABCB4 |
| 209620_s_at | AB005289 | 1 | ABCB7 |
| 214274_s_at | AI860341 | 1 | ACAA1 |
| 205355_at | NM_001609 | 1 | ACADSB |
| 219962_at | NM_021804 | 1 | ACE2 |
| 201715_s_at | NM_014977 | 1 | ACIN1 |
| 215227_x_at | BG035989 | 1 | ACP1 |
| 202135_s_at | NM_005735 | 1 | ACTR1B |
| 205260_s_at | NM_001107 | 1 | ACYP1 |
| 206833_s_at | NM_001108 | 1 | ACYP2 |
| 208268_at | NM_021777 | 1 | ADAM28 |
| 209321_s_at | AF033861 | 1 | ADCY3 |
| 208030_s_at | NM_001119 | 1 | ADD1 |
| 201773_at | NM_015339 | 1 | ADNP |
| 201281_at | NM_007002 | 1 | ADRM1 |
| 202144_s_at | NM_000026 | 1 | ADSL |
| 217729_s_at | NM_001130 | 1 | AES |
| 216609_at | AF065241 | 1 | AF065241 |
| 211071_s_at | BC006471 | 1 | AF1Q |
| 210111_s_at | AF277175 | 1 | AF277175 |
| 204333_s_at | NM_000027 | 1 | AGA |
| 203566_s_at | NM_000645 | 1 | AGL |
| 200850_s_at | NM_006621 | 1 | AHCYL1 |
| 202820_at | NM_001621 | 1 | AHR |
| 212980_at | AL050376 | 1 | AHSA2 |
| 204057_at | AI073984 | 1 | AI073984 |
| 212543_at | U83115 | 1 | AIM1 |
| 214102_at | AK023737 | 1 | AK023737 |
| 201675_at | NM_003488 | 1 | AKAP1 |
| 218580_x_at | NM_017900 | 1 | AKIP |
| 202139_at | NM_003689 | 1 | AKR7A2 |
| 215307_at | AL109722 | 1 | AL109722 |
| 216421_at | AL121886 | 1 | AL121886 |
| 216490_x_at | AL133267 | 1 | AL133267 |
| 201612_at | NM_000696 | 1 | ALDH9A1 |
| 218203_at | NM_013338 | 1 | ALG5 |
| 203545_at | NM_024079 | 1 | ALG8 |
| 214220_s_at | AW003635 | 1 | ALMS1 |
| 204976_s_at | AK023637 | 1 | AMMECR1 |
| 208722_s_at | BC001081 | 1 | ANAPC5 |
| 218769_s_at | NM_023039 | 1 | ANKRA2 |
| 218093_s_at | NM_017664 | 1 | ANKRD10 |
| 212211_at | AI986295 | 1 | ANKRD17 |
| 202442_at | NM_001284 | 1 | AP3S1 |
| 210278_s_at | AF155159 | 1 | AP4S1 |
| 213115_at | AL031177 | 1 | APG4A |
| 202512_s_at | AK001899 | 1 | APG5L |
| 202630_at | AA046411 | 1 | APPBP2 |
| 213892_s_at | AA927724 | 1 | APRT |
| 201526_at | NM_001662 | 1 | ARF5 |
| 218870_at | NM_018460 | 1 | ARHGAP15 |
| 213039_at | AB011093 | 1 | ARHGEF18 |
| 218964_at | NM_006465 | 1 | ARID3B |
| 221230_s_at | NM_016374 | 1 | ARID4B |
| 201229_s_at | BC000422 | 1 | ARIH2 |
| 201230_s_at | NM_006321 | 1 | ARIH2 |
| 218868_at | NM_020445 | 1 | ARP3BETA |
| 215457_at | AF070647 | 1 | ARPC1A |
| 207988_s_at | NM_005731 | 1 | ARPC2 |
| 213513_x_at | BG034239 | 1 | ARPC2 |
| 215684_s_at | AL096741 | 1 | ASCC2 |
| 212672_at | U82828 | 1 | ATM |
| 211755_s_at | BC005960 | 1 | ATP5F1 |
| 207508_at | NM_001689 | 1 | ATP5G3 |
| 209492_x_at | BC003679 | 1 | ATP5I |
| 202325_s_at | NM_001685 | 1 | ATP5J |
| 200818_at | NM_001697 | 1 | ATP5O |
| 214934_at | AW411030 | 1 | ATP9B |
| 209903_s_at | U49844 | 1 | ATR |
| 208833_s_at | AF119662 | 1 | ATXN10 |
| 214691_x_at | AU121431 | 1 | AU121431 |
| 222297_x_at | AV738806 | 1 | AV738806 |
| 221589_s_at | AW612403 | 1 | AW612403 |
| 202387_at | NM_004323 | 1 | BAG1 |
| 219667_s_at | NM_017935 | 1 | BANK1 |
| 202326_at | NM_006709 | 1 | BAT8 |
| 211833_s_at | U19599 | 1 | BAX |
| 200837_at | NM_005745 | 1 | BCAP31 |
| 210347_s_at | AF080216 | 1 | BCL11A |
| 219497_s_at | NM_018014 | 1 | BCL11A |
| 202315_s_at | NM_004327 | 1 | BCR |
| 213282_at | BE501952 | 1 | BE501952 |
| 213637_at | BE503392 | 1 | BE503392 |
| 215440_s_at | AL523320 | 1 | BEXL1 |
| 222026_at | BF437591 | 1 | BF437591 |
| 221847_at | BF665706 | 1 | BF665706 |
| 206255_at | NM_001715 | 1 | BLK |
| 207655_s_at | NM_013314 | 1 | BLNK |
| 201849_at | NM_004052 | 1 | BNIP3 |
| 212563_at | BG491842 | 1 | BOP1 |
| 203502_at | NM_001724 | 1 | BPGM |
| 213473_at | AL042733 | 1 | BRAP |
| 204531_s_at | NM_007295 | 1 | BRCA1 |
| 220059_at | NM_012108 | 1 | BRDG1 |
| 219177_at | NM_018321 | 1 | BRIX |
| 201458_s_at | NM_004725 | 1 | BUB3 |
| 209974_s_at | AF047473 | 1 | BUB3 |
| 202808_at | AK000161 | 1 | C10orf26 |
| 55662_at | H27225 | 1 | C10orf76 |
| 219067_s_at | NM_017615 | 1 | C10orf86 |
| 218213_s_at | NM_014206 | 1 | C11orf10 |
| 52164_at | AA065185 | 1 | C11orf24 |
| 218220_at | NM_021640 | 1 | C12orf10 |
| 44790_s_at | AI129310 | 1 | C13orf18 |
| 218940_at | NM_024558 | 1 | C14orf138 |
| 217768_at | NM_016039 | 1 | C14orf166 |
| 221932_s_at | AA133341 | 1 | C14orf87 |
| 201685_s_at | NM_014828 | 1 | C14orf92 |
| 208837_at | BC000027 | 1 | C15orf22 |
| 219439_at | NM_020156 | 1 | C1GALT1 |
| 212164_at | AL522296 | 1 | C1orf37 |
| 203960_s_at | NM_016126 | 1 | C1orf41 |
| 214214_s_at | AU151801 | 1 | C1QBP |
| 209422_at | AY027523 | 1 | C20orf104 |
| 218448_at | NM_017896 | 1 | C20orf11 |
| 219443_at | NM_017714 | 1 | C20orf13 |
| 217737_x_at | NM_016407 | 1 | C20orf43 |
| 217851_s_at | NM_016045 | 1 | C20orf45 |
| 218859_s_at | NM_016649 | 1 | C20orf6 |
| 202217_at | NM_004649 | 1 | C21orf33 |
| 218123_at | NM_017835 | 1 | C21orf59 |
| 221211_s_at | NM_020152 | 1 | C21orf7 |
| 218037_at | NM_024293 | 1 | C2orf17 |
| 221984_s_at | AL040896 | 1 | C2orf17 |
| 219137_s_at | NM_020194 | 1 | C2orf33 |
| 218518_at | NM_016603 | 1 | C5orf5 |
| 212176_at | AA902326 | 1 | C6orf111 |
| 208809_s_at | AL136632 | 1 | C6orf62 |
| 203259_s_at | BC001671 | 1 | C6orf74 |
| 41047_at | AI885170 | 1 | C9orf16 |
| 218992_at | NM_018465 | 1 | C9orf46 |
| 211984_at | AI653730 | 1 | CALM1 |
| 200622_x_at | AV685208 | 1 | CALM3 |
| 203538_at | NM_001745 | 1 | CAMLG |
| 218929_at | NM_017632 | 1 | CARF |
| 202402_s_at | NM_001751 | 1 | CARS |
| 213373_s_at | BF439983 | 1 | CASP8 |
| 200037_s_at | NM_016587 | 1 | CBX3 |
| 219174_at | NM_025103 | 1 | CCDC2 |
| 219470_x_at | NM_019084 | 1 | CCNJ |
| 204645_at | NM_001241 | 1 | CCNT2 |
| 200910_at | NM_005998 | 1 | CCT3 |
| 200812_at | NM_006429 | 1 | CCT7 |
| 206398_s_at | NM_001770 | 1 | CD19 |
| 209583_s_at | AF063591 | 1 | CD200 |
| 204581_at | NM_001771 | 1 | CD22 |
| 38521_at | X59350 | 1 | CD22 |
| 208650_s_at | BG327863 | 1 | CD24 |
| 208651_x_at | BG327863 | 1 | CD24 |
| 209771_x_at | AA761181 | 1 | CD24 |
| 216379_x_at | AK000168 | 1 | CD24 |
| 203593_at | NM_012120 | 1 | CD2AP |
| 213856_at | BG230614 | 1 | CD47 |
| 200984_s_at | X16447 | 1 | CD59 |
| 200985_s_at | NM_000611 | 1 | CD59 |
| 215925_s_at | AF283777 | 1 | CD72 |
| 205049_s_at | NM_001783 | 1 | CD79A |
| 205297_s_at | NM_000626 | 1 | CD79B |
| 206761_at | NM_005816 | 1 | CD96 |
| 205288_at | NM_003672 | 1 | CDC14A |
| 203468_at | NM_003674 | 1 | CDK10 |
| 212899_at | AB028951 | 1 | CDK11 |
| 204252_at | M68520 | 1 | CDK2 |
| 34210_at | N90866 | 1 | CDW52 |
| 218592_s_at | NM_017829 | 1 | CECR5 |
| 205642_at | NM_007018 | 1 | CEP1 |
| 209194_at | BC005334 | 1 | CETN2 |
| 215318_at | AL049782 | 1 | CG012 |
| 218628_at | NM_016053 | 1 | CGI-116 |
| 219030_at | NM_016058 | 1 | CGI-121 |
| 218102_at | NM_015954 | 1 | CGI-26 |
| 219590_x_at | NM_015958 | 1 | CGI-30 |
| 203721_s_at | NM_016001 | 1 | CGI-48 |
| 201569_s_at | NM_015380 | 1 | CGI-51 |
| 218642_s_at | NM_024300 | 1 | CHCHD7 |
| 208968_s_at | BC002568 | 1 | CIAPIN1 |
| 200999_s_at | NM_006825 | 1 | CKAP4 |
| 201897_s_at | BC001425 | 1 | CKS1B |
| 212752_at | AA176798 | 1 | CLASP1 |
| 219859_at | NM_014358 | 1 | CLECSF9 |
| 204085_s_at | NM_006493 | 1 | CLN5 |
| 204577_s_at | NM_024793 | 1 | CLUAP1 |
| 218111_s_at | NM_018686 | 1 | CMAS |
| 208912_s_at | BC001362 | 1 | CNP |
| 203642_s_at | NM_014900 | 1 | COBLL1 |
| 203073_at | NM_007357 | 1 | COG2 |
| 213243_at | AI052003 | 1 | COH1 |
| 201652_at | NM_006837 | 1 | COPS5 |
| 218328_at | NM_016035 | 1 | COQ4 |
| 200086_s_at | AA854966 | 1 | COX4I1 |
| 203663_s_at | NM_004255 | 1 | COX5A |
| 211025_x_at | BC006229 | 1 | COX5B |
| 201754_at | NM_004374 | 1 | COX6C |
| 213846_at | AA382702 | 1 | COX7C |
| 218142_s_at | NM_016302 | 1 | CRBN |
| 208670_s_at | AF109873 | 1 | CRI1 |
| 205081_at | NM_001311 | 1 | CRIP1 |
| 204349_at | BC005250 | 1 | CRSP9 |
| 220753_s_at | NM_015974 | 1 | CRYL1 |
| 221139_s_at | NM_015989 | 1 | CSAD |
| 201160_s_at | AL556190 | 1 | CSDA |
| 212073_at | AI631874 | 1 | CSNK2A1 |
| 203575_at | NM_001896 | 1 | CSNK2A2 |
| 213980_s_at | AA053830 | 1 | CTBP1 |
| 221742_at | AI472139 | 1 | CUGBP1 |
| 201372_s_at | AU145232 | 1 | CUL3 |
| 218970_s_at | NM_015960 | 1 | CUTC |
| 214974_x_at | AK026546 | 1 | CXCL5 |
| 215726_s_at | M22976 | 1 | CYB5 |
| 220999_s_at | NM_030778 | 1 | CYFIP2 |
| 221905_at | BF516433 | 1 | CYLD |
| 207786_at | NM_024514 | 1 | CYP2R1 |
| 216060_s_at | AK021890 | 1 | DAAM1 |
| 201624_at | NM_001349 | 1 | DARS |
| 218443_s_at | NM_018959 | 1 | DAZAP1 |
| 202428_x_at | NM_020548 | 1 | DBI |
| 211070_x_at | BC006466 | 1 | DBI |
| 201572_x_at | NM_001921 | 1 | DCTD |
| 208619_at | L40326 | 1 | DDB1 |
| 212690_at | AB018268 | 1 | DDHD2 |
| 208674_x_at | D29643 | 1 | DDOST |
| 203758_s_at | NM_018380 | 1 | DDX28 |
| 221699_s_at | AF334103 | 1 | DDX50 |
| 220482_s_at | NM_012139 | 1 | DELGEF |
| 221509_at | AB014731 | 1 | DENR |
| 206752_s_at | NM_004402 | 1 | DFFB |
| 203816_at | NM_001929 | 1 | DGUOK |
| 209549_s_at | BC001121 | 1 | DGUOK |
| 202481_at | NM_004735 | | DHRS3 |
| 202031_s_at | NM_015610 | 1 | DKFZP434J154 |
| 213701_at | AW299245 | 1 | DKFZp434N2030 |
| 222149_x_at | AL137398 | 1 | DKFZp434P162 |
| 212936_at | AI927701 | 1 | DKFZP564D172 |
| 208091_s_at | NM_030796 | 1 | DKFZP564K0822 |
| 221596_s_at | AL136619 | 1 | DKFZP564O0523 |
| 202537_s_at | NM_014043 | 1 | DKFZP564O123 |
| 210006_at | BC002571 | 1 | DKFZP564O243 |
| 213149_at | AW299740 | 1 | DLAT |
| 205677_s_at | NM_005887 | 1 | DLEU1 |
| 208810_at | AF080569 | 1 | DNAJB6 |
| 212467_at | AB014578 | 1 | DNAJC13 |
| 213088_s_at | BE551340 | 1 | DNAJC9 |
| 213092_x_at | AW241779 | 1 | DNAJC9 |
| 217917_s_at | NM_014183 | 1 | DNCL2A |
| 212538_at | AL576253 | 1 | DOCK9 |
| 221677_s_at | AF232674 | 1 | DONSON |
| 219452_at | NM_022355 | 1 | DPEP2 |
| 219373_at | NM_018973 | 1 | DPM3 |
| 208370_s_at | NM_004414 | 1 | DSCR1 |
| 218576_s_at | NM_007240 | 1 | DUSP12 |
| 208956_x_at | AB049113 | 1 | DUT |
| 209033_s_at | D86550 | 1 | DYRK1A |
| 207232_s_at | NM_014648 | 1 | DZIP3 |
| 221586_s_at | U15642 | 1 | E2F5 |
| 220942_x_at | NM_014367 | 1 | E2IG5 |
| 219787_s_at | NM_018098 | 1 | ECT2 |
| 208883_at | BF515424 | 1 | EDD |
| 209058_at | AB002282 | 1 | EDF1 |
| 209059_s_at | AB002282 | 1 | EDF1 |
| 212410_at | AI346431 | 1 | EFHA1 |
| 218438_s_at | NM_025205 | 1 | EG1 |
| 218696_at | NM_004836 | 1 | EIF2AK3 |
| 218287_s_at | NM_012199 | 1 | EIF2C1 |
| 212716_s_at | AW083133 | 1 | eIF3k |
| 208756_at | U36764 | 1 | EIF3S2 |
| 201437_s_at | NM_001968 | 1 | EIF4E |
| 214919_s_at | R39094 | 1 | EIF4EBP3, |
| | | | MASK-BP3 |
| 210839_s_at | D45421 | 1 | ENPP2 |
| 202596_at | BC000436 | 1 | ENSA |
| 212681_at | AI770004 | 1 | EPB41L3 |
| 202909_at | NM_014805 | 1 | EPM2AIP1 |
| 202776_at | NM_014597 | 1 | ERBP |
| 200043_at | NM_004450 | 1 | ERH |
| 218100_s_at | NM_018010 | 1 | ESRRBL1 |
| 202942_at | NM_001985 | 1 | ETFB |
| 205530_at | NM_004453 | 1 | ETFDH |
| 217838_s_at | NM_016337 | 1 | EVL |
| 215136_s_at | AL050353 | 1 | EXOSC8 |
| 32259_at | AB002386 | 1 | EZH1 |
| 202862_at | NM_000137 | 1 | FAH |
| 218504_at | NM_016044 | 1 | FAHD2A |
| 220643_s_at | NM_018147 | 1 | FAIM |
| 209405_s_at | BC002934 | 1 | FAM3A |
| 38043_at | X55448 | 1 | FAM3A |
| 201889_at | NM_014888 | 1 | FAM3C |
| 204283_at | NM_006567 | 1 | FARS1 |
| 211623_s_at | M30448 | 1 | FBL |
| 222119_s_at | AL117620 | 1 | FBXO11 |
| 212231_at | AB020682 | 1 | FBXO21 |
| 218432_at | NM_012175 | 1 | FBXO3 |
| 206759_at | NM_002002 | 1 | FCER2 |
| 210889_s_at | M31933 | 1 | FCGR2B |
| 203620_s_at | NM_014824 | 1 | FCHSD2 |
| 203115_at | AU152635 | 1 | FECH |
| 201540_at | NM_001449 | 1 | FHL1 |
| 214505_s_at | AF220153 | 1 | FHL1 |
| 221007_s_at | NM_030917 | 1 | FIPIL1 |
| 219117_s_at | NM_016594 | 1 | FKBP11 |
| 219118_at | NM_016594 | 1 | FKBP11 |
| 218003_s_at | M90820 | 1 | FKBP3 |
| 218349_s_at | AA824298 | 1 | FLJ10036 |
| 205510_s_at | NM_017976 | 1 | FLJ10038 |
| 219648_at | NM_018000 | 1 | FLJ10116 |
| 218067_s_at | NM_018011 | 1 | FLJ10154 |
| 218974_at | NM_018013 | 1 | FLJ10159 |
| 218004_at | NM_018045 | 1 | FLJ10276 |
| 217900_at | NM_018060 | 1 | FLJ10326 |
| 221934_s_at | BF941492 | 1 | FLJ10496 |
| 218314_s_at | AA024582 | 1 | FLJ10726 |
| 217884_at | NM_024662 | 1 | FLJ10774 |
| 218125_s_at | NM_018246 | 1 | FLJ10853 |
| 203941_at | NM_018250 | 1 | FLJ10871 |
| 53720_at | AI862559 | 1 | FLJ11286 |
| 213064_at | N64802 | 1 | FLJ11806 |
| 218341_at | NM_024664 | 1 | FLJ11838 |
| 219022_at | NM_022895 | 1 | FLJ12448 |
| 217866_at | NM_024811 | 1 | FLJ12529 |
| 46142_at | AI003763 | 1 | FLJ12681 |
| 218179_s_at | NM_021942 | 1 | FLJ12716 |
| 220199_s_at | NM_022831 | 1 | FLJ12806 |
| 44065_at | AI937468 | 1 | FLJ14827 |
| 219798_s_at | NM_019606 | 1 | FLJ20257 |
| 209672_s_at | AL136892 | 1 | FLJ20323 |
| 206583_at | NM_017776 | 1 | FLJ20344 |
| 221222_s_at | NM_017860 | 1 | FLJ20519 |
| 219751_at | NM_024860 | 1 | FLJ21148 |
| 218531_at | NM_025124 | 1 | FLJ21749 |
| 218262_at | NM_022762 | 1 | FLJ22318 |
| 219176_at | NM_024520 | 1 | FLJ22555 |
| 219880_at | NM_022907 | 1 | FLJ23053 |
| 220169_at | NM_024943 | 1 | FLJ23235 |
| 64432_at | W05463 | 1 | FLJ39616 |
| 218053_at | NM_017892 | 1 | FNBP3 |
| 202304_at | NM_014923 | 1 | FNDC3 |
| 203064_s_at | NM_004514 | 1 | FOXK2 |
| 209702_at | U79260 | 1 | FTO |
| 205324_s_at | NM_012280 | 1 | FTSJ1 |
| 218356_at | NM_013393 | 1 | FTSJ2 |
| 212847_at | AL036840 | 1 | FUBP1 |
| 214093_s_at | AA156865 | 1 | FUBP1 |
| 202231_at | NM_006360 | 1 | GA17 |
| 204618_s_at | NM_005254 | 1 | GABPB2 |
| 219815_at | NM_024637 | 1 | GAL3ST4 |
| 213049_at | BG436400 | 1 | GARNL1 |
| 214855_s_at | AL050050 | 1 | GARNL1 |
| 209729_at | BC001782 | 1 | GAS2L1 |
| 201738_at | NM_005875 | 1 | GC20 |
| 212139_at | D86973 | 1 | GCN1L1 |
| 208913_at | AA868560 | 1 | GGA2 |
| 200681_at | NM_006708 | 1 | GLO1 |
| 209883_at | AF288389 | 1 | GLT25D2 |
| 218913_s_at | NM_016573 | 1 | GMIP |
| 214157_at | AA401492 | 1 | GNAS |
| 202382_s_at | NM_005471 | 1 | GNPDA1 |
| 202106_at | NM_005895 | 1 | GOLGA3 |
| 201056_at | N53479 | 1 | GOLGB1 |
| 208843_s_at | BC001408 | 1 | GORASP2 |
| 209470_s_at | D49958 | 1 | GPM6A |
| 204793_at | NM_014710 | 1 | GPRASP1 |
| 201106_at | NM_002085 | 1 | GPX4 |
| 204396_s_at | NM_005308 | 1 | GRK5 |
| 201520_s_at | BF034561 | 1 | GRSF1 |
| 215438_x_at | BE906054 | 1 | GSPT1 |
| 201912_s_at | NM_002094 | 1 | GSPT1 |
| 202680_at | NM_002095 | 1 | GTF2E2 |
| 213357_at | AV701318 | 1 | GTF2H5 |
| 201338_x_at | NM_002097 | 1 | GTF3A |
| 215091_s_at | BE542815 | 1 | GTF3A |
| 218343_s_at | NM_012086 | 1 | GTF3C3 |
| 200075_s_at | BC006249 | 1 | GUK1 |
| 220936_s_at | NM_018267 | 1 | H2AFJ |
| 200853_at | NM_002106 | 1 | H2AFZ |
| 200080_s_at | AI955655 | 1 | H3F3A |
| 208630_at | AI972144 | 1 | HADHA |
| 201036_s_at | NM_005327 | 1 | HADHSC |
| 211569_s_at | AF001903 | 1 | HADHSC |
| 202042_at | NM_002109 | 1 | HARS |
| 203138_at | NM_003642 | 1 | HAT1 |
| 204018_x_at | NM_000558 | 1 | HBA1, HBA2 |
| 214414_x_at | T50399 | 1 | HBA2 |
| 209116_x_at | M25079 | 1 | HBB |
| 211696_x_at | AF349114 | 1 | HBB |
| 209274_s_at | BC002675 | 1 | HBLD2 |
| 202299_s_at | NM_006402 | 1 | HBXIP |
| 202300_at | NM_006402 | 1 | HBXIP |
| 215985_at | X92110 | 1 | HCG8 |
| 202957_at | NM_005335 | 1 | HCLS1 |
| 218620_s_at | NM_016173 | 1 | HEMK1 |
| 200093_s_at | N32864 | 1 | HINT1 |
| 218946_at | NM_015700 | 1 | HIRIP5 |
| 205671_s_at | NM_002120 | 1 | HLA-DOB |
| 203290_at | NM_002122 | 1 | HLA-DQA1 |
| 200679_x_at | NM_002128 | 1 | HMGB1 |
| 208808_s_at | BC000903 | 1 | HMGB2 |
| 200943_at | NM_004965 | 1 | HMGN1 |
| 200944_s_at | NM_004965 | 1 | HMGN1 |
| 209787_s_at | BC001282 | 1 | HMGN4 |
| 209068_at | D89678 | 1 | HNRPDL |
| 208990_s_at | AI912352 | 1 | HNRPH3 |
| 214918_at | AK024911 | 1 | HNRPM |
| 208766_s_at | BC001449 | 1 | HNRPR |
| 208713_at | BF724216 | 1 | HNRPUL1 |
| 203202_at | AI950314 | 1 | HRB2 |
| 203203_s_at | NM_007043 | 1 | HRB2 |
| 202098_s_at | NM_001535 | 1 | HRMT1L1 |
| 221564_at | AL570294 | 1 | HRMT1L1 |
| 206445_s_at | NM_001536 | 1 | HRMT1L2 |
| 209657_s_at | M65217 | 1 | HSF2 |
| 220839_at | NM_014168 | 1 | HSPC133 |
| 221570_s_at | AF201938 | 1 | HSPC133 |
| 217774_s_at | NM_016404 | 1 | HSPC152 |
| 218728_s_at | AK024569 | 1 | HSPC163 |
| 210211_s_at | AF028832 | 1 | HSPCA |
| 205133_s_at | NM_002157 | 1 | HSPE1 |
| 202602_s_at | NM_014500 | 1 | HTATSF1 |
| 212493_s_at | AI761110 | 1 | HYPB |
| 204744_s_at | NM_013417 | 1 | IARS |
| 210970_s_at | AF235049 | 1 | IBTK |
| 204868_at | NM_001545 | 1 | ICT1 |
| 202070_s_at | NM_005530 | 1 | IDH3A |
| 222285_at | AW134608 | 1 | IGHD |
| 209374_s_at | BC001872 | 1 | IGHM |
| 212827_at | X17115 | 1 | IGHM |
| 209341_s_at | AU153366 | 1 | IKBKB |
| 204116_at | NM_000206 | 1 | IL2RG |
| 221548_s_at | AY024365 | 1 | ILKAP |
| 212411_at | BE747342 | 1 | IMP4 |
| 205981_s_at | NM_001564 | 1 | ING1L |
| 48825_at | AA887083 | 1 | ING4 |
| 204552_at | AA355179 | 1 | INPP4A |
| 204202_at | NM_017604 | 1 | IQCE |
| 217908_s_at | NM_018442 | 1 | IQWD1 |
| 209075_s_at | AY009128 | 1 | ISCU |
| 205055_at | NM_002208 | 1 | ITGAE |
| 216178_x_at | AA215854 | 1 | ITGB1 |
| 202660_at | AA834576 | 1 | ITPR2 |
| 215791_at | AF003738 | 1 | ITSN1 |
| 212287_at | BF382924 | 1 | JJAZ1 |
| 210878_s_at | BC001202 | 1 | JMJD1B |
| 214861_at | AI341811 | 1 | JMJD2C |
| 200048_s_at | NM_006694 | 1 | JTB |
| 218570_at | NM_018095 | 1 | KBTBD4 |
| 45653_at | AW026481 | 1 | KCTD13 |
| 218823_s_at | NM_017634 | 1 | KCTD9 |
| 212733_at | AI798908 | 1 | KIAA0226 |
| 212735_at | AI798908 | 1 | KIAA0226 |
| 212474_at | D87682 | 1 | KIAA0241 |
| 212450_at | D87445 | 1 | KIAA0256 |
| 209256_s_at | AF277177 | 1 | KIAA0265 |
| 212855_at | D87466 | 1 | KIAA0276 |
| 212621_at | AB006624 | 1 | KIAA0286 |
| 214356_s_at | AI272899 | 1 | KIAA0368 |
| 212480_at | AB002374 | 1 | KIAA0376 |
| 202713_s_at | AA129755 | 1 | KIAA0391 |
| 212068_s_at | AB011087 | 1 | KIAA0515 |
| 212485_at | AU146596 | 1 | KIAA0553 |
| 212675_s_at | AB011154 | 1 | KIAA0582 |
| 34406_at | AB011174 | 1 | KIAA0602 |
| 201965_s_at | NM_015046 | 1 | KIAA0625 |
| 212200_at | AW274877 | 1 | KIAA0692 |
| 212311_at | AA522514 | 1 | KIAA0746 |
| 209553_at | BC001001 | 1 | KIAA0804 |
| 212546_s_at | AI126634 | 1 | KIAA0826 |
| 204568_at | NM_014924 | 1 | KIAA0831 |
| 205594_at | NM_014897 | 1 | KIAA0924 |
| 209654_at | BC004902 | 1 | KIAA0947 |
| 203831_at | NM_014925 | 1 | KIAA1002 |
| 200861_at | NM_016284 | 1 | KIAA1007 |
| 55872_at | AI493119 | 1 | KIAA1196 |
| 221736_at | BG236163 | 1 | KIAA1219 |
| 219520_s_at | NM_018458 | 1 | KIAA1280 |
| 207314_x_at | NM_006737 | 1 | KIR3DL2 |
| 217906_at | NM_014315 | 1 | KLHDC2 |
| 208975_s_at | BC003572 | 1 | KPNB1 |
| 204009_s_at | NM_004985 | 1 | KRAS2 |
| 200914_x_at | NM_004986 | 1 | KTN1 |
| 200915_x_at | NM_004986 | 1 | KTN1, |
| | | | TXNDC7 |
| 214709_s_at | Z22551 | 1 | KTN1, |
| | | | TXNDC7 |
| 200771_at | NM_002293 | 1 | LAMC1 |
| 212137_at | AV746402 | 1 | LARP |
| 34764_at | D21851 | 1 | LARS2 |
| 212446_s_at | AI658534 | 1 | LASS6 |
| 221515_s_at | BC001214 | 1 | LCMT1 |
| 202726_at | NM_002314 | 1 | LIG1 |
| 204357_s_at | NM_002314 | 1 | LIMK1 |
| 202386_s_at | NM_019081 | 1 | LKAP |
| 213527_s_at | AC002310 | 1 | LOC146542 |
| 213703_at | W95043 | 1 | LOC150759 |
| 221973_at | AI983904 | 1 | LOC150759 |
| 214801_at | W88821 | 1 | LOC163590 |
| 211325_x_at | U72518 | 1 | LOC171220 |
| 212866_at | AI081543 | 1 | LOC203069 |
| 213725_x_at | AI693140 | 1 | LOC283824 |
| 219043_s_at | NM_024065 | 1 | LOC285359, |
| | | | PDCL3 |
| 221797_at | AY007126 | 1 | LOC339229 |
| 50374_at | AA150503 | 1 | LOC339229 |
| 222000_at | AI915947 | 1 | LOC339448 |
| 218303_x_at | NM_016618 | 1 | LOC51315 |
| 218616_at | NM_020395 | 1 | LOC57117 |
| 203897_at | BE963444 | 1 | LOC57149 |
| 213346_at | BE748563 | 1 | LOC93081 |
| 218096_at | NM_018361 | 1 | LPAAT-e |
| 216250_s_at | X77598 | 1 | LPXN |
| 221640_s_at | AF274972 | 1 | LRDD |
| 211615_s_at | M92439 | 1 | LRPPRC |
| 209449_at | AF196468 | 1 | LSM2 |
| 202737_s_at | AA112507 | 1 | LSM4 |
| 211747_s_at | BC005938 | 1 | LSM5 |
| 204559_s_at | NM_016199 | 1 | LSM7 |
| 212248_at | AI886796 | 1 | LYRIC |
| 217536_x_at | M78162 | 1 | M78162 |
| 209014_at | AF217963 | 1 | MAGED1 |
| 220925_at | NM_021929 | 1 | MAK10 |
| 214703_s_at | AW954107 | 1 | MAN2B2 |
| 202670_at | AI571419 | 1 | MAP2K1 |
| 206854_s_at | NM_003188 | 1 | MAP3K7 |
| 202569_s_at | NM_002376 | 1 | MARK3 |
| 213671_s_at | AA621558 | 1 | MARS |
| 212064_x_at | AI471665 | 1 | MAZ |
| 209580_s_at | AL556619 | 1 | MBD4 |
| 201620_at | NM_003791 | 1 | MBTPS1 |
| 218440_at | NM_020166 | 1 | MCCC1 |
| 200978_at | NM_005917 | 1 | MDH1 |
| 209036_s_at | BC001917 | 1 | MDH2 |
| 212693_at | BE670928 | 1 | MDN1 |
| 218288_s_at | NM_021825 | 1 | MDS025 |
| 221706_s_at | BC006005 | 1 | MDS032 |
| 218061_at | NM_014623 | 1 | MEA |
| 202645_s_at | NM_000244 | 1 | MEN1 |
| 207098_s_at | NM_017927 | 1 | MFN1 |
| 204153_s_at | AI738965 | 1 | MFNG |
| 220189_s_at | NM_014275 | 1 | MGAT4B |
| 219001_s_at | NM_024345 | 1 | MGC10765 |
| 213104_at | AI799802 | 1 | MGC24381 |
| 218903_s_at | NM_024068 | 1 | MGC2731 |
| 200076_s_at | BC006479 | 1 | MGC2749 |
| 218714_at | NM_024031 | 1 | MGC3121 |
| 209191_at | BC002654 | 1 | MGC4083 |
| 202365_at | BC004815 | 1 | MGC5139 |
| 221580_s_at | BC001972 | 1 | MGC5306 |
| 200847_s_at | NM_016127 | 1 | MGC8721 |
| 200899_s_at | AK002091 | 1 | MGEA5 |
| 217871_s_at | BC000447 | 1 | MIF |
| 214246_x_at | AI859060 | 1 | MINK |
| 221824_s_at | AA770170 | 1 | MIR |
| 209845_at | AF117233 | 1 | MKRN1 |
| 204173_at | NM_002475 | 1 | MLC1SA |
| 202520_s_at | NM_000249 | 1 | MLH1 |
| 204206_at | NM_020310 | 1 | MNT |
| 212508_at | AK024029 | 1 | MOAP1 |
| 201994_at | NM_012286 | 1 | MORF4L2 |
| 215731_s_at | X98258 | 1 | MPHOSPH9 |
| 219162_s_at | NM_016050 | 1 | MRPL11 |
| 220526_s_at | NM_017971 | 1 | MRPL20 |
| 218339_at | NM_014180 | 1 | MRPL22 |
| 213897_s_at | AI832239 | 1 | MRPL23 |
| 209609_s_at | BC004517 | 1 | MRPL9 |
| 203800_s_at | BG254653 | 1 | MRPS14 |
| 221437_s_at | NM_031280 | 1 | MRPS15 |
| 212604_at | AI937794 | 1 | MRPS31 |
| 218654_s_at | NM_016071 | 1 | MRPS33 |
| 217942_at | NM_021821 | 1 | MRPS35 |
| 211783_s_at | BC006177 | 1 | MTA1 |
| 210212_x_at | BC002600 | 1 | MTCP1 |
| 216862_s_at | Z24459 | 1 | MTCP1 |
| 202309_at | NM_005956 | 1 | MTHFD1 |
| 216095_x_at | AF057354 | 1 | MTMR1 |
| 219822_at | NM_004294 | 1 | MTRF1 |
| 203199_s_at | N29717 | 1 | MTRR |
| 205145_s_at | NM_002477 | 1 | MYL5 |
| 222018_at | AI992187 | 1 | NACA |
| 204528_s_at | NM_004537 | 1 | NAP1L1 |
| 208753_s_at | BC002387 | 1 | NAP1L1 |
| 204749_at | NM_004538 | 1 | NAP1L3 |
| 218713_at | NM_024611 | 1 | NARG2 |
| 201517_at | BC001255 | 1 | NCBP2 |
| 218697_at | NM_016453 | 1 | NCKIPSD |
| 200610_s_at | NM_005381 | 1 | NCL |
| 217286_s_at | BC001805 | 1 | NDRG3 |
| 202298_at | NM_004541 | 1 | NDUFA1 |
| 209224_s_at | BC003674 | 1 | NDUFA2 |
| 217773_s_at | NM_002489 | 1 | NDUFA4 |
| 202785_at | NM_005001 | 1 | NDUFA7 |
| 208969_at | AF050641 | 1 | NDUFA9 |
| 202077_at | NM_005003 | 1 | NDUFAB1 |
| 218320_s_at | NM_019056 | 1 | NDUFB11 |
| 202839_s_at | NM_004146 | 1 | NDUFB7 |
| 203478_at | NM_002494 | 1 | NDUFC1 |
| 218101_s_at | NM_004549 | 1 | NDUFC2 |
| 201740_at | NM_004551 | 1 | NDUFS3 |
| 203189_s_at | NM_002496 | 1 | NDUFS8 |
| 203190_at | NM_002496 | 1 | NDUFS8 |
| 213331_s_at | AV700007 | 1 | NEK1 |
| 203413_at | NM_006159 | 1 | NELL2 |
| 218129_s_at | BC005316 | 1 | NFYB |
| 202475_at | NM_006326 | 1 | NIFIE14 |
| 212483_at | AB019494 | 1 | NIPBL |
| 203830_at | NM_022344 | 1 | NJMU-R1 |
| 202294_at | NM_005862 | 1 | NM_005862 |
| 217946_s_at | NM_016402 | 1 | NM_016402 |
| 219817_at | NM_016534 | 1 | NM_016534 |
| 218506_x_at | NM_018459 | 1 | NM_018459 |
| 218517_at | NM_024900 | 1 | NM_024900 |
| 212739_s_at | AL523860 | 1 | NME4 |
| 200875_s_at | NM_006392 | 1 | NOL5A |
| 202882_x_at | NM_016167 | 1 | NOL7 |
| 204791_at | NM_003297 | 1 | NR2C1 |
| 204651_at | AW003022 | 1 | NRF1 |
| 213061_s_at | AA643304 | 1 | NTAN1 |
| 213062_at | AA643304 | 1 | NTAN1 |
| 212605_s_at | AK025759 | 1 | NUDT3 |
| 212182_at | AB007956 | 1 | NUDT4 |
| 202900_s_at | NM_002532 | 1 | NUP88 |
| 213018_at | AI337901 | 1 | ODAG |
| 203569_s_at | NM_003611 | 1 | OFD1 |
| 219073_s_at | NM_017784 | 1 | OSBPL10 |
| 208717_at | BC001669 | 1 | OXA1L |
| 206637_at | NM_014879 | 1 | P2RY14 |
| 218131_s_at | AK024670 | 1 | p66alpha |
| 201545_s_at | NM_004643 | 1 | PABPN1 |
| 200816_s_at | NM_000430 | 1 | PAFAH1B1 |
| 208051_s_at | NM_006451 | 1 | PAIP1 |
| 209064_x_at | BF248165 | 1 | PAIP1 |
| 210466_s_at | BC002488 | 1 | PAI-RBP1 |
| 202759_s_at | BE879367 | 1 | PALM2-AKAP2 |
| 204715_at | NM_015368 | 1 | PANX1 |
| 212718_at | BF797555 | 1 | PAPOLA |
| 200006_at | NM_007262 | 1 | PARK7 |
| 203905_at | NM_002582 | 1 | PARN |
| 219033_at | NM_024615 | 1 | PARP8 |
| 213534_s_at | D50925 | 1 | PASK |
| 204004_at | AI336206 | 1 | PAWR |
| 205353_s_at | NM_002567 | 1 | PBP |
| 210825_s_at | AF130103 | 1 | PBP |
| 211941_s_at | BE969671 | 1 | PBP |
| 214177_s_at | AI935162 | 1 | PBXIP1 |
| 212694_s_at | NM_000532 | 1 | PCCB |
| 219737_s_at | NM_020403 | 1 | PCDH9 |
| 203378_at | AB020631 | 1 | PCF11 |
| 218260_at | NM_024050 | 1 | PCIA1 |
| 202174_s_at | NM_006197 | 1 | PCM1 |
| 218014_at | NM_024844 | 1 | PCNT1 |
| 205559_s_at | NM_006200 | 1 | PCSK5 |
| 212422_at | AL547263 | 1 | PDCD11 |
| 204025_s_at | NM_002598 | 1 | PDCD2 |
| 213581_at | BF446180 | 1 | PDCD2 |
| 203415_at | NM_013232 | 1 | PDCD6 |
| 204491_at | R40917 | 1 | PDE4D |
| 214129_at | AI821791 | 1 | PDE4DIP |
| 219575_s_at | NM_022341 | 1 | PDF, COG8 |
| 221811_at | BF033007 | 1 | PERLD1 |
| 219180_s_at | AI817074 | 1 | PEX26 |
| 205361_s_at | AI718295 | 1 | PFDN4 |
| 210908_s_at | AB055804 | 1 | PFDN5 |
| 204604_at | NM_012395 | 1 | PFTK1 |
| 222125_s_at | BC000580 | 1 | PH-4 |
| 212542_s_at | BF224151 | 1 | PHIP |
| 202738_s_at | BG149218 | 1 | PHKB |
| 202739_s_at | NM_000293 | 1 | PHKB |
| 203335_at | NM_006214 | 1 | PHYH |
| 209625_at | BC004100 | 1 | PIGH |
| 209998_at | BC001030 | 1 | PIGO |
| 202927_at | NM_006221 | 1 | PIN1 |
| 205632_s_at | NM_003558 | 1 | PIP5K1B |
| 213111_at | AB023198 | 1 | PIP5K3 |
| 218667_at | NM_022368 | 1 | PJA1 |
| 54051_at | H59033 | 1 | PKNOX1 |
| 205372_at | NM_002655 | 1 | PLAG1 |
| 213309_at | AL117515 | 1 | PLCL2 |
| 219024_at | NM_021622 | 1 | PLEKHA1 |
| 201682_at | NM_004279 | 1 | PMPCB |
| 213677_s_at | BG434893 | 1 | PMS1 |
| 218961_s_at | NM_007254 | 1 | PNKP |
| 205901_at | NM_006228 | 1 | PNOC |
| 209740_s_at | U03886 | 1 | PNPLA4 |
| 201115_at | NM_006230 | 1 | POLD2 |
| 217806_s_at | NM_015584 | 1 | POLDIP2 |
| 203366_at | NM_002693 | 1 | POLG |
| 212955_s_at | AL037557 | 1 | POLR2I |
| 218016_s_at | NM_018119 | 1 | POLR3E |
| 203782_s_at | NM_005035 | 1 | POLRMT |
| 213360_s_at | AA514622 | 1 | POM121, |
| | | | LOC340318 |
| 204839_at | NM_015918 | 1 | POP5 |
| 209482_at | BC001430 | 1 | POP7 |
| 205267_at | NM_006235 | 1 | POU2AF1 |
| 205661_s_at | NM_025207 | 1 | PP591 |
| 202494_at | NM_006112 | 1 | PP1E |
| 212750_at | AB020630 | 1 | PPP1R16B |
| 41577_at | AB020630 | 1 | PPP1R16B |
| 202165_at | BF966540 | 1 | PPP1R2 |
| 207830_s_at | NM_002713 | 1 | PPP1R8 |
| 221772_s_at | AI459157 | 1 | PPP2R2D |
| 201877_s_at | NM_002719 | 1 | PPP2R5C |
| 202432_at | NM_021132 | 1 | PPP3CB |
| 202741_at | AA130247 | 1 | PRKACB |
| 207957_s_at | NM_002738 | 1 | PRKCB1 |
| 209685_s_at | M13975 | 1 | PRKCB1 |
| 208694_at | U47077 | 1 | PRKDC |
| 220553_s_at | NM_018333 | 1 | PRPF39 |
| 209161_at | AI184802 | 1 | PRPF4 |
| 202127_at | AB011108 | 1 | PRPF4B |
| 209440_at | BC001605 | 1 | PRPS1 |
| 203401_at | NM_002765 | 1 | PRPS2 |
| 202529_at | NM_002766 | 1 | PRPSAP1 |
| 203089_s_at | NM_013247 | 1 | PRSS25 |
| 209337_at | AF063020 | 1 | PSIP1 |
| 202244_at | NM_002796 | 1 | PSMB4 |
| 209503_s_at | AF035309 | 1 | PSMC5 |
| 212296_at | NM_005805 | 1 | PSMD14 |
| 200830_at | NM_002808 | 1 | PSMD2 |
| 212219_at | D38521 | 1 | PSME4 |
| 218371_s_at | AA969958 | 1 | PSPC1 |
| 219293_s_at | NM_013341 | 1 | PTD004 |
| 201433_s_at | NM_014754 | 1 | PTDSS1 |
| 213795_s_at | AL121905 | 1 | PTPRA |
| 201166_s_at | NM_014676 | 1 | PUM1 |
| 216221_s_at | D87078 | 1 | PUM2 |
| 201608_s_at | NM_007062 | 1 | PWP1 |
| 201568_at | NM_014402 | 1 | QP-C |
| 202754_at | NM_015361 | 1 | R3HDM |
| 217763_s_at | AF183421 | 1 | RAB31 |
| 217764_s_at | AF183421 | 1 | RAB31 |
| 202373_s_at | AF255648 | 1 | RAB3-GAP150 |
| 218699_at | NM_003929 | 1 | RAB7L1 |
| 213313_at | AI922519 | 1 | RABGAP1 |
| 209181_s_at | U49245 | 1 | RABGGTB |
| 219151_s_at | NM_007081 | 1 | RABL2A, |
| | | | RABL2B |
| 207405_s_at | NM_002873 | 1 | RAD17 |
| 204199_at | NM_014636 | 1 | RALGPS1 |
| 220338_at | NM_018037 | 1 | RALGPS2 |
| 221809_at | AB040897 | 1 | RANBP10 |
| 211954_s_at | BC000947 | 1 | RANBP5 |
| 202582_s_at | AF306510 | 1 | RANBP9 |
| 218526_s_at | NM_014185 | 1 | RANGNRF |
| 209444_at | BC001851 | 1 | RAP1GDS1 |
| 205169_at | NM_005057 | 1 | RBBP5 |
| 212781_at | AK026954 | 1 | RBBP6 |
| 201092_at | NM_002893 | 1 | RBBP7 |
| 57540_at | AI823980 | 1 | RBKS |
| 204178_s_at | NM_006328 | 1 | RBM14 |
| 218117_at | NM_014248 | 1 | RBX1 |
| 201486_at | NM_002902 | 1 | RCN2 |
| 203898_at | AU154853 | 1 | RCP9 |
| 222203_s_at | AK023625 | 1 | RDH14 |
| 221532_s_at | AF309553 | 1 | REC14 |
| 210568_s_at | BC001052 | 1 | RECQL |
| 219041_s_at | NM_014374 | 1 | REPIN1 |
| 202296_s_at | NM_007033 | 1 | RER1 |
| 218428_s_at | NM_016316 | 1 | REV1L |
| 208070_s_at | NM_002912 | 1 | REV3L |
| 203659_s_at | NM_005798 | 1 | RFP2 |
| 202976_s_at | NM_014899 | 1 | RHOBTB3 |
| 202130_at | NM_003831 | 1 | RIOK3 |
| 214663_at | AB007941 | 1 | RIPK5 |
| 213397_x_at | AI761728 | 1 | RNASE4 |
| 218496_at | BG534527 | 1 | RNASEH1 |
| 202683_s_at | NM_003799 | 1 | RNMT |
| 200087_s_at | AK024976 | 1 | RNP24 |
| 212430_at | AL109955 | 1 | RNPC1 |
| 218462_at | NM_025065 | 1 | RPF1 |
| 222229_x_at | AL121871 | 1 | RPL26 |
| 218830_at | NM_016093 | 1 | RPL26L1 |
| 221593_s_at | BC001663 | 1 | RPL31 |
| 213687_s_at | BE968801 | 1 | RPL35A |
| 210034_s_at | AA582460 | 1 | RPL5 |
| 211542_x_at | BC004334 | 1 | RPS10 |
| 200095_x_at | AA320764 | 1 | RPS10 |
| 213890_x_at | AI200589 | 1 | RPS16 |
| 200949_x_at | NM_001023 | 1 | RPS20 |
| 200091_s_at | AA888388 | 1 | RPS25 |
| 200741_s_at | NM_001030 | 1 | RPS27 |
| 200017_at | NM_002954 | 1 | RPS27A |
| 218007_s_at | BC003667 | 1 | RPS27L |
| 208904_s_at | BF431363 | 1 | RPS28 |
| 204635_at | NM_004755 | 1 | RPS6KA5 |
| 205540_s_at | NM_016656 | 1 | RRAGB |
| 218088_s_at | AK023373 | 1 | RRAGC |
| 208456_s_at | NM_012250 | 1 | RRAS2 |
| 203704_s_at | AW118862 | 1 | RREB1 |
| 201975_at | NM_002956 | 1 | RSN |
| 212319_at | AB007857 | 1 | RUTBC1 |
| 201459_at | NM_006666 | 1 | RUVBL2 |
| 219598_s_at | NM_016104 | 1 | RWDD1 |
| 205087_at | NM_015485 | 1 | RWDD3 |
| 212438_at | BG252325 | 1 | RY1 |
| 213262_at | AI932370 | 1 | SACS |
| 203280_at | NM_014649 | 1 | SAFB2 |
| 209486_at | BC004546 | 1 | SAS10 |
| 218276_s_at | AI679398 | 1 | SAV1 |
| 213244_at | AI207792 | 1 | SCAMP4 |
| 205790_at | NM_003726 | 1 | SCAP1 |
| 212140_at | AB014548 | 1 | SCC-112 |
| 202541_at | BF589679 | 1 | SCYE1 |
| 218607_s_at | NM_018115 | 1 | SDAD1 |
| 218649_x_at | NM_004713 | 1 | SDCCAG1 |
| 218427_at | NM_006643 | 1 | SDCCAG3 |
| 200945_s_at | NM_014933 | 1 | SEC31L1 |
| 219349_s_at | NM_018303 | 1 | SEC5L1 |
| 201916_s_at | NM_007214 | 1 | SEC63 |
| 212630_at | AF055006 | 1 | SEC6L1 |
| 218265_at | NM_024077 | 1 | SECISBP2 |
| 219351_at | NM_014563 | 1 | SEDL |
| 221931_s_at | AV701173 | 1 | SEH1L |
| 204563_at | NM_000655 | 1 | SELL |
| 201194_at | NM_003009 | 1 | SEPW1 |
| 209723_at | BC002538 | 1 | SERPINB9 |
| 205352_at | NM_005025 | 1 | SERPINI1 |
| 205933_at | NM_015559 | 1 | SETBP1 |
| 216457_s_at | AK026080 | 1 | SF3A1 |
| 203818_s_at | NM_006802 | 1 | SF3A3 |
| 200685_at | AU146237 | 1 | SFRS11 |
| 200686_s_at | NM_004768 | 1 | SFRS11 |
| 214853_s_at | AI091079 | 1 | SHC1 |
| 205063_at | NM_003616 | 1 | SIP1 |
| 213600_at | AA425633 | 1 | SIPA1L3 |
| 219185_at | NM_012241 | 1 | SIRT5 |
| 203489_at | NM_006427 | 1 | SIVA |
| 200718_s_at | AA927664 | 1 | SKP1A |
| 207974_s_at | NM_006930 | 1 | SKP1A |
| 205234_at | NM_004696 | 1 | SLC16A4 |
| 203775_at | NM_014251 | 1 | SLC25A13 |
| 218989_x_at | NM_022902 | 1 | SLC30A5 |
| 213082_s_at | AJ005866 | 1 | SLC35D2 |
| 218928_s_at | NM_018964 | 1 | SLC37A1 |
| 202111_at | NM_003040 | 1 | SLC4A2 |
| 209884_s_at | AF047033 | 1 | SLC4A7 |
| 215043_s_at | X83301 | 1 | SMA3, SMA5 |
| 201784_s_at | NM_014267 | 1 | SMAP |
| 206544_x_at | NM_003070 | 1 | SMARCA2 |
| 218452_at | NM_014140 | 1 | SMARCAL1 |
| 201827_at | AF113019 | 1 | SMARCD2 |
| 218781_at | NM_024624 | 1 | SMC6L1 |
| 205596_s_at | AY014180 | 1 | SMURF2 |
| 202690_s_at | BC001721 | 1 | SNRPD1 |
| 203832_at | NM_003095 | 1 | SNRPF |
| 212777_at | L13857 | 1 | SOS1 |
| 221239_s_at | NM_030764 | 1 | SPAP1 |
| 217927_at | NM_014041 | 1 | SPC12 |
| 212526_at | AK002207 | 1 | SPG20 |
| 205861_at | NM_003121 | 1 | SPIB |
| 211704_s_at | AF356353 | 1 | SPIN2 |
| 212071_s_at | BE968833 | 1 | SPTBN1 |
| 218407_x_at | NM_013349 | 1 | SPUF |
| 209218_at | AF098865 | 1 | SQLE |
| 210959_s_at | AF113128 | 1 | SRD5A1 |
| 201247_at | BE513151 | 1 | SREBF2 |
| 205335_s_at | NM_003135 | 1 | SRP19 |
| 201273_s_at | NM_003133 | 1 | SRP9 |
| 202200_s_at | NM_003137 | 1 | SRPK1 |
| 218140_x_at | NM_021203 | 1 | SRPRB |
| 201138_s_at | NM_003142 | 1 | SSB |
| 201139_s_at | NM_003142 | 1 | SSB |
| 202591_s_at | NM_003143 | 1 | SSBP1 |
| 208666_s_at | BE866412 | 1 | ST13 |
| 208667_s_at | U17714 | 1 | ST13 |
| 209023_s_at | BC001765 | 1 | STAG2 |
| 212549_at | BE645861 | 1 | STAT5B |
| 211505_s_at | AL136601 | 1 | STAU |
| 208855_s_at | AF083420 | 1 | STK24 |
| 217934_x_at | NM_005861 | 1 | STUB1 |
| 203310_at | NM_007269 | 1 | STXBP3 |
| 221213_s_at | NM_017661 | 1 | SUHW4 |
| 202829_s_at | NM_005638 | 1 | SYBL1 |
| 219156_at | NM_018373 | 1 | SYNJ2BP |
| 200055_at | NM_006284 | 1 | TAF10 |
| 206613_s_at | NM_005681 | 1 | TAF1A |
| 221508_at | BC002756 | 1 | TAOK3 |
| 201263_at | NM_003191 | 1 | TARS |
| 209154_at | AF234997 | 1 | TAX1BP3 |
| 218466_at | NM_024682 | 1 | TBC1D17 |
| 203667_at | NM_004607 | 1 | TBCA |
| 203715_at | NM_003193 | 1 | TBCE |
| 213400_s_at | AV753028 | 1 | TBL1X |
| 209820_s_at | BC002361 | 1 | TBL3 |
| 216241_s_at | X57198 | 1 | TCEA1 |
| 202371_at | NM_024863 | 1 | TCEAL4 |
| 209153_s_at | M31523 | 1 | TCF3 |
| 222146_s_at | AK026674 | 1 | TCF4 |
| 222010_at | BF224073 | 1 | TCP1 |
| 222011_s_at | BF224073 | 1 | TCP1 |
| 203054_s_at | NM_022171 | 1 | TCTA |
| 203448_s_at | AI347136 | 1 | TERF1 |
| 212330_at | R60866 | 1 | TFDP1 |
| 218996_at | NM_013342 | 1 | TFPT |
| 214977_at | AK023852 | 1 | TG |
| 212910_at | W19873 | 1 | THAP11 |
| 213043_s_at | AI023317 | 1 | THRAP4 |
| 43544_at | AA314406 | 1 | THRAP5 |
| 218188_s_at | NM_012458 | 1 | TIMM13 |
| 203222_s_at | NM_005077 | 1 | TLE1 |
| 211077_s_at | Z25421 | 1 | TLK1 |
| 212997_s_at | AU151689 | 1 | TLK2 |
| 208942_s_at | U93239 | 1 | TLOC1 |
| 217979_at | NM_014399 | 1 | TM4SF13 |
| 208184_s_at | NM_003274 | 1 | TMEM1 |
| 209412_at | U61500 | 1 | TMEM1 |
| 218477_at | NM_014051 | 1 | TMEM14A |
| 221452_s_at | NM_030969 | 1 | TMEM14B |
| 202857_at | NM_014255 | 1 | TMEM4 |
| 209796_s_at | BC001027 | 1 | TMEM4 |
| 215346_at | BF664114 | 1 | TNFRSF5 |
| 206150_at | NM_001242 | 1 | TNFRSF7 |
| 48531_at | AA522816 | 1 | TNIP2 |
| 202561_at | AF070613 | 1 | TNKS |
| 210886_x_at | AB007457 | 1 | TP53AP1 |
| 220865_s_at | NM_014317 | 1 | TPRT |
| 213334_x_at | BE676218 | 1 | TREX2 |
| 210846_x_at | AF220130 | 1 | TRIM14 |
| 212544_at | AI131008 | 1 | TRIP3 |
| 202642_s_at | NM_003496 | 1 | TRRAP |
| 215735_s_at | AC005600 | 1 | TSC2 |
| 214606_at | BF129969 | 1 | TSPAN-2 |
| 212928_at | AL050331 | 1 | TSPYL4 |
| 217968_at | NM_003310 | 1 | TSSC1 |
| 36936_at | U58766 | 1 | TSTA3 |
| 201434_at | NM_003314 | 1 | TTC1 |
| 217964_at | NM_017775 | 1 | TTC19 |
| 210645_s_at | D83077 | 1 | TTC3 |
| 213174_at | BE675549 | 1 | TTC9 |
| 202266_at | NM_016614 | 1 | TTRAP |
| 209077_at | AL022313 | 1 | TXN2 |
| 201010_s_at | NM_006472 | 1 | TXNIP |
| 37577_at | U79256 | 1 | U79256 |
| 202151_s_at | NM_016172 | 1 | UBADC1 |
| 209115_at | AL117566 | 1 | UBE1C |
| 201343_at | BE621259 | 1 | UBE2D2 |
| 201344_at | BF196642 | 1 | UBE2D2 |
| 210024_s_at | AB017644 | 1 | UBE2E3 |
| 201002_s_at | NM_003349 | 1 | UBE2V1, Kua- |
| | | | UEV |
| 218082_s_at | NM_014517 | 1 | UBP1 |
| 205687_at | NM_019116 | 1 | UBPH |
| 212008_at | N29889 | 1 | UBXD2 |
| 218533_s_at | NM_017859 | 1 | UCKL1 |
| 209103_s_at | BC001049 | 1 | UFD1L |
| 212074_at | BE972774 | 1 | UNC84A |
| 206958_s_at | AF318575 | 1 | UPF3A |
| 206959_s_at | AF318575 | 1 | UPF3A |
| 214323_s_at | N36842 | 1 | UPF3A |
| 202090_s_at | NM_006830 | 1 | UQCR |
| 208909_at | BC000649 | 1 | UQCRFS1 |
| 208970_s_at | M14016 | 1 | UROD |
| 203031_s_at | NM_000375 | 1 | UROS |
| 200083_at | AA621731 | 1 | USP22 |
| 212388_at | AB028980 | 1 | USP24 |
| 220079_s_at | NM_018391 | 1 | USP48 |
| 2031171_s_at | NM_014871 | 1 | USP52 |
| 202745_at | NM_005154 | 1 | USP8 |
| 221514_at | BC001149 | 1 | UTP14A |
| 218495_at | NM_004182 | 1 | UXT |
| 207100_s_at | NM_016830 | 1 | VAMP1 |
| 214792_x_at | AI955119 | 1 | VAMP2 |
| 202550_s_at | AF160212 | 1 | VAPB |
| 212038_s_at | AL515918 | 1 | VDAC1 |
| 203683_s_at | NM_003377 | 1 | VEGFB |
| 208623_s_at | J05021 | 1 | VIL2 |
| 209950_s_at | BC004300 | 1 | VILL |
| 205844_at | NM_004666 | 1 | VNN1 |
| 204376_at | NM_014703 | 1 | VprBP |
| 220068_at | NM_013378 | 1 | VPREB3 |
| 212326_at | AB007922 | 1 | VPS13D |
| 218022_at | NM_016440 | 1 | VRK3 |
| 221998_s_at | BF062886 | 1 | VRK3 |
| 213048_s_at | W26593 | 1 | W26593 |
| 213598_at | W87688 | 1 | W87688 |
| 221725_at | AI962978 | 1 | WASF2 |
| 200609_s_at | NM_017491 | 1 | WDR1 |
| 209216_at | BC000464 | 1 | WDR45 |
| 212533_at | X62048 | 1 | WEE1 |
| 203112_s_at | NM_005663 | 1 | WHSC2 |
| 34225_at | AF101434 | 1 | WHSC2 |
| 205672_at | NM_000380 | 1 | XPA |
| 212160_at | AI984005 | 1 | XPOT |
| 213077_at | AL049305 | 1 | YTHDC2 |
| 219186_at | NM_020224 | 1 | ZBTB7 |
| 217594_at | R25849 | 1 | ZCCHC11 |
| 212655_at | AB011151 | 1 | ZCCHC14 |
| 212860_at | BG168720 | 1 | ZDHHC18 |
| 220261_s_at | NM_018106 | 1 | ZDHHC4 |
| 220206_at | NM_024772 | 1 | ZMYM1 |
| 207605_x_at | NM_024498 | 1 | ZNF117 |
| 37254_at | U09366 | 1 | ZNF133 |
| 209565_at | BC000832 | 1 | ZNF183 |
| 202778_s_at | NM_003453 | 1 | ZNF198 |
| 214823_at | AF033199 | 1 | ZNF204 |
| 219925_at | NM_007167 | 1 | ZNF258 |
| 211009_s_at | AF159567 | 1 | ZNF271 |
| 216399_s_at | AK025663 | 1 | ZNF291 |
| 211678_s_at | AF090934 | 1 | ZNF313 |
| 209538_at | U69645 | 1 | ZNF32 |
| 218079_s_at | NM_024835 | 1 | ZNF403 |
| 221626_at | AL136548 | 1 | ZNF506 |
| 204291_at | NM_014803 | 1 | ZNF518 |
| 221645_s_at | M27877 | 1 | ZNF83 |

Table 2: Genetic markers which are differentially expressed between probable multiple sclerosis which further converted to the diagnosis of definite MS during a two-years follow up and healthy controls are provided (the Probeset ID of the Affymetrix Gene Chip), along with the corresponding GenBank accession number (GenBank Acc. No.), the gene symbol and the direction of change in gene expression (“1”- upregulation; “−1” - downregulation). Note that the p values of the TNOM, Info and t-Test statistical tests all passed the 95% confidence level.

Sustained probable MS: Analysis of the signature of non-convertors—Analyzing of probable MS patients that did not convert to definite MS during the 2-year follow-up period as compared to healthy controls identified a specific gene expression signature of 503 most informative genes that is characteristic to these patients (Table 3, hereinbelow).

TABLE 3
Differentially expressed markers between probable multiple
sclerosis subjects which did not convert to the diagnosis of
definite multiple sclerosis (non-convertors) and healthy controls

| | GenBank | | Gene |
|---|---|---|---|
| Probeset | Acc. No. | Dir | Symbol |
| 212126_at | | −1 | BG391282 |
| 213002_at | AA770596 | −1 | AA770596 |
| 207593_at | NM_022169 | −1 | ABCG4 |
| 219935_at | NM_007038 | −1 | ADAMTS5 |
| 216678_at | AK000773 | −1 | AK000773 |
| 222252_x_at | AK023354 | −1 | AK023354 |
| 216746_at | AK024606 | −1 | AK024606 |
| 216774_at | AK025325 | −1 | AK025325 |
| 216336_x_at | AL031602 | −1 | AL031602 |
| 216822_x_at | AL359763 | −1 | AL359763 |
| 216813_at | AL512728 | −1 | AL512728 |
| 202920_at | NM_001148 | −1 | ANK2 |
| 215764_x_at | AA877641 | −1 | AP2A2 |
| 201168_x_at | NM_004309 | −1 | ARHGDIA |
| 213138_at | M62324 | −1 | ARID5A |
| 202208_s_at | BC001051 | −1 | ARL7 |
| 219996_at | NM_024708 | −1 | ASB7 |
| 215987_at | AV654984 | −1 | AV654984 |
| 222303_at | AV700891 | −1 | AV700891 |
| 222348_at | AW971134 | −1 | AW971134 |
| 222329_x_at | AW974816 | −1 | AW974816 |
| 205681_at | NM_004049 | −1 | BCL2A1 |
| 213281_at | BE327172 | −1 | BE327172 |
| 217921_at | BE543064 | −1 | BE543064 |
| 215775_at | BF084105 | −1 | BF084105 |
| 217591_at | BF725121 | −1 | BF725121 |
| 201235_s_at | BG339064 | −1 | BTG2 |
| 220509_at | NM_018605 | −1 | C13orf10 |
| 201309_x_at | NM_004772 | −1 | C5orf13 |
| 220614_s_at | NM_024694 | −1 | C6orf103 |
| 205476_at | NM_004591 | −1 | CCL20 |
| 205114_s_at | NM_002983 | −1 | CCL3, |
| | | | CCL3L1, |
| | | | MGC12815 |
| 201884_at | NM_004363 | −1 | CEACAM5 |
| 218177_at | NM_020412 | −1 | CHMP1.5 |
| 216016_at | AK027194 | −1 | CIAS1 |
| 216015_s_at | AK027194 | −1 | CIAS1 |
| 206207_at | NM_001828 | −1 | CLC |
| 213622_at | AI733465 | −1 | COL9A2 |
| 213504_at | W63732 | −1 | COPS6 |
| 204533_at | NM_001565 | −1 | CXCL10 |
| 203666_at | NM_000609 | −1 | CXCL12 |
| 209774_x_at | M57731 | −1 | CXCL2 |
| 207850_at | NM_002090 | −1 | CXCL3 |
| 214421_x_at | AV652420 | −1 | CYP2C9 |
| 218013_x_at | NM_016221 | −1 | DCTN4 |
| 221780_s_at | AF336851 | −1 | DDX27 |
| 220004_at | NM_018665 | −1 | DDX43 |
| 221293_s_at | NM_022047 | −1 | DEF6 |
| 213632_at | M94065 | −1 | DHODH |
| 33768_at | L19267 | −1 | DMWD |
| 215151_at | AB014594 | −1 | DOCK10 |
| 201044_x_at | AA530892 | −1 | DUSP1 |
| 201041_s_at | NM_004417 | −1 | DUSP1 |
| 204794_at | NM_004418 | −1 | DUSP2 |
| 213477_x_at | AL515273 | −1 | EEF1A1 |
| 201694_s_at | NM_001964 | −1 | EGR1 |
| 201693_s_at | NM_001964 | −1 | EGR1 |
| 200596_s_at | BE614908 | −1 | EIF3S10 |
| 204513_s_at | NM_014800 | −1 | ELMO1 |
| 210651_s_at | L41939 | −1 | EPHB2 |
| 205767_at | NM_001432 | −1 | EREG |
| 212106_at | AB020694 | −1 | ETEA |
| 201329_s_at | NM_005239 | −1 | ETS2 |
| 211307_s_at | U43677 | −1 | FCAR |
| 218814_s_at | NM_018252 | −1 | FLJ10874 |
| 220215_at | NM_024804 | −1 | FLJ12606 |
| 219397_at | NM_025147 | −1 | FLJ13448 |
| 218810_at | NM_025079 | −1 | FLJ23231 |
| 58367_s_at | AA429615 | −1 | FLJ23233 |
| 219617_at | NM_024766 | −1 | FLJ23451 |
| 206548_at | NM_024880 | −1 | FLJ23556 |
| 210414_at | AF169675 | −1 | FLRT1 |
| 210933_s_at | BC004908 | −1 | FSCN1 |
| 217370_x_at | S75762 | −1 | FUS |
| 209416_s_at | AF083810 | −1 | FZR1 |
| 215308_at | AF052148 | −1 | G22P1 |
| 209305_s_at | AF078077 | −1 | GADD45B |
| 219954_s_at | NM_020973 | −1 | GBA3 |
| 207034_s_at | NM_030379 | −1 | GLI2 |
| 205220_at | NM_006018 | −1 | GPR109B |
| 208524_at | NM_005290 | −1 | GPR15 |
| 209945_s_at | BC000251 | −1 | GSK3B |
| 212291_at | AI393355 | −1 | HIPK1 |
| 208026_at | NM_003540 | −1 | HIST1H4F |
| 208729_x_at | D83043 | −1 | HLA-B |
| 208812_x_at | BC004489 | −1 | HLA-C, |
| | | | HLA-B |
| 222126_at | AI247494 | −1 | HRBL, |
| | | | IRS3L |
| 206708_at | NM_002158 | −1 | HTLF |
| 36564_at | W27419 | −1 | IBRDC3 |
| 202637_s_at | AI608725 | −1 | ICAM1 |
| 215485_s_at | AA284705 | −1 | ICAM1 |
| 202638_s_at | NM_000201 | −1 | ICAM1 |
| 202438_x_at | BF346014 | −1 | IDS |
| 202081_at | NM_004907 | −1 | IER2 |
| 201631_s_at | NM_003897 | −1 | IER3 |
| 207901_at | NM_002187 | −1 | IL12B |
| 210118_s_at | M15329 | −1 | IL1A |
| 205067_at | NM_000576 | −1 | IL1B |
| 39402_at | M15330 | −1 | IL1B |
| 212657_s_at | AW083357 | −1 | IL1RN |
| 211506_s_at | AF043337 | −1 | IL8 |
| 202859_x_at | NM_000584 | −1 | IL8 |
| 208364_at | NM_001566 | −1 | INPP4A |
| 213146_at | AA521267 | −1 | JMJD3 |
| 41386_i_at | AB002344 | −1 | JMJD3 |
| 41387_r_at | AB002344 | −1 | JMJD3 |
| 201465_s_at | BC002646 | −1 | JUN |
| 203751_x_at | NM_005354 | −1 | JUND |
| 207141_s_at | U39196 | −1 | KCNJ3 |
| 212057_at | AA206161 | −1 | KIAA0182 |
| 215137_at | H92070 | −1 | KIAA0508 |
| 204403_x_at | NM_014719 | −1 | KIAA0738 |
| 206966_s_at | NM_016285 | −1 | KLF12 |
| 208467_at | NM_007249 | −1 | KLF12 |
| 204012_s_at | AL529189 | −1 | LCMT2 |
| 202068_s_at | NM_000527 | −1 | LDLR |
| 215462_at | AI978990 | −1 | LOC149478 |
| 216084_at | AL080137 | −1 | LOC389715 |
| 64899_at | AA209463 | −1 | LPPR2 |
| 205193_at | NM_012323 | −1 | MAFF |
| 219442_at | NM_024048 | −1 | MGC3020 |
| 209231_s_at | BC004191 | −1 | MGC3248 |
| 207984_s_at | NM_005374 | −1 | MPP2 |
| 210254_at | L35848 | −1 | MS4A3 |
| 202247_s_at | NM_004689 | −1 | MTA1 |
| 212452_x_at | AF113514 | −1 | MYST4 |
| 205669_at | NM_004540 | −1 | NCAM2 |
| 200855_at | AW771910 | −1 | NCOR1 |
| 204888_s_at | AF029729 | −1 | NEURL |
| 207535_s_at | NM_002502 | −1 | NFKB2 |
| 201502_s_at | NM_020529 | −1 | NFKBIA |
| 203927_at | NM_004556 | −1 | NFKBIE |
| 215720_s_at | AL031778 | −1 | NFYA |
| 220856_x_at | NM_014128 | −1 | NM_014128 |
| 207783_x_at | NM_017627 | −1 | NM_017627 |
| 208120_x_at | NM_031221 | −1 | NM_031221 |
| 202340_x_at | NM_002135 | −1 | NR4A1 |
| 210226_at | D85245 | −1 | NR4A1 |
| 211973_at | AW341200 | −1 | NUDT3 |
| 204435_at | NM_014778 | −1 | NUPL1 |
| 208274_at | NM_022375 | −1 | OCLM |
| 213131_at | R38389 | −1 | OLFM1 |
| 221344_at | NM_013936 | −1 | OR12D2 |
| 214637_at | BG437034 | −1 | OSM |
| 205432_at | NM_002557 | −1 | OVGP1 |
| 206880_at | NM_005446 | −1 | P2RXL1 |
| 215823_x_at | U64661 | −1 | PABPC1, |
| | | | PABPC3 |
| 204267_x_at | NM_004203 | −1 | PKMYT1 |
| 207717_s_at | NM_004572 | −1 | PKP2 |
| 204691_x_at | NM_003560 | −1 | PLA2G6 |
| 202924_s_at | AF006005 | −1 | PLAGL2 |
| 205934_at | NM_006226 | −1 | PLCL1 |
| 203471_s_at | NM_002664 | −1 | PLEK |
| 201489_at | BC005020 | −1 | PPIF |
| 37028_at | U83981 | −1 | PPP1R15A |
| 202014_at | NM_014330 | −1 | PPP1R15A |
| 205643_s_at | NM_004576 | −1 | PPP2R2B |
| 203317_at | NM_012455 | −1 | PSD4 |
| 204748_at | NM_000963 | −1 | PTGS2 |
| 206157_at | NM_002852 | −1 | PTX3 |
| 34478_at | X79780 | −1 | RAB11B |
| 205461_at | NM_006861 | −1 | RAB35 |
| 204543_at | NM_005312 | −1 | RAPGEF1 |
| 203750_s_at | NM_000964 | −1 | RARA |
| 209936_at | AF107493 | −1 | RBM5 |
| 216153_x_at | AK022897 | −1 | RECK |
| 209637_s_at | AF030111 | −1 | RGS12 |
| 221989_at | AW057781 | −1 | RPL10 |
| 215620_at | AU147182 | −1 | RREB1 |
| 205485_at | NM_000540 | −1 | RYR1 |
| 215670_s_at | AK022844 | −1 | SCAND2 |
| 217331_at | U63542 | −1 | SCC-112 |
| 208124_s_at | NM_004263 | −1 | SEMA4F |
| 213742_at | AW241752 | −1 | SFRS11 |
| 222169_x_at | N71739 | −1 | SH2D3A |
| 214623_at | AA845710 | −1 | SHFM3P1 |
| 220000_at | NM_003830 | −1 | SIGLEC5 |
| 210796_x_at | D86359 | −1 | SIGLEC6 |
| 203125_x_at | AF046997 | −1 | SLC11A2 |
| 208389_s_at | NM_004171 | −1 | SLC1A2 |
| 220091_at | NM_017585 | −1 | SLC2A6 |
| 210001_s_at | AB005043 | −1 | SOCS1 |
| 203372_s_at | NM_003877 | −1 | SOCS2 |
| 215223_s_at | W46388 | −1 | SOD2 |
| 215078_at | AL050388 | −1 | SOD2 |
| 217576_x_at | BF692958 | −1 | SOS2 |
| 210693_at | BC001788 | −1 | SPPL2B |
| 202021_x_at | AF083441 | −1 | SUI1 |
| 207684_at | NM_004608 | −1 | TBX6 |
| 221473_x_at | U49188 | −1 | TDE1 |
| 211769_x_at | BC006088 | −1 | TDE1 |
| 201109_s_at | AV726673 | −1 | THBS1 |
| 201110_s_at | NM_003246 | −1 | THBS1 |
| 214657_s_at | AU134977 | −1 | TncRNA |
| 207113_s_at | NM_000594 | −1 | TNF |
| 202643_s_at | AI738896 | −1 | TNFAIP3 |
| 202644_s_at | NM_006290 | −1 | TNFAIP3 |
| 206025_s_at | AW188198 | −1 | TNFAIP6 |
| 216042_at | AI275938 | −1 | TNFRSF25 |
| 212260_at | AL045800 | −1 | TNRC15 |
| 204080_at | NM_025077 | −1 | TOE1 |
| 212869_x_at | AI721229 | −1 | TPT1 |
| 211943_x_at | AL565449 | −1 | TPT1 |
| 214327_x_at | AI888178 | −1 | TPT1 |
| 212284_x_at | BG498776 | −1 | TPT1 |
| 220205_at | NM_013315 | −1 | TPTE |
| 213593_s_at | AW978896 | −1 | TRA2A |
| 205558_at | NM_004620 | −1 | TRAF6 |
| 213191_at | AF070530 | −1 | TRIF |
| 209013_x_at | AF091395 | −1 | TRIO |
| 207490_at | NM_025019 | −1 | TUBA4 |
| 201378_s_at | NM_014847 | −1 | UBAP2L |
| 215577_at | AU146791 | −1 | UBE2E1 |
| 211403_x_at | AF167079 | −1 | VCX-C, |
| | | | VCX2, |
| | | | VCX3, |
| | | | VCY, VCX |
| 214758_at | AL080157 | −1 | WDR21 |
| 218851_s_at | NM_018383 | −1 | WDR33 |
| 216036_x_at | AK001734 | −1 | WDTC1 |
| 222180_at | AU147889 | −1 | YES1 |
| 219312_s_at | NM_023929 | −1 | ZBTB10 |
| 203602_s_at | NM_003443 | −1 | ZBTB17 |
| 216350_s_at | X52332 | −1 | ZNF10 |
| 219228_at | NM_018555 | −1 | ZNF331 |
| 220836_at | NM_017757 | −1 | ZNF407 |
| 220086_at | NM_022466 | −1 | ZNFN1A5 |
| 43511_s_at | AI201594 | 1 | DKFZp762 |
| | | | M127 |
| 217480_x_at | M20812 | 1 | Ig kappa |
| | | | chain |
| 212993_at | AA114166 | 1 | XP_316923.1 |
| 212583_at | AB011132 | 1 | AB011132 |
| 209307_at | AB014540 | 1 | AB014540 |
| 217239_x_at | AF044592 | 1 | AF044592 |
| 210183_x_at | AF112222 | 1 | AF112222 |
| 217939_s_at | NM_017657 | 1 | AFTIPHILIN |
| 204057_at | AI073984 | 1 | AI073984 |
| 222273_at | AI419423 | 1 | AI419423 |
| 206513_at | NM_004833 | 1 | AIM2 |
| 214259_s_at | AI144075 | 1 | AKR7A2 |
| 209200_at | AL536517 | 1 | AL536517 |
| 218203_at | NM_013338 | 1 | ALG5 |
| 209425_at | AA888589 | 1 | AMACR |
| 202204_s_at | NM_001144 | 1 | AMFR |
| 218230_at | AL044651 | 1 | ARFIP1 |
| 201229_s_at | BC000422 | 1 | ARIH2 |
| 202564_x_at | NM_001667 | 1 | ARL2 |
| 203487_s_at | NM_015396 | 1 | ARMC8 |
| 214749_s_at | AK000818 | 1 | ARMCX6 |
| 213238_at | AI478147 | 1 | ATP10D |
| 208898_at | AF077614 | 1 | ATP6V1D |
| 212559_at | AU148827 | 1 | AU148827 |
| 219667_s_at | NM_017935 | 1 | BANK1 |
| 214836_x_at | BG536224 | 1 | BG536224 |
| 207655_s_at | NM_013314 | 1 | BLNK |
| 220059_at | NM_012108 | 1 | BRDG1 |
| 207369_at | Z97632 | 1 | BRS3 |
| 213410_at | AL050102 | 1 | C10orf137 |
| 219012_s_at | AK023651 | 1 | C11orf30 |
| 220240_s_at | NM_017905 | 1 | C13orf11 |
| 216263_s_at | AK022215 | 1 | C14orf120 |
| 213508_at | AA142942 | 1 | C14orf147 |
| 221932_s_at | AA133341 | 1 | C14orf87 |
| 52285_f_at | AW002970 | 1 | C18orf9 |
| 219283_at | NM_014158 | 1 | C1GALT2 |
| 217737_x_at | NM_016407 | 1 | C20orf43 |
| 221196_x_at | NM_024332 | 1 | C6.1A |
| 220329_s_at | NM_017909 | 1 | C6orf96 |
| 204480_s_at | NM_024112 | 1 | C9orf16 |
| 200622_x_at | AV685208 | 1 | CALM3 |
| 205034_at | NM_004702 | 1 | CCNE2 |
| 216379_x_at | AK000168 | 1 | CD24 |
| 200983_x_at | BF983379 | 1 | CD59 |
| 215925_s_at | AF283777 | 1 | CD72 |
| 202892_at | NM_004661 | 1 | CDC23 |
| 211804_s_at | AB012305 | 1 | CDK2 |
| 204661_at | NM_001803 | 1 | CDW52 |
| 219375_at | NM_006090 | 1 | CEPT1 |
| 213375_s_at | N80918 | 1 | CG018 |
| 218102_at | NM_015954 | 1 | CGI-26 |
| 33307_at | AL022316 | 1 | CGI-96 |
| 214426_x_at | BF062223 | 1 | CHAF1A |
| 220496_at | NM_016509 | 1 | CLEC2 |
| 202799_at | NM_006012 | 1 | CLPP |
| 222043_at | AI982754 | 1 | CLU |
| 219301_s_at | NM_014141 | 1 | CNTNAP2 |
| 212189_s_at | AK022874 | 1 | COG4 |
| 218328_at | NM_016035 | 1 | COQ4 |
| 213735_s_at | AI557312 | 1 | COX5B |
| 201597_at | NM_001865 | 1 | COX7A2 |
| 204920_at | AF154830 | 1 | CPS1 |
| 203633_at | BF001714 | 1 | CPT1A |
| 203804_s_at | NM_006107 | 1 | CROP |
| 203445_s_at | NM_005730 | 1 | CTDSP2 |
| 200932_s_at | NM_006400 | 1 | DCTN2 |
| 203785_s_at | NM_018380 | 1 | DDX28 |
| 209549_s_at | BC001121 | 1 | DGUOK |
| 202532_s_at | NM_000791 | 1 | DHFR |
| 220985_s_at | NM_030954 | 1 | DKFZP564A022 |
| 213647_at | D42046 | 1 | DNA2L |
| 221689_s_at | AB035745 | 1 | DSCR5 |
| 204841_s_at | NM_003566 | 1 | EEA1 |
| 206254_at | NM_001963 | 1 | EGF |
| 204410_at | NM_004681 | 1 | EIF1AY |
| 202461_at | NM_014239 | 1 | EIF2B2 |
| 208985_s_at | BC002719 | 1 | EIF3S1 |
| 208688_x_at | U78525 | 1 | EIF3S9 |
| 204505_s_at | NM_001978 | 1 | EPB49 |
| 203249_at | AB002386 | 1 | EZH1 |
| 218504_at | NM_016044 | 1 | FAHD2A |
| 222056_s_at | AA723370 | 1 | FAHD2A |
| 201863_at | NM_014077 | 1 | FAM32A |
| 38043_at | X55448 | 1 | FAM3A |
| 220408_x_at | NM_017569 | 1 | FAM48A |
| 209630_s_at | U87460 | 1 | FBXW2 |
| 210889_s_at | M31933 | 1 | FCGR2B |
| 217518_at | BF056029 | 1 | FER1L3 |
| 210298_x_at | AF098518 | 1 | FHL1 |
| 201540_at | NM_001449 | 1 | FHL1 |
| 208255_s_at | NM_012181 | 1 | FKBP8 |
| 219130_at | NM_019083 | 1 | FLJ10287 |
| 218179_s_at | NM_021942 | 1 | FLJ12716 |
| 221777_at | BE966197 | 1 | FLJ14827 |
| 219802_at | NM_024854 | 1 | FLJ22028 |
| 220169_at | NM_024943 | 1 | FLJ23235 |
| 205140_at | NM_003838 | 1 | FPGT |
| 204145_at | NM_004477 | 1 | FRG1 |
| 209729_at | BC001782 | 1 | GAS2L1 |
| 204220_at | NM_004877 | 1 | GMFG |
| 204000_at | NM_016194 | 1 | GNB5 |
| 218361_at | NM_018178 | 1 | GPP34R |
| 222155_s_at | AK021918 | 1 | GPR172A |
| 201106_at | NM_002085 | 1 | GPX4 |
| 201501_s_at | NM_002092 | 1 | GRSF1 |
| 203577_at | NM_001517 | 1 | GTF2H4 |
| 218343_s_at | NM_012086 | 1 | GTF3C3 |
| 221942_s_at | AI719730 | 1 | GUCY1A3 |
| 203817_at | W93728 | 1 | GUCY1B3 |
| 213515_x_at | AI133353 | 1 | HBG2 |
| 209273_s_at | BG387555 | 1 | HBLD2 |
| 204689_at | NM_001529 | 1 | HHEX |
| 215933_s_at | Z21533 | 1 | HHEX |
| 220387_s_at | NM_007071 | 1 | HHLA3 |
| 218525_s_at | NM_017902 | 1 | HIF1AN |
| 203932_at | NM_002118 | 1 | HLA-DMB |
| 205671_s_at | NM_002120 | 1 | HLA-DOB |
| 200904_at | X56841 | 1 | HLA-E |
| 213793_s_at | BE550452 | 1 | HOMER1 |
| 203914_x_at | NM_000860 | 1 | HPGD |
| 210112_at | U96721 | 1 | HPS1 |
| 217869_at | NM_016142 | 1 | HSD17B12 |
| 221791_s_at | BG167522 | 1 | HSPC016 |
| 221711_s_at | BC006244 | 1 | HSPC142 |
| 221622_s_at | AF246240 | 1 | HT007 |
| 207180_s_at | NM_006410 | 1 | HTAT1P2 |
| 209586_s_at | AF123539 | 1 | HTCD37 |
| 219209_at | NM_022168 | 1 | IFIH1 |
| 203595_s_at | N47725 | 1 | IFIT5 |
| 211868_x_at | AJ225092 | 1 | IGHG1 |
| 212592_at | AV733266 | 1 | IGJ |
| 221651_x_at | BC005332 | 1 | IGKC |
| 216207_x_at | AW408194 | 1 | IGKV1D-13 |
| 215121_x_at | AA680302 | 1 | IGLC2 |
| 48825_at | AA887083 | 1 | ING4 |
| 204202_at | NM_017604 | 1 | IQCE |
| 206493_at | NM_000419 | 1 | ITGA2B |
| 206494_s_at | NM_000419 | 1 | ITGA2B |
| 211945_s_at | BG500301 | 1 | ITGB1 |
| 216261_at | AI151479 | 1 | ITGB3 |
| 213483_at | AK025679 | 1 | KIAA0073 |
| 202503_s_at | NM_014736 | 1 | KIAA0101 |
| 212149_at | AW470003 | 1 | KIAA0143 |
| 212523_s_at | D63480 | 1 | KIAA0146 |
| 212733_at | AI798908 | 1 | KIAA0226 |
| 204876_at | NM_014699 | 1 | KIAA0296 |
| 76897_s_at | AA628140 | 1 | KIAA0674 |
| 212548_s_at | BF515124 | 1 | KIAA0826 |
| 207314_x_at | NM_006737 | 1 | KIR3DL2 |
| 201553_s_at | NM_005561 | 1 | LAMP1 |
| 203042_at | NM_002294 | 1 | LAMP2 |
| 221515_s_at | BC001214 | 1 | LCMT1 |
| 221274_s_at | NM_030805 | 1 | LMAN2L |
| 213408_s_at | AK024034 | 1 | LOC220686, |
| | | | PIK4CA |
| 216250_s_at | X77598 | 1 | LPXN |
| 35974_at | U10485 | 1 | LRMP |
| 210044_s_at | BC002796 | 1 | LYL1 |
| 201384_s_at | NM_005899 | 1 | M17S2 |
| 218573_at | NM_014061 | 1 | MAGEH1 |
| 206854_s_at | NM_003188 | 1 | MAP3K7 |
| 222036_s_at | AI859865 | 1 | MCM4 |
| 209199_s_at | L08895 | 1 | MEF2C |
| 202645_s_at | NM_000244 | 1 | MEN1 |
| 217043_s_at | U95822 | 1 | MFN1 |
| 217022_s_at | S55735 | 1 | MGC27165 |
| 201764_at | NM_024056 | 1 | MGC5576 |
| 214364_at | W84525 | 1 | MGC61716 |
| 213528_at | AL035369 | 1 | MGC9084 |
| 205612_at | NM_007351 | 1 | MMRN1 |
| 218853_s_at | NM_019556 | 1 | MOSPD1 |
| 218339_at | NM_014180 | 1 | MRPL22 |
| 203095_at | NM_002453 | 1 | MTIF2 |
| 210386_s_at | BC001906 | 1 | MTX1 |
| 203517_at | NM_006554 | 1 | MTX2 |
| 202960_s_at | NM_000255 | 1 | MUT |
| 203359_s_at | NM_012333 | 1 | MYCBP |
| 200027_at | NM_004539 | 1 | NARS |
| 202607_at | AL526632 | 1 | NDST1 |
| 209224_s_at | BC003674 | 1 | NDUFA2 |
| 202839_s_at | NM_004146 | 1 | NDUFB7 |
| 218101_s_at | NM_004549 | 1 | NDUFC2 |
| 217896_s_at | NM_024946 | 1 | NIP30 |
| 213682_at | AL036344 | 1 | NUP50 |
| 213018_at | AI337901 | 1 | ODAG |
| 205301_s_at | NM_016820 | 1 | OGG1 |
| 203351_s_at | NM_002552 | 1 | ORC4L |
| 37966_at | AA187563 | 1 | PARVB |
| 37965_at | AA181053 | 1 | PARVB |
| 216253_s_at | N73272 | 1 | PARVB |
| 213652_at | AU152579 | 1 | PCSK5 |
| 207414_s_at | NM_002570 | 1 | PCSK6 |
| 219180_s_at | AI817074 | 1 | PEX26 |
| 206390_x_at | NM_002619 | 1 | PF4 |
| 202739_s_at | NM_000293 | 1 | PHKB |
| 217097_s_at | AC004990 | 1 | PHTF2 |
| 207081_s_at | NM_002650 | 1 | PIK4CA |
| 202732_at | NM_007066 | 1 | PKIG |
| 201410_at | AI983043 | 1 | PLEKHB2 |
| 212719_at | AB011178 | 1 | PLEKHE1 |
| 201682_at | NM_004279 | 1 | PMPCB |
| 219317_at | NM_007195 | 1 | POLI |
| 202306_at | NM_002696 | 1 | POLR2G |
| 212955_s_at | AL037557 | 1 | POLR2I |
| 209382_at | U93867 | 1 | POLR3C |
| 209482_at | BC001430 | 1 | POP7 |
| 202884_s_at | T79584 | 1 | PPP2R1B |
| 200844_s_at | BE869583 | 1 | PRDX6 |
| 201805_at | NM_002733 | 1 | PRKAG1 |
| 207808_s_at | NM_000313 | 1 | PROS1 |
| 202529_at | NM_002766 | 1 | PRPSAP1 |
| 201316_at | AL523904 | 1 | PSMA2 |
| 204279_at | NM_002800 | 1 | PSMB9 |
| 201068_s_at | NM_002803 | 1 | PSMC2 |
| 200830_at | NM_002808 | 1 | PSMD2 |
| 202009_at | NM_007284 | 1 | PTK9L |
| 219178_at | NM_024638 | 1 | QTRTD1 |
| 220964_s_at | NM_030981 | 1 | RAB1B |
| 217763_s_at | AF183421 | 1 | RAB31 |
| 217764_s_at | AF183421 | 1 | RAB31 |
| 214552_s_at | AF098638 | 1 | RABEP1 |
| 203020_at | NM_014857 | 1 | RABGAP1L |
| 222077_s_at | AU153848 | 1 | RACGAP1 |
| 221809_at | AB040897 | 1 | RANBP10 |
| 202297_s_at | AF157324 | 1 | RER1 |
| 202296_s_at | NM_007033 | 1 | RER1 |
| 209882_at | AF084462 | 1 | RIT1 |
| 218301_at | NM_018226 | 1 | RNPEPL1 |
| 218583_s_at | NM_020640 | 1 | RP42 |
| 209773_s_at | BC001886 | 1 | RRM2 |
| 209486_at | BC004546 | 1 | SAS10 |
| 202084_s_at | NM_003003 | 1 | SEC14L1 |
| 200961_at | NM_012248 | 1 | SEPHS2 |
| 217756_x_at | NM_005770 | 1 | SERF2 |
| 205933_at | NM_015559 | 1 | SETBP1 |
| 204688_at | NM_003919 | 1 | SGCE |
| 213355_at | AI989567 | 1 | SIAT10 |
| 56256_at | AA150165 | 1 | SIDT2 |
| 219185_at | NM_012241 | 1 | SIRT5 |
| 201575_at | NM_012245 | 1 | SKIIP |
| 203775_at | NM_014251 | 1 | SLC25A13 |
| 203658_at | BC001689 | 1 | SLC25A20 |
| 215043_s_at | X83301 | 1 | SMA3, SMA5 |
| 207827_x_at | NM_007308 | 1 | SNCA |
| 206272_at | NM_006542 | 1 | SPHAR |
| 201273_s_at | NM_003133 | 1 | SRP9 |
| 202811_at | NM_006463 | 1 | STAMBP |
| 200028_s_at | NM_020151 | 1 | STARD7 |
| 208855_s_at | AF083420 | 1 | STK24 |
| 212990_at | AB020717 | 1 | SYNJ1 |
| 201259_s_at | AI768845 | 1 | SYPL |
| 221397_at | NM_023921 | 1 | TAS2R10 |
| 209152_s_at | M31523 | 1 | TCF3 |
| 219292_at | NM_018105 | 1 | THAP1 |
| 201447_at | AL567227 | 1 | TIA1 |
| 217979_at | NM_014399 | 1 | TM4SF13 |
| 221246_x_at | NM_018274 | 1 | TNS |
| 209917_s_at | BC002709 | 1 | TP53AP1 |
| 218095_s_at | BC003545 | 1 | TPARL |
| 220865_s_at | NM_014317 | 1 | TPRT |
| 203512_at | NM_014408 | 1 | TRAPPC3 |
| 217958_at | NM_016146 | 1 | TRAPPC4 |
| 204341_at | NM_006470 | 1 | TRIM16 |
| 210705_s_at | AF220028 | 1 | TRIM5 |
| 209778_at | AF007217 | 1 | TRIP11 |
| 215735_s_at | AC005600 | 1 | TSC2 |
| 221253_s_at | NM_030810 | 1 | TXNDC5 |
| 201588_at | NM_004786 | 1 | TXNL1 |
| 218289_s_at | NM_024818 | 1 | UBE1DC1 |
| 203281_s_at | NM_003335 | 1 | UBE1L |
| 218533_s_at | NM_017859 | 1 | UCKL1 |
| 203583_at | NM_014044 | 1 | UNC50 |
| 209268_at | AF165513 | 1 | VPS45A |
| 209452_s_at | AF035824 | 1 | VT11B |
| 220917_s_at | NM_025132 | 1 | WDR19 |
| 217784_at | NM_006555 | 1 | YKT6 |
| 220261_s_at | NM_018106 | 1 | ZDHHC4 |
| 202939_at | NM_005857 | 1 | ZMPSTE24 |
| 201541_s_at | NM_006349 | 1 | ZNHIT1 |

Table 3: Provided are genetic markers which are differentially expressed between subjects diagnosed with probable multiple sclerosis which did not convert to the diagnosis of definite MS (non-convertors) during a two-years follow up and healthy controls (the Probeset ID of the Affymetrix Gene Chip), along with the corresponding GenBank accession number (GenBank Acc. No.), the gene symbol and the direction (Dir) of change in gene expression (“1” - upregulation; “−1” - downregulation).
Note that the p values of the TNOM, Info and t-Test statistical tests all passed the 95% confidence level.

Gene expression pattern of subjects with definite diagnosis of multiple sclerosis—Table 4, hereinbelow, depict 722 genetic markers which are differentially expressed between subjects with a definite diagnosis of multiple sclerosis (both from relapse and remitting phases; blood samples were taken after the diagnosis of definite MS was confirmed, i.e., at least after the second neurological attack) and healthy controls.

TABLE 4
Differentially expressed markers between subjects with the
diagnosis of definite multiple sclerosis and healthy controls

| GenBank | | | | | |
|---|---|---|---|---|---|
| Accession | TNOM | Info | t-Test | | |
| No, | PValue | PValue | PValue | Dir | Gene Symbol |
| AI252582 | 5.31E−06 | 5.31E−06 | 2.46E−05 | −1 | ATP6V0E |
| NM_017686 | 5.31E−06 | 5.31E−06 | 1.44E−05 | −1 | GDAP2 |
| NM_006404 | 2.90E−05 | 4.28E−05 | 1.23E−05 | −1 | PROCR |
| NM_003916 | 1.49E−04 | 1.49E−04 | 8.21E−04 | −1 | AP1S2 |
| NM_015396 | 1.49E−04 | 1.49E−04 | 2.46E−03 | −1 | ARMC8 |
| NM_007212 | 1.49E−04 | 3.72E−05 | 5.28E−05 | −1 | RNF2 |
| AL138761 | 1.49E−04 | 1.49E−04 | 3.67E−03 | −1 | — |
| NM_020474 | 2.70E−04 | 1.90E−04 | 2.85E−02 | −1 | GALNT1 |
| N21138 | 2.70E−04 | 1.90E−04 | 2.98E−04 | −1 | RHOBTB3 |
| NM_015904 | 1.82E−03 | 1.47E−03 | 7.88E−03 | −1 | EIF5B |
| NM_003201 | 1.82E−03 | 1.47E−03 | 4.89E−03 | −1 | TFAM |
| NM_005263 | 1.82E−03 | 1.79E−03 | 1.67E−02 | −1 | GFI1 |
| NM_022335 | 1.82E−03 | 2.96E−03 | 1.64E−04 | −1 | — |
| NM_001222 | 1.82E−03 | 1.79E−03 | 3.87E−03 | −1 | SRP72 |
| BC002456 | 1.82E−03 | 2.96E−03 | 1.85E−04 | −1 | — |
| AW268817 | 1.82E−03 | 1.47E−03 | 3.21E−02 | −1 | CDC5L |
| AB044661 | 1.82E−03 | 3.63E−04 | 7.50E−05 | −1 | XAB1 |
| AK024044 | 1.82E−03 | 1.47E−03 | 8.77E−04 | −1 | SSA2 |
| D42055 | 1.82E−03 | 1.79E−03 | 2.32E−03 | −1 | NEDD4 |
| BF680255 | 1.82E−03 | 9.10E−04 | 4.33E−03 | −1 | — |
| AW238654 | 1.82E−03 | 1.47E−03 | 1.83E−03 | −1 | — |
| AK024584 | 1.82E−03 | 1.47E−03 | 2.95E−02 | −1 | — |
| L40992 | 1.82E−03 | 1.79E−03 | 2.40E−02 | −1 | RUNX2 |
| NM_014159 | 1.82E−03 | 1.47E−03 | 2.75E−03 | −1 | HYPB |
| AL137750 | 1.82E−03 | 1.47E−03 | 1.87E−02 | −1 | ETNK1 |
| AL567227 | 2.01E−03 | 2.88E−03 | 3.52E−02 | −1 | TIA1 |
| NM_014774 | 2.01E−03 | 2.88E−03 | 4.00E−02 | −1 | KIAA0494 |
| AU146275 | 2.01E−03 | 1.69E−03 | 1.55E−02 | −1 | ZNF161 |
| NM_002717 | 2.01E−03 | 1.69E−03 | 6.85E−03 | −1 | PPP2R2A |
| NM_004301 | 2.01E−03 | 2.88E−03 | 1.52E−03 | −1 | ACTL6A |
| BE622627 | 2.01E−03 | 1.69E−03 | 1.83E−02 | −1 | PIK3R3 |
| NM_005977 | 2.01E−03 | 2.55E−04 | 7.91E−04 | −1 | RNF6 |
| NM_003616 | 2.01E−03 | 2.88E−03 | 9.81E−03 | −1 | SIP1 |
| NM_005531 | 2.01E−03 | 1.69E−03 | 7.12E−04 | −1 | IFI16 |
| NM_005849 | 2.01E−03 | 2.88E−03 | 1.29E−03 | −1 | IGSF6 |
| NM_006065 | 2.01E−03 | 5.47E−04 | 1.08E−03 | −1 | SIRPB1 |
| NM_003452 | 2.01E−03 | 2.88E−03 | 1.16E−02 | −1 | ZNF189 |
| NM_003539 | 2.01E−03 | 2.88E−03 | 2.71E−03 | −1 | HIST1H4D |
| NM_006016 | 2.01E−03 | 1.69E−03 | 1.81E−03 | −1 | CD164 |
| AI332962 | 2.01E−03 | 1.69E−03 | 2.36E−03 | −1 | — |
| BC004421 | 2.01E−03 | 1.21E−03 | 1.18E−03 | −1 | ZNF330 |
| AB037701 | 2.01E−03 | 2.88E−03 | 5.57E−03 | −1 | SIP1 |
| AB000815 | 2.01E−03 | 2.88E−03 | 8.15E−04 | −1 | ARNTL |
| BC006403 | 2.01E−03 | 1.21E−03 | 1.38E−04 | −1 | NCK1 |
| AB037703 | 2.01E−03 | 2.88E−03 | 1.25E−02 | −1 | SIP1 |
| U90142 | 2.01E−03 | 1.21E−03 | 3.63E−04 | −1 | BTN2A1 |
| AF234262 | 2.01E−03 | 2.88E−03 | 1.75E−03 | −1 | MAP3K2 |
| M60725 | 2.01E−03 | 2.55E−04 | 1.06E−04 | −1 | RPS6KB1 |
| AI057093 | 2.01E−03 | 1.69E−03 | 1.21E−03 | −1 | RDX |
| AL079292 | 2.01E−03 | 2.88E−03 | 1.92E−02 | −1 | — |
| AB002347 | 2.01E−03 | 2.55E−04 | 2.15E−03 | −1 | C6orf133 |
| AL033377 | 2.01E−03 | 2.88E−03 | 4.63E−02 | −1 | GPR126 |
| AI811577 | 2.01E−03 | 1.69E−03 | 2.03E−03 | −1 | ZNF184 |
| AI860341 | 2.01E−03 | 1.69E−03 | 1.06E−03 | −1 | ACAA1 |
| AF052146 | 2.01E−03 | 2.88E−03 | 1.12E−03 | −1 | — |
| AK025174 | 2.01E−03 | 2.88E−03 | 4.88E−03 | −1 | GSDML |
| AL096729 | 2.01E−03 | 1.69E−03 | 2.80E−02 | −1 | GSTA1 |
| AK026712 | 2.01E−03 | 2.88E−03 | 4.93E−03 | −1 | — |
| AL049325 | 2.01E−03 | 2.88E−03 | 2.30E−02 | −1 | CCM1 |
| BC005316 | 2.01E−03 | 2.88E−03 | 2.52E−02 | −1 | NFYB |
| NM_016618 | 2.01E−03 | 5.47E−04 | 6.92E−03 | −1 | LOC51315 |
| NM_017423 | 2.01E−03 | 2.55E−04 | 6.86E−04 | −1 | GALNT7 |
| NM_018046 | 2.01E−03 | 2.88E−03 | 7.75E−03 | −1 | VG5Q |
| NM_018489 | 2.01E−03 | 1.69E−03 | 3.98E−04 | −1 | ASH1L |
| NM_018293 | 2.01E−03 | 1.69E−03 | 1.80E−03 | −1 | FLJ10997 |
| NM_018398 | 2.01E−03 | 1.69E−03 | 8.61E−03 | −1 | CACNA2D3 |
| NM_018042 | 2.01E−03 | 2.88E−03 | 1.97E−03 | −1 | FLJ10260 |
| NM_022488 | 2.01E−03 | 2.88E−03 | 5.03E−03 | −1 | APG3L |
| NM_030934 | 2.01E−03 | 1.69E−03 | 9.89E−03 | −1 | C1orf25 |
| AU157915 | 2.01E−03 | 2.88E−03 | 8.04E−03 | −1 | YTHDF3 |
| BG167522 | 2.01E−03 | 2.88E−03 | 2.61E−02 | −1 | HSPC016 |
| W72694 | 2.01E−03 | 2.55E−04 | 1.72E−03 | −1 | FAM26B |
| NM_002136 | 9.49E−03 | 8.40E−03 | 1.84E−02 | −1 | HNRPA1 |
| NM_005520 | 9.49E−03 | 8.40E−03 | 4.11E−03 | −1 | HNRPH1 |
| NM_020474 | 9.49E−03 | 1.37E−02 | 1.53E−02 | −1 | GALNT1 |
| NM_004048 | 9.49E−03 | 3.33E−03 | 1.95E−02 | −1 | B2M |
| NM_005415 | 9.49E−03 | 7.52E−03 | 2.69E−03 | −1 | SLC20A1 |
| AW051311 | 9.49E−03 | 1.56E−02 | 1.20E−02 | −1 | KPNA1 |
| AB020335 | 9.49E−03 | 7.52E−03 | 1.85E−02 | −1 | SEL1L |
| NM_002023 | 9.49E−03 | 1.37E−02 | 6.51E−03 | −1 | FMOD |
| NM_005103 | 9.49E−03 | 1.37E−02 | 1.21E−03 | −1 | FEZ1 |
| NM_000609 | 9.49E−03 | 7.52E−03 | 4.73E−02 | −1 | CXCL12 |
| NM_003759 | 9.49E−03 | 1.37E−02 | 2.07E−02 | −1 | SLC4A4 |
| NM_005589 | 9.49E−03 | 1.56E−02 | 1.03E−02 | −1 | ALDH6A1 |
| NM_005630 | 9.49E−03 | 1.56E−02 | 2.48E−02 | −1 | SLCO2A1 |
| NM_001046 | 9.49E−03 | 1.37E−02 | 2.06E−03 | −1 | SLC12A2 |
| NM_005388 | 9.49E−03 | 7.52E−03 | 2.40E−02 | −1 | PDCL |
| NM_005261 | 9.49E−03 | 8.40E−03 | 7.52E−03 | −1 | GEM |
| NM_014254 | 9.49E−03 | 1.56E−02 | 1.21E−02 | −1 | TMEM5 |
| NM_016656 | 9.49E−03 | 8.40E−03 | 7.06E−04 | −1 | RRAGB |
| NM_002147 | 9.49E−03 | 7.52E−03 | 1.86E−02 | −1 | HOXB5 |
| NM_004758 | 9.49E−03 | 1.37E−02 | 8.74E−03 | −1 | BZRAP1 |
| NM_002547 | 9.49E−03 | 1.56E−02 | 5.79E−03 | −1 | OPHN1 |
| NM_007366 | 9.49E−03 | 1.37E−02 | 8.98E−03 | −1 | PLA2R1 |
| NM_001340 | 9.49E−03 | 8.40E−03 | 3.60E−03 | −1 | CYLC2 |
| NM_015623 | 9.49E−03 | 7.52E−03 | 1.05E−02 | −1 | DKFZP564D166 |
| AF116710 | 9.49E−03 | 7.52E−03 | 1.10E−02 | −1 | RPS14 |
| BC000914 | 9.49E−03 | 7.52E−03 | 2.46E−03 | −1 | SFRS3 |
| U40763 | 9.49E−03 | 1.37E−02 | 7.34E−03 | −1 | PPIG |
| AB023200 | 9.49E−03 | 1.56E−02 | 4.79E−03 | −1 | C22orf19 |
| L07515 | 9.49E−03 | 3.33E−03 | 3.32E−04 | −1 | CBX5 |
| AB056663 | 9.49E−03 | 7.52E−03 | 1.00E−02 | −1 | ITCH |
| AW003989 | 9.49E−03 | 3.63E−04 | 4.13E−03 | −1 | C19orf2 |
| AF130104 | 9.49E−03 | 7.52E−03 | 1.37E−02 | −1 | — |
| BC002713 | 9.49E−03 | 7.52E−03 | 1.04E−02 | −1 | MXD4 |
| AF229180 | 9.49E−03 | 3.33E−03 | 4.12E−04 | −1 | AASS |
| BE737027 | 9.49E−03 | 7.52E−03 | 1.79E−03 | −1 | — |
| AI972268 | 9.49E−03 | 7.52E−03 | 1.65E−02 | −1 | PSME4 |
| AA148507 | 9.49E−03 | 2.52E−03 | 6.95E−04 | −1 | SLC7A1 |
| BG111635 | 9.49E−03 | 1.56E−02 | 2.16E−02 | −1 | CAST |
| AI022882 | 9.49E−03 | 1.37E−02 | 2.36E−03 | −1 | PAM |
| L43577 | 9.49E−03 | 7.52E−03 | 1.52E−02 | −1 | CXorf40 |
| BE312027 | 9.49E−03 | 8.40E−03 | 1.81E−03 | −1 | — |
| AK022846 | 9.49E−03 | 8.40E−03 | 1.60E−02 | −1 | INPP5B |
| BG260658 | 9.49E−03 | 7.52E−03 | 2.00E−02 | −1 | — |
| AW007137 | 9.49E−03 | 3.63E−04 | 2.91E−03 | −1 | — |
| AI970731 | 9.49E−03 | 1.37E−02 | 2.38E−02 | −1 | RPS7 |
| H71805 | 9.49E−03 | 1.56E−02 | 2.40E−03 | −1 | — |
| AV717561 | 9.49E−03 | 1.23E−02 | 1.55E−03 | −1 | — |
| AI613383 | 9.49E−03 | 1.56E−02 | 4.68E−03 | −1 | EEF1D |
| W87901 | 9.49E−03 | 1.37E−02 | 4.57E−03 | −1 | — |
| AK022065 | 9.49E−03 | 8.40E−03 | 1.10E−03 | −1 | RAB5A |
| AJ010395 | 9.49E−03 | 3.33E−03 | 1.27E−02 | −1 | DKC1 |
| AF113008 | 9.49E−03 | 2.52E−03 | 1.04E−03 | −1 | — |
| AL359578 | 9.49E−03 | 3.33E−03 | 1.21E−03 | −1 | — |
| AL049997 | 9.49E−03 | 8.40E−03 | 4.08E−02 | −1 | — |
| U80771 | 9.49E−03 | 1.56E−02 | 9.34E−03 | −1 | — |
| AF041811 | 9.49E−03 | 1.56E−02 | 1.10E−02 | −1 | NTRK3 |
| AW088547 | 9.49E−03 | 7.52E−03 | 3.59E−02 | −1 | — |
| NM_006717 | 9.49E−03 | 8.40E−03 | 2.15E−03 | −1 | SPIN |
| NM_018145 | 9.49E−03 | 1.56E−02 | 3.83E−03 | −1 | FLJ10579 |
| AA824298 | 9.49E−03 | 7.52E−03 | 3.28E−03 | −1 | FLJ10036 |
| NM_016649 | 9.49E−03 | 1.37E−02 | 2.29E−03 | −1 | C20orf6 |
| NM_018320 | 9.49E−03 | 1.56E−02 | 1.44E−02 | −1 | RNF121 |
| NM_003973 | 9.49E−03 | 8.40E−03 | 4.82E−03 | −1 | — |
| NM_017571 | 9.49E−03 | 8.40E−03 | 3.67E−03 | −1 | KIAA1212 |
| NM_022101 | 9.49E−03 | 1.37E−02 | 1.07E−02 | −1 | FLJ22965 |
| NM_014155 | 9.49E−03 | 7.52E−03 | 2.76E−02 | −1 | HSPC063 |
| NM_024312 | 9.49E−03 | 8.40E−03 | 1.51E−02 | −1 | MGC4170 |
| NM_024966 | 9.49E−03 | 3.33E−03 | 1.18E−03 | −1 | SEMA6D |
| NM_018655 | 9.49E−03 | 7.52E−03 | 1.39E−02 | −1 | LENEP |
| AF077053 | 9.49E−03 | 2.52E−03 | 2.54E−03 | −1 | TAF9L |
| BE972394 | 9.49E−03 | 1.56E−02 | 1.79E−03 | −1 | ZNF131 |
| BF195165 | 9.49E−03 | 1.56E−02 | 8.62E−03 | −1 | — |
| BF224259 | 1.74E−02 | 2.19E−02 | 2.51E−02 | −1 | SMNDC1 |
| BG168896 | 1.74E−02 | 2.72E−02 | 1.16E−02 | −1 | FNTA |
| NM_012248 | 1.74E−02 | 7.75E−03 | 2.21E−03 | −1 | SEPHS2 |
| BF739979 | 1.74E−02 | 2.72E−02 | 1.64E−02 | −1 | FLJ16518 |
| AF053641 | 1.74E−02 | 5.43E−03 | 2.99E−02 | −1 | CSE1L |
| BC002513 | 1.74E−02 | 2.72E−02 | 1.15E−02 | −1 | EIF2S1 |
| NM_001020 | 1.74E−02 | 5.43E−03 | 3.65E−02 | −1 | RPS16 |
| NM_003589 | 1.74E−02 | 2.19E−02 | 2.94E−02 | −1 | CUL4A |
| AL567227 | 1.74E−02 | 2.19E−02 | 1.49E−02 | −1 | TIA1 |
| NM_022037 | 1.74E−02 | 5.43E−03 | 1.66E−03 | −1 | TIA1 |
| NM_002806 | 1.74E−02 | 5.43E−03 | 4.98E−03 | −1 | PSMC6 |
| D42063 | 1.74E−02 | 2.72E−02 | 1.35E−02 | −1 | RANBP2 |
| AF185696 | 1.74E−02 | 2.19E−02 | 2.04E−02 | −1 | OSBP |
| NM_001560 | 1.74E−02 | 2.19E−02 | 4.92E−02 | −1 | IL13RA1 |
| AI984051 | 1.74E−02 | 5.47E−04 | 4.24E−05 | −1 | THRAP1 |
| AA156948 | 1.74E−02 | 2.19E−02 | 7.17E−03 | −1 | PRPF4B |
| NM_016553 | 1.74E−02 | 7.75E−03 | 2.86E−03 | −1 | NUP62 |
| AF302110 | 1.74E−02 | 5.43E−03 | 7.85E−03 | −1 | AASDHPPT |
| NM_003601 | 1.74E−02 | 2.19E−02 | 1.40E−02 | −1 | SMARCA5 |
| NM_006178 | 1.74E−02 | 5.43E−03 | 4.00E−03 | −1 | NSF |
| BC000365 | 1.74E−02 | 2.19E−02 | 8.40E−03 | −1 | GTF2H1 |
| AA834576 | 1.74E−02 | 5.43E−03 | 1.05E−02 | −1 | ITPR2 |
| NM_003903 | 1.74E−02 | 2.19E−02 | 9.52E−03 | −1 | CDC16 |
| NM_006460 | 1.74E−02 | 5.43E−03 | 7.32E−03 | −1 | HIS1 |
| NM_004661 | 1.74E−02 | 2.19E−02 | 4.09E−02 | −1 | CDC23 |
| NM_003850 | 1.74E−02 | 5.43E−03 | 1.17E−02 | −1 | SUCLA2 |
| NM_006421 | 1.74E−02 | 2.72E−02 | 4.86E−02 | −1 | ARFGEF1 |
| NM_000255 | 1.74E−02 | 2.19E−02 | 1.71E−02 | −1 | MUT |
| NM_005783 | 1.74E−02 | 5.43E−03 | 3.12E−02 | −1 | TXNDC9 |
| NM_013450 | 1.74E−02 | 5.43E−03 | 1.03E−02 | −1 | BAZ2B |
| NM_003194 | 1.74E−02 | 2.19E−02 | 3.67E−02 | −1 | TBP |
| NM_014663 | 1.74E−02 | 2.72E−02 | 1.21E−02 | −1 | JMJD2A |
| BG111661 | 1.74E−02 | 2.72E−02 | 4.74E−02 | −1 | GOLGA1 |
| AU157008 | 1.74E−02 | 2.72E−02 | 5.92E−03 | −1 | PSMD5 |
| NM_003674 | 1.74E−02 | 5.43E−03 | 3.21E−02 | −1 | CDK10 |
| NM_025137 | 1.74E−02 | 5.43E−03 | 1.19E−02 | −1 | FLJ21439 |
| NM_021645 | 1.74E−02 | 5.43E−03 | 3.23E−02 | −1 | UTP14C |
| NM_001114 | 1.74E−02 | 5.43E−03 | 3.34E−03 | −1 | ADCY7 |
| NM_005923 | 1.74E−02 | 1.18E−02 | 1.58E−03 | −1 | MAP3K5 |
| NM_002118 | 1.74E−02 | 2.19E−02 | 3.48E−02 | −1 | HLA-DMB |
| NM_007049 | 1.74E−02 | 2.72E−02 | 8.46E−03 | −1 | BTN2A1 |
| NM_005999 | 1.74E−02 | 2.19E−02 | 4.44E−02 | −1 | TSNAX |
| NM_006493 | 1.74E−02 | 2.19E−02 | 2.88E−02 | −1 | CLN5 |
| U16307 | 1.74E−02 | 5.43E−03 | 2.50E−03 | −1 | HRB2 |
| NM_004379 | 1.74E−02 | 2.19E−02 | 1.85E−03 | −1 | CREB1 |
| NM_002048 | 1.74E−02 | 2.19E−02 | 2.16E−02 | −1 | GAS1 |
| AB011092 | 1.74E−02 | 1.18E−02 | 1.68E−03 | −1 | ADCY9 |
| NM_004898 | 1.74E−02 | 2.19E−02 | 9.45E−03 | −1 | CLOCK |
| NM_003631 | 1.74E−02 | 2.43E−02 | 6.33E−03 | −1 | PARG |
| NM_002643 | 1.74E−02 | 2.72E−02 | 4.22E−02 | −1 | PIGF |
| NM_014950 | 1.74E−02 | 2.19E−02 | 9.23E−03 | −1 | ZBTB1 |
| NM_003838 | 1.74E−02 | 5.43E−03 | 1.77E−03 | −1 | FPGT |
| NM_006299 | 1.74E−02 | 5.43E−03 | 4.66E−02 | −1 | ZNF193 |
| AF082283 | 1.74E−02 | 2.19E−02 | 4.89E−02 | −1 | BCL10 |
| NM_007309 | 1.74E−02 | 2.72E−02 | 1.60E−02 | −1 | DIAPH2 |
| NM_020423 | 1.74E−02 | 2.72E−02 | 1.01E−02 | −1 | PACE-1 |
| NM_012135 | 1.74E−02 | 2.19E−02 | 4.11E−03 | −1 | FAM50B |
| NM_006300 | 1.74E−02 | 2.72E−02 | 3.98E−02 | −1 | ZNF230 |
| NM_006588 | 1.74E−02 | 2.19E−02 | 5.41E−03 | −1 | HSGP25L2G |
| NM_003265 | 1.74E−02 | 2.72E−02 | 4.50E−02 | −1 | TLR3 |
| NM_002158 | 1.74E−02 | 2.19E−02 | 1.63E−03 | −1 | HTLF |
| NM_001499 | 1.74E−02 | 2.72E−02 | 1.35E−02 | −1 | GLE1L |
| NM_000647 | 1.74E−02 | 5.47E−04 | 8.05E−04 | −1 | CCR2 |
| NM_004858 | 1.74E−02 | 2.19E−02 | 1.38E−03 | −1 | SLC4A8 |
| NM_015384 | 1.74E−02 | 5.47E−04 | 3.22E−03 | −1 | NIPBL |
| NM_003438 | 1.74E−02 | 5.47E−04 | 4.76E−04 | −1 | ZNF137 |
| NM_024986 | 1.74E−02 | 2.19E−02 | 9.65E−03 | −1 | FLJ12331 |
| NM_014812 | 1.74E−02 | 5.43E−03 | 3.21E−03 | −1 | KAB |
| NM_017522 | 1.74E−02 | 2.19E−02 | 4.19E−02 | −1 | LRP8 |
| NM_004324 | 1.74E−02 | 2.72E−02 | 3.10E−02 | −1 | BAX |
| AK024823 | 1.74E−02 | 2.19E−02 | 4.04E−03 | −1 | SUMO2 |
| AL570661 | 1.74E−02 | 2.72E−02 | 8.23E−03 | −1 | MCP |
| U72937 | 1.74E−02 | 2.19E−02 | 3.01E−02 | −1 | ATRX |
| AW073672 | 1.74E−02 | 5.43E−03 | 3.21E−03 | −1 | CTNND1 |
| BG534245 | 1.74E−02 | 4.17E−03 | 2.31E−03 | −1 | CSNK1A1 |
| AI659005 | 1.74E−02 | 7.75E−03 | 2.60E−03 | −1 | LGALS8 |
| BC005374 | 1.74E−02 | 2.19E−02 | 4.87E−02 | −1 | TXNDC4 |
| AF208043 | 1.74E−02 | 1.18E−02 | 2.83E−04 | −1 | IFI16 |
| BC002719 | 1.74E−02 | 2.72E−02 | 1.91E−02 | −1 | EIF3S1 |
| AF247168 | 1.74E−02 | 2.43E−02 | 3.04E−02 | −1 | NPD014 |
| AF006516 | 1.74E−02 | 2.19E−02 | 5.99E−03 | −1 | ABI1 |
| NM_001253 | 1.74E−02 | 1.18E−02 | 3.54E−03 | −1 | CDC5L |
| BC003600 | 1.74E−02 | 2.19E−02 | 8.59E−03 | −1 | LMO4 |
| AV701283 | 1.74E−02 | 2.19E−02 | 3.30E−03 | −1 | SEC22L1 |
| AI753638 | 1.74E−02 | 2.19E−02 | 2.93E−02 | −1 | OSBPL2 |
| AF165513 | 1.74E−02 | 2.19E−02 | 4.41E−03 | −1 | VPS45A |
| AF008442 | 1.74E−02 | 2.19E−02 | 8.61E−03 | −1 | POLR1C |
| AF112207 | 1.74E−02 | 2.19E−02 | 1.73E−03 | −1 | — |
| BE963245 | 1.74E−02 | 2.19E−02 | 4.82E−03 | −1 | FBXW11 |
| N25915 | 1.74E−02 | 2.72E−02 | 1.41E−02 | −1 | CUGBP1 |
| AK001280 | 1.74E−02 | 2.19E−02 | 2.35E−02 | −1 | HDGFRP3 |
| AL133600 | 1.74E−02 | 5.47E−04 | 1.41E−03 | −1 | STAM2 |
| AF182198 | 1.74E−02 | 2.72E−02 | 8.03E−03 | −1 | ITSN2 |
| AF176699 | 1.74E−02 | 2.72E−02 | 5.37E−03 | −1 | FBXL4 |
| U22815 | 1.74E−02 | 2.19E−02 | 1.20E−02 | −1 | PPFIA1 |
| BC001265 | 1.74E−02 | 2.19E−02 | 3.12E−03 | −1 | DJ462O23.2 |
| U89358 | 1.74E−02 | 2.19E−02 | 3.57E−02 | −1 | L3MBTL |
| AB034951 | 1.74E−02 | 2.72E−02 | 4.79E−02 | −1 | HSPA8 |
| BC002635 | 1.74E−02 | 2.19E−02 | 4.32E−02 | −1 | CSF2RA |
| AF274935 | 1.74E−02 | 2.19E−02 | 9.75E−03 | −1 | LOC54499 |
| BC005259 | 1.74E−02 | 2.19E−02 | 8.41E−03 | −1 | XRCC4 |
| AB002382 | 1.74E−02 | 2.19E−02 | 4.24E−03 | −1 | CTNND1 |
| BC006181 | 1.74E−02 | 2.19E−02 | 1.59E−02 | −1 | SFRS1 |
| AI359472 | 1.74E−02 | 2.19E−02 | 9.72E−03 | −1 | XTP2 |
| M27487 | 1.74E−02 | 2.72E−02 | 3.95E−02 | −1 | HLA-DPA1 |
| AU143855 | 1.74E−02 | 2.72E−02 | 2.08E−02 | −1 | PSME4 |
| BG111260 | 1.74E−02 | 2.19E−02 | 3.20E−03 | −1 | HIPK1 |
| AK022910 | 1.74E−02 | 5.43E−03 | 4.83E−04 | −1 | TNPO3 |
| AA195936 | 1.74E−02 | 5.43E−03 | 2.79E−02 | −1 | MGC21416 |
| AV715767 | 1.74E−02 | 2.19E−02 | 1.14E−02 | −1 | LIM |
| AV745949 | 1.74E−02 | 2.72E−02 | 2.35E−02 | −1 | SCAMP1 |
| D26069 | 1.74E−02 | 2.72E−02 | 1.34E−02 | −1 | CENTB2 |
| AL080111 | 1.74E−02 | 2.19E−02 | 1.71E−02 | −1 | NEK7 |
| AL562282 | 1.74E−02 | 2.19E−02 | 4.97E−02 | −1 | PP591 |
| BF970829 | 1.74E−02 | 2.19E−02 | 5.55E−03 | −1 | OSBPL8 |
| AI991252 | 1.74E−02 | 2.19E−02 | 2.36E−02 | −1 | BTN3A2 |
| AI742305 | 1.74E−02 | 4.17E−03 | 9.52E−05 | −1 | CHD9 |
| AA524345 | 1.74E−02 | 2.19E−02 | 1.02E−02 | −1 | SNX4 |
| AI752257 | 1.74E−02 | 2.72E−02 | 5.98E−03 | −1 | ZNF3 |
| BG548738 | 1.74E−02 | 2.72E−02 | 1.08E−02 | −1 | KIAA1040 |
| AB020684 | 1.74E−02 | 2.19E−02 | 1.15E−02 | −1 | KIAA0877 |
| AI040324 | 1.74E−02 | 2.19E−02 | 2.72E−03 | −1 | NCOA2 |
| AA284075 | 1.74E−02 | 2.72E−02 | 3.24E−03 | −1 | KNS2 |
| BE786164 | 1.74E−02 | 7.75E−03 | 2.14E−03 | −1 | — |
| AP000693 | 1.74E−02 | 1.18E−02 | 2.00E−03 | −1 | ZCWCC3 |
| N22548 | 1.74E−02 | 2.19E−02 | 1.10E−03 | −1 | — |
| BF970253 | 1.74E−02 | 2.19E−02 | 2.64E−02 | −1 | FLJ11806 |
| AV682436 | 1.74E−02 | 5.43E−03 | 1.97E−03 | −1 | PIK3C2A |
| Z78330 | 1.74E−02 | 5.43E−03 | 5.22E−03 | −1 | ACTR3 |
| AI041204 | 1.74E−02 | 5.47E−04 | 3.79E−03 | −1 | CAP350 |
| AL162056 | 1.74E−02 | 2.19E−02 | 4.90E−02 | −1 | KIAA1117 |
| AI823592 | 1.74E−02 | 1.18E−02 | 3.18E−03 | −1 | KIAA0423 |
| AI989567 | 1.74E−02 | 2.19E−02 | 3.94E−02 | −1 | SIAT10 |
| BF223370 | 1.74E−02 | 2.72E−02 | 2.52E−02 | −1 | MGC11332 |
| AW190088 | 1.74E−02 | 2.72E−02 | 3.63E−02 | −1 | ZNF307 |
| AI655015 | 1.74E−02 | 2.72E−02 | 1.05E−02 | −1 | DUSP7 |
| AA053830 | 1.74E−02 | 2.72E−02 | 3.29E−02 | −1 | CTBP1 |
| BE501352 | 1.74E−02 | 2.72E−02 | 9.70E−03 | −1 | DKFZp667G2110 |
| AB011097 | 1.74E−02 | 2.19E−02 | 9.50E−03 | −1 | ARTS-1 |
| M19720 | 1.74E−02 | 4.17E−03 | 1.92E−03 | −1 | — |
| AI912583 | 1.74E−02 | 4.17E−03 | 2.31E−03 | −1 | HRB2 |
| AA890010 | 1.74E−02 | 2.19E−02 | 1.46E−02 | −1 | — |
| AI376724 | 1.74E−02 | 5.43E−03 | 5.94E−04 | −1 | COX11 |
| AA209332 | 1.74E−02 | 2.19E−02 | 2.04E−02 | −1 | OPA1 |
| N58120 | 1.74E−02 | 2.19E−02 | 6.36E−03 | −1 | — |
| M34356 | 1.74E−02 | 2.19E−02 | 4.08E−02 | −1 | CREB1 |
| AK026142 | 1.74E−02 | 5.43E−03 | 4.71E−04 | −1 | ODAG |
| AI357539 | 1.74E−02 | 4.17E−03 | 1.80E−03 | −1 | LRCH3 |
| AI571996 | 1.74E−02 | 2.19E−02 | 3.71E−02 | −1 | STAM2 |
| AA830884 | 1.74E−02 | 2.19E−02 | 9.07E−03 | −1 | FMR1 |
| AA573862 | 1.74E−02 | 2.72E−02 | 2.11E−02 | −1 | HLA-A |
| AL121936 | 1.74E−02 | 7.75E−03 | 2.66E−03 | −1 | BTN2A1 |
| AC005614 | 1.74E−02 | 2.43E−02 | 2.52E−03 | −1 | LOC163131 /// |
| | | | | | LOC284323 |
| AL163248 | 1.74E−02 | 2.19E−02 | 1.33E−02 | −1 | ZNF294 |
| AK024456 | 1.74E−02 | 5.47E−04 | 5.10E−03 | −1 | FGD2 |
| AK001861 | 1.74E−02 | 4.17E−03 | 5.47E−04 | −1 | SNX13 |
| AK025663 | 1.74E−02 | 5.43E−03 | 1.00E−02 | −1 | ZNF291 |
| AK024606 | 1.74E−02 | 5.43E−03 | 1.73E−03 | −1 | — |
| AK025097 | 1.74E−02 | 2.19E−02 | 4.60E−02 | −1 | — |
| AF035299 | 1.74E−02 | 5.47E−04 | 5.00E−03 | −1 | DOK1 |
| X69383 | 1.74E−02 | 2.43E−02 | 1.30E−02 | −1 | — |
| S67289 | 1.74E−02 | 5.43E−03 | 3.46E−03 | −1 | CYBB |
| AI339732 | 1.74E−02 | 1.21E−03 | 1.55E−03 | −1 | CIAO1 |
| AA719797 | 1.74E−02 | 2.19E−02 | 4.46E−02 | −1 | OR7E18P |
| NM_014052 | 1.74E−02 | 2.19E−02 | 1.90E−03 | −1 | YWHAB |
| NM_014426 | 1.74E−02 | 2.72E−02 | 6.25E−03 | −1 | SNX5 |
| NM_018184 | 1.74E−02 | 4.17E−03 | 7.32E−03 | −1 | ARL10C |
| NM_016166 | 1.74E−02 | 2.19E−02 | 1.59E−02 | −1 | PIAS1 |
| NM_018442 | 1.74E−02 | 2.19E−02 | 2.87E−02 | −1 | IQWD1 |
| NM_018244 | 1.74E−02 | 2.19E−02 | 4.15E−02 | −1 | C20orf44 |
| NM_015153 | 1.74E−02 | 2.19E−02 | 1.15E−02 | −1 | PHF3 |
| NM_015961 | 1.74E−02 | 2.72E−02 | 3.91E−03 | −1 | SNF7DC2 |
| NM_018361 | 1.74E−02 | 2.19E−02 | 1.76E−02 | −1 | LPAAT-e |
| BC005316 | 1.74E−02 | 2.72E−02 | 4.18E−02 | −1 | NFYB |
| NM_018229 | 1.74E−02 | 2.19E−02 | 1.59E−02 | −1 | C14orf108 |
| AF195514 | 1.74E−02 | 5.47E−04 | 9.81E−04 | −1 | VPS4B |
| NM_024573 | 1.74E−02 | 5.43E−03 | 3.35E−03 | −1 | C6orf211 |
| NM_014028 | 1.74E−02 | 2.19E−02 | 4.90E−02 | −1 | OSTM1 |
| NM_001668 | 1.74E−02 | 2.19E−02 | 3.53E−02 | −1 | ARNT |
| NM_022494 | 1.74E−02 | 2.19E−02 | 9.09E−03 | −1 | ZDHHC6 |
| NM_022776 | 1.74E−02 | 5.47E−04 | 8.77E−04 | −1 | OSBPL11 |
| NM_022067 | 1.74E−02 | 2.19E−02 | 1.91E−02 | −1 | C14orf133 |
| NM_020640 | 1.74E−02 | 2.19E−02 | 1.07E−02 | −1 | RP42 |
| NM_018115 | 1.74E−02 | 1.18E−02 | 8.56E−04 | −1 | SDAD1 |
| NM_017646 | 1.74E−02 | 2.19E−02 | 9.56E−04 | −1 | TRIT1 |
| NM_003929 | 1.74E−02 | 2.72E−02 | 2.23E−02 | −1 | RAB7L1 |
| NM_017850 | 1.74E−02 | 7.75E−03 | 1.01E−03 | −1 | FLJ20508 |
| NM_012123 | 1.74E−02 | 2.72E−02 | 2.21E−02 | −1 | MTO1 |
| AK024569 | 1.74E−02 | 5.43E−03 | 1.05E−02 | −1 | HSPC163 |
| NM_023010 | 1.74E−02 | 2.19E−02 | 2.12E−03 | −1 | UPF3B |
| NM_025080 | 1.74E−02 | 5.43E−03 | 5.58E−03 | −1 | ASRGL1 |
| NM_017810 | 1.74E−02 | 2.72E−02 | 1.59E−02 | −1 | ZNF434 |
| NM_018569 | 1.74E−02 | 2.19E−02 | 8.22E−03 | −1 | C4orf16 |
| NM_019083 | 1.74E−02 | 2.72E−02 | 2.38E−02 | −1 | FLJ10287 |
| AF119868 | 1.74E−02 | 5.43E−03 | 1.89E−02 | −1 | KIAA1922 |
| NM_016020 | 1.74E−02 | 2.19E−02 | 1.58E−02 | −1 | TFB1M |
| NM_024638 | 1.74E−02 | 1.21E−03 | 1.92E−03 | −1 | QTRTD1 |
| NM_022168 | 1.74E−02 | 2.72E−02 | 9.45E−03 | −1 | IFIH1 |
| NM_024828 | 1.74E−02 | 2.19E−02 | 1.58E−03 | −1 | C9orf82 |
| NM_024546 | 1.74E−02 | 2.19E−02 | 1.30E−02 | −1 | C13orf7 |
| NM_017912 | 1.74E−02 | 2.72E−02 | 4.68E−02 | −1 | HERC6 |
| NM_017687 | 1.74E−02 | 2.19E−02 | 2.54E−03 | −1 | NHLRC2 |
| NM_024862 | 1.74E−02 | 7.75E−03 | 4.33E−03 | −1 | FBXO38 |
| NM_025027 | 1.74E−02 | 2.72E−02 | 3.08E−02 | −1 | ZNF606 |
| NM_025231 | 1.74E−02 | 4.17E−03 | 1.49E−03 | −1 | ZNF435 |
| NM_022147 | 1.74E−02 | 2.19E−02 | 3.26E−02 | −1 | IFRG28 |
| NM_004923 | 1.74E−02 | 2.72E−02 | 1.87E−02 | −1 | MTL5 |
| NM_024838 | 1.74E−02 | 5.43E−03 | 1.90E−02 | −1 | — |
| NM_024833 | 1.74E−02 | 2.19E−02 | 3.46E−02 | −1 | FLJ23506 |
| NM_007210 | 1.74E−02 | 1.21E−03 | 2.38E−03 | −1 | GALNT6 |
| NM_005774 | 1.74E−02 | 2.43E−02 | 2.15E−02 | −1 | ZNF224 |
| NM_016424 | 1.74E−02 | 2.19E−02 | 4.40E−03 | −1 | CROP |
| NM_018372 | 1.74E−02 | 2.72E−02 | 3.91E−02 | −1 | RIF1 |
| NM_013240 | 1.74E−02 | 2.19E−02 | 2.64E−03 | −1 | C21orf127 |
| NM_018327 | 1.74E−02 | 5.43E−03 | 2.08E−02 | −1 | C20orf38 |
| NM_015510 | 1.74E−02 | 2.72E−02 | 1.83E−02 | −1 | DKFZp566O084 |
| NM_018976 | 1.74E−02 | 5.43E−03 | 8.66E−03 | −1 | SLC38A2 |
| NM_030917 | 1.74E−02 | 2.19E−02 | 1.22E−02 | −1 | FIP1L1 |
| NM_030911 | 1.74E−02 | 1.21E−03 | 4.68E−04 | −1 | CDADC1 |
| NM_030963 | 1.74E−02 | 5.43E−03 | 1.52E−02 | −1 | RNF146 |
| AL120704 | 1.74E−02 | 2.19E−02 | 3.01E−02 | −1 | KPNA3 |
| BC000039 | 1.74E−02 | 1.21E−03 | 6.70E−04 | −1 | FAM26B |
| AF257135 | 1.74E−02 | 2.19E−02 | 3.46E−02 | −1 | WBSCR5 |
| AF274950 | 1.74E−02 | 5.43E−03 | 4.34E−02 | −1 | FLJ10637 |
| BC003073 | 1.74E−02 | 5.43E−03 | 1.21E−03 | −1 | FLJ10521 |
| AL565516 | 1.74E−02 | 2.19E−02 | 2.11E−02 | −1 | PANK3 |
| AI433464 | 1.74E−02 | 2.19E−02 | 3.87E−02 | −1 | PGM3 |
| NM_004251 | 1.74E−02 | 2.19E−02 | 3.14E−02 | −1 | RAB9A |
| AW162015 | 1.74E−02 | 2.72E−02 | 6.39E−03 | −1 | ZNF143 |
| AW002578 | 1.74E−02 | 2.19E−02 | 3.05E−02 | −1 | MGC3731 |
| AI983115 | 1.74E−02 | 2.72E−02 | 9.07E−03 | −1 | IL27RA |
| AW008921 | 1.74E−02 | 2.19E−02 | 3.59E−02 | −1 | SENP5 |
| AL137398 | 1.74E−02 | 2.72E−02 | 2.16E−03 | −1 | DKFZp434P162 |
| R43279 | 1.74E−02 | 4.17E−03 | 1.74E−03 | −1 | SNAPC3 |
| AW975638 | 1.74E−02 | 2.19E−02 | 4.47E−02 | −1 | — |
| AF070618 | 1.74E−02 | 2.19E−02 | 1.09E−02 | −1 | MRPS22 |
| U07139 | 1.74E−02 | 2.19E−02 | 4.38E−02 | −1 | CACNB3 |
| D29642 | 1.74E−02 | 5.47E−04 | 1.84E−02 | −1 | ARHGAP25 |
| AI458463 | 1.74E−02 | 5.43E−03 | 9.08E−03 | −1 | PACE-1 |
| Z48481 | 3.95E−02 | 2.49E−02 | 2.75E−02 | −1 | MMP14 |
| AV702810 | 3.95E−02 | 2.49E−02 | 6.09E−03 | −1 | SET |
| NM_016451 | 3.95E−02 | 4.88E−03 | 4.12E−03 | −1 | COPB |
| AF205218 | 3.95E−02 | 2.52E−03 | 1.02E−03 | −1 | IVNS1ABP |
| AW968555 | 3.95E−02 | 3.54E−02 | 4.32E−02 | −1 | TBL1X |
| NM_007214 | 3.95E−02 | 3.54E−02 | 3.59E−03 | −1 | SEC63 |
| NM_006838 | 3.95E−02 | 4.91E−02 | 1.87E−02 | −1 | — |
| BC000603 | 3.95E−02 | 3.54E−02 | 3.10E−03 | −1 | — |
| BE250417 | 3.95E−02 | 4.91E−02 | 9.76E−03 | −1 | ZMYND11 |
| AW183478 | 3.95E−02 | 9.26E−03 | 1.65E−02 | −1 | STK17A |
| NM_015641 | 3.95E−02 | 2.49E−02 | 1.28E−02 | −1 | TES |
| NM_014872 | 3.95E−02 | 4.91E−02 | 2.61E−02 | −1 | ZBTB5 |
| NM_014753 | 3.95E−02 | 4.91E−02 | 2.64E−02 | −1 | BMS1L |
| NM_001482 | 3.95E−02 | 9.26E−03 | 7.96E−03 | −1 | GATM |
| NM_000702 | 3.95E−02 | 2.49E−02 | 5.02E−03 | −1 | ATP1A2 |
| NM_006214 | 3.95E−02 | 3.54E−02 | 3.50E−02 | −1 | PHYH |
| BG034328 | 3.95E−02 | 4.88E−03 | 5.06E−03 | −1 | TFDP2 |
| BG252490 | 3.95E−02 | 4.91E−02 | 2.36E−02 | −1 | DNAJB4 |
| NM_016024 | 3.95E−02 | 4.91E−02 | 3.39E−02 | −1 | RBMX2 |
| NM_001353 | 3.95E−02 | 2.32E−02 | 6.14E−03 | −1 | AKR1C1 |
| NM_000428 | 3.95E−02 | 2.49E−02 | 1.45E−02 | −1 | LTBP2 |
| BF224146 | 3.95E−02 | 4.91E−02 | 3.64E−03 | −1 | TMEM5 |
| NM_002830 | 3.95E−02 | 3.54E−02 | 2.79E−02 | −1 | PTPN4 |
| NM_021077 | 3.95E−02 | 4.91E−02 | 4.77E−02 | −1 | NMB |
| NM_004944 | 3.95E−02 | 1.23E−02 | 3.94E−03 | −1 | DNASE1L3 |
| NM_016352 | 3.95E−02 | 9.26E−03 | 1.01E−02 | −1 | CPA4 |
| NM_003151 | 3.95E−02 | 3.54E−02 | 1.24E−02 | −1 | STAT4 |
| NM_002649 | 3.95E−02 | 3.54E−02 | 2.51E−02 | −1 | PIK3CG |
| NM_024506 | 3.95E−02 | 4.91E−02 | 1.05E−02 | −1 | GLB1L |
| NM_016436 | 3.95E−02 | 1.23E−02 | 2.13E−03 | −1 | C20orf104 |
| NM_001240 | 3.95E−02 | 2.32E−02 | 5.79E−03 | −1 | CCNT1 |
| NM_006610 | 3.95E−02 | 2.32E−02 | 3.17E−02 | −1 | MASP2 |
| NM_002385 | 3.95E−02 | 3.54E−02 | 4.24E−02 | −1 | MBP |
| BG494940 | 3.95E−02 | 4.91E−02 | 1.96E−02 | −1 | SSA2 |
| NM_014257 | 3.95E−02 | 1.75E−02 | 6.26E−03 | −1 | CD209L |
| NM_007068 | 3.95E−02 | 3.54E−02 | 3.77E−02 | −1 | DMC1 |
| AF208850 | 3.95E−02 | 4.91E−02 | 1.60E−02 | −1 | PTP4A2 |
| AF116710 | 3.95E−02 | 1.75E−02 | 9.09E−03 | −1 | RPS14 |
| BC001019 | 3.95E−02 | 1.23E−02 | 1.21E−02 | −1 | RPL39 |
| BC000734 | 3.95E−02 | 4.58E−02 | 1.57E−02 | −1 | EIF3S6 |
| AF226044 | 3.95E−02 | 4.91E−02 | 4.07E−02 | −1 | SNRK |
| AY008372 | 3.95E−02 | 3.54E−02 | 2.90E−02 | −1 | OSBPL3 |
| AF271775 | 3.95E−02 | 3.54E−02 | 5.26E−03 | −1 | LAT1-3TM |
| U79526 | 3.95E−02 | 3.54E−02 | 2.21E−02 | −1 | CMKLR1 |
| AF031137 | 3.95E−02 | 3.54E−02 | 9.11E−03 | −1 | NCR3 |
| AB006589 | 3.95E−02 | 2.49E−02 | 3.80E−02 | −1 | ESR2 |
| AF000381 | 3.95E−02 | 4.58E−02 | 9.92E−03 | −1 | — |
| BE466128 | 3.95E−02 | 3.54E−02 | 2.57E−02 | −1 | RBM25 |
| AI631874 | 3.95E−02 | 3.54E−02 | 7.58E−03 | −1 | CSNK2A1 |
| BG403834 | 3.95E−02 | 2.49E−02 | 2.54E−02 | −1 | KPNA6 |
| AW593213 | 3.95E−02 | 1.23E−02 | 1.83E−03 | −1 | KIAA1078 |
| BF214492 | 3.95E−02 | 7.66E−04 | 3.51E−03 | −1 | RPL5 |
| AA521269 | 3.95E−02 | 4.58E−02 | 1.11E−02 | −1 | — |
| BF739959 | 3.95E−02 | 1.23E−02 | 1.31E−02 | −1 | MFHAS1 |
| AW302047 | 3.95E−02 | 4.91E−02 | 6.80E−03 | −1 | — |
| AI252582 | 3.95E−02 | 2.49E−02 | 1.10E−03 | −1 | — |
| AI539361 | 3.95E−02 | 1.23E−02 | 1.39E−02 | −1 | — |
| NM_012081 | 3.95E−02 | 4.58E−02 | 1.28E−02 | −1 | ELL2 |
| AA400421 | 3.95E−02 | 4.91E−02 | 2.82E−02 | −1 | TWISTNB |
| AK023851 | 3.95E−02 | 4.88E−03 | 1.60E−03 | −1 | CAPN2 |
| BE000837 | 3.95E−02 | 1.23E−02 | 1.96E−03 | −1 | KIAA0220 |
| AL121891 | 3.95E−02 | 3.54E−02 | 3.37E−03 | −1 | UBCE7IP5 |
| AK023621 | 3.95E−02 | 9.26E−03 | 1.74E−02 | −1 | RHOBTB3 |
| S69182 | 3.95E−02 | 4.88E−03 | 3.02E−03 | −1 | — |
| AK026521 | 3.95E−02 | 3.54E−02 | 6.64E−03 | −1 | TAF1B |
| AL354872 | 3.95E−02 | 3.54E−02 | 4.77E−02 | −1 | CTH |
| NM_022731 | 3.95E−02 | 4.88E−03 | 4.52E−03 | −1 | NUCKS |
| NM_020239 | 3.95E−02 | 3.54E−02 | 1.25E−02 | −1 | CDC42SE1 |
| NM_022365 | 3.95E−02 | 3.54E−02 | 2.20E−02 | −1 | DNAJC1 |
| AV682567 | 3.95E−02 | 3.54E−02 | 4.39E−02 | −1 | MDS010 |
| NM_024779 | 3.95E−02 | 2.49E−02 | 2.19E−02 | −1 | PIP5K2C |
| NM_016052 | 3.95E−02 | 4.88E−03 | 2.26E−03 | −1 | CGI-115 |
| NM_022073 | 3.95E−02 | 4.91E−02 | 4.41E−02 | −1 | EGLN3 |
| NM_013281 | 3.95E−02 | 2.49E−02 | 3.12E−02 | −1 | FLRT3 |
| NM_016122 | 3.95E−02 | 3.54E−02 | 9.15E−03 | −1 | NY-REN-58 |
| NM_024084 | 3.95E−02 | 2.49E−02 | 8.27E−03 | −1 | MGC3196 |
| NM_017860 | 3.95E−02 | 3.54E−02 | 3.82E−03 | −1 | FLJ20519 |
| NM_023034 | 3.95E−02 | 2.32E−02 | 1.26E−02 | −1 | WHSC1L1 |
| NM_002548 | 3.95E−02 | 2.49E−02 | 2.42E−02 | −1 | OR1D2 |
| AL136733 | 3.95E−02 | 4.91E−02 | 1.29E−02 | −1 | UBAP1 |
| BC005934 | 3.95E−02 | 2.32E−02 | 1.83E−03 | −1 | FLJ21168 |
| AW303136 | 3.95E−02 | 4.88E−03 | 2.28E−03 | −1 | — |
| W87634 | 3.95E−02 | 2.49E−02 | 6.65E−03 | −1 | CXorf33 |
| AI669379 | 3.95E−02 | 4.91E−02 | 1.28E−02 | −1 | — |
| BG025063 | 3.95E−02 | 3.54E−02 | 3.63E−02 | −1 | — |
| NM_014622 | 1.49E−04 | 1.49E−04 | 2.75E−03 | 1 | LOH11CR2A |
| M87268 | 1.49E−04 | 1.49E−04 | 1.71E−03 | 1 | IGHG1 |
| NM_024588 | 1.49E−04 | 1.49E−04 | 4.18E−03 | 1 | FLJ23584 |
| NM_030926 | 1.49E−04 | 3.72E−05 | 2.14E−04 | 1 | ITM2C |
| NM_021173 | 2.70E−04 | 2.34E−04 | 1.25E−04 | 1 | POLD4 |
| NM_014403 | 2.70E−04 | 4.40E−04 | 1.20E−04 | 1 | SIAT7D |
| NM_004930 | 1.82E−03 | 9.10E−04 | 8.75E−04 | 1 | CAPZB |
| AF129756 | 1.82E−03 | 2.96E−03 | 1.39E−02 | 1 | BAT2 |
| AL096733 | 1.82E−03 | 2.96E−03 | 2.18E−03 | 1 | ATP6V0A1 |
| W74620 | 1.82E−03 | 1.47E−03 | 8.07E−04 | 1 | HNRPD |
| AC002544 | 1.82E−03 | 1.79E−03 | 2.14E−02 | 1 | LOC283970 |
| AF008937 | 1.82E−03 | 8.95E−05 | 4.76E−06 | 1 | STX16 |
| NM_006284 | 2.01E−03 | 2.88E−03 | 3.75E−02 | 1 | TAF10 |
| BF969352 | 2.01E−03 | 2.88E−03 | 6.33E−04 | 1 | ECE1 |
| AA675892 | 2.01E−03 | 1.69E−03 | 9.55E−04 | 1 | TOB1 |
| NM_004395 | 2.01E−03 | 2.88E−03 | 1.56E−02 | 1 | DBN1 |
| NM_000558 | 2.01E−03 | 2.88E−03 | 1.18E−03 | 1 | HBA1 /// HBA2 |
| NM_002412 | 2.01E−03 | 2.88E−03 | 2.62E−03 | 1 | MGMT |
| NM_003955 | 2.01E−03 | 1.69E−03 | 3.20E−02 | 1 | SOCS3 |
| NM_004089 | 2.01E−03 | 1.69E−03 | 4.98E−04 | 1 | DSIPI |
| AF022231 | 2.01E−03 | 2.88E−03 | 2.04E−03 | 1 | CTDSP2 |
| AL110191 | 2.01E−03 | 1.69E−03 | 4.57E−04 | 1 | DSIPI |
| M25079 | 2.01E−03 | 2.88E−03 | 1.68E−03 | 1 | HBB |
| L01639 | 2.01E−03 | 2.88E−03 | 5.25E−03 | 1 | CXCR4 |
| AF105974 | 2.01E−03 | 2.88E−03 | 1.36E−03 | 1 | HBA1 /// HBA2 |
| L07555 | 2.01E−03 | 2.88E−03 | 9.46E−03 | 1 | CD69 |
| AF167343 | 2.01E−03 | 2.88E−03 | 1.04E−02 | 1 | IL1RAP |
| AF116676 | 2.01E−03 | 2.88E−03 | 1.99E−02 | 1 | MYL4 |
| AF349114 | 2.01E−03 | 2.88E−03 | 1.56E−03 | 1 | HBB |
| AF349571 | 2.01E−03 | 2.88E−03 | 1.59E−03 | 1 | HBA1 /// HBA2 |
| BC005931 | 2.01E−03 | 2.88E−03 | 1.33E−03 | 1 | HBA1 |
| AJ225092 | 2.01E−03 | 2.88E−03 | 5.25E−03 | 1 | IGHG1 |
| AF348491 | 2.01E−03 | 2.88E−03 | 7.22E−03 | 1 | CXCR4 |
| T50399 | 2.01E−03 | 2.88E−03 | 1.29E−03 | 1 | HBA2 |
| AJ275355 | 2.01E−03 | 1.21E−03 | 5.42E−04 | 1 | MGC27165 |
| AJ249377 | 2.01E−03 | 2.88E−03 | 5.89E−03 | 1 | IGLJ3 |
| AF059180 | 2.01E−03 | 2.88E−03 | 1.99E−03 | 1 | HBB |
| AJ275374 | 2.01E−03 | 1.69E−03 | 3.02E−03 | 1 | — |
| V00489 | 2.01E−03 | 2.88E−03 | 1.63E−03 | 1 | HBA2 |
| NM_024299 | 2.01E−03 | 1.69E−03 | 1.98E−02 | 1 | C20orf149 |
| NM_020360 | 2.01E−03 | 1.69E−03 | 3.92E−03 | 1 | PLSCR3 |
| NM_017679 | 2.01E−03 | 1.21E−03 | 1.93E−04 | 1 | BCAS3 |
| AF230924 | 2.01E−03 | 2.88E−03 | 6.68E−03 | 1 | C6orf82 |
| AA523441 | 2.01E−03 | 2.88E−03 | 6.97E−04 | 1 | PEX16 |
| NM_005186 | 9.49E−03 | 1.56E−02 | 1.12E−02 | 1 | CAPN1 |
| BF304759 | 9.49E−03 | 7.52E−03 | 3.75E−02 | 1 | LRP1 |
| NM_001662 | 9.49E−03 | 8.40E−03 | 4.40E−03 | 1 | ARF5 |
| AF113019 | 9.49E−03 | 7.52E−03 | 3.36E−03 | 1 | SMARCD2 |
| NM_003025 | 9.49E−03 | 7.52E−03 | 2.68E−02 | 1 | SH3GL1 |
| NM_014846 | 9.49E−03 | 1.37E−02 | 4.68E−02 | 1 | KIAA0196 |
| NM_017458 | 9.49E−03 | 1.23E−02 | 2.56E−03 | 1 | MVP |
| NM_006243 | 9.49E−03 | 1.56E−02 | 4.40E−02 | 1 | PPP2R5A |
| NM_001985 | 9.49E−03 | 1.56E−02 | 3.40E−02 | 1 | ETFB |
| NM_016621 | 9.49E−03 | 1.37E−02 | 3.12E−02 | 1 | BHC80 |
| NM_004565 | 9.49E−03 | 1.23E−02 | 1.37E−03 | 1 | PEX14 |
| NM_005509 | 9.49E−03 | 1.56E−02 | 1.43E−02 | 1 | DMXL1 |
| NM_005224 | 9.49E−03 | 9.26E−03 | 8.52E−03 | 1 | ARID3A |
| NM_003070 | 9.49E−03 | 1.56E−02 | 1.21E−02 | 1 | SMARCA2 |
| AI810484 | 9.49E−03 | 3.63E−04 | 4.25E−03 | 1 | CBFA2T2 |
| AF226990 | 9.49E−03 | 1.37E−02 | 2.39E−02 | 1 | HLA-G |
| U78774 | 9.49E−03 | 7.52E−03 | 1.03E−02 | 1 | NFYC |
| U71088 | 9.49E−03 | 3.63E−04 | 1.69E−03 | 1 | MAP2K5 |
| AA085748 | 9.49E−03 | 1.56E−02 | 1.69E−02 | 1 | LOC149603 |
| AB011126 | 9.49E−03 | 8.40E−03 | 2.72E−03 | 1 | FNBP1 |
| BF570210 | 9.49E−03 | 1.37E−02 | 4.19E−03 | 1 | PNPLA2 |
| AI343248 | 9.49E−03 | 1.37E−02 | 7.03E−03 | 1 | SRP46 |
| AA126728 | 9.49E−03 | 7.52E−03 | 2.86E−02 | 1 | ICAM2 |
| N36926 | 9.49E−03 | 8.40E−03 | 1.13E−02 | 1 | GNA11 |
| AA602532 | 9.49E−03 | 8.40E−03 | 1.41E−02 | 1 | CLN2 |
| BE898639 | 9.49E−03 | 8.40E−03 | 1.18E−02 | 1 | ADD1 |
| AV724215 | 9.49E−03 | 1.56E−02 | 2.07E−02 | 1 | — |
| AK001574 | 9.49E−03 | 1.37E−02 | 3.28E−03 | 1 | GORASP1 |
| BF034906 | 9.49E−03 | 8.40E−03 | 2.75E−03 | 1 | PL6 |
| AL121981 | 9.49E−03 | 3.33E−03 | 1.69E−03 | 1 | DLG1 |
| NM_024531 | 9.49E−03 | 7.52E−03 | 1.12E−02 | 1 | GPR172A |
| NM_016274 | 9.49E−03 | 8.40E−03 | 1.75E−03 | 1 | CKIP-1 |
| NM_004542 | 9.49E−03 | 7.52E−03 | 1.25E−02 | 1 | NDUFA3 |
| NM_007254 | 9.49E−03 | 1.37E−02 | 4.31E−03 | 1 | PNKP |
| NM_015711 | 9.49E−03 | 1.37E−02 | 1.96E−03 | 1 | GLTSCR1 |
| NM_022350 | 9.49E−03 | 1.56E−02 | 4.57E−02 | 1 | LRAP |
| NM_017915 | 9.49E−03 | 1.56E−02 | 3.46E−02 | 1 | FLJ20641 |
| AK026970 | 9.49E−03 | 7.52E−03 | 1.53E−03 | 1 | STX16 |
| AF078847 | 9.49E−03 | 1.37E−02 | 3.27E−02 | 1 | GTF2H2 |
| AI828531 | 9.49E−03 | 1.56E−02 | 1.01E−02 | 1 | WIZ |
| NM_001344 | 1.74E−02 | 2.19E−02 | 2.80E−02 | 1 | DAD1 |
| NM_006184 | 1.74E−02 | 2.19E−02 | 3.03E−03 | 1 | NUCB1 |
| NM_002743 | 1.74E−02 | 2.19E−02 | 8.32E−03 | 1 | PRKCSH |
| NM_007245 | 1.74E−02 | 2.43E−02 | 3.73E−03 | 1 | ATXN2L |
| NM_015853 | 1.74E−02 | 2.19E−02 | 7.03E−03 | 1 | LOC51035 |
| NM_003367 | 1.74E−02 | 2.72E−02 | 3.92E−02 | 1 | USF2 |
| NM_025195 | 1.74E−02 | 2.19E−02 | 4.92E−03 | 1 | TRIB1 |
| BE675849 | 1.74E−02 | 2.19E−02 | 4.29E−02 | 1 | C9orf60 |
| BC000436 | 1.74E−02 | 2.19E−02 | 1.14E−02 | 1 | ENSA |
| NM_006732 | 1.74E−02 | 2.19E−02 | 1.84E−02 | 1 | FOSB |
| NM_019058 | 1.74E−02 | 2.72E−02 | 1.70E−02 | 1 | DDIT4 |
| NM_020248 | 1.74E−02 | 2.19E−02 | 2.01E−02 | 1 | CTNNBIP1 |
| NM_003364 | 1.74E−02 | 2.72E−02 | 3.03E−02 | 1 | UPP1 |
| NM_016294 | 1.74E−02 | 2.19E−02 | 9.73E−03 | 1 | PPP6C |
| NM_014569 | 1.74E−02 | 2.19E−02 | 1.31E−02 | 1 | ZFP95 |
| NM_005354 | 1.74E−02 | 2.72E−02 | 3.91E−02 | 1 | JUND |
| NM_004994 | 1.74E−02 | 2.72E−02 | 3.80E−03 | 1 | MMP9 |
| NM_021127 | 1.74E−02 | 2.19E−02 | 4.47E−02 | 1 | PMAIP1 |
| NM_002460 | 1.74E−02 | 2.19E−02 | 4.63E−02 | 1 | IRF4 |
| NM_002201 | 1.74E−02 | 2.72E−02 | 3.88E−03 | 1 | ISG20 |
| NM_004073 | 1.74E−02 | 2.19E−02 | 1.47E−02 | 1 | PLK3 |
| NM_005738 | 1.74E−02 | 2.19E−02 | 4.15E−02 | 1 | ARL4A |
| NM_014716 | 1.74E−02 | 2.19E−02 | 1.32E−02 | 1 | CENTB1 |
| BG035761 | 1.74E−02 | 5.43E−03 | 1.26E−02 | 1 | SOCS3 |
| NM_018134 | 1.74E−02 | 2.19E−02 | 3.27E−02 | 1 | IQCC |
| NM_000519 | 1.74E−02 | 2.19E−02 | 2.23E−02 | 1 | HBD |
| NM_003811 | 1.74E−02 | 1.18E−02 | 1.57E−02 | 1 | TNFSF9 |
| NM_005191 | 1.74E−02 | 2.19E−02 | 8.99E−03 | 1 | CD80 |
| NM_017528 | 1.74E−02 | 4.17E−03 | 9.17E−04 | 1 | WBSCR22 |
| NM_002620 | 1.74E−02 | 2.72E−02 | 4.54E−02 | 1 | PF4V1 |
| NM_000423 | 1.74E−02 | 2.19E−02 | 7.31E−04 | 1 | KRT2A |
| AL578551 | 1.74E−02 | 2.19E−02 | 4.25E−03 | 1 | RNF10 |
| U91543 | 1.74E−02 | 2.72E−02 | 1.55E−02 | 1 | CHD3 |
| BC004242 | 1.74E−02 | 2.19E−02 | 6.76E−03 | 1 | HNRPUL1 |
| U03886 | 1.74E−02 | 2.72E−02 | 2.05E−02 | 1 | PNPLA4 |
| M36172 | 1.74E−02 | 2.19E−02 | 3.03E−02 | 1 | MYL4 |
| U20498 | 1.74E−02 | 2.19E−02 | 6.66E−03 | 1 | CDKN2D |
| BC000383 | 1.74E−02 | 4.17E−03 | 3.83E−02 | 1 | WTAP |
| BC006383 | 1.74E−02 | 5.43E−03 | 4.42E−04 | 1 | GPAA1 |
| AY026505 | 1.74E−02 | 5.43E−03 | 8.99E−04 | 1 | KIF2C |
| U17074 | 1.74E−02 | 2.19E−02 | 1.23E−02 | 1 | CDKN2C |
| D87858 | 1.74E−02 | 2.19E−02 | 3.94E−02 | 1 | FCAR |
| AI827941 | 1.74E−02 | 5.43E−03 | 1.32E−03 | 1 | MYH9 |
| BE968833 | 1.74E−02 | 2.72E−02 | 2.71E−02 | 1 | SPTBN1 |
| AK021419 | 1.74E−02 | 2.19E−02 | 2.12E−02 | 1 | SMARCB1 |
| D50918 | 1.74E−02 | 5.43E−03 | 3.23E−03 | 1 | |
| H65865 | 1.74E−02 | 2.72E−02 | 1.69E−02 | 1 | FLJ13910 |
| M62324 | 1.74E−02 | 5.43E−03 | 7.50E−03 | 1 | ARID5A |
| AW168132 | 1.74E−02 | 2.19E−02 | 3.40E−03 | 1 | KIAA0404 |
| AA514622 | 1.74E−02 | 2.19E−02 | 3.47E−02 | 1 | POM121 /// |
| | | | | | LOC340318 |
| AL578583 | 1.74E−02 | 5.47E−04 | 1.21E−03 | 1 | CORT |
| AA022949 | 1.74E−02 | 2.19E−02 | 1.18E−02 | 1 | — |
| BG485135 | 1.74E−02 | 2.72E−02 | 1.23E−02 | 1 | — |
| BG540628 | 1.74E−02 | 2.19E−02 | 1.73E−02 | 1 | — |
| BG482805 | 1.74E−02 | 2.19E−02 | 1.53E−02 | 1 | — |
| AC007842 | 1.74E−02 | 2.19E−02 | 4.29E−02 | 1 | LOC163131 |
| BG325734 | 1.74E−02 | 2.72E−02 | 1.53E−02 | 1 | MAPKAPK2 |
| AW404894 | 1.74E−02 | 2.72E−02 | 1.37E−02 | 1 | — |
| W46388 | 1.74E−02 | 2.72E−02 | 1.58E−02 | 1 | SOD2 |
| AL008730 | 1.74E−02 | 4.17E−03 | 3.33E−03 | 1 | C6orf4 |
| X58851 | 1.74E−02 | 2.19E−02 | 4.04E−02 | 1 | MYL4 |
| L14482 | 1.74E−02 | 2.19E−02 | 2.57E−02 | 1 | POU6F1 |
| AL050332 | 1.74E−02 | 2.19E−02 | 1.57E−03 | 1 | LYPLA2P1 |
| D84143 | 1.74E−02 | 2.19E−02 | 5.78E−03 | 1 | — |
| AJ224869 | 1.74E−02 | 2.19E−02 | 7.85E−03 | 1 | CXCR4 |
| AL022067 | 1.74E−02 | 2.72E−02 | 2.51E−02 | 1 | PRDM1 |
| AL512687 | 1.74E−02 | 5.43E−03 | 1.56E−03 | 1 | NOMO2 /// |
| | | | | | NOMO1 /// |
| | | | | | NOMO3 |
| D84140 | 1.74E−02 | 2.19E−02 | 4.15E−03 | 1 | — |
| AF043583 | 1.74E−02 | 2.19E−02 | 1.59E−02 | 1 | — |
| AI042030 | 1.74E−02 | 5.43E−03 | 3.57E−03 | 1 | SMC1L1 |
| NM_005746 | 1.74E−02 | 2.72E−02 | 9.92E−03 | 1 | PBEF1 |
| NM_005746 | 1.74E−02 | 2.72E−02 | 1.28E−02 | 1 | PBEF1 |
| AL523965 | 1.74E−02 | 2.19E−02 | 1.86E−02 | 1 | C6orf106 |
| NM_017874 | 1.74E−02 | 2.19E−02 | 2.42E−03 | 1 | C20orf27 |
| NM_022452 | 1.74E−02 | 5.43E−03 | 1.14E−02 | 1 | FBS1 |
| NM_024324 | 1.74E−02 | 2.19E−02 | 2.83E−03 | 1 | — |
| NM_024535 | 1.74E−02 | 2.19E−02 | 1.05E−03 | 1 | CORO7 |
| NM_023948 | 1.74E−02 | 2.19E−02 | 1.92E−02 | 1 | MOSPD3 |
| NM_022341 | 1.74E−02 | 5.43E−03 | 1.97E−03 | 1 | PDF /// COG8 |
| NM_016633 | 1.74E−02 | 2.19E−02 | 2.74E−02 | 1 | ERAF |
| NM_012447 | 1.74E−02 | 2.72E−02 | 1.41E−02 | 1 | STAG3 |
| NM_020533 | 1.74E−02 | 2.19E−02 | 4.81E−02 | 1 | MCOLN1 |
| NM_019891 | 1.74E−02 | 2.19E−02 | 2.86E−02 | 1 | ERO1LB |
| NM_018113 | 1.74E−02 | 2.19E−02 | 4.51E−02 | 1 | LIMR |
| NM_017774 | 1.74E−02 | 5.43E−03 | 7.44E−03 | 1 | CDKAL1 |
| NM_024669 | 1.74E−02 | 2.72E−02 | 3.93E−03 | 1 | FLJ11795 |
| NM_014076 | 1.74E−02 | 7.75E−03 | 7.57E−04 | 1 | GPR97 |
| N39536 | 1.74E−02 | 5.43E−03 | 1.95E−03 | 1 | NOMO2 /// |
| | | | | | NOMO1 /// |
| | | | | | NOMO3 |
| L14754 | 1.74E−02 | 2.19E−02 | 2.49E−02 | 1 | IGHMBP2 |
| Y14330 | 1.74E−02 | 2.43E−02 | 4.44E−03 | 1 | JAG2 |
| U88964 | 1.74E−02 | 2.19E−02 | 1.28E−02 | 1 | ISG20 |
| AL096779 | 1.74E−02 | 2.72E−02 | 2.66E−02 | 1 | C22orf4 |
| AC005954 | 1.74E−02 | 4.17E−03 | 4.12E−03 | 1 | TJP3 |
| AF022991 | 1.74E−02 | 2.72E−02 | 1.02E−02 | 1 | PER1 |
| AB002328 | 1.74E−02 | 2.72E−02 | 1.88E−02 | 1 | CABIN1 |
| AI150117 | 1.74E−02 | 2.72E−02 | 1.52E−02 | 1 | TOPORS |
| AI743331 | 1.74E−02 | 2.19E−02 | 7.64E−03 | 1 | C20orf67 |
| L38487 | 3.95E−02 | 3.54E−02 | 1.21E−02 | 1 | ESRRA |
| BC002356 | 3.95E−02 | 4.58E−02 | 8.37E−03 | 1 | NUCB1 |
| NM_000177 | 3.95E−02 | 7.66E−04 | 3.31E−03 | 1 | GSN |
| NM_000018 | 3.95E−02 | 4.91E−02 | 1.50E−02 | 1 | ACADVL |
| NM_002332 | 3.95E−02 | 3.54E−02 | 2.62E−02 | 1 | LRP1 |
| AI920976 | 3.95E−02 | 3.54E−02 | 1.14E−02 | 1 | RERE |
| AL523776 | 3.95E−02 | 9.26E−03 | 1.65E−02 | 1 | OTUB1 |
| NM_003097 | 3.95E−02 | 4.91E−02 | 4.68E−02 | 1 | SNURF /// |
| | | | | | SNRPN |
| NM_006295 | 3.95E−02 | 7.66E−04 | 4.08E−03 | 1 | VARS2 |
| NM_001540 | 3.95E−02 | 2.49E−02 | 2.73E−02 | 1 | HSPB1 |
| NM_003040 | 3.95E−02 | 2.52E−03 | 5.86E−03 | 1 | SLC4A2 |
| NM_015049 | 3.95E−02 | 3.54E−02 | 5.05E−03 | 1 | ALS2CR3 |
| BC005003 | 3.95E−02 | 4.91E−02 | 2.77E−02 | 1 | NFYC |
| BC000120 | 3.95E−02 | 2.49E−02 | 1.09E−02 | 1 | GTF2F1 |
| NM_005334 | 3.95E−02 | 3.54E−02 | 1.79E−02 | 1 | HCFC1 |
| NM_004216 | 3.95E−02 | 3.54E−02 | 1.04E−02 | 1 | DEDD |
| NM_006700 | 3.95E−02 | 1.23E−02 | 8.01E−04 | 1 | FLN29 |
| NM_004514 | 3.95E−02 | 2.49E−02 | 2.54E−02 | 1 | FOXK2 |
| NM_005077 | 3.95E−02 | 3.54E−02 | 5.69E−03 | 1 | TLE1 |
| NM_005641 | 3.95E−02 | 4.91E−02 | 9.70E−03 | 1 | TAF6 |
| NM_003704 | 3.95E−02 | 7.66E−04 | 3.50E−03 | 1 | C4orf8 |
| NM_006547 | 3.95E−02 | 3.54E−02 | 2.21E−02 | 1 | IMP-3 |
| AL136771 | 3.95E−02 | 2.32E−02 | 2.43E−03 | 1 | ELL |
| NM_000265 | 3.95E−02 | 4.88E−03 | 2.45E−03 | 1 | NCF1 |
| NM_002555 | 3.95E−02 | 9.26E−03 | 2.18E−03 | 1 | SLC22A18 |
| NM_003043 | 3.95E−02 | 3.54E−02 | 2.64E−02 | 1 | SLC6A6 |
| NM_014813 | 3.95E−02 | 1.23E−02 | 3.47E−03 | 1 | LRIG2 |
| NM_001157 | 3.95E−02 | 4.88E−03 | 5.60E−03 | 1 | ANXA11 |
| NM_006865 | 3.95E−02 | 4.58E−02 | 1.11E−02 | 1 | LILRA3 |
| NM_004258 | 3.95E−02 | 7.66E−04 | 3.58E−02 | 1 | IGSF2 |
| AF052179 | 3.95E−02 | 3.54E−02 | 3.45E−02 | 1 | ARF1 |
| BE674658 | 3.95E−02 | 2.32E−02 | 2.24E−02 | 1 | FLJ13052 |
| BC003381 | 3.95E−02 | 3.54E−02 | 1.54E−03 | 1 | KIAA0217 |
| K02920 | 3.95E−02 | 9.26E−03 | 7.34E−03 | 1 | GBA |
| D87454 | 3.95E−02 | 4.91E−02 | 4.30E−02 | 1 | KIAA0265 |
| U66879 | 3.95E−02 | 1.75E−02 | 3.88E−03 | 1 | BAD |
| AB011179 | 3.95E−02 | 2.32E−02 | 3.44E−03 | 1 | NCDN |
| D37781 | 3.95E−02 | 4.91E−02 | 1.33E−02 | 1 | PTPRJ |
| U22815 | 3.95E−02 | 2.49E−02 | 2.64E−03 | 1 | PPFIA1 |
| U81802 | 3.95E−02 | 2.49E−02 | 7.85E−03 | 1 | PIK4CB |
| BC004516 | 3.95E−02 | 9.26E−03 | 4.07E−02 | 1 | MAX |
| AF056322 | 3.95E−02 | 2.49E−02 | 2.77E−02 | 1 | SP100 |
| AF234161 | 3.95E−02 | 4.91E−02 | 5.17E−03 | 1 | CIZ1 |
| U90940 | 3.95E−02 | 4.91E−02 | 4.23E−02 | 1 | FCGR2C |
| AL046054 | 3.95E−02 | 2.32E−02 | 1.78E−02 | 1 | PTOV1 |
| AI762552 | 3.95E−02 | 4.91E−02 | 9.59E−03 | 1 | HNRPDL |
| BF974389 | 3.95E−02 | 3.54E−02 | 7.94E−03 | 1 | MTVR1 |
| AW237172 | 3.95E−02 | 2.32E−02 | 2.21E−02 | 1 | JMJD2B |
| AA877910 | 3.95E−02 | 1.23E−02 | 3.15E−03 | 1 | ATP2A3 |
| AW157619 | 3.95E−02 | 9.26E−03 | 3.57E−03 | 1 | CES2 |
| AW451236 | 3.95E−02 | 2.49E−02 | 2.11E−02 | 1 | TCEB3 |
| BE856549 | 3.95E−02 | 3.54E−02 | 4.99E−02 | 1 | KIAA0974 |
| AW072388 | 3.95E−02 | 1.75E−02 | 4.60E−03 | 1 | NCF1 |
| AK025604 | 3.95E−02 | 3.54E−02 | 9.44E−03 | 1 | M-RIP |
| AL096741 | 3.95E−02 | 4.58E−02 | 5.49E−03 | 1 | ASCC2 |
| AK025271 | 3.95E−02 | 3.54E−02 | 4.30E−02 | 1 | CHCHD3 |
| AB035175 | 3.95E−02 | 2.32E−02 | 2.57E−03 | 1 | IGHG1 |
| NM_016145 | 3.95E−02 | 4.91E−02 | 1.95E−02 | 1 | PTD008 |
| BE891920 | 3.95E−02 | 2.49E−02 | 4.78E−02 | 1 | ARPC4 |
| NM_024293 | 3.95E−02 | 9.26E−03 | 3.73E−03 | 1 | C2orf17 |
| NM_014164 | 3.95E−02 | 3.54E−02 | 1.73E−03 | 1 | FXYD5 |
| AL561281 | 3.95E−02 | 3.54E−02 | 4.57E−02 | 1 | MAP4K4 |
| NM_006342 | 3.95E−02 | 3.54E−02 | 3.08E−02 | 1 | TACC3 |
| NM_017684 | 3.95E−02 | 3.54E−02 | 1.76E−02 | 1 | VPS13C |
| NM_022089 | 3.95E−02 | 2.32E−02 | 4.84E−03 | 1 | HSA9947 |
| NM_018053 | 3.95E−02 | 2.49E−02 | 2.04E−02 | 1 | FLJ10307 |
| NM_024604 | 3.95E−02 | 4.91E−02 | 3.07E−02 | 1 | FLJ21908 |
| NM_018694 | 3.95E−02 | 3.54E−02 | 4.96E−02 | 1 | ARL6IP4 |
| NM_019096 | 3.95E−02 | 4.58E−02 | 6.10E−03 | 1 | GTPBP2 |
| AB016531 | 3.95E−02 | 2.52E−03 | 1.67E−02 | 1 | PEX16 |
| AI660075 | 3.95E−02 | 3.54E−02 | 3.63E−02 | 1 | LRCH4 |
| AF052151 | 3.95E−02 | 4.88E−03 | 7.99E−03 | 1 | MTVR1 |
| AI744083 | 3.95E−02 | 3.54E−02 | 6.98E−03 | 1 | MOSPD2 |

Table 4: Provided are genetic markers which are differentially expressed between subjects diagnosed with definite multiple sclerosis and healthy controls (the Probeset ID of the Affymetrix Gene Chip), along with the corresponding GenBank accession number (GenBank Acc. No.), the gene symbol and the direction (Dir) of change in gene expression (“1” - upregulation; “−1” - downregulation).
Note that the p values of the TNOM, Info and t-Test statistical tests all passed the 95% confidence level.

### Example 3

### Identification of Genes which Expression Pattern is Characteristics to Probable Multiple Sclerosis Subjects which Further Convert to Definite Multiple Sclerosis

Probable vs. definite gene expression patterns—To identify genes which expression pattern, i.e., upregulation or downregulation is characteristics to probable multiple sclerosis subjects who further convert to definite multiple sclerosis (within a 2 years period), the PBMC expression pattern of genes differentially expressed between definite RRMS/healthy controls (722 genetic markers shown in Table 4, hereinabove) was compared to the expression pattern of probable MS who converted to definite MS (12 patients, converted within 2-years)/healthy controls (1517 genetic markers shown in Table 2, hereinabove).

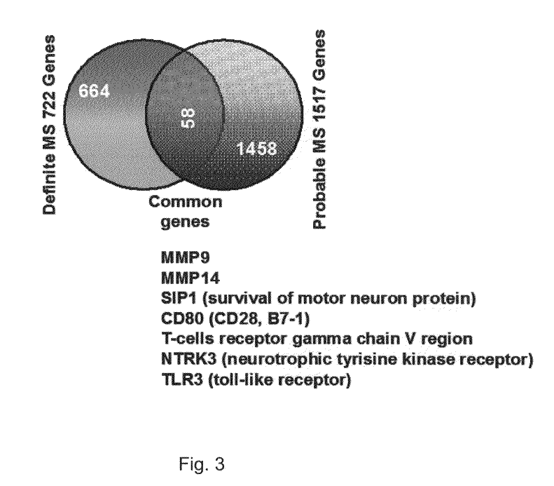

This intersection disclosed 58 universal genes that characterize probable (who are predisposed to develop definite MS) and definite MS disease (FIG. 3 and Table 5, hereinbelow). This signature included MMP genes: MMP9, MMP14; antigen presenting genes like: B7-1 (CD80, CD28); T-cells receptor genes and neuron survival genes: SIP1, TCRγV.

TABLE 5
Universal expression pattern of markers common to subjects with
probable MS diagnosis which later converted to definite diagnosis
of MS and subjects with definite diagnosis of MS

| | SEQ | | | | |
|---|---|---|---|---|---|
| | ID | GenBank | | Fold | |
| Probeset | NO: | Accession No. | Dir | Change | Gene Symbol |
| 203335_at | 1 | NM_006214 | −1 | 2.0 | PHYH |
| 216746_at | 2 | AK024606 | −1 | 2.1 | AK024606 |
| 217377_x_at | 3 | AF041811 | −1 | 2.3 | NTRK3 |
| 214274_s_at | 4 | AI860341 | −1 | 2.4 | ACAA1 |
| 204151_x_at | 5 | NM_001353 | −1 | 2.6 | AKR1C1 |
| 203468_at | 6 | NM_003674 | −1 | 2.9 | CDK10 |
| 218303_x_at | 7 | NM_016618 | −1 | 2.9 | LOC51315 |
| 204221_x_at | 8 | U16307 | −1 | 2.9 | HRB2 |
| 218129_s_at | 9 | BC005316 | −1 | 3.1 | NFYB |
| 212073_at | 10 | AI631874 | −1 | 3.2 | CSNK2A1 |
| 218607_s_at | 11 | NM_018115 | −1 | 3.2 | SDAD1 |
| 206271_at | 12 | NM_003265 | −1 | 3.4 | TLR3 |
| 217908_s_at | 13 | NM_018442 | −1 | 3.5 | IQWD1 |
| 160020_at | 14 | Z48481 | −1 | 3.7 | MMP14 |
| 217381_s_at | 15 | X69383 | −1 | 4.1 | TCR |
| 218349_s_at | 16 | AA824298 | −1 | 4.8 | FLJ10036 |
| 221007_s_at | 17 | NM_030917 | −1 | 5.0 | FIP1L1 |
| 218728_s_at | 18 | AK024569 | −1 | 5.7 | HSPC163 |
| 218096_at | 19 | NM_018361 | −1 | 6.2 | LPAAT-e |
| 213980_s_at | 20 | AA053830 | −1 | 6.4 | CTBP1 |
| 222149_x_at | 21 | AL137398 | −1 | 6.6 | DKFZp434P162 |
| 221222_s_at | 22 | NM_017860 | −1 | 6.8 | FLJ20519 |
| 202660_at | 23 | AA834576 | −1 | 8.1 | ITPR2 |
| 210235_s_at | 24 | U22815 | −1 | 8.3 | PPFIA1 |
| 205540_s_at | 25 | NM_016656 | −1 | 8.8 | RRAGB |
| 204682_at | 26 | NM_000428 | −1 | 9.0 | LTBP2 |
| 218859_s_at | 27 | NM_016649 | −1 | 12.2 | C20orf6 |
| 218699_at | 28 | NM_003929 | −1 | 26.5 | RAB7L1 |
| 205063_at | 29 | NM_003616 | −1 | 31.3 | SIP1 |
| 214085_x_at | 30 | AI912583 | −1 | 66.8 | HRB2 |
| 216399_s_at | 31 | AK025663 | −1 | 82.9 | ZNF291 |
| 219575_s_at | 32 | NM_022341 | 1 | 2.0 | PDF, COG8 |
| 216330_s_at | 33 | L14482 | 1 | 2.1 | POU6F1 |
| 203222_s_at | 34 | NM_005077 | 1 | 2.1 | TLE1 |
| 203064_s_at | 35 | NM_004514 | 1 | 2.2 | FOXK2 |
| 207176_s_at | 36 | NM_005191 | 1 | 2.2 | CD80 |
| 213360_s_at | 37 | AA514622 | 1 | 2.2 | POM121 |
| 205920_at | 38 | NM_003043 | 1 | 2.4 | SLC6A6 |
| 206544_x_at | 39 | NM_003070 | 1 | 2.4 | SMARCA2 |
| 218961_s_at | 40 | NM_007254 | 1 | 2.7 | PNKP |
| 201526_at | 41 | NM_001662 | 1 | 2.9 | ARF5 |
| 203936_s_at | 42 | NM_004994 | 1 | 3.0 | MMP9 |
| 207908_at | 43 | NM_000423 | 1 | 3.1 | KRT2A |
| 202942_at | 44 | NM_001985 | 1 | 3.3 | ETFB |
| 211696_x_at | 45 | AF349114 | 1 | 4.3 | HBB |
| 218037_at | 46 | NM_024293 | 1 | 4.9 | C2orf17 |
| 209116_x_at | 47 | M25079 | 1 | 5.3 | HBB |
| 204018_x_at | 48 | NM_000558 | 1 | 6.4 | HBA1, HBA2 |
| 200055_at | 49 | NM_006284 | 1 | 6.5 | TAF10 |
| 202596_at | 50 | BC000436 | 1 | 6.6 | ENSA |
| 201827_at | 51 | AF113019 | 1 | 7.2 | SMARCD2 |
| 214414_x_at | 52 | T50399 | 1 | 7.6 | HBA2 |
| 202768_at | 53 | NM_006732 | 1 | 14.0 | FOSB |
| 215684_s_at | 54 | AL096741 | 1 | 14.9 | ASCC2 |
| 212071_s_at | 55 | BE968833 | 1 | 21.8 | SPTBN1 |
| 202111_at | 56 | NM_003040 | 1 | 28.8 | SLC4A2 |
| 212413_at | 57 | D50918 | 1 | 105.1 | D50918 |
| 209740_s_at | 58 | U03886 | 1 | 106.7 | PNPLA4 |

Table 5: Genetic markers which are differentially expressed between probable multiple sclerosis which further developed definite diagnosis of MS and healthy controls are provided (the Probeset ID of the Affymetrix Gene Chip), along with the corresponding GenBank accession number, the gene symbol, the SEQ ID NO., the direction of change in gene expression (“1”- upregulation; “−1” - downregulation) and the fold change in subjects diagnosed with probable MS (who are predisposed to develop definite MS) as compared to healthy control (probable MS/healthy control).
Note that the p values of the TNOM, Info and t-Test statistical tests all passed the 95% confidence level.

Determination of the prediction power of selected genes which differentiate between probable MS subjects who are predisposed to develop definite MS and healthy controls—To evaluate the power of each of the 58 differentiating genes (SEQ ID NOs:1-58) identified in this study to predict the predisposition of a probable MS subject to develop a definite MS diagnosis, the study sample of 40 probable patients was randomly divided into 80% of the subjects as a “training set” and 20% (to confirm) of the subjects as a “test set” and a model was build using the SVM based on RBF kernel. For each of the differentiating genes the predictability of the training set on the test set was computed and the average error following 25 permutations was calculated. Genes with the lowest average error were selected, then, for each selected gene, the remaining genes were added one after the other, by selecting the next gene such that the average error after 25 repeats of the group of genes including the new gene has the lowest average error as compared to the addition of another gene. This process was repeated 57 times for each additional gene added to the previous group of genes. The resulting average error for each gene combination is depicted in Table 6, hereinbelow, wherein the first gene in row number 1 (SEQ ID NO:4) exhibits the best predictive power (error average of “0.21”) as a single gene.

TABLE 6

| Row | SEQ | | | | |
|---|---|---|---|---|---|
| Num- | ID | | Gene Bank | Error | Gene |
| ber | NO: | Probeset ID | ID | Average | Symbol |
| 1 | 4 | 214274_s_at | AI860341 | 0.216363636 | ACAA1 |
| 2 | 16 | 218349_s_at | NM_017975 | 0.216363636 | FLJ10036 |
| 3 | 5 | 204151_x_at | NM_001353 | 0.247272727 | AKR1C1 |
| 4 | 56 | 202111_at | NM_003040 | 0.230909091 | SLC4A2 |
| 5 | 20 | 213980_s_at | AA053830 | 0.223636364 | CTBP1 |
| 6 | 3 | 217377_x_at | AF041811 | 0.158181818 | NTRK3 |
| 7 | 1 | 203335_at | NM_006214 | 0.169090909 | PHYH |
| 8 | 10 | 212073_at | AI631874 | 0.176363636 | CSNK2A1 |
| 9 | 57 | 212413_at | D50918 | 0.169090909 | 6-Sep |
| 10 | 24 | 210235_s_at | U22815 | 0.194545455 | PPFIA1 |
| 11 | 14 | 160020_at | Z48481 | 0.214545455 | MMP14 |
| 12 | 49 | 200055_at | NM_006284 | 0.201818182 | TAF10 |
| 13 | 13 | 217908_s_at | NM_018442 | 0.181818182 | PC326 |
| 14 | 37 | 213360_s_at | AA514622 | 0.163636364 | na |
| 15 | 6 | 203468_at | NM_003674 | 0.165454545 | CDK10 |
| 16 | 47 | 209116_x_at | M25079 | 0.158181818 | HBB |
| 17 | 50 | 202596_at | BC000436 | 0.163636364 | ENSA |
| 18 | 21 | 222149_x_at | AL137398 | 0.154545455 | — |
| 19 | 46 | 218037_at | NM_024293 | 0.143636364 | MGC3035 |
| 20 | 8 | 204221_x_at | U16307 | 0.165454545 | GLIPR1 |
| 21 | 26 | 204682_at | NM_000428 | 0.174545455 | LTBP2 |
| 22 | 2 | 216746_at | AK024606 | 0.169090909 | — |
| 23 | 15 | 217381_s_at | X69383 | 0.172727273 | — |
| 24 | 51 | 201827_at | AF113019 | 0.167272727 | SMARCD2 |
| 25 | 44 | 202942_at | NM_001985 | 0.170909091 | ETFB |
| 26 | 19 | 218096_at | NM_018361 | 0.167272727 | LPAAT-e |
| 27 | 17 | 221007_s_at | NM_030917 | 0.176363636 | FIP1L1 |
| 28 | 25 | 205540_s_at | NM_016656 | 0.176363636 | RRAGB |
| 29 | 33 | 216330_s_at | L14482 | 0.161818182 | POU6F1 |
| 30 | 48 | 204018_x_at | NM_000558 | 0.174545455 | HBA1 |
| 31 | 36 | 207176_s_at | NM_005191 | 0.161818182 | CD80 |
| 32 | 34 | 203222_s_at | NM_005077 | 0.158181818 | TLE1 |
| 33 | 12 | 206271_at | NM_003265 | 0.132727273 | TLR3 |
| 34 | 29 | 205063_at | NM_003616 | 0.136363636 | SIP1 |
| 35 | 23 | 202660_at | AA834576 | 0.132727273 | — |
| 36 | 11 | 218607_s_at | NM_018115 | 0.150909091 | FLJ10498 |
| 37 | 45 | 211696_x_at | AF349114 | 0.149090909 | HBB |
| 38 | 53 | 202768_at | NM_006732 | 0.16 | FOSB |
| 39 | 41 | 201526_at | NM_001662 | 0.156363636 | ARF5 |
| 40 | 40 | 218961_s_at | NM_007254 | 0.130909091 | PNKP |
| 41 | 31 | 216399_s_at | AK025663 | 0.154545455 | ZNF291 |
| 42 | 58 | 209740_s_at | U03886 | 0.143636364 | DXS1283E |
| 43 | 27 | 218859_s_at | NM_016649 | 0.147272727 | C20orf6 |
| 44 | 43 | 207908_at | NM_000423 | 0.132727273 | KRT2A |
| 45 | 35 | 203064_s_at | NM_004514 | 0.132727273 | ILF1 |
| 46 | 30 | 214085_x_at | AI912583 | 0.16 | HRB2 |
| 47 | 52 | 214414_x_at | T50399 | 0.156363636 | HBA1 |
| 48 | 55 | 212071_s_at | BE968833 | 0.147272727 | SPTBN1 |
| 49 | 7 | 218303_x_at | NM_016618 | 0.16 | LOC51315 |
| 50 | 9 | 218129_s_at | NM_006166 | 0.161818182 | NFYB |
| 51 | 42 | 203936_s_at | NM_004994 | 0.167272727 | MMP9 |
| 52 | 28 | 218699_at | BG338251 | 0.145454545 | RAB7L1 |
| 53 | 54 | 215684_s_at | AL096741 | 0.170909091 | FLJ21588 |
| 54 | 32 | 219575_s_at | NM_022341 | 0.167272727 | COG8 |
| 55 | 22 | 221222_s_at | NM_017860 | 0.178181818 | FLJ20519 |
| 56 | 18 | 218728_s_at | NM_014184 | 0.181818182 | HSPC163 |
| 57 | 38 | 205920_at | NM_003043 | 0.190909091 | SLC6A6 |
| 58 | 39 | 206544_x_at | NM_003070 | 0.194545455 | SMARCA2 |

Table 6: Shown are the average errors of predicting a probability of a probable MS subject to develop the diagnosis of definite MS within a 2-years period based on a model computed for each gene or a group of genes in the MS training set group. The ascending order of genes reflects combinations of genes, where each row includes the gene specified in that row and in all preceding rows. For example, the average error presented in row number 4 reflects the average error in predicting a probability of a probable MS subject to develop the diagnosis of definite MS within a 2-years period using the group of genes described in rows 1, 2, 3 and 4 (i.e., SEQ ID NOs: 4, 16, 5 and 56).
Probe set ID = Affymetrix ID.

As shown in Table 6 hereinabove, the predictive power of each set of genes was evaluated using the MS training and test sets of samples. The polynucleotide exhibiting the best predictive power in determining the probability of a probable MS subject to convert to the diagnosis of definite MS was the polynucleotide set forth by SEQ ID NO:4 (GenBank Accession No. AI860341; row No. 1 in Table 6), in which the average error between the test and training groups was 0.216. Similarly, the combination genes set forth by SEQ ID NOs:4 and 16 (GenBank Accession No. NM_{—}017975; row No. 2 in Table 6) displayed a predictive power with 0.216 average error. Another exemplary combination, which provides an even higher prediction power (with a smaller average error) is shown in row number 6 in Table 6, in which the combination of the polynucleotide sequences set forth in SEQ ID NOs:4, 16, 5, 56, 20 and 3 displayed a high predictive power with 0.158 average error. Yet another exemplary combination, which provides an even higher prediction power (with a smaller average error) is shown in row number 35 in Table 6, in which the combination of the polynucleotide sequences set forth in SEQ ID NOs:4, 16, 5, 56, 20, 3, 1, 10, 57, 24, 14, 49, 13, 37, 6, 47, 50, 21, 46, 8, 26, 2, 15, 51, 44, 19, 17, 25, 33, 48, 36, 34, 12, 29 and 23 displayed a high predictive power with 0.132 average error. Thus, this analysis enables one skilled in the art to select a group of polynucleotides which can give the best predictive power for prediction of the probability of a subject diagnosed with probable MS (after the first neurological attack) to develop the diagnosis of definite MS within 2 years.

### CONCLUSIONS

1. PBMC gene expression signature distinguished probable MS patients from healthy subjects.

2. Patients that experience a second relapse and converted to definite MS during 2 years of follow-up period have a specific gene expression signature.

3. Patients with probable and definite MS demonstrate a universal gene expression signature.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

### REFERENCES

### Additional References are Cited in Text

1. Anderson D W, Ellenberg J H, Leventhal C M, et al. Revised estimate of the prevalence of multiple sclerosis in the United States. Ann Neurol. 1992 March; 31(3):333-6.
2. Weinshenker B C. Natural history of multiple sclerosis. Ann Neurol. 1994; 36 Suppl:S6-11.
3. Confavreux C, Vukusic S, Moreau T, et al. Relapses and progression of disability in multiple sclerosis. N Engl J. Med. 2000 Nov. 16; 343(20):1430-8.
4. Poser C M, Paty D W, Scheinberg L, et al. New diagnostic criteria for multiple sclerosis: guidelines for research protocols. Ann Neurol. 1983 March; 13(3):227-31.
5. Oksenberg J R, Baranzini S E, Barcellos L F, et al. Multiple sclerosis: genomic rewards. J. Neuroimmunol. 2001 Feb. 15; 113(2):171-84.
6. Compston A, Coles A. Genetic analysis of multiple sclerosis. Curr Neurol Neurosci Rep. 2002 May; 2(3):259-66.
7. Achiron A, Barak Y. Multiple sclerosis-from probable to definite diagnosis: a 7-year prospective study. Arch Neurol. 2000 July; 57(7):974-9.
8. O'Riordan J I, Tompson A J, Kingsley D P, et al. The prognostic value of brain MRI in clinically isolated syndromes of the CNS. A 10-year follow-up. Brain. 1998 March; 121 (Pt 3):495-503.
9. Morrissey S P, Miller D H, Kendall B E, et al. The significance of brain magnetic resonance imaging abnormalities at presentation with clinically isolated syndromes suggestive of multiple sclerosis: a 5-year follow-up study. Brain. 1993; 116:135-146.
10. Graves D J. Powerful tools for genetic analysis come of age. Trends Biotechnol. 1999; 17 :127-134.
11. Duggan D J, Bittner M, Chen Y, et al. Expression profiling using cDNA microarrays. Nat Genet 1999; 21(1 Suppl):10-14.
12. Greenberg S A. DNA microarray gene expression analysis technology and its application to neurological disorders. Neurology 2001; 57:755-61.
13. Whitney L W, Becker K G, Tresser N J, Caballero-Ramos C I, Munson P J, Prabhu V V, Trent J M, McFarland H F, Biddison W E. Analysis of gene expression in multiple sclerosis lesions using cDNA microarrays. Ann Neurol. 1999; 46(3): 425-8.
14. Lock C B, Heller R A. Gene microarray analysis of multiple sclerosis lesions. Trends Mol Med 2003; 9: 535-41.
15. Ramanathan M, Weinstock G, Nguyen L T, et al. In vivo gene expression revealed by cDNA arrays: the pattern in relapsing remitting multiple sclerosis patients compared with normal subjects. J Neuroimmunol 2001; 116:213-219.
16. Achiron, A., Gurevich, M., Friedman, N., Kaminski, N. and Mandel, M. Blood transcriptional signatures of multiple sclerosis: unique gene expression of disease activity. Ann Neurol 2004; 55: 410-7.
17. Kurtzke J F. Rating neurologic impairment in multiple sclerosis an expanded disability status scale (EDSS). Neurology 1983; 33:1444-1452.
18. Achiron A, Gicquel S, Miron S, Faibel M. Brain MRI lesion load quantification in multiple sclerosis: a comparison between automated multispectral and semi-automated thresholding computer-assisted techniques. Magn Reson Imaging 2002; 20:713-20.
19. Furey T S, Cristianini N, Duffy N, Bednarski D W, Schummer M, Haussler D. Support vector machine classification and validation of cancer tissue samples using microarray expression data. Bioinformatics 2000; 16:906-14.
20. Statnikov A, Aliferis C F, Tsamardinos I, Hardin D, Levy S. A comprehensive evaluation of multicategory classification methods for microarray gene expression cancer diagnosis. Bioinformatics 2005; 21:631-43.
21. Ben-Dor A, Bruhn L, Friedman N, Nachman I, Schummer M, Yakhini Z. Tissue classification with gene expression profiles. J Comput Biol 2000; 7: 559-83.

1. A method of determining a probability of a subject diagnosed with probable multiple sclerosis to develop definite multiple sclerosis, comprising determining in a cell of the subject a level of expression of at least one polynucleotide sequence selected from the group consisting of SEQ ID NOs:4, 16, 5, 56, 20, 3, 1, 10, 57, 24, 14, 49, 13, 37, 6, 47, 50, 21, 46, 8, 26, 2, 15, 51, 44, 19, 17, 25, 33, 48, 36, 34, 12, 29, 23, 11, 45, 53, 41, 40, 31, 58, 27, 43, 35, 30, 52, 55, 7, 9, 42, 28, 54, 32, 22, 18, 38, and 39, wherein an alteration above a predetermined threshold in said level of expression of said at least one polynucleotide sequence in said cell of the subject relative to a level of expression of said at least one polynucleotide sequence in a reference cell is indicative of the probability of the subject diagnosed with probable multiple sclerosis to develop definite multiple sclerosis.

2. A method of treating a subject diagnosed with probable multiple sclerosis, comprising:
(a) determining the probability of the subject diagnosed with probable multiple sclerosis to develop definite multiple sclerosis according to the method of claim 1, and;
(b) selecting a treatment regimen based on said probability;
thereby treating the subject diagnosed with probable multiple sclerosis.

3.-4. (canceled)

5. A probeset comprising a plurality of oligonucleotides and no more than 500 oligonucleotides wherein each of said plurality of oligonucleotides is capable of specifically recognizing at least one polynucleotide sequence selected from the group consisting of SEQ ID NOs:4, 16, 5, 56, 20, 3, 1, 10, 57, 24, 14, 49, 13, 37, 6, 47, 50, 21, 46, 8, 26, 2, 15, 51, 44, 19, 17, 25, 33, 48, 36, 34, 12, 29, 23, 11, 45, 53, 41, 40, 31, 58, 27, 43, 35, 30, 52, 55, 7, 9, 42, 28, 54, 32, 22, 18, 38, and 39.

6. The probeset of claim 5, wherein each of said isolated nucleic acid sequences or said plurality of oligonucleotides is bound to a solid support.

7. The probeset of claim 5, wherein said plurality oligonucleotides are bound to said solid support in an addressable location.

8. The method of claim 1, wherein said reference cell is of an unaffected subject.

9. The method of claim 8, wherein said alteration is upregulation of said expression level of said at least one polynucleotide sequence in said cell of the subject relative to said reference cell, whereas said at least one polynucleotide sequence is selected from the group consisting of SEQ ID NOs: 32-58.

10. The method of claim 9, wherein said probability of the subject diagnosed with probable multiple sclerosis to develop definite multiple sclerosis is higher than about 75%.

11. The method of claim 8, wherein said alteration is downregulation of said expression level of said at least one polynucleotide sequence in said cell of the subject relative to said reference cell, whereas said at least one polynucleotide sequence is selected from the group consisting of SEQ ID NOs: 1-31.

12. The method of claim 11, wherein said probability of the subject diagnosed with probable multiple sclerosis to develop definite multiple sclerosis is higher than about 75%.

13. The method of claim 1, wherein said detecting said level of expression is effected using an RNA detection method.

14.-15. (canceled)

16. The method of claim 1, wherein said at least one polynucleotide sequence is as set forth by the polynucleotide sequences of SEQ ID NOs:4, 16, 5, 56, 20, 3, 1, 10, 57, 24, 14, 49, 13, 37, 6, 47, 50, 21, 46, 8, 26, 2, 15, 51, 44, 19, 17, 25, 33, 48, 36, 34, 12, 29, 23, 11, 45, 53, 41, 40, 31, 58, 27, 43, 35, 30, 52, 55, 7, 9, 42, 28, 54, 32, 22, 18, 38, and 39.

17. The method of claim 1, wherein said cell of the subject is a blood cell.

18. The method of claim 1, wherein said detecting said level of expression is effected at the protein level.

19. A kit for determining a probability of a subject diagnosed with probable multiple sclerosis to develop definite multiple sclerosis, comprising the probeset of claim 5 and a reference cell.

20. The kit of claim 19, further comprising at least one reagent suitable for detecting hybridization of said plurality of oligonucleotides and at least one RNA transcript corresponding to said at least one polynucleotide sequence selected from the group consisting of SEQ ID NOs:4, 16, 5, 56, 20, 3, 1, 10, 57, 24, 14, 49, 13, 37, 6, 47, 50, 21, 46, 8, 26, 2, 15, 51, 44, 19, 17, 25, 33, 48, 36, 34, 12, 29, 23, 11, 45, 53, 41, 40, 31, 58, 27, 43, 35, 30, 52, 55, 7, 9, 42, 28, 54, 32, 22, 18, 38, and 39.

21. The kit of claim 19, further comprising packaging materials packaging said at least one reagent and instructions for use in determining the probability of a subject diagnosed with probable multiple sclerosis to develop definite multiple sclerosis.


SEQUENCE LISTING (58 sequences)

SEQ ID NO: 1 - DNA, 1578 bp, Homo sapiens
gcccgctgcggtaaatggggcagaggccgggaggggtgggggttccccgcgccgcagccatggagcagcttcgcgccgccgcccgtctgcagattgttctgggccacctcggccgcccctcggccggggctgtcgtagctcatcccacttcagggactatttcctctgccagtttccatcctcaacaattccagtatactctggataataatgttctaaccctggaacagagaaaattttatgaagaaaatgggtttctagtaatcaaaaatcttgtacctgatgccgatattcaacgctttcggaatgagtttgaaaaaatctgcagaaaggaggtgaaaccattaggattaacagtaatgagagatgtgaccatttcgaaatccgaatatgctccaagtgagaagatgatcacgaaggtccaggatttccaggaagataaggagctcttcagatactgcactctccccgagattctgaaatatgtggagtgcttcactggacctaatattatggccatgcacacaatgttgataaacaaacctccagattctggcaagaagacgtcccgtcaccccctgcaccaggacctgcactatttccccttcaggcccagcgatctcatcgtttgcgcctggacggcgatggagcacatcagccggaacaacggctgtctggttgtgctcccaggcacacacaagggctccctgaagccccacgattaccccaagtgggaggggggagttaacaaaatgttccacgggatccaggactacgaggaaaacaaggcccgggtgcacctggtgatggagaagggcgacactgttttcttccatcctttgctcatccacggatctggtcagaataaaacccagggattccggaaggcaatttcctgccatttcgccagtgccgattgccactacattgacgtgaagggcaccagtcaagaaaacatcgagaaggaagttgtaggaatagcacataaattctttggagctgaaaatagcgtgaacttgaaggatatttggatgtttcgagctcgacttgtgaaaggagaaagaaccaatctttgaaatagccatctgctataactctttcaacagaaaaccaaaaccaaacgaaatgtctaaggaaaatgttttcttaatgagatgatgtaaccttttctatcacttgttaaaagcagaaaacatgtatcaggtacttaattgcatagagttagttttgcagcacaatggtgttgctttaatggaaaaaaaaaacagtaaaagtgaaatattactgttttaaggaaaactaatttagggtggcagccaataaaggtggttggtgtctaatttaagtgttaaatcaatttctttcattcagttagctctttacccaagaagaagtgaatgatttggagcttagggtatgttttgtatcccctttctgataaacccattccctaccaattttatgtcataagagatttttttcccccaaatctagaacaatgtataatacatttcacatctagtcaagggcataggaacggtgtcatggagtccaaataaagtggatattcctgctcgg

SEQ ID NO: 2 - DNA, 1995 bp, Homo sapiens
aactcctgggctcaagtgatcctcctgccttagcctcctaaagggctgagattacaggtgtgagccaccacgcctggccaagaagagagttttaaatgttaataaattggcctgtgataacactgaattatattgggtttcaagaaatcacaaaaaaagcaacacctccctgctgtcagcagctcatagaatggtgggctagacataaacgtatacaaggaaattgccacattgtaagaaaatgtcctattagtggcacatactcatggccatggaagagcaaagaagaggagatagaaagagatagggtgaaggggggaggacaggaaacccagtcaacttttagttcatgtcttttaaaataaggaagagactctaatcagaagttgacaagtgtcaccttccaatattgtgactttctcaatatcacagaggccagggttttaagaaaaatcactctgcgaagatgacactgactttctcttaatctcccctacctcatcttcccaaaaagattaactgaactaatagacttcaaacttctggcctaacttccagtttacaggaaatcatgaatgagagaaactgaacagcctcaacaggaaacaatcagacacatcctccatgtgggatgtcctccaaggcaagtggcctggtctctttctaaagtcaatgtcaaaaaaaaaaaaaaaaaatcccaggcacagtggccttacacttgtaatcccagcactttgtgaagccaaggccattggatcacctgaggtcaggagttcgaggccagcctaaccaatgtggtaaaaccctgtctctactaaaaatacaaaaattagctgggcgtgttggtgggcacaagtaatcccacctactcggaaggctgagacaggagaatcacttgaacccaggaggcggaggttacagtgagccaatatagcaccattgccctccagcctgggtgacaagagcaaaacgccatctcaaaaaaaaaaaaaaaaaaaaaaagtgagagaactgttctagatgctagattaaacataaaagagtttaggactacaatatgaattgcaatgcatgagcacctgtgtatctgaatttaggaagaaaaaatcgccataaaagacaatttggagacaactagatagatttaaatatggactggataccagatataatgagaaaattatcgttcatttttcatattttgaaatgttagtgtctttatgtacgagaatgcccttactattaggaaacagatgctcaagaacttaggccaggcacagtggctcatgcctataatctgaatacttgggaggctgaggcaggaggatcacttgagtctgagaccagcctgggcaacatggtaagatttctctctatagaaataaaattaaaattagccaggtgtggtggcatgtgcctatagttccagctacttgggaggctgaggtgggaggattagttgagcccagaagttcgaggctgcagtgagctatgattgcaccactgccctccagcctgggtgacatagtgagaccctgccttaaaatatatatagagagagagggtaagcatcatgacatctccttaccctgattaatctcaaactgttcagcaaaaaacatatatatatatatacacacatccgcataccttcgcatacacatatacacacacaaatagagaaagcaaatgcaggctgggcacggttgctcacacctataatcccaacactttgggaggccaaggcaggcagatcacctgagatcaggagttcgagaccagcctggccaacatggcaaaaccccgtctctactaaatatacaaaaaagttagctggatgtagtggcgggcgcctgtaatcccagctactcggggggctaaggcaggagaatcacttgaacccgagaggcgggggttgcagtgaacggagatggcgccactgcattccagcctgggtgacagagcgagatgattgtctcaaaaaaaaaaaaaaaa

SEQ ID NO: 3 - DNA, 1403 bp, Homo sapiens
ttttttttttttttctttttttttccttttttcagtgttgtatgtgtagcaggcacttgagtttatatgaagatgttcgcttcagtcaaggatggaaggagttgtgaggtggaagggagatgtgaggcagggagagaggaggcaacagagtatgaattcatgaccaccagccaccactagccaagaatgtccaggtagattggggtggccttccccaaagcatggaggattttgtagatctccttgatgttcaaccgctgctgtggttccctctgccagcaccccagcatgacatcgtacacctctttggggcagactcggggccgctccaaaacacgaccttgggtaatgcactcaatgacctccgtgtttgagagttggaaccatggctgctttccataggtgaagatctcccagaggatcaccccgaagctccatacatcactctctgtagtgaacttccggtacatgatgctttcaggaggcatccagcgaatggggagcatggtgtgtcctcccaccctgtaataatccgtgctgtagacatctctggacatgccgaagtccccaatcttcactagcagattcgctccaaccaggcagttcctggtggccaggtctcggtgcacaaagtgctgggaggccaggtacaccatacccgaggcgatctgactggcaatgtggagcatttgggagagccccagctcacccttggcctggcgtggctgtccatccacaaggatcattgcatctggcccatgggccctgaggaacttattcaggtctccatgcttcatgtattcaaagaccatgatgagggggtccccatcgccgcacactccatagaacttgacaatgtgctcatgctgcaggttggtgagcagctcggcctccctctggaaatccttccgggcagccagggtgggatccttcagggccttcacagccacaagcatcttgtccttggtcgggctgaggttgtagcactcggccaggaagacctttccaaaggctccctcacccagttctcgcttcagcacgatgtctctcctcttaatgtgctgcacatctgctattctcccaatgggcatggcgtgctcttcaggcggggagacagagaccatgatgtggttcatgtaagccaggtcttcccgatgagagaggttgatgggcttcccttccctatgcagcccgtcctcggagagcctggactgtttgaaatccacggagtgccgggggttcaggatcagagggtgcatgatggggctgggcatcagctggatcacgcgtgtgctctcctgccgggggctggatggcttcgggtgggactcggaggacgctgggcagtgattattctccatgggagacactgacagagggtaggactcctggtggttgttctc

SEQ ID NO: 4 - DNA, 423 bp, Homo sapiens
  misc_feature (97)..(97): n is a, c, g, or t
ttttttttaagataaattcatgcacttttactatgcccattgcacttctcatccatggatttgccttgccttaagaattaaccatggccttgtggcntgggtgacccaggntgcttttatcttgcacagctaaagagggtctgatgggtggctcaacaccccacccactcctataccatgtcatcagtgttcacattttccaaatgagttgaagtgccttgatctgtccactgccaccaccaccagtgctgagttttcccatgtggttttgcttttgtggtgttactgccttgctgctagagcagcaggactgtctgcgtagcgcctccagcctgggacctcactcagttcccagggtattcaaagacggcagcggctcccattccagtcccgatgcacatggacaccactccgtatgccctc

SEQ ID NO: 5 - DNA, 1207 bp, Homo sapiens
ccagaaatggattcgaaatatcagtgtgtgaagctgaatgatggtcacttcatgcctgtcctgggatttggcacctatgcgcctgcagaggttcctaaaagtaaagctttagaggccaccaaattggcaattgaagctggcttccgccatattgattctgctcatttatacaataatgaggagcaggttggactggccatccgaagcaagattgcagatggcagtgtgaagagagaagacatattctacacttcaaagctttggtgcaattcccatcgaccagagttggtccgaccagccttggaaaggtcactgaaaaatcttcaattggattatgttgacctctaccttattcattttccagtgtctgtaaagccaggtgaggaagtgatcccaaaagatgaaaatggaaaaatactatttgacacagtggatctctgtgccacgtgggaggccgtggagaagtgtaaagatgcaggattggccaagtccatcggggtgtccaacttcaaccgcaggcagctggagatgatcctcaacaagccagggctcaagtacaagcctgtctgcaaccaggtggaatgtcatccttacttcaaccagagaaaactgctggatttctgcaagtcaaaagacattgttctggttgcctatagtgctctgggatcccaccgagaagaaccatgggtggacccgaactccccggtgctcttggaggacccagtcctttgtgccttggcaaaaaagcacaagcgaaccccagccctgattgccctgcgctaccagctacagcgtggggttgtggtcctggccaagagctacaatgagcagcgcatcagacagaacgtgcaggtgtttgaattccagttgacttcagaggagatgaaagccatagatggcctaaacagaaatgtgcgatatttgacccttgatatttttgctggcccccctaattatccattttctgatgaatattaacatggagggcattgcatgaggtctgccagaaggccctgcgtgtggatggtgacacagaggatggctctatgctggtgactggacacatcgcctctggttaaatctctcctgcttggtgatttcagcaagctacagcaaagcccattggccagaaaggaaagacaataattttgttttttcattttgaaaaaattaaatgctctctcctaaagattcttcacctaaaaaa

SEQ ID NO: 6 - DNA, 1883 bp, Homo sapiens
gtgagccaccgcccccagcctggcctggcatttctttgagttcaggaagtgtgacaaggatttggacacccagaaataagcgtgtcgagaagagcacaagcagaggatccagcgctcggcatggcggagccagatctggagtgcgagcagatccgtctgaagtgtattcgtaaggagggcttcttcacggtgcctccggaacacaggctgggacgatgccggagtgtgaaggagtttgagaagctgaaccgcattggagagggtacctacggcattgtgtatcgggcccgggacacccagacagatgagattgtcgcactgaagaaggtgcggatggacaaggagaaggatggcatccccatcagcagcttgcgggagatcacgctgctgctccgcctgcgtcatccgaacatcgtggagctgaaggaggtggttgtggggaaccacctggagagcatcttcctggtgatgggttactgtgagcaggacctggccagcctcctggagaatatgccaacacccttctcggaggctcaggtcaagtgcatcgtgctgcaggtgctccggggcctccagtatctgcacaggaacttcattatccacagggacctgaaggtttccaacttgctcatgaccgacaagggttgtgtgaagacagcggatttcggcctggcccgggcctatggtgtcccagtaaagccaatgacccccaaggtggtcactctctggtaccgagcccctgaactgctgttgggaaccaccacgcagaccaccagcatcgacatgtgggctgtgggctgcatactggccgagctgctggcgcacaggcctcttctccccggcacttccgagatccaccagatcgacttgatcgtgcagctgctgggcacgcccagtgagaacatctggccgggcttttccaagctgccactggtcggccagtacagcctccggaagcagccctacaacaacctgaagcacaagttcccatggctgtcggaggccgggctgcgcctgctgcacttcctgttcatgtacgaccctaagaaaagggcgacggccggggactgcctggagagctcctatttcaaggagaagcccctaccctgtgagccggagctcatgccgacctttccccaccaccgcaacaagcgggccgccccagccacctccgagggccagagcaagcgctgtaaaccctgacggtgggcctggcacacgcctgtattcccacaccaggtcttccgatcagtggtgtctgtgaagggtgccgcgagccaggctgaccaggcgcccgggatccagctcatccccttggctgggaacatcctccactgacttcctcccactgtctgccctgaacccactgctgcccccagaaaaaggccgggtgacaccgggggctcccagcccgtgcaccctggaagggcaggtctggcggctccatccgtggctgcaggggtctcatgtggtcctcctcgctatgttggaaatgtgcaaccactgcttcttgggaggagtggtgggtgcagtccccccgctgtctttgagttgtggtggaccgctggcctgggatgagagggcccagaagaccttcgtatcccctctcagtcgcccggggctgtcccgtgcatgggttggctgtggggaccccaggtgggcctggcaggactccagatgaggacaagagggacaaggtatggggtgggagccacaattgaggataccccgagctaccaggagagccctgggctggaggctgagctggatccctgctccccacacggaggacccaacaggaggccgtggctctgatgctgagcgaagctataggctcttgttggataaaagcttttttaacagaaaaaaaaaaaaaaaaaaaa

SEQ ID NO: 7 - DNA, 1774 bp, Homo sapiens
tgttcctgcctctccgccacctccaccgcggcttatcccctggctggcggcgttggcggggcgggggacagtagttgtagacgcccgcccctgcctcagagaagataccacattgaatgaacaaagactgaactttgtttacagataccttttataaaagatgtctgtggagtagtgttacagttcaaataacaaaggattttgcattatgagatgagtgagagtggagaattggacaaaaattgatttttgatctctgaattgtggtttttagctcaccagttattgtgtagtctcactatttgaaaattcatgctgaaaaactgagtgaatatgatcagatttctccaccaaattctcagcctgataaggaaaatcccgttttgtcaacctaatgaagcattcaaagaagacatatgactcttttcaagatgaacttgaagattatattaaagtacagaaagccagaggcttagagccaaagacttgtttcagaaagatgaaaggggactatttggaaacctgtgggtacaaaggagaggttaattccagacccacgtatagaatgtttgaccagagactcccatctgaaaccatccagacctacccaagatcatgcaatattccacaaacagtggaaaatcggttgcctcagtggttaccagcccatgacagcagattgagactagactctctgagctactgtcagttcacgagggactgtttctcagaaaaaccagtacccctgaactttaatcaacaagaatatatttgtggctcacatggtgtagaacatagagtttacaagcacttctcctcagataacagtaccagtactcatcaagccagtcacaaacagatacatcagaagaggaaaaggcacccagaggaaggcagagaaaaatcagaggaggagcggtctaagcataagagaaaaaaaagctgcgaggaaattgacttagacaaacacaagagcatccaaagaaagaaaacagaggtggaaatagaaaccgtacatgtcagtacagaaaagcttaagaatcgaaaggagaaaaaaagccgagatgtagtctctaagaaagaggaacgtaagcgtacaaaaaagaaaaaggaacaaggccaagaaaggacagaggaggaaatgctttgggaccagtctattcttggattttgaagctttcaaagttggttctcccaaagttaaattgaaaaaataggtgagagcttggttttatgatatccgtgttcataccacttttcttatgtgaataggttctttaacttctaacaaaggcctagtaaacaaagtgtttagcatgcttgctctccaacacagaaattgcttttcctcattttctaaaagcattattacattttttgaacatatagtgtaatttcctttaatgaaagtgactctgcttttattcatcaaattgctttgatggtggaaatattttctgttgggaggttatttattttaaattggaggattaatgacctttgcacaatctgtttcttgattgggtttgttatagttttgagttgggtattttatgttcattggtttttctctgtgaagcaatttttttctcctttattagatctaacttgcagtgtattttctaggctggaaagtggaaaatgaaatatattatgatcttaggttacataaagtttctaaagtttcaaagagtcttgatacaaaatcagtttatattctgaaaatatttataataaagtattctaatttctaaaaaaaaaaaaaaaaaaaaaa

SEQ ID NO: 8 - DNA, 1597 bp, Homo sapiens
ggctgagcactcaggcaatcacactctcagaaactgcggcggctctggactgcagcctccaaggctccatgccagacaaagctatgcgtagtcacacttgctacaatagcctggatggtttcttttgtctccaattattcacacacagcaaatattttgccagatatcgaaaatgaagatttcatcaaagactgcgttcgaatccataacaagttccgatcagaggtgaaaccaacagccagtgatatgctatacatgacttgggacccagcactagcccaaattgcaaaagcatgggccagcaattgccagttttcacataatacacggctgaagccaccccacaagctgcacccaaacttcacttcactgggagagaacatctggactgggtctgtgcccattttttctgtgtcttccgccatcacaaactggtatgacgaaatccaggactataacttcaagactcggatatgcaaaaaagtctgtggccactacactcaggttgtttgggcagatagttacaaagttggctgcgcagttcaattttgccctaaagtttctggctttgacgctctttccaatggagcacattttatatgcaactacggaccaggagggaattacccaacttggccatataagagaggagccacctgcagtgcctgccccaataatgacaagtgtttggacaatctctgtgttaacgacagcgagaccaagtcaaacgttactactatgttgtatatcaggctggcccatatatccacgtaacagatacacttctctctttctcattgttaattcagtaattctaatactgtctgttataattaccattttggtacagcacaagtaccctaatttagttcttttggactaatacaattcaggaaagaaaaaacccaaaaaccaacctcattcacatatggctttttttttaacccaataacaattaggtgtaccttcctatttaaacatttcataaaaaaatatatgttatagcaatactcttactcaaaagaagaaatttcctaactctatcagataaactcatctttagtataaataagcattatttgcaggttgcctacaggtggacttttagtaagtaacctaacccatgtttcagcttctaaatctcgaaaatgagcaaggtacaggtacagtagcacatttttaggtgattcttagtaactccagtagccttcattagttaaaaacattattattttttgcatgctgcttcgactctaaatatcttgttttccctgtctttttggtttactacttccccagattcagaacagaggagtaactaggggatctgattttagaggcctaattttctgttcatggactgttaaaagtaaaaccaaactttcaaaagggataaacctaaatatttacttgttatcattagagagggaacatcaaatgctggcactatatacatacgatcagcctgattatgatatcacaatggtcgtaatgtatacaaagacttatataccactttctcgtataaatttttcaaaaaatacaataataatataatttataaagaacactcttctatgaacaaccaccaccacca

SEQ ID NO: 9 - DNA, 734 bp, Homo sapiens
ccaaccagggctgcattggaggttgaaatcacaaagattagacacctttttagataggtgttcttcagcaccactgacaacacggttctgacagtatttcatgacaatggacggcgacagctctacaacagatgcttctcaactaggaatctctgcagactatattggaggaagtcattatgttatacagcctcatgatgatactgaggacagcatgaatgatcatgaagacacaaatggttcaaaagaaagtttcagagaacaagatatatatcttccaatagcaaacgtggctaggataatgaaaaatgccatacctcaaacgggaaagattgcaaaagatgccaaagaatgtgttcaagaatgtgtaagtgagttcatcagttttataacatctgaagcaagtgaaaggtgccatcaagagaaacggaaaacaatcaatggagaagatattctctttgctatgtctactttaggctttgacagttatgtggaacctctgaaattataccttcagaaattcagagaggctatgaaaggagaaaagggaattggtggagcagtcacagctacagatggactaagtgaagagcttacagaggaggcatttactaaccagttaccagctggcttaataaccacagacggtcaacaacaaaatgttatggtttacacaacatcatatcaacagatttctggtgtccagcaaattcagttttcatgatctgaagaaa

SEQ ID NO: 10 - DNA, 4423 bp, Homo sapiens
  misc_feature (316)..(316): n is a, c, g, or t
gggggtttccgcttccggcagcagcggctgcagcctcgctctggtccctgcggctggcggccgagccgtgtgtctcctcctccatcgccgccatattgtctgtgtgagcagaggggagagcggccgccgccgctgccgcttccaccacagctctatcaaggcttgtcaagcagtgtgctcatcacatggtaaatcatgcagcgtggaacctcataaaatctccaagaaacatcattcacccatactgactagtttcacatctctttgtttgaagaaaacaggtctgaaacaaggtcttacccccagctgcttctgnaacacagtgactgccagatctccaaacatcaagtccagctttgtccgccaacctgtctgacatgtcgggacccgtgccaagcagggccagagtttacacagatgttaatacacacagacctcgagaatactgggattacgagtcacatgtggtggaatggggaaatcaagatgactaccagctggttcgaaaattaggccgaggtaaatacagtgaagtatttgaagccatcaacatcacaaataatgaaaaagttgttgttaaaattctcaagccagtaaaaaagaagaaaattaagcgtgaaataaagattttggagaatttgagaggaggtcccaacatcatcacactggcagacattgtaaaagaccctgtgtcacgaacccccgccttggtttttgaacacgtaaacaacacagacttcaagcaattgtaccagacgttaacagactatgatattcgattttacatgtatgagattctgaaggccctggattattgtcacagcatgggaattatgcacagagatgtcaagccccataatgtcatgattgatcatgagcacagaaagctacgactaatagactggggtttggctgagttttatcatcctggccaagaatataatgtccgagttgcttcccgatacttcaaaggtcctgagctacttgtagactatcagatgtacgattatagtttggatatgtggagtttgggttgtatgctggcaagtatgatctttcggaaggagccatttttccatggacatgacaattatgatcagttggtgaggatagccaaggttctggggacagaagatttatatgactatattgacaaatacaacattgaattagatccacgtttcaatgatatcttgggcagacactctcgaaagcgatgggaacgctttgtccacagtgaaaatcagcaccttgtcagccctgaggccttggatttcctggacaaactgctgcgatatgaccaccagtcacggcttactgcaagagaggcaatggagcacccctatttctacactgttgtgaaggaccaggctcgaatgggttcatctagcatgccagggggcagtacgcccgtcagcagcgccaatatgatgtcagggatttcttcagtgccaaccccttcaccccttggacctctggcaggctcaccagtgattgctgctgccaacccccttgggatgcctgttcncagctgccnntggcgctcagcagtaacggccctatctgtctcctgatgcctgagcagaggtgggggagtccaccctctccttgatgcagnttgcgcntggcggggaggggtgaaacacttcagaagcaccgtgtctgaaccgttgcttgtggatttatagtagttcagtcataaaaaaaaaattataatagnctgannntcttttttcttttttttttnaactcgaacttttcataactcaggggattccctgaaaaattacctgcaggtggaatatttcatggacaaatttttttttctcccctcccaaatttagttcctcatcacaaaagaacaaagataaaccagcctcaatcccggctgctgcatttaggtggagacttcttcccattcccaccattgttcctccaccgtcccacactttagggggttggtatctcgtgctcttctccagagattacaaaaatgtagcttctcaggggaggcaggaagaaaggaaggaaggaaagaaggaagggaggacccaatctataggagcagtggactgcttgctggtcgcttacatcactttactccataagcgcttcagtggggttatcctagtggctcttgtggaagtgtgtcttagttacatcaagatgttgaaaatctacccaaaatgcagacagatactaaaaacttctgttcagtaagaatcatgtcttactgatctaaccctaaatccaactcatttatacttttatttttagttcagtttaaaatgttgataccttccctcccaggctccttaccttggtcttttccctgttcatctcccaacatgctgtgctccatagctggtaggagagggaaggcaaaatctttcttagttttctttgtcttggccattttgaattcatttagttactgggcataacttactgctttttacaaaagaaacaaacattgtctgtacaggtttcatgctagagctaatgggagatgtggccacactgacttccattttaagctttctaccttcttttcctccgaccgtccccttccctcacatgccatccagtgagaagacctgctcctcagtcttgtaaatgtatcttgagaggtaggagcagagccactatctccattgaagctgaaatggtagacctgtaattgtgggaaaactataaactctcttgttacagccccgccaccccttgctgtgtgtatatatataatactttgtccttcatatgtgaaagatccagtgttggaattctttggtgtaaataaacgtttggttttatttatcaannnnaaannnaannnnnntgtgtaaaggtcttgctgggtgggtgtctcatgttcacatctgaggggcctgcagccctgtaccgtggaggcttcccaaggcccccattttatacacccctcgttcgacccatggtaccgggcagagcagagaggccttaaaaaaaaagcaccacaagccaaagcgtctctggggattaaggatcctttgccataaaactcatttgtgtctcagcagtgcagggatnggggatggaaaaagtcgccagttttngtgtgtnngtgtgtgtgtgtaaagtggactttccactgtaatccaaccacctaagtttatcaggtgcttcactgaggaagcctagttttttaagcacaatagcaaaaccatcagctctgtattttctcctgttatttcattacagtagctgcttgtgggaactaggaaaaattcttccaacatattttaaggcctaaaatcttagttccccattctcctaccttatagattcacaggcctttctcgcctaggcatcatagataaacgtaattgtttggggagttgaatttaatgaacttatctaactttgtaacccatcttggctttagtaactttatcaaggtggtggctttagtgaatatantggtaaactttagaggacgctaaagcctccttttatagcgcttctcaacggtagggagagctgaagggaaaacattctgactgtgtggcagggtgattttcttccttatgaccacttacagtggatatttattgtacttgacccttttatgccctagaatgctgtgagggttaccatgttgaatttgtgcagaagctaaaagcaccagatgtgccagagatgcaatttgtgattatgtttgcactggattgtgatttgaacaggacacttataactaatgagttctttcttttgaggtggggagagggttgtaaatcaagacttcataccctatccttgtagctcggaaattgaggtgtagcttaggctgatgcggagagctgcagacagctggacctccttacacccatcagaccttggaataaaccgaaggtgaatgcagaggactctgaggtgccacccaagagccttttaaaataaatagaagtttttaaaaatgctacttaagagtcactgcagaagcatggagagagagaaaaaaaaaaaagacccaattgggtggggggtgggggggaggcaagggtttacagagtaacaacttacgtgttgttgtaaattacctcatctgtataccttgtattgggacactatttntcagcactacctgtgtctgcattgcttagatggcaggaaatggaatggccaacatgaaaggttatgttggcaattactctgaaagattttttaaagtatttttaaactttgtttgaagtggtccctcccctcttcctctgcttttttttaagtttgaagccagaaggccctccccaccttgctttattggttgtaattgtattgtaattggtatgactaaaccataactgtgctgggaagaagttgtgccatgaaggtctttaattttttttaataaaaacatttaaataaatgaaannnnnnnnnannnaaa

SEQ ID NO: 11 - DNA, 2755 bp, Homo sapiens
caaataaacccagcaaaggaactagcagagctggtgatgtttatggcacagattagtcactgctacccagagtacctaagtaattttcctcaagaggtgaaagatcttctctcctgcaatcataccgtattggatccagatctgcgaatgacattttgcaaagctttgatcttgctgagaaataagaatctcatcaatccatcaagcctgctagaactcttctttgaactttttcgttgccatgataaacttctgcgaaagactttatacacacatattgtgactgatatcaagaatataaatgcaaaacacaagaacaataaagtgaatgtagtattgcaaaatttcatgtacaccatgttaagagatagcaatgcaaccgcagccaagatgtctttagatgtaatgattgaactctacagaaggaacatctggaatgatgcaaaaactgtcaatgttatcacaactgcatgtttctctaaggtcaccaagatattagttgccgctttgacattctttcttgggaaagatgaagatgaaaaacaggacagtgactccgaatctgaggatgatggaccaacagcaagagacctgctagtacaatatgctacagggaagaaaagttccaaaaacaagaaaaagttggaaaaggcaatgaaagtgctcaagaaacaaaaaaagaagaaaaaaccagaggtgtttaacttttcagccattctcttgattcatgatccccaagattttgcggaaaaactactaaagcagcttgagtgctgtaaggagaggtttgaagtgaagatgatgctcatgaaccttatctccagattggtgggaattcatgagcttttcctcttcaatttctatccctttttgcaaaggtttctgcagccccaccaaagagaagtaaccaagatccttctgtttgctgcacaagcatctcatcacctagtacccccagagattattcaatcattgcttatgactgtggcaaacaattttgttaccgacaagaactctggagaagtcatgacagtaggaatcaatgctataaaggagataacagctcgatgtcctctggccatgactgaagaacttctccaagacctggctcagtataaaacacacaaggataagaatgtaatgatgtctgctagaactttgattcacctcttccgaacactgaatcctcagatgctgcagaagaaattccggggtaagcctacagaggcctccatagaagcaagagtacaagaatatggagaattagatgctaaagattacattccaggagcagaagttctggaagttgagaaagaagagaatgctgaaaatgatgaagatggatgggaaagtaccagtctcagtgaggaggaggatgctgatggtgaatggattgatgtgcaacactcttccgatgaagaacagcaagaaatctccaagaagctgaacagcatgcccatggaggagcggaaggccaaagctgcagccatcagcactagccgagttttaactcaggaagacttccagaaaatccgcatggcccaaatgagaaaagaacttgatgctgcccccgggaaatgccagaagaggaaatacattgaaatagacagtgatgaagagcccaggggtgaattactttctcttcgggacattgaacgccttcataaaaagccaaagtctgacaaagagacaagactagcaactgcaatggctggaaagacagaccgaaaagaatttgtgaggaagaaaaccaaaacaaatccattttccagttcgacaaataaagagaagaaaaaacagaagaactttatgatgatgcggtatagccagaatgtccggtcaaaaaataagcgttccttccgagaaaaacagttggcactacgagatgcacttttgaaaaagagaaaaagaatgaagtaacttcctggcaagttttccattcctagaagaatgctaagtttgtgtccttgctctgaaaattggtaaatcaagcatgtttgtttacattaaaaagtccagacacactgtattgtgaaaactgctgaacatgtggcagcaattttgtgtttttattttggagacggctaatggtaggaatgttaatgtaaatagtggtggtaatgtaaaatcatttcatttatcattcatgcaaaaaaaagtatgtattgagtgcctattcattgtcactgtagatgcaaaacgaatgagctgtaaccccttcactcaaggcattgacagcttagctgtgagggtggacacacatatgtgtatttacagtgcagtgtaaatagttctgtagtagaggttaactccatatttctgtgaggtcactgaggccttatggactaactctgtgaggataggagttatatattcttataagacaaaacaaaacaggacaatgttacaagagtaagaggttcttacttgtacataggctttcctgctgaaaacaggcccctgctgtacagattttgggtacataatttagctcttttagtcagtccaagagatttaagtgaccccccccccccccgtgttttttttgtttttgtttttgttttgaatgccatgtaaaggctttttggttaagacctcacttttaaaactgccttaagtataaatagtacctttggaatatatttagttcatcatttgagctgccttcatactggtttcctcagccttccttcagcctgtaatattttcagcccactgtttaccttgtctcaataaaaggtttctaatgccaaat

SEQ ID NO: 12 - DNA, 3029 bp, Homo sapiens
gcggccgcgtcgacgaaatgtctggatttggactaaagaaaaaaggaaaggctagcagtcatccaacagaatcatgagacagactttgccttgtatctacttttgggggggccttttgccctttgggatgctgtgtgcatcctccaccaccaagtgcactgttagccatgaagttgctgactgcagccacctgaagttgactcaggtacccgatgatctacccacaaacataacagtgttgaaccttacccataatcaactcagaagattaccagccgccaacttcacaaggtatagccagctaactagcttggatgtaggatttaacaccatctcaaaactggagccagaattgtgccagaaacttcccatgttaaaagttttgaacctccagcacaatgagctatctcaactttctgataaaacctttgccttctgcacgaatttgactgaactccatctcatgtccaactcaatccagaaaattaaaaataatccctttgtcaagcagaagaatttaatcacattagatctgtctcataatggcttgtcatctacaaaattaggaactcaggttcagctggaaaatctccaagagcttctattatcaaacaataaaattcaagcgctaaaaagtgaagaactggatatctttgccaattcatctttaaaaaaattagagttgtcatcgaatcaaattaaagagttttctccagggtgttttcacgcaattggaagattatttggcctctttctgaacaatgtccagctgggtcccagccttacagagaagctatgtttggaattagcaaacacaagcattcggaatctgtctctgagtaacagccagctgtccaccaccagcaatacaactttcttgggactaaagtggacaaatctcactatgctcgatctttcctacaacaacttaaatgtggttggtaacgattcctttgcttggcttccacaactagaatatttcttcctagagtataataatatacagcatttgttttctcactctttgcacgggcttttcaatgtgaggtacctgaatttgaaacggtcttttactaaacaaagtatttcccttgcctcactccccaagattgatgatttttcttttcagtggctaaaatgtttggagcaccttaacatggaagataatgatattccaggcataaaaagcaatatgttcacaggattgataaacctgaaatacttaagtctatccaactcctttacaagtttgcgaactttgacaaatgaaacatttgtatcacttgctcattctcccttacacatactcaacctaaccaagaataaaatctcaaaaatagagagtgatgctttctcttggttgggccacctagaagtacttgacctgggccttaatgaaattgggcaagaactcacaggccaggaatggagaggtctagaaaatattttcgaaatctatctttcctacaacaagtacctgcagctgactaggaactcctttgccttggtcccaagccttcaacgactgatgctccgaagggtggcccttaaaaatgtggatagctctccttcaccattccagcctcttcgtaacttgaccattctggatctaagcaacaacaacatagccaacataaatgatgacatgttggagggtcttgagaaactagaaattctcgatttgcagcataacaacttagcacggctctggaaacacgcaaaccctggtggtcccatttatttcctaaagggtctgtctcacctccacatccttaacttggagtccaacggctttgacgagatcccagttgaggtcttcaaggatttatttgaactaaagatcatcgatttaggattgaataatttaaacacacttccagcatctgtctttaataatcaggtgtctctaaagtcattgaaccttcagaagaatctcataacatccgttgagaagaaggttttcgggccagctttcaggaacctgactgagttagatatgcgctttaatccctttgattgcacgtgtgaaagtattgcctggtttgttaattggattaacgagacccataccaacatccctgagctgtcaagccactacctttgcaacactccacctcactatcatgggttcccagtgagactttttgatacatcatcttgcaaagacagtgccccctttgaactctttttcatgatcaataccagtatcctgttgatttttatctttattgtacttctcatccactttgagggctggaggatatctttttattggaatgtttcagtacatcgagttcttggtttcaaagaaatagacagacagacagaacagtttgaatatgcagcatatataattcatgcctataaagataaggattgggtctgggaacatttctcttcaatggaaaaggaagaccaatctctcaaattttgtctggaagaaagggactttgaggcgggtgtttttgaactagaagcaattgttaacagcatcaaaagaagcagaaaaattatttttgttataacacaccatctattaaaagacccattatgcaaaagattcaaggtacatcatgcagttcaacaagctattgaacaaaatctggattccattatattggttttccttgaggagattccagattataaactgaaccatgcactctgtttgcgaagaggaatgtttaaatctcactgcatcttgaactggccagttcagaaagaacggataggtgcctttcgtcataaattgcaagtagcacttggatccaaaaactctgtacattaaatttatttaaatattcaattagcaaaggagaaactttctcaatttaaaaagttctatggcaaatttaagttttccataaaggtgttataatttgtttattcatatttgtaaatgattatattctatcacaattacatctcttctaggaaaatgtgtctccttatttcaggcctatttttgacaattgacttaattttacccaaaataaaacatataagcacgcaaaaaaaaaaaaaaaaaa

SEQ ID NO: 13 - DNA, 2727 bp, Homo sapiens
aatattttataccaacgttgagcaagatgcagaaaccaacagacaatgccaatttacgtgtcattatggaactacttatgagattatgactgtacccaatgacccttacacttttctctcttgtggtgaagatggaactgttaggtggtttgatacacgcatcaaaactagctgcacaaaagaagattgtaaagatgatattttaattaactgtcgacgtgctgccacgtctgttgctatttgcccaccaataccatattaccttgctgttgggttgttctgacagctcagtacgaatatatgatcggcgaatgctgggcacaagagctacagggaattatgcaggtcgagggactactggaatggttgcccgttttattccttcccatcttaataataagtcctgcagagtgacatctctgtgttacagtgaagatggtcaagagattctcgttagttactcttcagattacatatatctttttgacccgaaagatgatacagcacgagaacttaaaactccttctgcgggaaggagagaaggagaagagttgcgacaaccaccagttaagcgtttgagacttcgtggtgattggtcagatactggacccagagcaaggccggagagtgaacgagaacgagatggagagccagagtccccaatgtgtccattgatgccagagaatgtcctgatatgttatcaagatggtttgaagaagcaagtgaggttgcacaaagcaatagaggacgaggaagatctcgacccagaggtggaacaagtcaatcagatatttcaactcttcctacggtcccatcaagtcctgatttggaagtgagtgaaactgcaatggaagtagatactccagctgaacaatttcttcagccttctacatcctctacaatgtcagctcaggctcattcgacatcatctcccacagaaagccctcattctactcctttgctatcttctccagacagtgaacaaaggcagtctgttgaggcatctggacaccacacacatcatcagtctgattctccttcttctgtggttaacaaacagctcggatccatgtcacttgacgagcaacaggataacaataatgaaaagctgagccccaaaccagggacaggtgaaccagttttaagtttgcactacagcacagaaggaacaactacaagcacaataaaactgaactttacagatgaatggagcagtatagcatcaagttctagaggaattgggagccattgcaaatctgagggtcaggaggaatctttcgtcccacagagctcagtgcaaccaccagaaggagacagtgaaacaaaagctcctgaagaatcatcagaggatgtgacaaaatatcaggaaggagtatctgcagaaaacccagttgagaaccatatcaatataacacaatcagataagttcacagccaagccattggattccaactcaggagaaagaaatgacctcaatcttgatcgctcttgtggggttccagaagaatctgcttcatctgaaaaagccaaggaaccagaaacttcagatcagactagcactgagagtgctaccaatgaaaataacaccaatcctgagcctcagttccaaacagaagccactgggccttcagctcatgaagaaacatccaccagggactctgctcttcaggacacagatgacagtgatgatgacccagtcctgatcccaggtgcaaggtatcgagcaggacctggtgatagacgctctgctgttgcccgtattcaggagttcttcagacggagaaaagaaaggaaagaaatggaagaattggatactttgaacattagaaggccgctagtaaaaatggtttataaaggccatcgcaactccaggacaatgataaaagaagccaatttctggggtgctaactttgtaatgagtggttctgactgtggccacattttcatctgggatcggcacactgctgagcatttgatgcttctggaagctgataatcatgtggtaaactgcctgcagccacatccgtttgacccaattttagcctcatctggcatagattatgacataaagatctggtcaccattagaagagtcaaggatttttaaccgaaaacttgctgatgaagttataactcgaaacgaactcatgctggaagaaactagaaacaccattacagttccagcctctttcatgttgaggatgttggcttcacttaatcatatccgagctgaccggttggagggtgacagatcagaaggctctggtcaagagaatgaaaatgaggatgaggaataataaactctttttggcaagcacttaaatgttctgaaatttgtataagacatttattatatttttttctttacagagctttagtgcaattttaaggttatggtttttggagtttttccctttttttgggataacctaacattggtttggaatgattgtgtgcatgaatttgggagattgtataaaacaaaactagcagaatgtttttaaaactttttgccgtgtatgaggagtgctagaaaatgcaaagtgcaatattttccctaaccttcaaatgtgggagcttggatcaatgttgaagaataattttcatcatagtgaaaatgttggttcaaataaatttctacacttgccatttgcatgtttgttgctttctaattaaagaaactggttgttttaagatcaaaaaaaaaaaaaaaaaaaaaaaaaaaaaa

SEQ ID NO: 14 - DNA, 3555 bp, Homo sapiens
  misc_feature (37)..(37): n is a, c, g, or t
gcacagcgcgactaagcagaagaaagatcaaaaaccnggaaaagaggagaagagcaaacaggcactttgaggaacaatcccctttaactcnaagccgnacagcngtctaggaattcaagttcagtgcctaccgaagacaaaggcgccccgagggagtggcggtgcgaccccagggcgtgggcccggccgcggagccnacactgcccggctgacccggtggtctcggaccatgtctcccgccccaagacccncccgttgtctcctgctccccctgctcacgctcggcaccgcgctcgcctccctcggctcggcccaaagcagcagcttcagccccgaagcnnnnnnnnnnnnnnnnnnnnnnnnnnnncccggggacctacgtacccacacacagnnnnnnnnnnnnnnnnnctcagcggccatcgctgccatgcagaagttttacggcttgcannnnnnnnnnnnnnnnnnnnnnnnnnnnnnnnnnnnnnnnnnnnnnnnnnnnnnnnnngttccagacaagtttggggcnnnnnnnnnnnnnnnnnnnnnnnnnnnnnnctacgccatccagggtctcaaatggcaacataatgaaatcactttctgcatccagaattacacccccaaggtgggcgagtatgccacnnnnnnnnnnnnnnnnnnnnnnnnnnnnnnnnnnnnnnnnnnnnnnnnnnnnnnnttccgcgaggtgccctatgcctacatccgtgagggccatgagnnnnnnnnnnnnnnnnnnnnnnnntttgccgagggcttccatggcgacagcacgcccttcgatggtgagggcggcttcctggcccatgcctacttcccaggccccaannnnnnnnnnnnnnnccactttgactctgccgagccttggactgtcaggaatgaggatctgaatggaaannnnnnnnnnnnnnnnnnnnnnnacgagctgggccatgccctggggctcgagcattccagtgacccctcggccatcatggcacccttttaccagtggatggacacggagaattttgtgnnnnnnnnnnnnnnnnnnnnnnncatccagcaactttatgggggtgagtcagggttccccaccaagatgccccctcaacccaggactacctcccggccttctgttcctgataaacccaaaaaccccacctatgggcccaacatctgtgacgggaactttgacaccgtggccatgctccgaggggagatgtttgtcttcaaggagcgctggttctggcgggtgaggaataaccaagtgatggatggatacccaatgcccattggccagttctggcggggcctgcctgcgtccatcaacactgcctacgagaggaaggatggcaaattcgtcttcttcaaaggagacaagcattgggtgtttgatgaggcgtccctggaacctggctaccccaagcacattaaggagctgggccgagggctgcctaccgacaagnnnnnnnnnnnnnnnttctggatgcccaatggaaagacctacttcttccgtggaaacaagtactaccgtttcaacgaagagctcagggcagtggatagcgagtaccccaagaacatcaaagtctgggaagggatccctgagtctcccagagggtcattcatgggcagcgatgaagtcttcacttacttctacaaggggaacaaatactggaaattcaacaaccagaagctgaaggtagaaccgggctaccccaagtcagccctgagggactggatgggctgcccatcgggaggccggccggatgaggggactgaggaggagacggaggtgatcatcattgaggtggacgaggagggcggcggggcggtgagcgcggctgccgtggtgctgcccgtgctgctgctgctcctggtgctggcggtgggccttgcagtcttcnnnnnnnnnnnnnnngggacccccaggcgactgctctactgnnnnnnnnnnnnnnnngacaaggtctgacgcccanccgccggcccgcccactcctaccacaaggactttgcctctgaaggccagtggcagcaggtggtggtgggtgggctgntcccatcgtcccgagccccctccccgcagcctccttgcttctctctgtcccctggctggcctccttcaccctgaccgcctccctccctcctgccccggcattgcatcttccctagataggtcccctgagggctgagtgggagggcggccctttccagcctctgcccctcaggggaaccctgtagctttgtgtctgtccagccccatctgaatgtgttgggggctctgcacttgaaggcaggaccctcagacctcgctggtaaaggtcaaatggggtcatctgctccttttccatcccctgacataccttaacctcnnnnnnnnnnnnnnnnnnnnnnnnnnnnnnnnnnnnnnnnnnnnnnnnnnnnnnnnnnnnnnnnnnnnnnnnnnnnnnnnnnnnnnggctaaaaggaatctaatcttgttgagggtagagaccctgagacagtgtgagggggtggggactgccaagccaccctaagaccttgggaggaaaactcagagagggtcttcgttgctcagtcagtcaagttcctcggagatctgcctctgcctcacctaccccagggaacttccaagaaggagcctgagccactggggactaagtgggcagaagaaacccttggcagccctgtgcctctcgaatgttagccttggatggggctttcacagttagaagagctgaaaccaggggtgcagctgtcaggtagggtggggccggtgggagaggcccgggtcagagccctgggggtgagcctgaaggccacagagaaagaaccttgcccaaactcaggcagctggggctgaggcccaaaggcagaacagccagagggggcaggaggggaccaaaaaggaaaatgaggacgtgcagcagcattggaaggctggggccnggcagnccaggnnaanncnancagggggccancagggtgggctngtggagctctcaggaagggccctgaggaaggcacacttgctcctgttggtccctgtccttgctgcccaggcagngtggaggggaaggnnnnnnnagnnagnnnnagnnncagagaaggcanncannngnggaatgaggggcttcacgagaggccacagggcctggctggccacgctgtcccnnnnnnnnnnnnnnnnnnnnnnnnnnnnnnnnnnnnngctcgcttaggctgggtccacgcttccctggtgccagcacccctcaagcctgtctcaccagtggcctgccctctcgctcccccacccagcccacccattgaagtctccttgggccaccaaaggtggtggccatggtaccggggacttgggagagtgagacccagtggagggagcaagaggagagggatgtcgggggggtggggcacggggtaggggaaatggggtgaacggtgctggcagttcggctagatttctgtcttgtttgtttttttgttttgtttaatgtatatttttattataattattatatatgaattccaaaaaaaaaaaaaaaaaaaa

SEQ ID NO: 15 - DNA, 213 bp, Homo sapiens
tgggaggaaggcttgggagtcccctaagacacatcttcagtcacttctctctgcctgtgactcaggaagaccagctcctcctactgtcttctgtgctagggatcacttccttgttgagtggggccggagttttaagaggatcttctgctcctcctcatctggtccctttccttccaaggcccccgagaggaaggcatgcggtgggccctactg

SEQ ID NO: 16 - DNA, 1670 bp, Homo sapiens
gggcaaaccctttgaaaaatattctaaatgaaaatgacatagtattcatagtggaaaaagtgcctttagaaaaggaagaaacaagtcatattgaagaacttcaatctgaagaaactgccatatctgatttctctactggcgaaaatgttggaccacttgctttaccagttgggaaggcaaggcagttaattggactttacaccatggctcacaatcctaatatgacccatttgaagattaatctgcctgttactgcccttccccccctttgggtaagatgtgacagttcagatcctgaaggtacttgttggctaggagctgagcttatcacaacaaacaacagcattacaggaattgtcttatatgtggtcagttgtaaagctgataaaaattattctgtaaatcttgaaaacctaaaaaatttacacaagaaaagacatcacttgtctactgtaacatccaaaggctttgcccagtatgagctctttaagtcctctgccttggatgatacaatcacagcatcacaaactgcgatcgctttggatatttcctggagtcctgtggatgagattcttcaaatccctccactctcttcaactgcaatctgaatattaaagtggaatcaggagagcccagaggtcctttgaatcatctctacagagaactgaaatttcttcttgttttggctgatggtttgaggactggtgtcactgaatggctcgagcccctggaagcaaaatctgctgttgaacttgttcaggaatttctgaatgacttaaataagctggatggatttggtgattctacaaaaaaagacactgaggttgagaccttgaagcatgacactgctgcagtcgatcgttccgtcaagcgtcttttcaaagttcggagtgatcttgattttgctgagcaactgtggtgcaaaatgagcagtagtgtgatttcataccaagactaggtgaagtgtttcacattgatcatccagagtctacaacgtggtgatatacagccatggctccatagtggaagtaacagtttactaagtaagctcattcatcagtcttatcatggaaccatggacacagtttctctcagtgggactattccagttcaaatgcttttggaaattggtttggacaaactaaagaaagattatatcagttttttcataggtcaggaacttgcatctttgaatcatttggaatacttcattgctccatcagtagatatacaagaacaggtttatcgtgtccaaaaactccaccatattctagaaatattagtcagttgcatgcctttcattaaatctcaacatgaactcctcttttctttaacacagatctgcataaagtattacaaacaaaatcctcttgatgagcaacacatttttcagctgccagtcagaccaactgctgtaaagaacttatatcaaagtgagaagccacagaaatggagagtggaaatatatagtggtcaaaagaagattaagacagtttggcaactgagtgacagctcacccataggccatctgaattttcacaaacctgatttttcggaattaacactaagcggtagcctggaagaaaggatattctttactaacatggttacctgcagccaggtgcatttcaagtgaagtgtgctgatgaagtcctctataagcacaagcc

SEQ ID NO: 17 - DNA, 1914 bp, Homo sapiens
cggacgcgtgggcggacgcgtggggccttcctgggattggagtctcgagctttcttcgttcgttcgccggcgggttcgcgcccttctcgcgcctcggggctgcgaggctggggaaggggttggagggggctgttgatcgccgcgtttaagttgcgctcggggcggccatgtcggccggcgaggtcgagcgcctagtgtcggagctgagcggcgggaccggaggggatgaggaggaagagtggctctatggcgatgaaaatgaagttgaaaggccagaagaagaaaatgccagtgctaatcctccatctggaattgaagatgaaactgctgaaaatggtgtaccaaaaccgaaagtgactgagaccgaagatgatagtgatagtgacagcgatgatgatgaagatgatgttcatgtcactataggagacattaaaacgggagcaccacagtatgggagttatggtacagcacctgtaaatcttaacatcaagacagggggaagagtttatggaactacagggacaaaagtcaaaggagtagaccttgatgcacctggaagcattaatggagttccactcttagaggtagatttggattcttttgaagataaaccatggcgtaaacctggtgctgatctttctgattattttaattatgggtttaatgaagatacctggaaagcttactgtgaaaaacaaaagaggatacgaatgggacttgaagttataccagtaacctctactacaaataaaattacggtacagcagggaagaactggaaactcagagaaagaaactgcccttccatctacaaaagctgagtttacttctcctccttctttgttcaagactgggcttccaccgagcaggagattacctggggcaattgatgttatcggtcagactataactatcagccgagtagaaggcaggcgacgggcaaatgagaacagcaacatacaggtcctttctgaaagatctgctactgaagtagacaacaattttagcaaaccacctccgtttttccctccaggagctcctcccactcaccttccacctcctccatttcttccacctcctccgactgtcagcactgctccacctctgattccaccaccgggttttcctcctccaccaggcgctccacctccatctcttataccaacaatagaaagtggacattcctctggttatgatagtcgttctgcacgtgcatttccatatggcaatgttgcctttccccatcttcctggttctgctccttcgtggcctagtcttgtggacaccagcaagcagtgggactattatgccagaagagagaaagaccgagatagagagagagacagagacagagagcgagaccgtgatcgggacagagaaagagaacgcaccagagagagagagagggagcgtgatcacagtcctacaccaagtgttttcaacagcgatgaagaacgatacagatacagggaatatgcagaaagaggttatgagcgtcacagagcaagtcgagaaaaagaagaacgacatagagaaagacgacacagggagaaagaggaaaccagacataagtcttctcgaagtaatagtagacgtcgccatgaaagtgaagaaggagatagtcacaggagacacaaacacaaaaaatctaaaagaagcaaagaaggaaaagaagcgggcagtgagcctgcccctgaacaggagagcaccgaagctacacctgcagaataggcatggttttggccttttgtgtatattagtaccagaagtagatactataaatcttgttatttttctggataatgtttaagaaatttaccttaaatcttgttctgtttgttagtatgaaaagttaactttttttccaaaataaaagagtgaatttttcatgttaagttaaaaaaaaaaaaaaaaa

SEQ ID NO: 18 - DNA, 652 bp, Homo sapiens
ggacggaaggagcggcggcgacggaggaggaggatggaggcggtggtgttcgtcttctctctcctcgattgttgcgcgctcatcttcctctcggtctacttcataattacattgtctgatttagaatgtgattacattaatgctagatcatgttgctcaaaattaaacaagtgggtaattccagaattgattggccataccattgtcactgtattactgctcatgtcattgcactggttcatcttccttctcaacttacctgttgccacttggaatatatatcgatacattatggtgccgagtggtaacatgggagtgtttgatccaacagaaatacacaatcgagggcagctgaagtcacacatgaaagaagccatgatcaagcttggtttccacttgctctgcttcttcatgtatctttatagtatgatcttagctttgataaatgactgaagctggagaagccgtggttgaagtcagcctacactacagtgcacagttgaggagccagagacttcttaaatcatccttagaaccgtgaccatagcagtatatattttcctcttggaacaaaaaactatttttgctgtatttttaccatataaagtatttaaaaaacaggaaaaaaaaaaaaaaaaaaaaaaaaaaaaaa

SEQ ID NO: 19 - DNA, 3346 bp, Homo sapiens
acgtactccatgcgctacctgctgcccagcgtcgtgctcctgggcacggcgcccacctacgtgttggcctggggggtctggcggctgctctccgccttcctgcccgcccgcttctaccaagcgctggacgaccggctctactgcgtctaccagagcatggtgctcttcttcttcgagaattacaccggggtccagatattgctatatggagatttgccaaaaaataaagaaaatataatatatttagcaaatcatcaaagcacagttgactggattgttgctgacatcttggccatcaggcagaatgcgctaggacatgtgcgctacgtgctgaaagaagggttaaaatggctgccattgtatgggtgttactttgctcagcatggaggaatctatgtaaagcgcagtgccaaatttaacgagaaagagatgcgaaacaaggtgcagagctacgtggacgcaggaactccaatgtatcttgtgatttttccagaaggtacaaggtataatccagagcaaacaaaagtcctttcagctagtcaggcatttgctgcccaacgtggccttgcagtattaaaacatgtgctaacaccacgaataaaggcaactcacgttgcttttgattgcatgaagaattatttagatgcaatttatgatgttacggtggtttatgaagggaaagacgatggagggcagcgaagagagtcaccgaccatgacggaatttctctgcaaagaatgtccaaaaattcatattcacattgatcgtatcgacaaaaaagatgtcccagaagaacaagaacatatgagaagatggctgcatgaacgtttcgaaatcaaagataagatgcttatagaattttatgagtcaccagatccagaaagaagaaaaagatttcctgggaaaagtgttaattccaaattaagtatcaagaagactttaccatcaatgttgatcttaagtggtttgactgcaggcatgcttatgaccgatgctggaaggaagctgtatgtgaacacctggatatatggaaccctacttggctgcctgtgggttactattaaagcatagacaagtagctgtctccagacagtgggatgtgctacattgtctatttttggcggctgcacatgacatcaaattgtttcctgaatttattaaggagtgtaaataaagccttgttgattgaagattggataatagaatttgtgacgaaagctgatatgcaatggtcttgggcaaacatacctggttgtacaactttagcatcggggctgctggaagggtaaaagctaaatggagtttctcctgctctgtccatttcctatgaactaatgacaacttgagaaggctgggaggattgtgtattttgcaagtcagatggctgcatttttgagcattaatttgcagcgtatttcactttttctgttattttcaatttattacaacttgacagctccaagctcttattactaaagtatttagtatcttgcagctagttaatatttcatcttttgcttatttctacaagtcagtgaaataaattgtatttaggaagtgtcaggatgttcaaaggaaagggtaaaaagtgttcatggggaaaaagctctgtttagcacatgattttattgtattgcgttattagctgattttactcattttatatttgcaaaataaatttctaatatttattgaaattgcttaatttgcacaccctgtacacacagaaaatggtataaaatatgagaacgaagtttaaaattgtgactctgattcattatagcagaactttaaatttcccagctttttgaagatttaagctacgctattagtacttccctttgtctgtgccataagtgcttgaaaacgttaaggttttctgttttgttttgtttttttaatatcaaaagagtcggtgtgaaccttggttggaccccaagttcacaagatttttaaggtgatgagagcctgcagacattctgcctagatttactagcgtgtgccttttgcctgcttctctttgatttcacagaatattcattcagaagtcgcgtttctgtagtgtggtggattcccactgggctctggtccttcccttggatcccgtcagtggtgctgctcagcggcttgcacgcagacttgctaggaagaaatgcagagccagcctgtgctgcccactttcagagttgaactctttaagcccttgtgagtgggcttcaccagctactgcagaggcattttgcatttgtctgtgtcaagaagttcaccttctcaagccagtgaaatacagacttaatttgtcatgactgaacgaatttgtttatttcccattaggtttagtggagctacacattaatatgtatcgccttagagcaagagctgtgttccaggaaccagatcacgatttttagccatggaacaatatatcccatgggagaagacctttcagtgtgaactgttctatttttgtgttataatttaaacttcgatttcctcatagtcctttaagttgacatttctgcttactgctactggatttttgctgcagaaatgtatcagtggcccacattaaacataccagttggatcatgataagcaaaatgaaagaaataatgattaagggaaaattaagtgactgtgttacactgcttctcccatgccagagaataaactctttcaagcatcatctttgaagagtcgtgtggtgtgaattggtttgtgtacattagaatgtatgcacacatccatggacactcaggatatagttggcctaataatcggggcatgggtaaaacttatgaaaatttcctcatgctgaattgtaattttctcttacctgtaaagtaaaatttagatcaattccatgtctttgttaagtacagggatttaatatattttgaatataatgggtatgttctaaatttgaactttgagaggcaatactgttggaattatgtggattctaactcattttaacaaggtagcctgacctgcataagatcacttgaatgttaggtttcatagaactatactaatcttctcacaaaaggtctataaaatacagtcgttgaaaaaaattttgtatcaaaatgtttggaaaattagaagcttctccttaacctgtattgatactgacttgagttattttctaaaattaagagccgtatacctacctgtaagtcttttcacatatcatttaaacttttgtttgtattattactgatttacagcttagttattaatttttctttataagaatgccgtcgatgtgcatgcttttatgttttttagaaaagggtgtgtttggatgaaagtaaaaaaaaaaataaaatctttcactgtctct

SEQ ID NO: 20 - DNA, 459 bp, Homo sapiens
  misc_feature (12)..(13): n is a, c, g, or t
tttttttttttnntttttttttttttgtatgttggaacgtgccccagaatgaggcagttggcaaacttctcaggacaatgaatccttcccgtttttctttttatgccacacagtgcattgttttttctacctgcttgtcttatttttagaataatttagaaaaacaaaacaaaggctgtttttcctaattttggcatgaacccccccttgttccaaatgaagacggcatcacgaagcagctccaaaaggaaaagcttgggcggtgcccagcgtgcccgctgcccatcgacgtctgtcctggggacgtggagggtggcagcgtccccgcctgcaccagtgccgtcctgctgatgtggtaggctagcaatattttggttaaaatcatgtttgtgactgtaaccatttgtatgaattattttaaagaaataaaaatcctggaaagagnnannnnnnccagca

SEQ ID NO: 21 - DNA, 2574 bp, Homo sapiens
  misc_feature (200)..(200): n is a, c, g, or t
cggcacgagggtttgatctaatcttaatcacagtgagctcttcattagcacaatatgtggtttgccccaagtgtgcactatttaatactttgtaatatgccaccaagagtactgacatttagagttgtttaaaggccgagaactggaaacagcctttccctcattttctgtgtattggtgatgggagtaataacattttnggggagctttttaaatttcacagaagaggaaagttgcctgctctggcaggtatgtgcaagatagagtgtgtttcatttgttctgttgccaagaattagtgctgtactattgtagttcctttaggatttgtatgtgctctgggctcatgaagatattgcatcatgagcttcagcagttgtactcttttttgatgacctaaaaagggcttatttctgaggaatgaaaggttcccatcattgactanggatgtggaaaacctttcctagcttagagcatttgtatctatattttaaagtcagagttcatgttacctgttttaatcacatgactgcatgtcccagtacacaaaanggcactggttggcattcttcttaatgtatttagtaaagatcagaagaaatcctttaagagttnaaatgtccctggaacangcatacaggctctagtcaagaatgaattagagtgaaggaaagctgtgtgacacctggcattcctctgttcatggagcttctttgaggcttgaagattgattttaccatctagaccactctgcctattcttcaaccaccttggttactttgacataggaattgacttcttttccttgaatggaaaacactttgaaataataataaacattgttataaactaananatgtgagagtgcttagttgaaacaaaaaggagttttagtagacagtattatactatctttgaaaatcaaggagaagtttatgcaacttaaaatgtgtacaaactgcagngcaatctactgttnggtgaatgtcagtgtattatcaggaaacatgtctatacaatcacangagttatatttcctcacaaacttctttgtgaagagngaaatgngtttctgntacctctgggnttcancttacgggncatattttgngcangnattnatgtgattgntgcctatgcntgnatgaatngaatgaanntttcnagttngnacattgcctnaaatcnataacttgatgatgcttgggaaagactcaacagttaaaacttcatgaagttctaatgtctgtgttccaaaacacatcacattattaggatgtagggagatatgtatgtgtgctccctggggtggggatttctagttactagaccatctccatttttagcanttggcatcctcatgatacttttataaatacgacattaacaggagagcagcagtacgattttgccgatggaataacagatttgccggcaatcactgaaagagtgcaaatatcgggtccttgtgacttcaacggactcttccaaattgtatgaatgtatcaatgtattagataaacccagtttcagaatgataaagaaaaaatgttagaccaaataatgcggctagttaacagtggtacgatttctagcccgtggntttaaaatgcacttaaagtcctgtccttgccttttattttctgaacttgatgttttngcattctttgagttcagtttaaagacaactacgagcatctgtaaccaatctgacaataatgtgttcatcaggtgcctntggnattaaatcacatactggcatatttaagctgaatgtcaatctggaaaataaattgactgtattaacggaaataccactctttgtgtagatatttgtcgtatattgaagaaaaagctaaaaagaatggaaatcgcatgactataacttaagtctttcttcaaagtgcatgcagtcttttgcgatacctcattcagccaagtattggtattcttcctcattcggtataaggcagctttcaatttgcttagagggcaacattggaaggttagagttcatcagaaacagaattctaaaatgtgagttcaattcaataaatttgaatttctgtaggaagaatcaaatcaccgatttaaagattgcaatatataataatcatttttaaagtattggattaaatctgataggttttccagaaatgaacaaaaatcagctctaaaaccaaagctgatttttagaaaatttgaaaatgtaaatcagccctatccatactatagtttctctaaaactttatctgaaagagtcattttaaaataactattaaacaatgtaactgctatcttaatgttctgaaataagttaaaacattttaaaatatgaatactgtaaaggaaataaacggtgggaaggaaaagtagagaaagaaatgccaattccagtccaaagctttatttgccaagttttcttagaatgaattttaccaatttatgaattcttgtaagcggaatgtaaaacggaaatactgaaagacttttgcctaaagtggcattattgactgctggtgtgatgctactgtaatgtaataaattattaagttgttgcaaaaaaaaaaaaaaa

SEQ ID NO: 22 - DNA, 1904 bp, Homo sapiens
ggaagggagggagcgaaggtaggaggcagggcttgcctcactggccaccctcccaaccccaagagcccagccccatggtccccgccgccggcgcgctgctgtgggtcctgctgctgaatctgggtccccgggcggcgggggcccaaggcctgacccagactccgaccgaaatgcagcgggtcagtttacgctttgggggccccatgacccgcagctaccggagcaccgcccggactggtcttccccggaagacaaggataatcctagaggacgagaatgatgccatggccgacgccgaccgcctggctggaccagcggctgccgagctcttggccgccacggtgtccaccggctttagccggtcgtccgccattaacgaggaggatgggtcttcagaagagggggttgtgattaatgccggaaaggatagcaccagcagagagcttcccagtgcgactcccaatacagcggggagttccagcacgaggtttatagccaatagtcaggagcctgaaatcaggctgacttcaagcctgccgcgctcccccgggaggtctactgaggacctgccatgccatctcctgaggatctgcggctggtgctgatgccctttccgggcgccttcgagttggggcgctgagccagctccgcacggagcacaagccttgcacctatcaacaatgtccctgcaaccgacttcgggaagagtgccccctggacacaagtctctgtactgacaccaactgtgcctctcagagcaccaccagtaccaggaccaccactacccccttccccaccatccacctcagaagcagtcccagcctgccacccgccagcccctgcccagccctggctttttggaaacgggtcaggattggcctggaggatatttggaatagcctctcttcagtgttcacagagatgcaaccaatagacagaaaccagaggtaatggccacttcatccacatgaggagatgtcagtatctcaacctctcttgccctttcaatcctagcgcccactagatatttttagtacagaaaaacaaaactggaaaacacattgtttggtcttgtgtttctttacagaggtacctgagggaggagagacataaatcccttcatccctaagactgaactatgtaactagcagcctctggcttgttttctactccctgtccctcaggataaaatgttgatattgctcattttcctcatttccaacattgttttaaaacaagtacttcttttacaggcttgaaaaatctcaaataaacgctaagaaaagggagtaggaagaacaaggagttgagcccttgaaagatgacagtggtcttcttgccttcatgcttggccctctctcctcaaaagggcaatgttggtacaaaattccatctcagccactttcgaggagttatcttcattagctatatccatcctttaatccaacacacacctgcaatgattactgtgcaactattttgcttaattttttatttgaaaaaatgtatttaaaagtccaacaactttttaatataaattacgactctcaaacccattcccatcactttattagtgatggtagcatacatattagagaaggtagctaaaggcaagagagcaccaaaggaaaaagactgtccaaagaacaggtattagaatgaggccgaagatcacggtgaccagagatttctaggagtctctaacctttccaccctatcctgttaaccctttagatctctagtataacactcaggctactgaggtattttagagcaacaagctgggttactttcagagcaaccagcttgactggaactgagagtaaattgggaatgtatgaccaatcttagaccctgaaaaatggcagaaaatacatggaaatttgaaaaaaaaaaaaaaaaaa

SEQ ID NO: 23 - DNA, 12609 bp, Homo sapiens
  misc_feature (35)..(37): n is a, c, g, or t
ttttttttttttttttttttttttttttttttttnnnttatacacatccaattttattaacattcacttataaattatctgcccttgtcatatattggaatcgtgttttttaggcaaatataacaatcctctactgcaggagagaaagtcttcttggtgatatgagaaatactggtgtgataatttcataaattattcaagtctgtggaaacactccagttcagagaagggtacctggnatttgttttcagctttctcnattttcacatgcacgttaggctatctggttttgatagtgcgtgttttcttctattaagtgagatagctttctcaataattatatcagaaacagcagcagtgagtgactagagggcctctactggcctcaggcattttctaaaaaccggaattagctgtcaaattttgagaatacagagcttcccacggcctcccccatccctgcatttggtgtgcacctcacaatttcatgcaatttagtgacttgccaagctcctgcaggcatctgtgtttgtgctgaagatgtctgtctcctctgcggtgcaccgtgtcgtgtatcacagcatattggaatgggtctgtaggtaggaatagctgatttgttattcaggatggctttaacagccgctggaaggaacacaccatcttatggtagttgctcttccacacagctttgttctctgcatgggtgangggtatctctggggtccatcaactattacatcatgatgcctgtgcaatatttacttgcttacgtccaatctactgggtccctgaagaataagcttttttcttcttcttagctaatatttacccctccttttctctaatgatagagtactgggaagcagaaatcccnnnntncnccttctctcacnaactccccagcctccccactcattctcagtcctggggactgggggctagtggctgagtgtgggctatactgccttgctgtttgcaatgaaacaattgtaaaataaagagagcagccaaaacagggagttatgagctgagaatatgggaataagcacacaattagttctgacgaaagaaatatcttcctgatgttattaacaatctactagaaattgtgtcctctttaccaacagaaaaatattgatgtgcaagaaaataagcttatttctaacttnattttttaagnggtagaanntntnntnnnttttgcatacatacaatttatttatttatattatagccctgtatgatacaagcattttctcatctaaaagtgttattttaactcagaaaacatactngcattaagctcttgagcctcagaatggagcacagagggaacaaaacccttttgaaattaaaaatattattcagcagaaactgatacttttgaaccaatatgaatttgaaataactaaatattttttcaaaatctcccttaacatcaangttcaagatgattgcatttattttgatgtcactatataaatacaaggatgttaactgatactcacctaagtcaaatattccagttgcattaacaaacatacacataacaaatataaatttatatcaaatgtcaaatttccccattatcttctcctcttcttcattaggaatttgatgttctgaaaagtgccttttttgtctgcttcgattttttgttgctgttttttttttttttttgctttataatttctaagcatttaggcagaaaagaaagaggcattaaaaatacaactcagaaatatttaaattttgatgaaataagagtcttgttagttgcagtctcttttgaatactttccttgggagaaaatatgctgtgaaacagtgatctgccactacanagtggttggggacattaggtcatttctagaaagcaattatatacacctaatggcttattccctanaaaatgaactattatggtgtcagaaaaggctagatactttatcatctgaaagacaggttgctcagatgtatacaaatgaatttattctcttgacctctagttcagatgtcagaaatattttgtactaaaacagagataggttcctatattggaatatgttcaatatttactacctttctcttctattctctatccataaaactctgcctgattaaaatattttaaagtgtactttttacctgatactgttaatagcattgatgatcctggattttaaagatggctgctttagtctactttgattttgggaaagccattttaaacaaacaaatatgtggtatcatttaaagttttataaagaattaagagtaactgaatgacagctgaatttctcatgtatttattgactgcctcctaggttcaaagttctgtgctgcatgcagacatatcaaaatttcttatatctttggatcatgaatgtcagggtctttcagctcctggtcagaaaaaatactgtcatgtcagagtgaggaattcttagtatgaggtggtgctggggagtaccagtagcttcagttcaaaaattcattgctctgggtcccaacagctttgttccaagtctacatcttaacagtatgtggaaggattggttcaggcttcctttatattctacatagatcttgatttgggatttttaggaaccaatagcttaagctatgttagaacaatccaaaagctttaaaaattgtcagagcaaaccctatagcaaaccctatctagaccttaaaccttggcgttgataactgagaatttaaagactctggaggcagtgttttgttcctgtgggggagaggccaggtcaacttgcagagtatctcccagagtctggtatcaagtgtcttcactccctgtcacatcacttttttcccaacactttcattatccatattttgctctgctattccaaatgtgtagtctctaagaataacctgaacacnccaggttattctctccctcgacaaattctgagtgcatgatttaatacaaaaacaatttgaataagagatttttatcttgctacattttgaaaagcccttacagatacagttttaaatatgtaattgaaatgtggcattttcttgagtattcaatacatttcttggatttttttttcctaactgcaggaacagcatacataaaaacgctcgggtcctgaagcaattctttaagcacagcaaacgttgagtcanntggctttcactccatgtttataaaaggcaatgccaccatcataagtttctcaataattcctatcacttntaagcattttcatgtattaatctcaatatattttaatagcatacatatggtctcaaaaaaccctttcctggggcttactgttggctttttattcccccagatcttcttaaaatcttagcctatacatgacttagaacatgtttttaacaagttatatgaaaatctccttttgtgctccacattgacagagcctgtctgaatcccagagaaggcaatcacatcctgttatgttgtcagtgtgagaggcaaaagctccaaagtactaagttcctagagctgtaaatccattgtgcttcatgttataaataaagaaattgccctgtgacagctacagtttggaagcattatcatttctcttttcanctctcacttttaatcttttcagttattttccttagtaaaacttagccaccaaagtatgacttcttgtgtcacaaacctatatgccacaattaaaagaaagaaattccagccaaccatgtcagtggctgtagtcagcatttaagacaaaactgactcctatcgatttagcagaagagagttctccctgccccgtgtctccttccatcctgccgctccctgccctctccagccttttgggcaagccttttcttcctgatgcattgcgaatgtgtgatgtacgctttctagcagatgcatttgaggttaggtcttcgcaaccatgaaaacacagttataaattgttctcggagctaacccactcaacatcttggcacttggttgtttttaacttttcaacaataggcactgttcactcccctccatctcagttctttattcagtgatcaggcaggacactgatgaaaggctagtcacggcttccccccatggtatcagtgtggtggcatgtgatgattcacatggggtgtgtttgatccgaggaagcccagtctctgcttattcttcctttgttctgtcatctgctccttgagctccgccagctgacccgacagctgtttgaccagactcatggtcgattccaacttctcctgaaggctccgaatttcattttgctcactgtcgccttcattgctaacgagggacatggctcgcatccgaggaaaccaatccaaattcttctccacaatcatttgagccacataactttcaggtccagtgtattctgttgngtctttaactttcaccaggactatgaagtacaaataatgccacatattgtgttctgacttaatgtgctcctcaaatgaanccgttttattatcaaacttgtctctctcaagtccacagatgaaacaagttgtctttagaatttcttcttttttctgtttttcgcttctgagatcagcaaaagtatcgatgataacaccaaaaatcaagttcagaacaataatgataacaatgaaataaaaaagaaggtcataaaccactcgggcagcaaacaanggctcatctttcgatggccttcttagcacatcccccacaccaccgccattcntgaggccctggttcagcacggtgacaatgcacataaggagagtgtcacacgtcctttcaattccatcttcatactcttcatcagctgtatttgaagctggaattgtgggtgaacagttctccttggcacatgcttccatcatggtagttaaagtcatagtaggcacttgatgactgcctgtaacaggagttcggtttttcagcctatcaacttccatagtgaagtcatccttcaaaaaaaggaacccaataatggaaaacaggtagacgaggatgagagccaggactgcagttagaataatagagcggccatttcgtgtgacactttttatgacattcagcaaagtctcttccctgtacaccaaatcaaaaagcaggaagctatagaagaattcatggacaaaaaggcccagcatgcaaaccaggacatacgccacgtgatagagaaaggccatatccaggatgactgctcggtacccacgggtgaacgtgccacgatttccaacnaaactcaccagaaaaacaattttattacaaagattagctgcaccaagaagtattaatgtaggcccaagacctattgtatatattgatctgagcattattgatacaagaaacggccgaatacccacaggcttggagaagaaaaacagcatagatgtgcagatcgcaactgctatccaaagaagaaccgagaacaatggagaaagtgtaccttcatctccatcatccccaaatgggtagaagagagcaacagctaaattgatgaacacagccaggttgaaggaaatgctcccccagagagagatgtgcctcgagaaccagaacagtgcagggttattcctgattttcttctgccacttcatttcattgtagagatcttctgtttgctggaaaaagtcattcactttacttccttgttcatccctttcagttgtattgaacacacggcacttggattctcgagtgaggtattcacatatattggggacaggaaaaactatttgttccatggtcctatcatgccggacaatctcaatctgtgcagtgtggttggcataatactttaaggcttcatctccttcatctggatccgatcctggtttgagcatctgctgcaacagtttattgtggcgggccaactgatgggccagaatatagatattgtgtccaacatcttttggagaaacaccatcatctccaccctcatcatccccatggtcacattccaatccttggttataggcattcttcatcacatccaccagttctctgggtctcatgttaaaaagaattctttctgcattctcactgtcatgtctgctttccataatggccagcaaaagtttagatgcattgttctttagctggagcaccaggtccattcggtatttaccaagagggtttatgtcattcagaatcaaagcaatgatgatatcaatcccattagactcatgtgtagcgatacaggtctgattttcatggcaagggccctggcaatactcagtcaagctctccaggttctggttgaccagcgctacattcttctcattgatgtagagacccaacaggcccaggccaccggttgtacttccacaaatgcagtccagaaactgaagggtctcacagactaggttgtaatttgttttgttgttttgattcctcaagaagttctgcaattcccggttgtgattctcacacagtaactgaagaaatctcagtattggctgcatgatggcaattgcgggactcattgttacttcctctgcggatttttcctcagtgtttcccgcttctggtcctgtgcacataatgtctatttctggatccatttctcttctgtatacacaatatgctttggatgttgctgaagaagcttctgttaattgccctttcattccctcttttaaatgtagtgttgaatctcttactctcattcgtggaccagatgtcatcaattcattgtcatcgtccctttttttgttacctaaatctatggtattaactgtcactgttgatcttatttctttctgagcagccttcattcgatcatagagaactttaaagaatttttctgactttttttgttcatgcaactgctggtagaaagaatactgtgtttgtgtatttcctccttcaagcaaggcaatgccgaggaaaatgccttctgaaaaaattctgtcatttttggtgttcactataacatcgatgacaagttctgatgcaccttctttatccagcagacactgaatgtctgacattgatatccccatcttatctgaatcttgtccagaaaagcttcctcccacctgtgcagttttggagtaggctcctgataggtgtccattcacaccaatactataatcacctttaaagtatcgattcagaagtatctttcttaatgtgttaccctcttccacaaagctgtctttcttctctaacatttctcgtaatgtctgaagaattttaatgcacagtttttcttctttctccattagtttctttgtatgattaatcaacttcgacatgaaagcgccacatcttattcttgcatcgcttccctcagggaacagcagttctggactgtacaatacatcaaccaacactgagaattcagcctgcatcattgggctgaactggtgctccaaggaggccactacatcctgtaacttttcaataatatttctgtaatcccaagcaggccctccaagagcttccttaaagcgtggcccagagcgagctgatagtctccaacccattgctgctctctgcaccatatttgaatggctcttcatgaaaagagtattaacttggctgtccaaatccactggaatggcaattccacgattttttgccacttcagccaaagttctgatacaggattccactgaggctttctgcgctgggtttggccaggtgcaattgtaaattctgaaggcagattgcagtagctgaataaaaactggctgatgtgtctggaggctggtactattgtctgaaaagggagaattaaagaagccgctcacaatattcattattgactcagtaacacacttttccaaaaagatgtctgcatgtttcctgtctgtagttgtgttgcaaacccttgccatatccaccaagaagttctcaaataatttccaaatgtggttacttgtatagatttctttcatttccacttcagtgtcaacataacagtgattaacaaagttcacataagcaattttaacctcagggatgcagtcgtcatgggtcaccaccctcactatgtcgtccagcgggagaagggaattacacttgatttcagtgtagacatttttcccctctgtgcatgctgccagcaactccaccagggtgatgtggtaggctaaggggccactctcatcccctcggtctctctctgaacacatcatatggagaaggattggaaatgatgctctatcattgtaaaatatcagcacgtcttcacccccatttatcaactctgtcattaccatatcctggcatttcttcacatatttaccatctgcttttacaattgtttgcaaaaacctcaggtactccacgtggcggccatgtgtctcaatgcagtgcacaaanngntgtacaantctctcgctaatttcgtngcacagatggtaatngttcatgaagatgtgccgcatggtttctgcttcaaggagacctggagttaaaaacaaattcagatgtttatgaagaagaacttgattctgtggatttcctcgacagaaattctgcagaaatgtatgggctagattcattacttcattcatcttttcatcattcttttcatagggtatctgcagaagatccaacaccaccgaatgcgcccccatatttttcagtaatcgttgatgttgattccgacactttttattctgcacacagagtttacttagcctgatcaaaatctcctttacaatccggtanttattgctcttgttgctgtcaatctgaggtttctttgttccatcctgcactggacttaaaatgtttgattcctcaattggctcttcaccacctttcacttgactttcccctatttctccattctcatagttgctgctcttctccacccatagctcagacttttctactgtcagtcgaagctggtctagatctgccttgatttgcttgtagttatctacgtcttgnnnngnnancagtaattgcacctgcttaaatgcctgtaaaacctctgccctctggctgaagtgcttaaacaacagctgcagggctccagacagcaaaggcgngtagtcgtgcatgatcagatgaatgaggacccgtaaaaacgtcctgcctccttcatcgtcaagttgaactggatttttttcttttcttcccgcaaacatagtttctgcctgagctgcaatttcatctatatcaggaacaatagctgatggtagtaaagtgtctggagatccactggcagatgtctccgcattgtcattgtcctctccnaactccttctnatatattgacagcatatatgagatcctataatccagtctgacactcaggataaactgcaaaatctcaatnntntnnannnnngngtccatcacagtcacatcctcntgctcggtggggctcccttgcttgctcgggtggatgctgggtggcacatccggcacgctcatggggaagatggagcctctactgagtaccatctgggtcatcatctctcccaccccatgaatggttctcatcacattgtttcctccatcttgaaatttgcttaatctttcaaagtatgatgacatgggggcctgtacaatgtctaaaatagccagaagtgttcttgttagccttaataactcactgaaactataaaatccaaagtatataagattccgagccaagtggaccacctcaaatgtcagtttatttttttctttatccccaaaaggaaagggctggtttacaacttctttcaaatattcttcaacaaattccattgtcagggnnaatttcctcttcatatcatttctggaagagtctgttatagaatcatattcatgaattgtgatctttgtggggatttctgtccagagcctggcatagcgaacaggcaccacggactcctggggatcccggtcaacgtgcatgtggagcatgaggcgacagaaggacgctcggaggtcgaacggcaggctctcatccgacacacaccgcaggatcaggtctacagacagctgtgtagaaatctggtttatggccagatactggcgatccaagcacatccttgcaaagaggtttagctggtacctgtaataggtaagaacttctaagtcagctttggtgccttcttttgcctcttgagcaaggtgcctgatagctttgccatgaggttccttgttgctgtcaatccaatagagccaaacttcttcatcatcaatgtcatctgaaaggatggagctctccatggggttgtctgcttgcattgagaccaccttagtttgaatgagaatgtctgcattgcctggactcaacataaatttacagatgagttcttgagttacagggatagcagtggtattagacacacacagatctgacaaataatccaaaaaccttggctcccgatttctcctgagtaaactgacaaatgtttctatttcttttgctgtgatatgtttctctagtagttttctgttgttgtgcaacaaagntgtgatagtatcttctgccaaaatatcatagccaatctgggactgcatgacacagaaattcttagcaatatattcctgatttttccggtaatcctgctgcgagtgtctcaggacgcggtaacagagccgcagcatgtacttgtagggtgcatatctttgatcccccagatcttcaagtctcagcatcgagccttctcctgctttctctttaaagggtgctttaagaattccaaatacctgtgccagtatgttttgttccctcatcaatttttgacgctctcggtttggcttagtgataaccacatccagaacttcttgtccattattaggcacatcagcaacaaagaatatgagatcttccaataatttggttacaaacctcctttcattctgagttattgtgccgttttctagctttttaactgtggtcgctagtactttattggcatcattggcaaagtctaagtctcgaacttcagacagtggaacagacacgattgcgaacgcttctttatcttctttggtttggcaggttccaatctttaacataacaggcctctcttcatctgtgtctatggggatactagtactggttacccatgtgttggtgcataaatgccttaaccgaacatatgagttccttggaaccaggcagtcagctctctgaagagttgtggcatctagttcaaaaagggatgcaatgtcattgccatgcgggactgaaaccaaagtatacatnatcttctcccctgcctggcgttttttctttgaagttggagggactccatctctcacattttttccttcattttgggcatctcgataatcaggattaagctctgcagctaaatagtttccagttgcaagatgcttaaatctgaacaagctgttccactgtcctgcacccccacggcatgggtcatgatgaaccacctctatttcccagagtgctttagaactagtagcagaagtagctgntngnnncaaggtcgtacgaaggaaaatgtgctgttttttctcatattcatcacaagtcaaaaacttctcttgttccgcatgaaataatctaacaacgtcccctccttttaatacatcctctcgataggaactatatttcatgaataaagtgattttccagctggtgttgcaattgacagcattcacctctttacaccctgggttatcaagaagctctatgttgctggcatgtagtggctgccctgcattcacaggcatcaaaacaactttatctcctacaacaatattgtcaccctcgcttctcagtttccagaatggatgaatataaaaccaagacccttcatttcctgcagcatccaaggacacacgcatggcattcttctccagtaaagcaggtaatctcttgttgacagtaagatatttgttgctttttatatgcagtagttgtataacattactgtattttacaatttctcccaacagtttcttattctccgattcattttgtttttgttccagttctgcagcgtgctgtagtttcttcagcaaggctgcctcggtgtggttcccttgtttggcttgctttgctttccaatattgcttctgggcagaatatctgttcatagggcacaccttgaaaaggcagtctctgaacttcttgggagggttggcaaggtccccggcctctgggtgcaccacacatctgtcatccactaaccccaaggtgctgatgaagccgttgaccgagccctccgcgtacagggacacgatgtcccctatgtagaggaagctggacattttctcagtcatgctgcttcatgctgcttcatgttccacagtggacgtccctcttcttccctgcgccctcgccgccctctctccagggagccgccgcggcagaagcggatcggatcgcgggactacagcggccaagagccacggcggaggcacggcccgagccactgagcgtcgcgctcagccgtggtgcgcgccggggaagccagaccggggccaagcgccagctcgggacaccccgcgaagaggcagcccaggcgcccagagaagccgcagccgccgcccgctccccggcaggtttcctgttcctttctgaagttttctctccaacacctttgctcctcctcctcctccttccctctccgctcccacctcgtgtccactccctgctctgcgaccgctcggccgtcacagcgccggcgggagctggaag

SEQ ID NO: 24 - DNA, 3976 bp, Homo sapiens
gaattccgggcccccggccgtccctcttaaggctcttcctccgctccgccagtgtccggccgcgggccggccttagtgactggggcggcgggccccggggccgcggcgtggggcgggcaggcggacgccggccgcgggctgctttcgtcggctcccaagctctcccggagcgagcagccgcccgcgagccgccgcggagcctcctcgcccgctcccgccggcgagcaagatgatgtgcgaggtgatgccgaccatcagcgaagcagaaggcccccctggaggaggtggaggccatggttccggctccccttcacagccagatgcagattcacattttgaacagttgatggtctccatgctagaagaaagggaccgccttcttgatacactgagagagactcaagaaacgctggccttaacccaggggaagttacacgaggttggtcatgaaagagattccttgcagagacagctcaacacggcacttccacaggagttcgcagcacttactaaagaactcaatgtatgcagggaacagctccttgaaagggaagaagaaattgctgaactgaaagcagaaaggaataacaccaggctgctgttagagcatttggaatgccttgtctccaggcatgagcggtctcttaggatgaccgtggtgaagagacaagcgcagtctccagcaggcgtgtccagcgaagtggaagtgctgaaagcactgaagtccttatttgaacaccacaaagctctggatgaaaaggtgagagagcgattacgagtagcacttgaaagatgtagtttgttagaagaggaattaggtgccacacacaaagagctaatgattcttaaagaacagaataatcagaaaaaaactctaacagatggagtgctggacataaaccatgaacaagaaaatacaccaagcacgagtggaaagagatcttctgatggttctttaagccacgaggaagaccttgctaaagtaattgagctccaagaaatcataagtaagcagtcaagggaacagagccaaatgaaagaacgcctggcttccctttccagtcatgtgacagaactggaagaggatctggacacggctagaaaagatctcatcaaatctgaagaaatgaacacaaaattgcaacgagatgtccgtgaagccatggcccaaaaggaagatatggaagagagaatcactactcttgaaaaacgctacctcgctgcacagcgtgaagccacatctgtgcatgacctcaatgataaacttgaaaatgaaattgcaaataaagattctatgcatcgacagactgaagataaaaaccgccagttacaggagcgcttggaattggcagagcaaaagctgcaacagacactgaggaaggcagagacgctcccggaggtggaggcggagctggcccagagggtggcagcgctttccaaggctgaagagagacacggcaacattgaagaaaggttacgacagatggaagcacagttggaggagaagaatcaagaactgcagcgggcaaggcaaagagaaaaaatgaacgaagaacataataaacgtttatcagacactgttgacaagctgctttcagaatctaatgagaggcttcaacttcatcttaaagagagaatggctgctttggaagataagaactctcttttaagagaagttgaaagtgcaaaaaagcagttagaagaaacacaacacgataaggatcagcttgtcctaaacattgaagcactgagggctgaactagaccacatgagactaagaggtgcttcacttcatcatggccgaccccacttgggcagtgtcccagatttcaggttccccatggcagacggccacacagactcctacagcaccagtgcagtgctgcggcgcccacagaaaggccggctggcagccctgcgagatgagccttccaaggtacaaactcttaatgagcaggattgggaacgtgcccagcaagctagtgtcttggcaaatgtagcacaagcattcgagagtgatgctgacgtgtctgatggtgaagatgacagggacactctcctcagctcagttgacctgctatcgcccagcgggcaggccgacgcgcacacactagccatgatgcttcaggagcagctggacgccatcaacaaagagatcaggttgattcaggaagaaaaagaaaatacagagcagcgggcagaggagattgaaagtcgagttggcagtggaagtctagacaatcttggtcgttttagatcaatgagctccattcccccctaccctgcttcctcgcttgctagctcctcccctccgggcagtgggcgctccaccccacgaaggatccctcacagcccagctcgggaagtggacagactgggcgtcatgacccttttgccaccttccagagaagaggtacgagatgacaagacaaccataaagtgtgaaacctccccgccttcctccccgagagcccttcggttagaccggctgcacaaaggggcgctgcacaccgtcagccacgaggacatcagggacataaggaactccacaggctcccaggatggtcccgtgagcaaccccagcagtagcaacagtagccaggactcgctccacaaagccccaaagaagaaaggcattaagtcctccattggccgcttgtttggcaagaaagaaaagggccgacctggacaaactggcaaagaagcattaggacaagctggtgtttccgagacggataactcatctcaggatgccttgggacttagcaaattggggggacaggctgaaaaaaatcgtaaacttcaaaaaaagcatgaattgctggaggaagcccggagacaaggtttaccttttgcccaatgggacgggccaacggttgtggtctggctagagctctgggttgggatgccagcctggtatgtggctgcctgccgagcaaacgtgaaaagcggggccatcatgtcggccctgtccgacacagagatccagcgtgagattggcatcagcaaccccctgcacaggctgaagctgaggctggccatccaggagatcatgtcgctgaccagcccgtctgccccgcccacatctagaacgacactcgcctatggggacatgaaccacgagtggatcggcaacgagtggctccccagcctgggcctcccccagtaccgcagctacttcatggagtgccttgtagacgccaggatgctggaccacttgaccaagaaagaccttcgagggcagctgaaaatggtcgacagttttcacagaaacagtttccagtgtggaattatgtgcctgagaaggttaaattatgaccggaaagaactggaaagaaaaagagaagaaagtcagagtgaaataaaagacgtgcttgtttggagcaatgatcgagtgattcgctggatcctgtcaattggccttaaagaatatgcaaacaatcttatagagagtggtgttcacggagcacttctggccttagatgaaaccttcgacttcagtgcactggcactgctgttacagatcccgacgcagaacacacaggctcgtgctgtcttggaaagagaatttaacaaccttttggtcatggggactgatagaaggtttgatgaagatgatgataaaagctttaggagagcaccttcatggagaaaaaagtttagaccaaaggacattcgtggcttagctgctgggtcagcagagactctccctgcaaacttccgggtgacttcttctatgtcttccccctctatgcagccaaagaagatgcagatggacggtatgtgatgggtcacactaacctgtcacttgttgggagcatgagcagctttctgtctggaacattaataatgatctaaaacggcctatttaatatgttacaaggcacttgagtatggttgcatgtccaaatataaatgtttttaaattaactctaacatttgtttataaaagtttaaccataataatagaattttt

SEQ ID NO: 25 - DNA, 1579 bp, Homo sapiens
cagctgaggcgttctccgcttagtgcctacggcatgggtcagctctttctgccacctccgccttcgcggtcctcacctctttgcgggtgaagttaccttttgggtttccagaccttgggggcctgaggcaaactcggggacgaacccctaacccacctctcttttctgtcattctccaactcttactttgtaccacggaatcacttggccttaaagctagccagctcggccgtgccttgaggccaggttcgagagataaaggtaaccgtggggaaagcggccccccagtggacaaaggcggaggcaagaatagagcaggcctgagggccataggcgatgagaataggcagttgaggggtgaaaaagaaaacaaacaaacaacaacaacaaaccctgaagagtactgaagatttagaagggactggaaaggacttgttgcgcaatggaagaatctgactctgagaaaacgacggagaaagaaaatctggggccgagaatggatccaccactaggggaaccggaaggatcgcttgggtgggtgctaccaaatacagccatgaagaaaaaggtgctgttgatgggtaaaagtgggtctggtaagaccagcatgaggtctattatctttgcaaattatattgccagagacacacgtcgccttggcgcaacaatactagaccgtatacatagtcttcaaattaatagcagtttgagcacctactctctcgtagactctgttggaaatacaaagacatttgatgtagaacattctcatgttcgatttctgggaaacctggtattgaacctgtgggattgtggtgggcaagacaccttcatggaaaattatttcactagccaacgggacaacatcttccgaaatgtggaggttctgatttatgtctttgatgtggagagccgcgaactggaaaaggacatgcactattaccaatcatgcctggaggccattctgcagaattctccagatgccaaaatattttgcttggtacacaaaatggatctggtacaggaggatcaacgggacctgatttttaaagagcgagaagaagatttgaggcgtttgtctcgcccattggaatgttcttgtttccgaacatctatctgggatgaaaccctctataaggcttggtccagcatagtttatcagctgattcccaatgttcagcagctggaaatgaacctaaggaattttgctgaaattatcgaagctgatgaagtacttctttttgagagagctacttttctggtaatttctcactatcagtgtaaagagcagcgtgatgcccatagatttgagaaaataagcaacattattaagcagttcaagctgagctgcagcaagctggctgcctctttccagagtatggaagtcaggaactctaacttcgctgctttcattgacatctttacatccaacacttatgtgatggttgtagtgtctgatccgtccattccttctgcagctactctgatcaacatccgcaatgccaggaaacactttgaaaagctggaaagagtggatggaccaaagcagtgtcttctcatgcgctaaacattagtagta

SEQ ID NO: 26 - DNA, 7017 bp, Homo sapiens
cctcgctccctctccggtaatgagggggctgagctgtccctccgaggagggggcctggtgtggataaaagagacgaaaaagccgggggaggtttccaaaaataaaaccgtccgggtccccttcagacggctgcaggcacagggaggaggcgcgaaggtgcagcagccgtgcgagcccagctggagtaggagcgcggactcgaggctcggggcgcgcagccctcgttccgccgagagccgggcccccagtcggccgcttcagggccccctagactcagagaagctggccgccgggcggggccgggagaacagcccgcgggcgtccagcgtgccgaccacaaagctcttcgcggtgcccgcgcgcaccactctccagccgccccgcgccatgaggccgcggaccaaagcccgcagcccggggcgcgccctgcggaacccctggagaggcttcctgccgctcaccctggctctcttcgtgggcgcgggtcatgcccaaagggaccccgtagggagatacgagccggctggtggagacgcgaatcgactgcggcgccctgggggcagctacccggcagcggctgcagccaaggtgtacagtctgttccgggagcaggacgcgcctgtcgcgggcttgcagcccgtggagcgggcccagccgggctgggggagccccaggaggcccaccgaggcggaggccaggaggccgtcccgcgcgcagcagtcgcggcgtgtccagccacctgcgcagacccggagaagcactcccctgggccagcagcaaccagcaccccggacccgggccgcgccggctctcccacgcctggggaccccacagcggtctggggctgcgcccccaaccccgccgcgagggcggctcacggggaggaacgtctgcgggggacagtgctgcccaggatggacaacagcaaacagcaccaaccactgtatcaaacccgtttgcgagccgccgtgccagaaccggggctcctgcagccgcccgcagctctgtgtctgccgctctggtttccgtggagcccgctgcgaggaggtcattcccgatgaggaatttgacccccagaactccaggctggcacctcgacgctgggccgagcgttcacccaacctgcgcaggagcagtgcggctggagagggcaccttggccagagcacagccgccagcaccacagtcgccgcccgcaccacagtcgccaccagctgggaccctgagtggcctcagccagacccacccttcccagcagcacgtggggttgtcccgcactgtccgacttcacccgactgccacggccagtagccagctctcttccaacgccctgcccccgggaccaggccttgagcagagagatggcacccaacaggcggtacctctggagcacccctcatccccctgggggctgaacctcacggagaaaatcaagaagatcaagatcgtcttcactcccaccatctgcaagcagacctgtgcccgtggacactgtgccaacagctgtgagaggggcgacaccaccaccctgtacagccagggcggccatgggcacgatcccaagtctggcttccgcatctatttctgccagatcccctgcctgaacggaggccgctgcatcggcagggacgaatgctggtgccccgccaactccaccgggaagttctgccacctgcctatcccgcagccggacagggagcctccagggagggggtcccgccccagggccttgctggaagccccactgaagcagtccactttcacactgccgctctccaaccagctggcctccgtgaacccctccctggtgaaggtgcacattcaccacccacccgaggcctcagtgcagatccaccaggtggcccaggtgcggggcggggtggaggaggccctagtggagaacagcgtggagaccagacccccgccctggctgcctgccagccctggccacagcctctgggacagcaacaacatccctgctcggtctggagagccccctcggccactgcccccagcagcacccaggcctcgaggactgctgggccggtgttacctgaacactgtgaacggacagtgtgccaaccctctgctggagctgactacccaggaggactgctgtggcagtgtgggagccttctggggggtgactttgtgtgccccatgcccacccagaccagcctccccggtgattgagaatggccagctggagtgtcctcaggggtacaagagactgaacctcactcactgccaagatatcaacgagtgcttgaccctgggcctgtgcaaggacgcggagtgtgtgaataccaggggcagctacctgtgcacatgcagacctggcctcatgctggatccatcgcggagccgctgtgtgtcggacaaggcaatctccatgctgcagggactgtgctaccggtcgctggggcccggcacctgcaccctgcctttggcccagcggatcaccaagcagatatgctgctgcagccgcgtgggcaaagcatggggcagcgagtgtgagaaatgccctctgcctggcacagaggccttcagagagatctgccctgccggccacggctacacctacgcgagctccgacatccgcctgtccatgaggaaagccgaggaggaggaactggcaaggcccccaagggagcaagggcagaggagcagcggggcactgcccgggccagcagagaggcagcccctccgggtcgtcacggacacctggcttgaggccgggaccatccctgacaagggtgactctcaggctggccaggtcacgaccagtgtcactcatgcacctgcctgggtcacagggaatgccacaaccccaccaatgcctgaacaggggattgcagagatacaggaagaacaagtgaccccctccaccgatgtgctggtgaccctgagcaccccaggcattgacagatgcgctgctggagccaccaacgtctgtggccctggaacctgcgtgaacctccccgatggatacagatgtgtctgcagccctggctaccagctgcaccccagccaggcctactgcacagatgacaacgagtgtctgagggacccctgcaagggaaaagggcgctgcatcaaccgcgtggggtcctactcctgcttctgctaccctggctacactctggccacctcaggggcgacacaggagtgtcaagatatcaatgagtgtgagcagccaggggtgtgcagcggggggcagtgcaccaacaccgagggctcgtaccactgcgagtgtgatcagggctacatcatggtcaggaaaggacactgccaagatatcaacgaatgccgtcaccccggtacctgccctgatgggagatgcgtcaattcccctggctcctacacttgtctggcctgtgaggagggctaccggggccagagtgggagctgtgtagatgtgaatgagtgtctgactcccggggtctgtgcccatggaaagtgcaccaacctagaaggctccttcagatgctcttgtgagcagggctatgaggtcacctcagatgagaagggctgccaagatgtggatgagtgtgccagccgggcctcatgccccacaggcctctgcctcaacacggagggctccttcgcctgctctgcctgtgagaacgggtactgggtgaatgaagacggcactgcctgtgaagacctagatgagtgtgccttcccgggagtctgcccctccggagtctgcaccaacacggctggctccttctcctgcaaggactgcgatgggggctaccggcccagccccctgggtgactcctgtgaagatgtggatgaatgtgaagacccccagagcagctgcctgggaggcgagtgcaagaacactgtgggctcctaccagtgcctctgtccccagggcttccagctggccaatggcaccgtgtgtgaggatgtgaatgagtgcatgggggaggagcactgcgcaccccacggcgagtgcctcaacagccacgggtctttcttctgtctgtgcgcgcctggcttcgtcagcgcagaggggggcaccagctgccaggatgtggacgagtgtgccaccacagacccgtgtgtgggagggcactgtgtcaacaccgagggctccttcaactgtctatgtgagactggcttccagccctccccagagagtggagagtgtgtggatattgacgagtgtgaggactatggagacccggtgtgtggcacctggaagtgtgaaaacagccctggctcctaccgctgtgttctgggctgccagcctggcttccacatggccccgaacggagactgcattgacatagacgagtgcgccaacgacaccatgtgtggcagccacggcttctgtgacaacactgatggctccttccgctgcctctgtgaccagggcttcgagatctctccctcaggctgggactgtgtggatgtgaacgagtgtgagcttatgctggcggtatgtggggccgcgctctgtgagaacgtggagggctccttcctgtgcctctgtgccagtgacctggaggagtacgatgcccaggaggggcactgccgcccacggggggctggaggtcagagtatgtctgaggccccaacgggggaccatgccccggcccccacccgcatggactgctactccgggcagaagggccatgcgccctgctccagtgtcctgggccggaacaccacacaggctgaatgctgctgcacccagggcgctagctggggagatgcctgtgacctctgcccgtctgaggactcagctgaattcagcgagatctgccctagtggaaaaggctacattcctgtggaaggagcctggacgtttggacagaccatgtacacagatgcggatgagtgtgtgatattcgggcctggtctctgcccgaacggccggtgcctcaacaccgtgcctggttatgtctgcctgtgcaatcccggcttccactacgatgcttcccacaagaagtgtgaggatcacgatgagtgccaggacctggcctgtgagaatggcgagtgcgtcaacacggagggctccttccactgcttctgcagccccccgctcaccctggacctcagccagcagcgctgcatgaacagcaccagcagcacggaggacctccctgaccacgacatccacatggacatctgctggaaaaaagtcaccaatgatgtgtgcagcgaacccctgcgtgggcaccgcaccacctacacggaatgctgctgccaggacggcgaggcctggagccagcagtgtgctctgtgtcccccgaggagctctgaggtctatgctcagctgtgcaacgtggctcgcattgaggcagagcgggaggccggggtccacttccggccaggctatgagtatggccccgggcccgatgacctgcactacagcatctatggcccagatggggcccccttctacaactacctgggccccgaggacaccgtccctgagcctgccttccccaacacagccggtcactcagcggaccgcacacccatccttgagtctcctttgcagccctcagaactccagccccactacgtggccagccatccagagcccccagccggcttcgaagggcttcaggcggaggagtgcggcatcctgaacggctgtgagaatggccgctgtgtgcgcgtgcgggagggctacacctgtgactgttttgagggcttccagctggatgcggcccacatggcctgcgtagatgtgaatgagtgtgatgacttgaacgggcctgctgtgctctgtgtccatggttactgcgagaacacagagggctcctaccgctgccactgctccccgggatatgtggctgaggcagggcccccccactgcactgccaaggagtagcagtcaggggtcagtgtggcaactacctggaaatggcctccagtcacaggcaggggccttgaggatgatttcctagctgggaagacaccgtgacatcaggccagaggtttccaatcagccttgcctgctttcatctctcccagcttagcctctggctgtaagcttcggtcattgcctccatgcccttgcttggctcaagcaccaccaatcgctttaatgcttcagccaccgcatgaggccctgtccaccacctttcctggccttgctatgggatgcttaccaaaggatggccctcatccaccctcccaagctgtgcgagcatgcaaggccccatggcctcacactgcagacacccctttccagccacaatccaccatcatcctgacgatcccacaactgggacagaggctacatctgccctagggaggtccttcagaatctgtggagcaagaaaggatttggggaagcttggggactgactccagagccccctcctaagaaccatcaccaccactcagccaatctgttctgggccctgattttgccacacctccatcctgtagcccattctctgaccccaaggagtggcagaagatcccttcactcagagaagcaaggctgatattagcttgttgaatgtaagagacacaaatgaagaagaacaaagagcctgagaaagcagcaagaggacatgatgaaaaatacgtggagttgatgagaaaggggagccaaggctttatacgtctaaagaaaatattcagtagctgaatccgcccagtgatagcctgtgggcaccagcagcaagggctgccatgggatacagcacccatctacaaagacctctattacataaacactgcttcttacaggaaacaaacctcttctgggatctccttttgtgaaaaccagtttgatgtgctaaaagtaaaaagtctattttccagtgtggtcttgttcagaagcagccagatttccaatgttgtttttcccctccactcagaaacccctgccctttcccttcagaaaacgatggcaggcattcctctgagtttacaagcagagactcactccaacccaaactagctgggagttcagaaccatggtggaataaagaaatgtgcatctggtccaaaaaaaaaaaaaaaaaaaaaaaaaaaaaaaaaa

SEQ ID NO: 27 - DNA, 1484 bp, Homo sapiens
tggctgcgagaagacgacagaagacattagactcaggcacctctgaaattgtgaaatctcccagaatcgagtgttctaagacaagaagagaaatgcaatcagtggttcaactcataatgacaagagacagtgatggttatgaaaactcaacagatggtgaaatgtgtgacaaagatgctctggaggaagattcagaaagcgttagtgaaataggaagtgatgaggaatctgaaaatgaaattacaagtgttggtagagcttcaggtgatgacgatggaagtgaagatgatgaagaggaggatgaagatgaagaggaggatgaagatgaggatagtgaggatgatgataaaagtgacggtggccctgatcttgcaaggggtaaaggaaatatagaaactagttctgaagatgaagatgatacggcagatttgtttccagaagaatctggttttgagcatgcttggagagaattagataaagatgctcctcgtgctgatgagattacacgtcgattagtagtttgtaacatggactgggatagattaaaggcaaaagatttgctggctctgttcaattcatttaaacccaaaggaggtgtaatattttccgtcaagatatatccttcagaatttagaaaggagaggatgaaggaagagcaagttcaaggaccagtagagctattaagtattcctgaagatgccccagaaaaagactggacgtctagagaaaaattgagagattatcaattcaaacgactgaagtactattatgcagtagtagactgtgattctccggaaacagctagtaaaatttatgaggattgtgatggcctggaatttgaaagtagttgttctttcatagatctaaggtatataccagatgatattacttttgatgatgagcctaaggatgcagcctcagaagtgaatttaacagcatataaaccaaaatatttcacttctgctgcaatgggaacatcaacggtggaaatcacttgggatgagactgatcatgaaagaattacaatgctcaacaggaagtttaaaaaggaagagcttttggacatggattttcaagcctacttagcttcctctagtgaagacgaagaggagatagaagaggagctacaaggtgatgatggagtcaatgtagaagaagatgggaaaacaaagaaaagtcagaaggatgatgaagagcaaattgctaaatacaggcagctcttgcaggttattcaagaaaaagaaaagaaaggcaaagaaaatgatatggaaatggaaattaaatgggttccaggtcttaaagaaagtgcagaagagatggtcaaaaacaaattggaaggaaaggataaactgaccccttgggaacaatttttagaaaagaagaaagagaaaaaaagactgaaaaggaaacagaaggctcttgctgaagaggccagtgaagaggaacttccctcttggatgttgatttgaatgacccatactttg

SEQ ID NO: 28 - DNA, 3237 bp, Homo sapiens
  misc_feature (17)..(17): n is a, c, g, or t
aaatgtagtgccacagnaaccctgcacgtgacgcttgngtcntnntggnanatanaggcgcgngnnngnnngtctagggaatcgaggtgccggctgcnnntnncnnnnnannngcccagacaggcggtgggtgggaatgcctcacttcagtttgaagagggtccggatccaaaggggttaaaacgagcgacccccgatccccgaccacacttcccgcctccctaaaacgcacaccccgctagccatgggcngccgcgaccnccngttcaaagtgctggtggtgggggacgccgcagtgggcaagacgtcgctggtgcagcgatattcccaggacagcttcagcaaacactacaagtccacggtgggagtggattttgctctgaaggttctccagtggtctgactacgagatagtgcggcttcagctgtgggatattgcagggcaggagcgcttcacctctatgacacgattgtattatcgggatgcctctgcctgtgttattatgtttgacgttaccaatgccactaccttcagcaacagccagaggtggaaacaggacctagacagcaagctcacactacccaatggagagccggtgccctgcctgctcttggccaacaagtgtgatctgtccccttgggcagtgagccgggaccagattgaccggttcagtaaagagaacggtttcacaggttggacagaaacatcagtcaaggagaacaaaaatattaatgaggctatgagagtcctcattgaaaagatgatgagaaattccacagaagatatcatgtctttgtccacccaaggggactacatcaatctacaaaccaagtcctccagctggtcctgctgctagtagtgtttggcttattttccatcccagttctgggaggtcttttaagtctcttccctttggttgcccacctgaccattttattaagtanatttgaanngncnnnngncnnntgtccagtaaggaggcccattgtcacttagaaaagacanctggaacccatgtgcatttctgcatctcctggattagcctttcacatgttgctgactcacattagtgccagttagtgccttcggtgtaagatcttctcatcagccctcaatttgtgatccggaattttgtgagaaggattagaaatcagcacctgcgttttagagatcataattctcacctacttctgagcttatttttccatttgatattcattgatatcatgacttccaattgagaggaaaatgagatcaaatgtcatttcccaaatttcttgtaggccgttgtttcagattctttctgtcttggaatgtaaacatctgattctggaatgcagaaggaggggtctgggcatctgtggatttttggctactagaagtgtcccagaagtcactgtatttttgaaacttctaacgtcataattaagtttctcttgtcttggcatcaagaatagtcaagttttttggccgggcatggtggctcatgcctgtaatcccagcacttggggaggccaaggcaggcggatcacatgaggccaggaattcgagaccaacctggtcagcatggcaaaaccccgtctctactaaaagtacaaaaattagccaggcgtgatggcacgtgtctgtaatcccagctactctggagactgaggtgggagaatcgcttgagactgggaggcagaggttgcagtgaaccgagatcatgccaccgcacttcagcctgggtgacagagaaggactccgtctcaaaaaaaaaanaaaaaagaatagtcatttttaaactacctatctcatgcaatgaaagcattttcttccacaaagagcttaatcctcatgataggattgcctagtgtctcccatttgcaggtttctgggttgatgtcttaatgcataatactgcaagtgacatcagctggctgtgatgcttcgaaataggtctgctcctcacagctttgggaatctgaatggaagaagaaaagagagaagttaacaacctccactggggcaactttgtgaacangtaggcacttagtcataggaaacatattatgtgcaggtcctagcctggggtaggaaagtagatagacagaaaatcattaggtaatttaagtactaaattgggcagggctttttagtatcaaatcactactagaccgtttaatttgttaaattatctctaggatggtgatttataacctacccaaagttatcgatattcttactaaactctgaggcctgaagttctgtgatagaccttaaataagtgtcctaagtcagtggttcccaaatctggctggtcgggaatacctgggaagtttgttaaaattttttaaaaatgttttaagatttttgggtcctgagccaggcgtggtggctcacacctgtaatcccagcactttgggaggctgaggcaggtggatcgcctgaggtcaggagttcaagatcaacctggccaacatactgaaaccccgtctctactaaaaataagaaaaattagctgggcgtggtggcgggcacctgtaatcccagctacttgggaggctgaggcaggagaatcacttgaacctgggagttagaggttgcagtgagctgagatcacaccattgcgcttcagncctgggcaacaagagtgaaactccatctccaaaaaaaaaaaacaaaaagaaaaagattttggggtcccgacctcaaacctactgaatcagaatttctagggatgaagcctaggaatgtgttgttgttttcagagcttccctggtgattgtgataagcctggtttggaaaccattgctggagaactttgtaaagatacagagacccagaccttttgtatttacatttaaatacaaatacaaatcctgggtttctatatgttctgttagcttttcaggtgattctgctacacagacgttgaaaaccactgccctaagaaagagatcagaggccacatatcagagagaaaagggaccaaaccttcggtggtttgttgtgtgtcgttttaatgccaattattttaacttgcacagtcttctgaaaccttgtattaatagttctcttttgtattaccattttcaggtagggttttgatcactatgattctgaagataatagtgaaatagtgaatttcattgatatgaagagataattgattttcattcattggtttgaacacctgcaaaatcacaaataaatgagaactaagtcttgtaaaaaaaaaanaaaaaaaaaaaaaaa

SEQ ID NO: 29 - DNA, 1285 bp, Homo sapiens
  misc_feature (25)..(25): n is a, c, g, or t
taacgctccctaaactgccacttgntcagctccgcgcctaaggtgtctattagtgcgcctgcgctgtgacctagaatgggcgcatgcgccgagcggaactggctggtttgaaaaccatggcgtgggtaccagcggagtccgcagtggaagagttgatgcctcggctattgccggtagagccttgcgacttgacggaaggtttcgatccctcggtacccccgaggacgcctcaggaatacctgaggcgggtccagatcgaagcagctcaatgtccagatgttgtggtagctcaaattgacccaaagaagttgaaaaggaagcaaagtgtgaatatttctctttcaggatgccaacccgcccctgaaggttattccccaacacttcaatggcaacagcaacaagtggcacagttttcaactgttcgacagaatgtgaacaaacatagaagtcactggaaatcacaacagttggatagtaatgtgacaatgccaaaatctgaagatgaagaaggctggaagaaattttgtctgggtgaaaagttatgtgctgacggggctgttggaccagccacaaatgaaagtcctggaatagattatgtacaaattggttttcctcccttgcttagtattgttagcagaatgaatcaggcaacagtaactagtgtcttggaatatctgagtaattggtttggagaaagagactttactccagaattgggaagatggctttatgctttattggcttgtcttgaaaagcctttgttacctgaggctcattcactgattcggcagcttgcaagaaggtgctctgaagtgaggctcttagtggatagcaaagatgatgagagggttcctgctttgaatttattaatctgcttggttagcaggtattttgaccaacgtgatttagctgatgagccatcttgatgtagctgatctctcagggatagaagatatttctcatgaaggcagcctaactctgaggaaaacaatgccaattcaagtacagatttcaacacatcttcaacactatgtgaagggttcacatcttaacctgtgcaattcagattgatactcagaatatgggttgatttgaatatctgaaatatcaatggaaaatcccactcagtttttgatgaacagtttgaacagttttctgtaatcaagcagcttgcatagaaattgtatgatgaaattttacataggttcttggtgctgttttgttctttttttgttttttgttgttttgttatttacttatatacatataaaattttattgaaaat

SEQ ID NO: 30 - DNA, 738 bp, Homo sapiens
  misc_feature (38)..(38): n is a, c, g, or t
ttttttttttttttttttttttttttttttttttggtnggtggtggttgttcatagaagagtgttctttataaattatattattattgtattttttgaaaaatttatactagaaagtggtatatatgtctttgtatacattacgaccattgtgattatccattaaatccagtgtctgatgctattggttagtaatgatgtcccagcatttgatgttccctctctaatgataacaagtaaatatttaggtttatcccttttgaaagtttggttttacttttaacagtccatgaacagaaaattaaggcctctaaaatcagatcccctagttactcctctgttctgaatctggggaagtagtaaaccaaaaagacagggaaaaccagatatttagagtcgaagcagcatgcaaaaaataataatgtttttaactaatgaaggctactggagttactaagaatcacctaaaaatgtgctactgtaccttgctcattttgcagatttagaagctgaaacatgggttaggttacttactaaaagtccacctgtggcaacctgcaaataatgcttatttatactaaagatgagtttatctgatagagttaggaaatttcntcttttgagtaagagtattgctataacatatatttttttctgaaatgttttaaaatagaagtacacctaattgttattggttaaaaaaaaagccatatgtgaatgaggttggtttttgggtttt

SEQ ID NO: 31 - DNA, 2352 bp, Homo sapiens
tttttttttttttttttttttgaaaacagaataagttactttattacaagaacaaaacagacttgctaattttatgtctcctttcacccatatcacaaatgcaaagtatatattatcataagatggaaattaccaaactccaagatactctgcataaatgaaataaactcaacttgcaaaattagcaagtagaacattcaagtatctacagtcattataaatagtgtaaagtacatgccatatctacagtgtgggaagagtataaacatgggaaaatgagttcttaaggaacaattagaatgtttgtttggacaatttaaaatattaacaagggtactcaaatacagaatcaaccaaaacatttattttttctcttttttcaagaaaaactgtcgagcttcttcccaggcctgctgaggaaatctgttagccagctcaagatagtcttggggaccaaggcatttccctttgggttggtaaggctggttttccgcttgacctggagtctgtgccaaatcctgaatgaaagtggccagtaaaacacagctcatctcttgctccagaatgatcttgttctgatggttgttgtaacaagcagcgataagtgaagggaacagtactttgatcagccgtgggtcactgaaatactggaagggcaactggcagagcttctgcagcactgtggggtggcggccggactgcacgatcacctgaaaagcaggcagatgaagagctgcaaagctgttgaagaaacgtaaactctgaatggccacttggatggtattttgagtgtaattctccttgggactggcagtgctggggtccaagatggtgccatggaagaggacacagtagagcatatgaagaactccagccaggtcggttgcctgaagagcagctgtcagccctgtgggatcctggcgattattgtcaaatatgctgtatgaccttccagtgacagcaaagcacagtgtacacattgcatgtaagagtcctgcggcatgctgcagaaatatggccatcttgggattctcatccactgggccttgcaccgagaggaagcaggcacacagtttgtcaatcagacccatgttcaccacgtagctgataaggtcctgaactcgattgttaaaaggatcaccttgtgaggttttgtttttcatttcctgtgttggtatttttggggtagctggctggcagtttccatctggtcgattggcaatcaggcagcccaaaaccacagcactgactttgagaagtccagttgtcaagccttcaaaaacttgtttatttgtatttctccccaaaatagtattattttcatctggaacataaaccgtcaactggtgtatcaggaggtccattaagaaggtaatcttgttactaaacagaacatcactgcagttttctgaacagttattgcaggtgaggttgtaaacattgattgcattgcagagagacttaggaggaattcttaaaactgtgttcacatttgtggctgggactactgcttgaaggatgtgttcaagggctgttaatccaccagcagcttgaaatgcaatctgatctgccacattctctttttccagtattctagtgatctctcctagggtccgatccaaagcagacactttattgtttgcccatgagccactgtcttgaacttgtacttgttttagaagatctttggctaatcgctgaagctttgctttataaggtgaatcagagccagaatttttggtttccattaaactctcatattccttagccctgaagttcatccgggcttttatctttttggctttttttttatttttttgccgctcttctccatctttcaaagcttctgctggagctgtactttcaaccacaatgtcaataatgtacttcttcaaggaaagatgctccacttcttcatctgacagttcacgcccctggatgctggtattctctctcacggcttgctggtgttttctccctttaacatggctaaaaagatatacctctgaagagatcaggacattgcagagagaacactgcttctttctttcataaggggtcagtttgggggcataatcagtatttgcatgtcgcccactgcttagctcagcagctttttcttttctttgttcaatctgttccatgtgccttcgaatactttcatcatgcttgagctgaatttttttctgtaactcttccatagcttcttgttgagcagctgtgagtgctgccaatcgttcttccctgtctctagctctttcccgggctgcatcctcacgggttttttccttttcttgcctctgttgttcaattcgggcttcttgttctttcctcttcattaacaattcttct

SEQ ID NO: 32 - DNA, 1180 bp, Homo sapiens
ccgggtcactgttaagggggccgccatggcccggctgtggggcgcgctgagtctttggccactgtgggcggccgtgccgtggggcggggcggcagccgtcggtgtccgggcttgcagctccacggccgccccggacggcgtcgagggcccggcgctgcggcgctcctattggcgccacctgaggcgtctggtgctgggtcctcccgaaccgccgttctcgcacgtgtgccaagtcggggacccggtgctgcgcggcgtggcggccccggtggagcgggcgcagctaggcgggcccgagctgcagcggctgacgcaacggctggtccaggtgatgcggcggcggcgctgcgtgggcctaagcgcgccgcagctgggggtgccgcggcaggtgctggcgctggagctccccgaggcgctgtgtcgggagtgcccgccccgccagcgcgcgctccgccaaatggagcccttccccctgcgcgtgttcgtgaaccccagcctgcgagtgcttgacagccgcctggtcacctttcccgagggctgcgagagcgtcgccggcttcctggcctgcgtgccccgcttccaggcggtgcagatctcagggctggaccccaatggagaacaggtggtgtggcaggcgagcgggtgggcagcccgcatcatccagcacgagatggaccacctgcagggctgcctgtttattgacaaaatggacagcaggacgttcacaaacgtctattggatgaaggtgaatgactaaagctttgctactggggctgaggattccggataccaagacgcaaacactttcactttgagctgggcaaatcttacttggcatcaacttggatggctcgcatatgacaggaaactggattgccaaggcatggcagactgagctggaggaagatgtcagaaatgtttgccctgaaatcagttacggacaaaatgttggctacaacttgaggagaaaaatcacccccaaaggagtggacatttcctaacaattctgtatggaggaaggtggggtaattgcattcgtctgcagtagacacgagttcctcggacctgtataatctcccaaagccaaggtttggtaataatgtagtccccaaatacctgaaagctgtctttaaaaatgcaggtaagcatgtgactggcaaaaaaaaaaaaaaaaaaaaaa

SEQ ID NO: 33 - DNA, 1899 bp, Homo sapiens
ccaagccagtggacaccccccccacagatcaccgtccagcctgcagcgttcgcatttagcccaggaatcatcagtgctgcttccctcgggggacagacccagatcctggggtccctcactacagctccagtcattaccagcgccattcccagcatgccagggatcagcagtcagatcctcaccaatgctcagggacaggttattggaacccttccatgggtagtgaactcagctagtgtggcggccccagcaccagcccaaagcctgcaggtccaggccgtgaccccccagctgttgttgaacgcccagggccaggtgattgcgaccctggctagcagccccctgcctccacctgtggctgtccggaagccaagcacacctgagtcccctgctaagagtgaggtgcagcccatccagcccacaccaaccgtgccccagcctgctgtggtcattgccagcccagctccagccgccaagccatctgcctctgctcctatcccaattacctgctcagagacccccaccgtcagccagttggtgtccaagccacatactccaagtctggatgaggatgggatcaacttagaagagatccgggagtttgccaagaactttaagatccggcggctctcgctgggccttacacagacccaggtgggtcaggctctgactgcaacggaaggtccagcctacagccagtcagccatctgccggttcgagaagctagacatcacacccaagagtgcccagaagctaaagccggtgctggaaaagtggctaaacgaagctgaactgcggaaccaggaaggccagcagaacctgatggagtttgtgggaggcgagccctccaagaaacgcaaacgccgcacctccttcaccccccaggccatagaggctctcaatgcctattttgagaagaacccactgcccacaggccaggagatcactgaaattgctaaggagctcaactacgaccgtgaggtagtgcgggtctggttctgcaatcggcgccagacgctcaagaacaccagcaagctgaacgtctttcagatcccttagggctcagccctggccctgtgttctagcactttgtccatttcccgtggcatccggctgcagccactgccatgacagcacctgtcattttgccacgtgcagctgtgctcaccccaggtcatcagactccaccgtgtgcatgtgcatcaatgtccctcttttctcccacacatctcacatcatggggaggccagagggggccacacgagagctccaggctctgggctggtcactccgaagaagaggatttgtgacgtcacttagagaagcaccttgctagcatggtttctgaagggtgaattctggtggggaaccagaaactccctgtctttggggcagggctaaagcagctcctaaggaccactggccattagctcttgcttttgatggcattctctttccaccttgtcttctcctttgctcctctgtgttagtgtggcaggtatgacaactcatccagtggaaacacagcctcacactgcccttccgccccccacactttgcctgcaggtgcaccgaaagacctgggagataaaattcaaaaaagtgtgatgtgctgctcagaaggtcagactccatgtctgccttgacctcaaggtcagaaggttcccaaacccctggggctggaacatgggatctcctcttccacctcttcctggttcctttgcggggaaaattgcactaaaacagaaccttttcttaatccatgttggaaggaagcaacagtgaactctacctgttctggagttctcctgggtctccagaaggttgggaatttagaaaataaggctgttctttcatattttaatttaatatatgtcaatggccatccctcccacaaaa

SEQ ID NO: 34 - DNA, 2352 bp, Homo sapiens
acagagccccgccgccgccagagcgatgttcccgcagagccggcacccgacgccgcaccaggctgcaggccagcccttcaagttcactatcccggagtccctggaccggattaaagaggaattccagttcctgcaggcgcagtatcacagccttaaattggaatgtgagaaactggcaagtgaaaagacagaaatgcagaggcactatgtgatgtattatgaaatgtcatatggattaaacattgaaatgcacaaacagactgaaatcgccaagagattgaatacgatttgtgcacaagtcatcccatttctgtctcaggaacatcaacaacaggtggcccaggctgttgaacgtgccaaacaggtgaccatggcagagttgaatgccatcatcgggcagcagcagttgcaagctcagcatctttctcatggccacggacccccagttccccttacgcctcacccttcgggacttcagcctcctggaatcccgcccctcgggggcagtgccggccttcttgcgctgtctagtgctctgagtgggcagtctcacttggcaataaaagatgacaagaagcaccacgatgcagagcaccacagagacagagagccgggcacaagtaattccctcctggtcccagacagtctaagaggcacagataaacgcagaaatggacctgaattttccaatgacatcaagaaaaggaaggtggatgataaggactccagccactatgacagtgatggtgacaaaagcgatgacaacttagttgtggatgtgtctaatgaggacccttcttctccgcgagcaagccctgcccactcgccccgggaaaatggaatcgacaaaaatcgcctgctaaagaaggatgcttctagcagtccagcttccacggcctcctcggcaagttccacttctttgaaatccaaagaaatgagcttgcatgaaaaagccagcacgcctgttctgaaatccagcacaccaacgcctcggagcgacatgccaacgccgggcaccagcgccactccaggcctccgtccaggtctcggcaagcctccagccatagaccccctcgttaaccaagcggcagctggcttgaggacacccctggcagtgcccggcccatatcctgctccttttgggatggtcccccacgctggcatgaacggcgagctgaccagcccaggcgctgcctacgccagtttacacaacatgtcgccccagatgagcgccgcagccgcccgcggccgccgtggtcggtacgggcgctcccccatggtggggtttgatcctccccctcacatgagagtacctaccattcctccaaacctggcaggaatccctggggggaaacctgcatactccttccacgttactgcagacggtcagatgcagcctgtcccttttcccccgacgcccctcatcggacccggaatcccccggcatgctcgccagatcaacaccctcaaccacggggaggtggtgtgcgctgtgaccatcagcaaccccacgagacacgtgtacacaggcgggaagggctgcgtcaaggtctgggacatcagccaccctggcaataagagccctgtctcccagctcgactgtctgaacagagacaattatatccgttcctgtaaattgctacccgatggctgcactctcatagtgggaggggaagccagtactttgtccatttgggacctggcggctccaaccccgcgcatcaaggcggagctgacgtcctcggcccccgcctgctacgccctggccatcagccccgattccaaggtctgcttctcatgctgcagcgacggcaacatcgctgtgtgggatctgcacaaccagacactagtgaggcaattccagggccacacagacggagccagctgtattgacatttctaatgatggcaccaagctctggacgggtggtttggacaacacagtcaggtcctgggacctgcgcgaggggcggcagctgcagcagcacgacttcacctcccagatcttctccctggggtactgccccaccggggagtggctggcagtgggcatggagagcagcaatgtggaggtgctgcacgtgaacaagcctgacaagtaccagctgcacctgcatgagagctgcgtgctgtccctgaaatttgcttactgtggtaaatggtttgtgagtactggaaaagataacctcctcaatgcttggcggaccccctatggagccagcatattccagtccaaagagtcctcgtcagtgcttagctgtgacatctctgtggatgataagtacatagtcactggctcgggggacaagaaggctacagtctatgaagtcatctactgaaaacattatgtggt

SEQ ID NO: 35 - DNA, 3059 bp, Homo sapiens
gccccccccccacgcctcctcccctcctcccgcccgcgcgtgctcccgcccctcgccgccgctcgctcgctcgccggccggcctccgctcggccccctccctcagctccggtgcgcggcggccgacgacccgcggcctgggcctcggcgcgccaccggcgcccgcgcggagcggcccgggggccctcacgcaggcccatggcggcggcgcggcgcgctctcgggccgggcaccacgcgggcgcggggccgggggcgcggggccgggggcgggtcccgccgggcgctgggccgtgggccgcctggagggccgcgagttcgagtatctgatgaagaagcgctcggtgaccatcggccgcaactcgtcgcagggctcggtggacgtgagcatgggccactcgagcttcatctcccggcgccacctcgagatcttcacgcccccgggcggcgcggccatggcggggccgctccggagctgccgcccgcgcagcccaggcccgacgccggcggcgacttctacctgcgctgcttgggcaagaacggggtattcgtggacggcgtgttccagaggcgcggggcgccgccgctgcagctgccgcgcgtgtgcacattcaggttcccgagcacaaacatcaagataacgttcactgccctgtccagcgagaagagagagaagcaggaggcgtctgagtctccagtgaaggccgtacagccacacatctcgcccctgaccatcaacattccagacaccatggcccacctcatcagccctctgccctcccccacgggaaccatcagcgctgcaaactcctgcccctccagcccccggggagcggggtcttcagggtacaaggtgggccgagtgatgccatctgacctcaatttaatggctgacaactcacagcctgaaaatgaaaaggaagcttcaggtggagacagcccgaaggatgattcaaagccgccttactcctacgcgcagctgatagttcaggcgattacgatggctcccgacaaacagctcaccctgaacgggatttatacacacatcactaaaaattatccctactacaggactgcggacaagggctggcagaattcaattcgccacaatctctctctgaatcgttatttcatcaaagtgccgcgttcccaggaagaaccaggcaaaggctcgttctggaggatagacccagcctctgaaagcaaattaatagaacaggcttttaggaaacgacggcctaggggcgtgccctgctttagaacccctctgggaccgctctcttctaggagtgccccagcctctcccaatcacgcgggagtgctgtctgctcactctagtggcgcccagacccctgagagcctgtcgagggaaggttcgccggcccccctggagcctgagcctggcgctgcacagcccaaactcgctgtcatccaggaagcccggtttgcccagagcgccccagggtcacctctgtccagtcagccagtcttaatcaccgtccagcggcagctaccacaggccatcaagcctgtcacctacactgtggccaccccagtgaccacctcgacctcccagccacccgtcgtgcagacggttcacgtcgtccaccagatcccagcggtgtcggtcaccagtgtggccggactggccccagcgaacacgtacactgtctctggacaagctgtggtcaccccggcagccgtgctggcccctcctaaggcagaggcccaggagaatggagaccacagggaagtcaaagtgaaagtagagcctattcccgccattggccacgccacgctcggcactgccagccggatcattcagacggcacagaccaccccggtccagacggtgaccatagtacaacaggcacctctaggtcaacaccagctaccaataaaaactgtaacacaaaacggcactcacgtggcatcagtccccactgcggtccacggccaggtgaacaatgccgcggcgagtcctttgcacatgttggcaacacacgcatccgcatcggcctccctgcccacaaagcgccacaacggtgaccagccggagcagccggagctgaagcggatcaagacagaagacggcgagggcatcgtcattgccctgagcgtggacacgccaccggcagccgtaagggaaaagggtgtccagaactagcgaccgggagagcttttctttaacgatatcaactctgtggtgccaaaaggagacgcggcctcccgccagcactcgggggtgcagggccctgtggttggacttcacctctcagcactgaaaacccaaaacccagctggccttaacactccttaaagacagaagtcacacttgaacaaaacccacacacaacaaaacctgatttgggagacggtgtctccactgagcacctgctgggctgagcttctacctacgagtgaaactctgtcctcccgcgaggaccaggcatcgctgtgtgaggacggcacggccagcgcctgctgtgagtgggtctcccaagactaggcctcaggacgcggggggagccatccccgccgccctcacaggacccaccaggcagcggagacatgtggaattagagtattttgaggtgtcctttctttacaaaataatggggtcttgggcatttcacatcactccatttctactgagactttcagaatcacacaggccctttccgtggatttcatttggggcaaagaaacaacatagttttgtttttgttttcagcctatggaatgatttccttttgtctgtcttgttcaagttcagacgaagctactctggcatctgcacatttccgtgttacagcagctgcctgatgaattttatccacctccatttcagcatgtggctcgcgtggacaggtggacggacgctgtggccgcatggaaccttgagaacccagggacgagccagtgccgggaaggaactgccgggactcaccgagctgcacttaactgttctctttctggctattttttgttgtttgtttctttgtgttgactttgtccctggcaaaattttccactctgagtaaaacaagtctcggaattc

SEQ ID NO: 36 - DNA, 1549 bp, Homo sapiens
aaaccctctgtaaagtaacagaagttagaaggggaaatgtcgcctctctgaagattacccaaagaaaaagtgatttgtcattgctttatagactgtaagaagagaacatctcagaagtggagtcttaccctgaaatcaaaggatttaaagaaaaagtggaatttttcttcagcaagctgtgaaactaaatccacaacctttggagacccaggaacaccctccaatctctgtgtgttttgtaaacatcactggagggtcttctacgtgagcaattggattgtcatcagccctgcctgttttgcacctgggaagtgccctggtcttacttgggtccaaattgttggctttcacttttgaccctaagcatctgaagccatgggccacacacggaggcagggaacatcaccatccaagtgtccatacctcaatttctttcagctcttggtgctggctggtctttctcacttctgttcaggtgttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgataacctgctcccatcctgggccattaccttaatctcagtaaatggaatttttgtgatatgctgcctgacctactgctttgccccaagatgcagagagagaaggaggaatgagagattgagaagggaaagtgtacgccctgtataacagtgtccgcagaagcaaggggctgaaaagatctgaaggtagcctccgtcatctcttctgggatacatggatcgtggggatcatgaggcattcttcccttaacaaatttaagctgttttacccactacctcaccttcttaaaaacctctttcagattaagctgaacagttacaagatggctggcatccctctcctttctccccatatgcaatttgcttaatgtaacctcttcttttgccatgtttccattctgccatcttgaattgtcttgtcagccaattcattatctattaaacactaatttgag

SEQ ID NO: 37 - DNA, 1584 bp, Homo sapiens
  misc_feature (308)..(311): n is a, c, g, or t
ttttttttttttttttttttttttttacgatagatcacagttttttattcttaatgtcacaagcaggagaaaaatctcacattcatactaaaaattccaactagactcaacaggaatgaagtctctatttgtaatggaaagtcccagcctcccgctgccgtccagtgtgtgtactgtacacatccacactcactctcactcagggttcccggaccggctgtcctgcctgcggaactgaggtaaacaagctcaggtactgacactaggagggtctaccttacataaggtacaggtagaagcttgattgnnnnctaggcccaggcccacccagaccctccaatcctaacaggtatttaggcttgaggttcactccctcctcagctgcacacgcagccaggtataacactcgccctcagtcacaacggggagggggcaccggttacatctacatcacattatttataaaataagaattacatttcatataacatggccagaaggagctctagtcccccaggaaagctgccggggacagcatttgagcctcttctttgcacaggcataacttaacnatacagctaattcctagttaatagcatttatacttaaccacctcaatgaaccaagcttgaaggaatttaaaaggcaatttagcttaaatacaaaaataaatttttgttaaaaaacgtttaaatatttttttcttttaatttagacacacgcattcatacttctcccaaagaggttgggcgtgacagcaaggcgcttgggcctggggtatgtggtttcagaaggacggaaggaaaggatgggctgcagagggccctgtttgggaaaaataggattttaaaaatatggttcattaatttaggttttctaacatctactttgggntgacgtagcctccagtgaggtcagttaagtnggacagaaaccgcagagggaagaggtctttgcttcccctgggcccattctccctggctgccagcccttgaagtcagaacaccatgggaaaattcaggagtcggcacttgtagccgtcaagtggtgctactttccacattcaattagcgaggccccagaaaacttgaaacaggaagtccggcactaccaacatgccgccactcatacaactcaactcttcctccaaactcgattcaaagagcaatacacagagcgtcagacaaaggaacacaggacaaggcttgaaacatacaatcctggagaggcagacgagggcccaatggggccaaagggtagctgggcgaggaggcagctgaagcagcggtgggcatcctgcctgctccaagagcttctcccttcacccctggctctgccaggctaacagtcttgagccaccaagctctgaggggccaaactggcagagggttggagatgaaacattgattcaggcttggcaggaagaaaacggcaggagcagcacccactggggtctccaaggcaaagctagattcaagggcatttaaaaaggagggaatcctgcaaggaaccggtgagggacacagcagcaagaaatggggaaacagagtccccagaagggaccagaggaagagaaccacttct

SEQ ID NO: 38 - DNA, 3969 bp, Homo sapiens
gaattccgaaagcaaggagatggccaccaaggagaagctgcagtgtctgaaagatttccacaaggacatggtgaagccctcaccagggaagagcccaggcacgcggcctgaggacgaggctgagggaaaacctccgcagagggagaagtggtctagcaagatcgactttgtgctctctgtggctggcggcttcgtgggcttgggcaacgtctggcgcttcccgtacctctgctacaagaatggtggaggtgcgtttctcataccgtattttattttcctgtttgggagcggcctgcctgtgtttttcttggagatcatcataggccagtacacctctgaagggggcatcacctgctgggaaaagatctgccccttgttctctggtatcggctatgcctccgttgtaattgtgtccctcctgaatgtctactacatcgtcatcctggcctgggccacatactacctgttccagtccttccagaaggagctgccctgggcacactgcaaccacagctggaacacacctcactgcatggaggacaccatgcgcaagaacaagagtgtctggatcaccatcagctccaccaacttcacctcccctgtcatcgagttctgggagcgcaacgtgctgagcttgtcccctggaatcgaccacccaggctctctgaaatgggacctcgctctctgccttcttttagtctggctagtgtgtttcttctgcatctgcaagggcgtcaggtccactgggaaggtcgtctacttcacagccacttttccattcgccatgctcctggtgctgctggtccgagggctgacgctgccgggcgcgggccgaggcatcaagttctatctgtatcctgacatcacccgccttgaggacccacaggtgtggattgacgctgggactcagatattcttctcttatgccatctgcctgggggctatgacctcgctggggagctacaacaagtacaagtataactcgtacagggactgtatgctgctgggatgcctgaacagtggtaccagttttgtgtctggcttcgcaattttttccatcctgggcttcatggcacaagagcaaggggtggacattgctgatgtggctgagtcaggtcctggcctggccttcattgcctacccaaaagctgtgacaatgatgccgctgcccacattttggtccattcttttttttattatgcttctcttgcttggactggatagccagtttgttgaagttgaaggacagatcacatccttggttgatctttacccatccttcctaaggaagggttatcgtcgggaaatcttcatcgccttcgtgtgtagcatcagctacctgctggggctgacgatggtgacggagggtggcatgtatgtgtttcagctctttgactactatgcagctagcggtgtatgccttttgtgggttgcattctttgaatgttttgttattgcctggatatatggaggtgataacctttatgatggtattgaggacatgattggctatcggcccgggccctggatgaagtacagctgggtgatcactccagttctctgtgttggatgtttcatcttctcgctcgtcaagtacgtacccctgacctacaacaaaacatacgtgtccccaacttgggccattgggctgggctggagcctggccctttcctccatgctctgcgttcccttggtcatcgtcatccgcctctgccagactgaggggccgttccttgtgagagtcaagtacctgctgaccccaagggaacccaaccgctgggctgtggagcgcgagggagccacaccttacaactctcgcaccgtcatgaacggcgctctcgtgaaaccgacccacatcattgtggagaccatgatgtgagctctctcgggtcgacggggccggcggctttcctgctgtttactaacattagattcacataggaccaggtttacagagctttatatttgcactaggatttttttttttttgtaattgtcacagaaaatgtaattgtgggtatgtgtgcgtgcgtgtgtgtgtgtgtgtgtgtgtatcgtgtgtgtgtgttttgttttgatttgggggatattttgtacaaaaagaaaacccacgggaagatgtccgtggagaggcagagctttcatactgaattagatgtattttatgggaatttggtaaatttttctttgtatttttttttttacatataagtatatatacacttagagattgtcatatacttttaccacttgaattgatcttcttgccagcaatagatctcattttcaaaagcaattcttcggtgctgtgtagctggcagaaagttctgtccagtaaacgcaggatggaattttcctgggactctacacccatcttaaggtggtataccttccaaatcctggttcagatggaagaaatagcaggagagaggacccattagctggcagacccaggggaagaaaggagggctgtgaggagatacctcattaaacttggcttagtgaagaagagagatgccaaaggaatgaaccaacccttcacataaaggagactggctgaagctgaatgaggaggccctatagcagaagtctgattctaagagcagtagaaacttgtaccagaagcaaaatcccacttttaattttgagatggtgagtggatagtcagtagaccgtcagaaccactggccagagagggagctgctagagatccaagaaggctggcaggaatgaggctcacaactcagcctcgcaagaggtggcagaggcacaggaggccacagtccttcctggggcattccaggcagagaaggagcagaggctctcccggcaggagctggggtctcagggctcagatgagtctgttgcatttgaatggggtcatagcaggttctggtcattccccaagcaacatctcagcatctcttaaagttgcctgcaggaatgaagcatgacatacctgttgagggactaggggagtggtggggaggtgagtggaccaaaggatataggccccaggcatgcagatgggcccggtgtcggggaggggtgctttctttcctcatctccccactccccactctcagcctgggagactcctgccaagccctcattaaagatgccaccctgggctgccctggcacctagcaaggcacaccaagaacagcttttgagtcgtatcctccactggggaagtgctcccagttcagaacaagggcagcccgtggtgctgacctaggatataacaaagctcttcacttcaaaacccctgcaatagctgggtttacagacatttaccacctggggacccaaaagagaaggcctaggagagttttctagaaggttgggattgtcagggtcctggccccccagaactggcttgatcaagggccttatgtggagcagaggttgtctctgaaccaggagagaaggtactatacctttcaaatccccagggcagacacacccccacccagcccctatttggacctaaactgtgccatttgaacagtcacttccaagctcagtctaaatgaaaccgaaacgtgaccacgcacaaaggcagtcactgctcgaggggtgcagaccgcagaattttcacagcaggggctcttggaactctggaaacccccttcttaaatttgggaggaggagtatgcctttggtgtccccctcccaagggcaattctgaaccccatctttggcaggcatacatatttcactgtttccaaagctatctactctgccaaacaacacccagtcctattccaaactctcaacgattctatcttgttcctgtttttctatgtatttatggttgccgtttgtgtctgatttgattttactgttttttccctgattttatggagtagcattgtgacctgttttcctttgtcttatataactttagtaaactaaccactgtcaatgattgagggcaggtggcacgtggggaagagggcggaattc

SEQ ID NO: 39 - DNA, 5257 bp, Homo sapiens
aaaaattttctgttaccaaattttacaacttctaataagactactataactttatgtaaactgatgaagatgtgctgattaacatattctgtgatatggtttacaacttttaatcataattgtccatgattttggaatgctgttatttatcagtaaatgtaaaatatttgaggcatttagccatacacacactagaactttttaaaactttgtcctatagtgtaattataaactgatgactattatcttcatacattgagtcttcatgcatcaatgaaatgaaaaatataggagtagatgtccacgcccacagaccctggtgcgatgccccacccagggccttcgccggggcctgggccttcccctgggccaattcttgggcctagtccaggaccaggaccatccccaggttccgtccacagcatgatggggccaagtcctggacctccaagtgtctcccatcctatgccgacgatggggtccacagacttcccacaggaaggcatgcatcaaatgcataagcccatcgatggtatacatgacaaggggattgtagaagacatccattgtggatccatgaagggcactggtatgcgaccacctcacccaggcatgggccctccccagagtccaatggatcaacacagccaaggttatatgtcaccacacccatctccattaggagccccagagcacgtctccagccctatgtctggaggaggcccaactccacctcagatgccaccaagccagccgggggccctcatcccaggtgatccgcaggccatgagccagcccaacagaggtccctcacctttcagtcctgtccagctgcatcagcttcgagctcagattttagcttataaaatgctggcccgaggccagcccctccccgaaacgctgcagcttgcagtccaggggaaaaggacgttgcctggcttgcagcaacaacagcagcagcaacagcagcagcagcagcagcagcagcagcagcagcagcagcaacagcagccgcagcagcagccgccgcaaccacagacgcagcaacaacagcagccggcccttgttaactacaacagaccatctggcccggggccggagctgagcggcccgagcaccccgcagaagctgccggtgcccgcgcccggcggccggccctcgcccgcgccccccgcagccgcgcagccgcccgcggccgcagtgcccgggccctcagtgccgcagccggccccggggcagccctcgcccgtcctccagctgcagcagaagcagagccgcatcagccccatccagaaaccgcaaggcctggaccccgtggaaattctgcaagagcgggaatacagacttcaggcccgcatagctcataggatacaagaactggaaaatctgcctggctctttgccaccagatttaagaaccaaagcaaccgtggaactaaaagcacttcggttactcaatttccagcgtcagctgagagaggaggtggtggcctgcatgcgcagggacacgaccctggagacggctctcaactccaaagcatacaaacggagcaagcgccagactctgagagaagctcgcatgaccgagaagctggagaagcagcagaagattgagcaggagaggaaacgccgtcagaaacaccaggaatacctgaacagtattttgcaacatgcaaaagattttaaggaatatcatcggtctgtggccggaaagatccagaagctctccaaagcagtggcaacttggcatgccaacactgaaagagagcagaagaaggagacagagcggattgaaaaggagagaatgcggcgactgatggctgaagatgaggagagttatagaaaactgattgatcaaaagaaagacaggcgtttagcttaccttttgcagcagaccgatgagtatgtagccaatctgaccaatctggtttgggagcacaagcaagcccaggcagccaaagagaagaagaagaggaggaggaggaagaagaaggctgaggagaatgcagagggtggggagtctgccctgggaccggatggagagcccatagatgagagcagccagatgagtgacctccctgtcaaagtgactcacacagaaaccggcaaggttctgttcggaccagaagcacccaaagcaagtcagctggacgcctggctggaaatgaatcctggttatgaagttgcccctagatctgacagtgaagagagtgattctgattatgaggaagaggatgaggaagaagagtccagtaggcaggaaaccgaagagaaaatactcctggatccaaatagcgaagaagtttctgagaaggatgctaagcagatcattgagacagctaagcaagacgtggatgatgaatacagcatgcagtacagtgccaggggctcccagtcctactacaccgtggctcatgccatctcggagagggtggagaaacagtctgccctcctaattaatgggaccctaaagcattaccagctccagggcctggaatggatggtttccctgtataataacaacttgaacggaatcttagccgatgaaatggggcttggaaagaccatacagaccattgcactcatcacttatctgatggagcacaaaagactcaatggcccctatctcatcattgttcccctttcgactctatctaactggacatatgaatttgacaaatgggctccttctgtggtgaagatttcttacaagggtactcctgccatgcgtcgctcccttgtcccccagctacggagtggcaaattcaatgtcctcttgactacttatgagtatattataaaagacaagcacattcttgcaaagattcggtggaaatacatgatagtggacgaaggccaccgaatgaagaatcaccactgcaagctgactcaggtcttgaacactcactatgtggcccccagaaggatcctcttgactgggaccccgctgcagaataagctccctgaactctgggccctcctcaacttcctcctcccaacaatttttaagagctgcagcacatttgaacaatggttcaatgctccatttgccatgactggtgaaagggtggacttaaatgaagaagaaactatattgatcatcaggcgtctacataaggtgttaagaccatttttactaaggagactgaagaaagaagttgaatcccagcttcccgaaaaagtggaatatgtgatcaagtgtgacatgtcagctctgcagaagattctgtatcgccatatgcaagccaaggggatccttctcacagatggttctgagaaagataagaaggggaaaggaggtgctaagacacttatgaacactattatgcagttgagaaaaatctgcaaccacccatatatgtttcagcacattgaggaatcctttgctgaacacctaggctattcaaatggggtcatcaatggggctgaactgtatcgggcctcagggaagtttgagctgcttgatcgtattctgccaaaattgagagcgactaatcaccgagtgctgcttttctgccagatgacatctctcatgaccatcatggaggattattttgcttttcggaacttcctttacctacgccttgatggcaccaccaagtctgaagatcgtgctgctttgctgaagaaattcaatgaacctggatcccagtatttcattttcttgctgagcacaagagctggtggcctgggcttaaatcttcaggcagctcatacagtggtcatctttgacagcgactggaatcctcatcaggatctgcaggcccaagaccgagctcaccgcatcgggcagcagaacgaggtccgggtactgaggctctgtaccgtgaacagcgtggaggaaaagatcctcgcggccgcaaaatacaagctgaacgtggatcagaaagtgatccaggcgggcatgtttgaccaaaagtcttcaagccacgagcggagggcattcctgcaggccatcttggagcatgaagaggaaaatgaggaagaagatgaagtaccggacgatgagactctgaaccaaatgattgctcgacgagaagaagaatttgacctttttatgcggatggacatggaccggcggagggaagatgcccggaacccgaaacggaagccccgtttaatggaggaggatgagctgccctcctggatcattaaggatgacgctgaagtagaaaggctcacctgtgaagaagaggaggagaaaatatttgggagggggtcccgccagcgccgtgacgtggactacagtgacgccctcacggagaagcagtggctaagggccatcgaagacggcaatttggaggaaatggaagaggaagtacggcttaagaagcgaaaaagacgaagaaatgtggataaagatcctgcaaaagaagatgtggaaaaagctaagaagagaagaggccgccctcccgctgagaaactgtcaccaaatccccccaaactgacaaagcagatgaacgctatcatcgatactgtgataaactacaaagatagttcagggcgacagctcagtgaagtcttcattcagttaccttcaaggaaagaattaccagaatactatgaattaattaggaagccagtggatttcaaaaaaataaaggaaaggattcgtaatcataagtaccggagcctaggcgacctggagaaggatgtcatgcttctctgtcacaacgctcagacgttcaacctggagggatcccagatctatgaagactccatcgtcttacagtcagtgtttaagagtgcccggcagaaaattgccaaagaggaagagagtgaggatgaaagcaatgaagaggaggaagaggaagatgaagaagagtcagagtccgaggcaaaatcagtcaaggtgaaaattaagctcaataaaaaagatgacaaaggccgggacaaagggaaaggcaagaaaaggccaaatcgaggaaaagccaaacctgtagtgagcgattttgacagcgatgaggagcaggatgaacgtgaacagtcagaaggaagtgggacggatgatgagtgatcagtatggacctttttccttggtagaactgaattccttcctcccctgtctcatttctacccagtgagttcatttgtcatataggcactgggttgtttctatatcatcatcgtctataaactagctttaggatagtgccagacaaacatatgatatcatggtgtaaaaaacacacacatacacaaatatttgtgaccaaatgggcctcaaagattcagattgaaacaaacaaaaagctttt

SEQ ID NO: 40 - DNA, 1635 bp, Homo sapiens
atgggcgaggtggaggccccgggccgcttgtggctcgagagcccccctggggaggcgccccccatcttcctgccctcggacgggcaagccctggtcctgggcaggggacccctgacccaggttacggaccggaagtgctccagaactcaagtggagctggtcgcagatcctgagacccggacagtggcagtgaaacagctgggagttaacccctcaactaccgggacccaggagttgaagccggggttggagggctctctgggggtgggggacacactgtatttggtcaatggcctccacccactgaccctgcgctgggaagagacccgcacaccagaatcccagccagatactccgcctggcacccctctggtgtcccaagatgagaagagagatgctgagctgccgaagaagcgtatgcggaagtcaaaccccggctgggagaacttggagaagttgctagtgttcaccgcagctggggtgaaaccccagggcaaggtggctggctttgatctggacgggacgctcatcaccacacgctctgggaaggtctttcccactggccccagtgactggaggatcttgtacccagagattccccgtaagctccgagagctggaagccgagggctacaagctggtgatcttcaccaaccagatgagcatcgggcgcgggaagctgccagccgaggagttcaaggccaaggtggaggctgtggtggagaagctgggggtccccttccaggtgctggtggccacgcacgcaggcttgtaccggaagccggtgacgggcatgtgggaccatctgcaggagcaggccaacgacggcacgcccatatccatcggggacagcatctttgtgggagacgcagccggacgcccggccaactgggccccggggcggaagaagaaagacttctcctgcgccgatcgcctgtttgccctcaaccttggcctgcccttcgccacgcctgaggagttctttctcaagtggccagcagccggcttcgagctcccagcctttgatccgaggactgtctcccgctcagggcctctctgcctccccgagtccagggccctcctgagcgccagcccggaggtggttgtcgcagtgggattccctggggccgggaagtccacctttctcaagaagcacctcgtgtcggccggatatgtccacgtgaacagggacacgctaggctcctggcagcgctgtgtgaccacgtgtgagacagccctgaagcaagggaaacgggtcgccatcgacaacacaaacccagacgccgcgagccgcgccaggtacgtccagtgtgcccgagccgcgggcgtcccctgccgctgcttcctcttcaccgccactctggagcaggcgcgccacaacaaccggtttcgagagatgacggactcctctcatatccccgtgtcagacatggtcatgtatggctacaggaagcagttcgaggccccaacgctggctgaaggcttctctgccatcctggagatcccgttccggctatgggtggagccgaggctggggcggctgtactgccagttctccgagggctgagccccgcccagctcccctccacaataaacgctgtttctccttgaaaaaaaaaaaaaaaaaaaaaaaaaa

SEQ ID NO: 41 - DNA, 978 bp, Homo sapiens
ccgcgtcggtgcccgcgcccctccccgggccccgccatgggcctcaccgtgtccgcgctcttttcgcggatcttcgggaagaagcagatgcggattctcatggttggcttggatgcggctggcaagaccacaatcctgtacaaactgaagttgggggagattgtcaccaccatcccaaccataggcttcaatgtagaaacagtggaatataagaacatctgtttcacagtctgggacgtgggaggccaggacaagattcggcctctgtggcggcactacttccagaacactcagggcctcatctttgtggtggacagtaatgaccgggagcgggtccaagaatctgctgatgaactccagaagatgctgcaggaggacgagctgcgggatgcagtgctgctggtatttgccaacaagcaggacatgcccaacgccatgcccgtgagcgagctgactgacaagctggggctacagcacttacgcagccgcacgtggtatgtccaggccacctgtgccacccaaggcacaggtctgtacgatggtctggactggctgtcccacgagctgtcaaagcgctaaccagccaggggcaggcccctgatgcccggaagctcctgcgtgcatccccgggatgaccagactcccggactcctcaggcagtgccctttcctcccacttttcctcccccatagccacaggcctctgctcctgctcctgcctgcatgttctctctgttgttggagcctggagccttgctctctgggcacagaggggtccactctcctgcctgctgggacctatggaaggggcttcctggccaaggccccctcttccagaggaggagcagggatctgggtttccttttttttttctgttttgggtgtactctaggggccaggttgggagggggaaggtgagggcttcgggtggtgctataatgtggcactggatcttgagtaataaatttgctgtggtttg

SEQ ID NO: 42 - DNA, 2334 bp, Homo sapiens
agacacctctgccctcaccatgagcctctggcagcccctggtcctggtgctcctggtgctgggctgctgctttgctgcccccagacagcgccagtccacccttgtgctcttccctggagacctgagaaccaatctcaccgacaggcagctggcagaggaatacctgtaccgctatggttacactcgggtggcagagatgcgtggagagtcgaaatctctggggcctgcgctgctgcttctccagaagcaactgtccctgcccgagaccggtgagctggatagcgccacgctgaaggccatgcgaaccccacggtgcggggtcccagacctgggcagattccaaacctttgagggcgacctcaagtggcaccaccacaacatcacctattggatccaaaactactcggaagacttgccgcgggcggtgattgacgacgcctttgcccgcgccttcgcactgtggagcgcggtgacgccgctcaccttcactcgcgtgtacagccgggacgcagacatcgtcatccagtttggtgtcgcggagcacggagacgggtatcccttcgacgggaaggacgggctcctggcacacgcctttcctcctggccccggcattcagggagacgcccatttcgacgatgacgagttgtggtccctgggcaagggcgtcgtggttccaactcggtttggaaacgcagatggcgcggcctgccacttccccttcatcttcgagggccgctcctactctgcctgcaccaccgacggtcgctccgacggcttgccctggtgcagtaccacggccaactacgacaccgacgaccggtttggcttctgccccagcgagagactctacacccgggacggcaatgctgatgggaaaccctgccagtttccattcatcttccaaggccaatcctactccgcctgcaccacggacggtcgctccgacggctaccgctggtgcgccaccaccgccaactacgaccgggacaagctcttcggcttctgcccgacccgagctgactcgacggtgatggggggcaactcggcgggggagctgtgcgtcttccccttcactttcctgggtaaggagtactcgacctgtaccagcgagggccgcggagatgggcgcctctggtgcgctaccacctcgaactttgacagcgacaagaagtggggcttctgcccggaccaaggatacagtttgttcctcgtggcggcgcatgagttcggccacgcgctgggcttagatcattcctcagtgccggaggcgctcatgtaccctatgtaccgcttcactgaggggccccccttgcataaggacgacgtgaatggcatccggcacctctatggtcctcgccctgaacctgagccacggcctccaaccaccaccacaccgcagcccacggctcccccgacggtctgccccaccggaccccccactgtccacccctcagagcgccccacagctggccccacaggtcccccctcagctggccccacaggtccccccactgctggcccttctacggccactactgtgcctttgagtccggtggacgatgcctgcaacgtgaacatcttcgacgccatcgcggagattgggaaccagctgtatttgttcaaggatgggaagtactggcgattctctgagggcagggggagccggccgcagggccccttccttatcgccgacaagtggcccgcgctgccccgcaagctggactcggtctttgaggagccgctctccaagaagcttttcttcttctctgggcgccaggtgtgggtgtacacaggcgcgtcggtgctgggcccgaggcgtctggacaagctgggcctgggagccgacgtggcccaggtgaccggggccctccggagtggcagggggaagatgctgctgttcagcgggcggcgcctctggaggttcgacgtgaaggcgcagatggtggatccccggagcgccagcgaggtggaccggatgttccccggggtgcctttggacacgcacgacgtcttccagtaccgagagaaagcctatttctgccaggaccgcttctactggcgcgtgagttcccggagtgagttgaaccaggtggaccaagtgggctacgtgacctatgacatcctgcagtgccctgaggactagggctcccgtcctgctttgcagtgccatgtaaatccccactgggaccaaccctggggaaggagccagtttgccggatacaaactggtattctgttctggaggaaagggaggagtggaggtgggctgggccctctcttctcacctttgttttttgttggagtgtttctaataaacttggattctctaaccttt

SEQ ID NO: 43 - DNA, 2427 bp, Homo sapiens
agcctgtgactttcctccctggacaaaggcatcatgagttgtcagatctcttgcaaatctcgaggaagaggaggaggtggaggaggattccggggcttcagcagcggctcagctgtggtgtctggtggaagccggagatcaacttccagcttctcctgcttgagccgccatggtggtggtggcgggggcttcggtggaggcggctttggcagtcggagtcttgttggccttggagggaccaagagcatctccattagtgtggctggaggaggtggtggctttggcgccgctggtggatttggtggcagaggaggtggttttggaggcggcagcggctttggaggcggcagcggctttggaggtggcagcggcttcagtggtggtggtttcggtggaggcggctttggtggaggccgctttggaggttttgggggccctggtggtgttggaggtttagggggtcctggtggctttgggcctggaggataccctggtggcatccacgaagtctctgtcaaccagagcctcctgcagcctctcaacgtgaaagttgacccagagatccagaatgtgaaggcccaagagcgtgagcagatcaaaactctcaacaacaaatttgcctccttcattgacaaggtgcggttcttggagcagcagaaccaggtgttacagaccaaatgggagctgctacaacaaatgaatgttggcacccgccccatcaacctggagcccatcttccaggggtatatcgacagcctcaagagatatctggatgggctcactgcagaaagaacatcacagaattcagagctgaataacatgcaggatcttgtggaggattataagaagaagtatgaggatgaaatcaataagcgcacagctgctgagaatgattttgtgacgcttaaaaaggacgtggacaatgcctacatgataaaggtggagttgcagtccaaggtggacctgctgaaccaggaaattgagtttctgaaagttctctatgatgcggagatatcccagatacatcagagtgtcactgacaccaacgtcatcctctccatggacaacagccgcaacctggacttggatagcatcatcgccgaggtcaaggcccagtatgaggagatcgcccagaggagcaaggaagaagcggaggccctgtaccacagcaagtatgaggagctccaggtgactgtcgggagacatggagacagcctgaaagagatcaagatagagatcagcgagctgaaccgcgtgatccagaggctgcagggggagatcgcacatgtgaagaagcagtgtaagaatgtgcaagatgccatcgcagatgccgagcagcgtggggagcatgccctcaaggatgccaggaacaagttgaatgacctggaggaggccctgcagcaggccaaggaggacttggcgcggctgctgcgtgactaccaggagctgatgaacgtgaagctggccctagatgtggagatcgccacctaccgcaaactgctggagggcgaggagtgcaggatgtctggagacctcagcagcaatgtgactgtgtctgtgacaagcagcaccatttcatcaaatgtggcatccaaggctgcctttggaggttctggaggtagagggtccagttccggaggaggatacagctctggaagcagcagttatggctctggaggccgacagtctggctccagaggcggtagtggaggaggaggttctatctctggaggaggatatggctctggcggtggttctggaggaagatacggatctggtggtggctctaagggagggtccatctctggaggaggatatggctctggaggtggaaaacacagctctggaggtggctctagaggaggctccagctctggaggaggatatggctctggaggtgggggttctagctctgtaaagggtagctcaggtgaagcttttggttccagcgtgaccttctcttttagataaagatgagcccccaccaccaccgactctcccaacccagactctcccactccagaatgtagaagcctgtctctgtacctctaactggcagcaagttaaatttttgtcatttatctctgatggcactttgagggaaaagaatgtccacatacagtttttgaaagatcttctctccaaaccagttagttagagccagtgacgcctctgtgttctggggcggaatctgtgctgtctaggtttgtgcttctagccatgcccattcccgcccccaccatgcctctttgcattgcccattttccagatgtgtattctgttgaggacccaggcccatccagggatttcatctctaagcctggcagtgctggggggaaatgtgtttctgtgtatatagctcctcttgtccactctgctttcggaagtgctgtggtctgggggtcttcataataaacctcatttgca

SEQ ID NO: 44 - DNA, 852 bp, Homo sapiens
caccctgtaagtggctgcggcgggaagatggcggagctgcgcgtgctcgtagctgtcaagagggtcatcgactacgccgtgaagatccgagtgaagcctgacaggaccggtgtggtcacggatggtgtgaagcactccatgaaccccttctgtgagatcgcggtggaggaggctgtgcggctcaaggagaagaagctggtgaaggaggtcatcgccgtcagctgtgggcctgcacagtgccaggagacgattcgtaccgccctggccatgggtgcagaccgaggtatccacgtggaggtgcccccagcagaagcagaacgcttgggtcccctgcaggtggctcgggtcctggccaagctggcagagaaggagaaggtggacctggtgctgctgggcaaacaggccatcgatgatgactgtaaccagacagggcagatgacagctggatttcttgactggccacagggcacattcgcctcccaggtgacgctggagggggacaagttgaaagtggagcgggagatcgatgggggcctggagaccctgcgcctgaagctgccagctgtggtgacagctgacctgaggctcaacgagccccgctacgccacgctgcccaacatcatgaaagccaagaagaagaagatcgaggtgatcaagcctggggacctgggtgtggacctgacctccaagctctctgtgatcagtgtggaggacccgccccagcgcacggccggcgtcaaggtggagaccactgaggacctggtggccaagctgaaggagattgggcggatttgagcccctcccagagatggcaataaaactgactctcaacatcaaaaaaaaaaaaaaaaa

SEQ ID NO: 45 - DNA, 581 bp, Homo sapiens
acaactgtgttcactagcaacctcaaacagacaccatggtgcacctgactcctgaggagaagtctgccgttactgccctgtggggcaaggtgaacgtggatgaagttggtggtgaggccctgggcaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcaggctcctgggcaacgtgctggtctgtgtgccggcccatcactttggcaaagaattcacccaaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattc

SEQ ID NO: 46 - DNA, 2446 bp, Homo sapiens
ctgggtctgggtctgagcctaggcatgagtgaggccaccagtgaggcagaggaggaggcggccacggccgaggcggtgggacgcctggccacgacgctgtggctgcggctccgcggctgggaggcggtgctggcggcggcgcagcggttgctggtgtgggagaagccgctgcacagcctggtcacggcggccgcgctcaacggcctcttctggttgctgtcttcctcgtccctccggcccttcttcctactcagcgtctcacttttggcctattttctgctggatctctggcagcctcgctttctccctgacgtttcagcatcatccccagaggagccacactctgacagtgagggtgcggggtcaggcgcccggccgcacctgctgagtgtgcccgagttgtgcagatacctggctgagagctggctcaccttccagattcacctgcaggagctgctgcagtacaagaggcagaatccagctcagttctgcgttcgagtctgctctggctgtgctgtgttggctgtgttgggacactatgttccaggaattatgatttcctacattgtcttgttgagtatcctgctgtggcccctggtggtttatcatgagctgatccagaggatgtacactcgcctggagcccctgctcatgcagctggactacagcatgaaggcagaagccaatgccctgcatcacaaacacgacaagaggaagcgtcaggggaagaatgcacccccaggaggtgatgagccactggcagagacagagagtgaaagcgaggcagagctggctggcttctccccagtggtggatgtgaagaaaacagcattggccttggccattacagactcagagctgtcagatgaggaggcttctatcttggagagtggtggcttctccgtatcccgggccacaactccgcagctgactgatgtctccgaggatttggaccagcagagcctgccaagtgaaccagaggagaccctaagccgggacctaggggagggagaggagggagagctggcccctcccgaagacctactaggccgtcctcaagctctgtcaaggcaagccctggactcggaggaagaggaagaggatgtggcagctaaggaaaccttgttgcggctctcatcccccctccactttgtgaacacgcacttcaatggggcagggtcccccccagatggagtgaaatgctcccctggaggaccagtggagacactgagccccgagacagtgagtggtggcctcactgctctgcccggcaccctgtcacctccactttgccttgttggaagtgacccagccccctccccttccattctcccacctgttccccaggactcaccccagcccctgcctgcccctgaggaagaagaggcactcaccactgaggactttgagttgctggatcagggggagctggagcagctgaatgcagagctgggcttggagccagagacaccgccaaaaccccctgatgctccacccctggggcccgacatccattctctggtacagtcagaccaagaagctcaggccgtggcagagccatgagccagccgttgaggaaggagctgcaggcacagtagggcttcctggctaggagtgttgctgtttcctcctttgcctaccactctggggtggggcagtgtgtggggaagctggctgtcggatggtagctattccaccctctgcctgcctgcctgcctgctgtcctgggcatggtgcagtacctgtgcctaggattggttttaaatttgtaaataattttccatttgggttagtggatgtgaacagggctagggaagtccttcccacagcctgcgcttgcctccctgcctcatctctattctcattccactatgccccaagccctggtggtctggccctttctttttcctcctatcctcagggacctgtgctgctctgccctcatgtcccacttggttgtttagttgaggcactttataatttttctcttgtcttgtgttcctttctgctttatttccctgctgtgtcctgtccttagcagctcaaccccatcctttgccagctcctcctatcccgtgggcactggccaagctttagggaggctcctggtctgggaagtaaagagtaaacctggggcagtgggtcaggccagtagttacactcttaggtcactgtagtctgtgtaaccttcactgcatccttgccccattcagcccggcctttcatgatgcaggagagcagggatcccgcagtacatggcgccagcactggagttggtgagcatgtgctctctcttgagattaggagcttccttactgctcctctgggtgatccaagtgtagtgggaccccctactagggtcaggaagtggacactaacatctgtgcaggtgttgacttgaaaaataaagtgttgattggctagaaaaaaaaaaaaaaaaaaaaaa

SEQ ID NO: 47 - DNA, 468 bp, Homo sapiens
  misc_feature (6)..(6): n is a, c, g, or t
atggtncanntnacnccngtggagaagtcngcngtnacngcnctntggggnaaggtnaangtggatgaagnnggnggngaggccctgggcagnctgctngtggtctacccttggacccagaggttcttngantcnttnggggatctgnnnacnccngangcagttatgggcaaccctaaggtgaaggctcatggcaagaaagtgctcggtgcctttagtgatggcctggctcacctggacaacctcaagggcacctttgccacactgagtgagctgcactgtgacaagctncangtggatcctgagaacttcaggctnctnggcaacgtgntngtctgngtgctggcccatcactttggcaaagaattcaccccaccagtgcangcngcctatcagaaagtggtngctggtgtngctaatgccctggcccacaagtatcactaagctngcnttnttgntgtccaattt

SEQ ID NO: 48 - DNA, 576 bp, Homo sapiens
actcttctggtccccacagactcagagagaacccaccatggtgctgtctcctgccgacaagaccaacgtcaaggccgcctggggtaaggtcggcgcgcacgctggcgagtatggtgcggaggccctggagaggatgttcctgtccttccccaccaccaagacctacttcccgcacttcgacctgagccacggctctgcccaggttaagggccacggcaagaaggtggccgacgccctgaccaacgccgtggcgcacgtggacgacatgcccaacgcgctgtccgccctgagcgacctgcacgcgcacaagcttcgggtggacccggtcaacttcaagctcctaagccactgcctgctggtgaccctggccgcccacctccccgccgagttcacccctgcggtgcacgcctccctggacaagttcctggcttctgtgagcaccgtgctgacctccaaataccgttaagctggagcctcggtggccatgcttcttgccccttgggcctccccccagcccctcctccccttcctgcacccgtacccccgtggtctttgaataaagtctgagtgggcggc

SEQ ID NO: 49 - DNA, 756 bp, Homo sapiens
cctctcccaccggcccgatgagctgcagcggctccggcgcggaccccgaggcggcgccggcctccgccgcctcggccccgggccccgcgcccccggtctcggctcccgccgcgctgccctccagcaccgccgcggagaacaaggccagccccgcggggacagcggggggacctggggctggagcagctgctgggggcacgggacccttggcggcgcgggccggggagccagctgagcggcgtggggcggctccggtgtcggcgggtggcgcggcgcccccggagggggccatatctaacggggtttacgtactgccgagcgcggccaacggagacgtgaagcccgtggtgtccagcacgcctttggtggacttcttgatgcagctggaagattacacgcctacgatcccagatgcagtgactggttactacctgaaccgtgctggctttgaggcctcagacccacgcataattcggctcatctccttagctgcccagaaattcatctcagatattgccaatgatgccctacagcactgcaaaatgaagggcacggcctccggcagctcccggagcaagagcaaggaccgcaagtacactctaaccatggaggacttgacccctgccctcagcgagtatggcatcaatgtgaagaagccgcactacttcacctgagccacccaacctaaatgtacttatctgtccccatgtccccacaccagcctgttttcataataaactttattgtgacaggcta

SEQ ID NO: 50 - DNA, 1211 bp, Homo sapiens
ggcacgaggggagccgaagcagcagcgcaggttgtccccgtttcccctcccccttcccttctccggttgccttcccgggccccttacactccacagtcccggtcccgccatgtcccagaaacaagaagaagagaaccctgcggaggagaccggcgaggagaagcaggacacgcaggagaaagaaggtattctgcctgagagagctgaagaggcaaagctaaaggccaaatacccaagcctaggacaaaagcctggaggctccgacttcctcatgaagagactccagaaagggcaaaagtactttgactcaggagactacaacatggccaaagccaagatgaagaataagcagctgccaagtgcaggaccagacaagaacctggtgactggtgatcacatccccaccccacaggatctgccccagagaaagtcctcgctcgtcaccagcaagcttgcgggtggccaagttgaatgatgctgcccggggctctgccagatcctgagacgcttcccctccctgccccacccgggtcctgtgctggctcctgccccttcctgcttttgcagccaggggtcaggaggtggctcgggtgtgggctggagaggcagaagccctttcctgttggtgtcccagcacatggagccccttgggctgagcaccaagaccttgaaccttttttgttttaccttttttccaaataacagttgggagaaatatcaatgaaattctgggggtgggggtggggttgaaagggtggggtgggagatatggaggagtatgaattagggcttggagttggtaaaaacattcctgactatccttcttaaccacgtggctgatgtggggtaggtatgaggggaaggaagtggagtagcctaatgaaaaggggttctagttgagctctgtagataaatgccttgtttcagtgtggttggagacctggtgtcagataaaagaaactccatccgcacagacagatgcaaacagctcctctagttctgcagagctagttgagaactcaacattaatcattttaaaaagtactgtccttgaaatagatttgctgtgggaagaagggcagtgagtgtgggagaaaggagccgtgagcgtggggaaccccacagagcccaaaggactttttcagtattcgaaataaacaaaacaaaaacccatgaaaaaacccaacccagaaaaaaaaaaaaaaaaaaaa

SEQ ID NO: 51 - DNA, 1216 bp, Homo sapiens
ctgaagacccagagagatttcatgctcagttttagcaccgacccccaggacttcatccaggaatggctccgttcccagcgccgagacctcaagatcatcactgatgtgattggaaatcctgaggaggagagacgagctgctttctaccaccagccctgggcccaggaagcagtaggcaggcacatctttgccaaggtgcagcagcgaaggcaggaactggaacaggtgctgggaattcgcctgacctaactgctcagggatctttcttcccagccctggagcctggagggagaccaccctctgggtccttgctggggccgcagacacgtaggctggggtgaggagtgtctgctgtcaccctctactctccagctttagttttataaatgtagtgataggattccttgttgcttggtccccaaagccttatactttttgcattggctttaattgggttcagcagatgcctcctctgcccccctgcaggcaggcccaagtaggactgctggaggctgtgctttgacattgtaagacatttccgaaccaaaggctgctgggtttgcatgtttacagactccccctggggcgagggtcagagctggctctggggagctgggctaggaagaggaggtgcagcctagactcttcctagcctttctaaaccaaagttctttgccattcctacaagcccagccttgctgctggttttttcctttcctttgggtatttgcactattttgggagcaagttttctatgtgggagccactttttttgtacaggggtaagttgggggttttcagggagcctgttaggtgcctccttcttttctttcctcaatctatgcaagcggctctggccgccatcatctcctgggatgccagagggctgcctctccagcggcttgggccggggaggggacactccagttctctagcatggcctgaggtatggggtatgtgcatgtggaggccagggtaaggtgaatggggaggctgggaggactggtgttgccctttggagcttggtgaggagggtgggcctagggcttggcgagtgccacttttggcaggtttggaaatttccaaataaatccttttgtctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 52 - DNA, 366 bp, Homo sapiens
  misc_feature (19)..(19): n is a, c, g, or t
ttttttttttttttgccgnccactcagactttattcaaagaccaggaagggccggtgcaaggaggggaggagggcccgttgganngccnagcnngnnnnnngaacggctaccgaggctccagcttaacggtatttggaggtcagcacggtgctcacagaagccaggaacttntccagggaggcgtgcaccncaggggttgaactcggcggggaggtgggcggccagggtcaccagcaggcagtggcttaggagcttgaagttgaccngggtccacccnaagnttntgcgcgtgcaggtcgntcagggcggacagcgcgtttgggcatntcgtccacgtgcgccacggctttgggtcaagcgcgttc

SEQ ID NO: 53 - DNA, 3775 bp, Homo sapiens
cattcataagactcagagctacggccacggcagggacacgcggaaccaagacttggaaacttgattgttgtggttcttcttgggggttatgaaatttcattaatcttttttttttccggggagaaagtttttggaaagattcttccagatatttcttcattttcttttggaggaccgacttactttttttggtcttctttattactcccctccccccgtgggacccgccggacgcgtggaggagaccgtagctgaagctgattctgtacagcgggacagcgctttctgcccctgggggagcaacccctccctcgcccctgggtcctacggagcctgcactttcaagaggtacagcggcatcctgtgggggcctgggcaccgcaggaagactgcacagaaactttgccattgttggaacgggacgttgctccttccccgagcttccccggacagcgtactttgaggactcgctcagctcaccggggactcccacggctcaccccggacttgcaccttacttccccaacccggccatagccttggcttcccggcgacctcagcgtggtcacaggggcccccctgtgcccagggaaatgtttcaggctttccccggagactacgactccggctcccggtgcagctcctcaccctctgccgagtctcaatatctgtcttcggtggactccttcggcagtccacccaccgccgcggcctcccaggagtgcgccggtctcggggaaatgcccggttccttcgtgcccacggtcaccgcgatcacaaccagccaggacctccagtggcttgtgcaacccaccctcatctcttccatggcccagtcccaggggcagccactggcctcccagcccccggtcgtcgacccctacgacatgccgggaaccagctactccacaccaggcatgagtggctacagcagtggcggagcgagtggcagtggtgggccttccaccagcggaactaccagtgggcctgggcctgcccgcccagcccgagcccggcctaggagaccccgagaggagacgctcaccccagaggaagaggagaagcgaagggtgcgccgggaacgaaataaactagcagcagctaaatgcaggaaccggcggagggagctgaccgaccgactccaggcggagacagatcagttggaggaagaaaaagcagagctggagtcggagatcgccgagctccaaaaggagaaggaacgtctggagtttgtgctggtggcccacaaaccgggctgcaagatcccctacgaagaggggcccgggccgggcccgctggcggaggtgagagatttgccgggctcagcaccggctaaggaagatggcttcagctggctgctgccgcccccgccaccaccgcccctgcccttccagaccagccaagacgcaccccccaacctgacggcttctctctttacacacagtgaagttcaagtcctcggcgaccccttccccgttgttaacccttcgtacacttcttcgtttgtcctcacctgcccggaggtctccgcgttcgccggcgcccaacgcaccagcggcagtgaccagccttccgatcccctgaactcgccctccctcctcgctcggtgaactctttagacacacaaaacaaacaaacacatgggggagagagacttggaagaggaggaggaggaggagaaggaggagagagaggggaagagacaaagtgggtgtgtggcctccctggctcctccgtctgaccctctgcggccactgcgccactgccatcggacaggaggattccttgtgttttgtcctgcctcttgtttctgtgccccggcgaggccggagagctggtgactttggggacagggggtgggaaggggatggacacccccagctgactgttggctctctgacgtcaacccaagctctggggatgggtggggaggggggcgggtgacgcccaccttcgggcagtcctgtgtgaggatgaagggacgggggtgggaggtaggctgtggggtgggctggagtcctctccagagaggctcaacaaggaaaaatgccactccctacccaatgtctcccacacccaccctttttttggggtgcccaggttggtttcccctgcactcccgaccttagcttattgatcccacatttccatggtgtgagatcctctttactctgggcagaagtgagcccccccttaaagggaattcgatgcccccctagaataatctcatccccccacccgacttcttttgaaatgtgaacgtccttccttgactgtctagccactccctcccagaaaaactggctctgattggaatttctggcctcctaaggctccccaccccgaaatcagcccccagccttgtttctgatgacagtgttatcccaagaccctgccccctgccagccgaccctcctggccttcctcgttgggccgctctgatttcaggcagcaggggctgctgtgatgccgtcctgctggagtgatttatactgtgaaatgagttggccagattgtggggtgcagctgggtggggcagcacacctctggggggataatgtccccactcccgaaagcctttcctcggtctcccttccgtccatcccccttcttcctcccctcaacagtgagttagactcaagggggtgacagaaccgagaagggggtgacagtcctccatccacgtggcctctctctctctcctcaggaccctcagccctggcctttttctttaaggtcccccgaccaatccccagcctaggacgccaacttctcccaccccttggcccctcacatcctctccaggaaggcagtgaggggctgtgacatttttccggagaagatttcagagctgaggctttggtacccccaaacccccaatatttttggactggcagactcaaggggctggaatctcatgattccatgcccgagtccgcccatccctgaccatggttttggctctcccaccccgccgttccctgcgcttcatctcatgaggatttctttatgaggcaaatttatattttttaatatcggggggtggaccacgccgccctccatccgtgctgcatgaaaaacattccacgtgccccttgtcgcgcgtctcccatcctgatcccagacccattccttagctatttatccctttcctggtttccgaaaggcaattatatctattatgtataagtaaatatattatatatggatgtgtgtgtgtgcgtgcgcgtgagtgtgtgagcgcttctgcagcctcggcctaggtcacgttggccctcaaagcgagccgttgaattggaaactgcttctagaaactctggctcagcctgtctcgggctgacccttttctgatcgtctcggcccctctgattgttcccgatggtctctctccctctgtcttttctcctccgcctgtgtccatctgaccgttttcacttgtctcctttctgactgtccctgccaatgctccagctgtcgtctgactctgggttcgttggggacatgagattttattttttgtgagtgagactgagggatcgtagatttttacaatctgtatctttgacaattctgggtgcgagtgtgagagtgtgagcagggcttgctcctgccaaccacaattcaatgaatccccgacccccctaccccatgctgtacttgtggttctctttttgtattttgcatctgaccccggggggctgggacagattggcaatgggccgtcccctctccccttggttctgcactgttgccaataaaaagctcttaaaaacgc

SEQ ID NO: 54 - DNA, 1970 bp, Homo sapiens
  misc_feature (853)..(853): n is a, c, g, or t
tttttttttttgaaagggtcaaaaattttattagcaggactttttgtgtttttgaatatacaggtttcctgctgtccagggtaaaggggaagtggtgtcttgtggcccgaggtttggggcgcttgccttgggactccatccccatctctttcccgctagcgcagctgggggaaggtgcctgcttgccggccccacggattcttcggctgtggcataaggcactgtgtgttctgcaggaaggcgctcatggctggctggtagatgagaggcggccttctcaggggctgcaaagctgaggctgttgaggggttgagttgaacttggggcccctagtgaggaggccccaggcgatgggagcacggcctggtgagtctggtgccgctgcctccccactggccctgcaccaggtctcaggatgggatcatgcctttgctcctcttgcggtcggccatggttctccggttgtggttggctcttgtcgccttgttggcttccttcttcctgcgttcctgggttgtctcgcggctctgcccatggcctcgggggctgccggccactgctgttgagctgtcatgccggtaccctttcttggcgagaaaggccatgcgcctggcttctgccttctctctcagcactgcagggtcctgaacaaaatggtcgggcttgggagcctcctcgtcagcatcgtcttcctcatcgtcgtcatcctcctcctgcccttctctaggcactttggttctcagcacctgagggatggtgaatggcctgcggctgatgagctcgtcatcagagtctgcatcattggcgcccacctggttgccatcgtatgtgtcatcgtactcatcctcgtagntagacactgtggtagggcaggctctcgcctggctgcagtggcacctgcaatggcataagcatgggtctggggggctcaggggctggcctggtttccctggcccagcagagacctggccttggtggnctacctcctccaccaccacgctgtactgctcgtagcgctgccgctgtgccgccactgcacgcttgtcgtncagcaaactccncgtgttttcctccttcctggtgctcttgcccttgtgcacccggctcaggtctactgagtccctgctgaacacatcaaactcgtcattctggaagacgttgtggcgagacgtcagcaggggtgtagggtctggtttcatttctctgtctaggttgcggtccagctggctgagggtgggggccagccgctcctccaggatattgttgatcacctgctctgggtcgtagtggtagtactccaggcaggccaggatgaagccctcaccaaggtctggcagcaggtccttcacttgggagatgagagagtccagttccaccccacacatggcagggcccacagccgcggctgctcccatgcactcctcttcctccgagttctccggatgtgatgatgcttgactgactgcctctgctgtcaccgtgaccccgttaggctccccattaggctcctcaatcaccgatgggtctttagcatctgtggctttccgtctgtccaccccttcccatgcactctcgactncctggaggatgtaggcagtccgcgtctcgtccaanactgatgaggcctgctgnagcaagctgatgtcttcggccacggggaagagtgcattcatagtccccggaggaacctcttctcctgcagcaaggagctgaagatctgaaggaactcttcgatgaagccctgaatgttgtcacagctgctttctaggatgggaaggaggcagatctggttcaggatgatgtggaaaatctccattagcttcttcctggaatgggagagcctctgccacaggtcaccaagaagcctgtnacaaaantcgtgcttctggaaggtctggcaagccaaagggaagatatccaaaaaggcccaaagtgtggtgcaggtatnaaaaaggtagagaacaatgtcc

SEQ ID NO: 55 - DNA, 3869 bp, Homo sapiens
  misc_feature (889)..(890): n is a, c, g, or t
cctggtttctctacatactaaccccatgtctctctggactcacacctggctcaggccttccagaccaaactcatacttcctcctcaccagaagtttccgaatctcacactagagaagatttgtcacctttaaagtccactcacattattttgactcagggttttgttttttgtctatttcccctcctgcaactctcctttaaaagtgaaaagagagagaatggtttaagtagtctgttgctcataagcacgtgtctgcagtctgtcacttgcaaggccttggtgatcaccatgcagcacttgtgcgcacacacacccccacaagtcttttttcctaataatccattttccacattgccatgagggtaaagggttccaatcaaaaccaccattaaactgcatagtttctagtcttccctcagccctgatgctaattatgatagtttgcatagacataacttaaacttgtgattctccctccttccagagctttttgtagccctttttccccccaaaattagttttgttcaaaatcaagcattgggcccagccattaagataagacctaataagtgagagatacagttcccaagagtttacagatccaggtgttcacatggtggtggccagtctaccgtttatcttgtcaaggttgttactgctaatttggtgtcactttggtcttgggcagcggtggtgggcgacgtatctaatgagcgtgccattgacttctccatgtttgtctgtgtacgaggggtgcaggttgtctgtaatccactaaagtgtcagccacacccagctttagtaactcgctaatcgcacaccctgggtatatcagccaggggctgctacagttaatggtacacagttgatggtacacaggctgaaanngataangaatcaagaccctactacatttccaaagccgaaaagccatgagggttacaatttaaatgagcttgggaaggaannnncccgaagacgtctccagcctgaagggtcagagccgctggaagcctggctgtctctgggctttctcgtctcagtgcagcaaggcaccgtgatcaaccaacccgtgatggggaaggccctggttctgtctttgtttgggggtaaatagctgtgtggtgttgagtgactgtgattggcttttccagctcctttaacacaaactacaacccactcacgatacaacttcggcagatgtggtcaaattgatgtttgttgacttgcctcatttccttccagctcttaagttccaaagtctaactgggatggtagatgtgaaatgcatgattgtcgtcctaatttctgcatgtgtttatgacaatgtgttgaggggagggggaggagagccagagagcattttcaggaaccgaggtctttccttgtcttactctgggggaatgccatgtngnctctgatagagttgtctatcggtctgtttgtgaatgccctttcgcttaaacttccagcccttctggtggcatccttggttgtaaggtggctgctgtataacgtccactctctttcatatttaatatgtgtgattgttactgttgtctttttgttttaaagtaaccataagaggagatgatctgagttatatttattgatcagaggaatagagtgcagtaaaactcatactggcatttcgcttttctcatttccctgtaaatgggcgggtattaatccattcccaccttctaatgtccttgatgtccagctggctttgtgggtgtgcaatggttttactgtactgcctcgatctgtgccaggcatttatacacagccacatgtgcgagtcaggtagagcgtatgtggtcacccccatggctcacggagttagctgaatggcctctccgctgtccctagatggcagaaacggtggacacaagcgaaatggtcaacggcgctacagaacaaaggacgagctctaaagagtccagccccatcccctccccgacctctgatcgtaaagccaagactgccctcccagcccagagtgccgccaccttaccagccagaacccaggagacaccttcggcccagatggaaggcttcctcaatcggaaacacgagtgggaggcccacaataagaaagcctcaagcaggtcctggcacaatgtttattgtgtcataaataaccaagaaatgggtttctacaaagatgcnaagactgctgcttctggaattccctaccacagcgaggtccctgtgagtttgaaagaagctgtctgcgaagtggcccttgattacaaaaagaagaancncgtattcaagctaagactaaatgatggcaatgagtacctcttccaagccnaagacgatgaggaaatgaacacatggatccaggctatctcttccgccatctcctctgataaacacgaggtgtctgccagcacccagagcacgccagcatccagccgcgcgcagaccctccccaccagcgtcgtcaccatcaccagcgagtccagtcccggcaagcgggaaaaggacaaagagaaagacaaagagaagcggttcagcctttttggcaaaaagaaatgaactcctttccttcacctcctgcccttctcttaccttttcagtgaaattccagcatgcaagctcagaaccaacacattactctctgtgcctaatgttcctcaatgtggttgattttttttttttttnnaatttatagagcatttcggggggggntgggggaaacacacctaaacactttatctccaagttacaaaagtttgaggtgcagagggaaggccagatttttttttnaatgaaattatatagattagatctcagtatttaaactgttcctcaattttgtgaggctgtgttggaaataacccgcctctagtgctgttggtatgcaaggcagcggtgcttaatcaatatttcctgtgctcaccagaggcaaaatgtaccaatatcctgacaccattctctctccatttacttctggtggttaccctgactcttgactcttagaagtgcccgagatggggctaacctttattaaacagatcgcatattatgatcttgctgcagccacagtgcagctccacattaactctacagaccaaaccatttgtatctggcatcacttactaacacacgacatgcggcttttctgcatcaactgctatgacggttaagaatgtcagtatacaagaaggaatagaaaactgatactgttttaaataatctgtaatttcaatttttttttttttttngctgaaatacattatattgtacgtttgagataattctagntacaaagtataataaaactagatngtataataaaccctttaaatcattggtaagtgtacaagtggtggnaactgaagcatttactggnacaaagtaatgttnactctaatggttacttgctcgtgcgttgnnccacactgtgttataatttgcttcatttccttgctatttgatacatagtgtgcatttctctgtcactgtaactattgtaatgacaaattttcatcttactgcacaatcaaaatgacattgataggaatgaactccagaggctgggcctgaacagggaggtggtcgctcaggcctggtgctcagtcgtacgacctgtacctctcaacttttgccctatctgttaaatatatgctatgtcattaaatgcttttaaatctaaaaaaaaaaaaaaaaaaanaaaaaaaannnnnngngtgtgcatgcccagtagggaaactgcaaagggagaaatgacaaacaagaaacattttacaaccagtctgggctcacttttgcattttttatgcatgtctggtgcacaagctttgaaaactacagcaaacagtaataaatgtgactgttttgtagttataaaaaaaa

SEQ ID NO: 56 - DNA, 3964 bp, Homo sapiens
agtccagagcgagcgggttatgcctccgcctcgttgccctgaatgccgcagcgacacaggaaagggctaagattcggccatgagcagcgcccctcggctgcccgccaagggcgcagattctttctgtacgccagagccagagagcttgggccctgggacgcctgggttccccgagcaggaggaagacgaacttcaccgcaccctgggcgtggagcggtttgaggagatcctacaggaggccgggtctcgtggaggggaggagccaggccgcagctatgggatggaagactttgagtaccgccgccagtcctcccaccacatccatcacccactgtccacccacctgcctccgggtgcacgccgacgcaagacaccccagggcccaggacggaagcctcgaaggcgcccgggagcctccccgactggagaaaccccgaccattgtggagggggaggaagatgaggatgaggccagcgaggctgagggggcccgggctctcactcagccgtcccctgtctccacaccctcctcggtgcagttctttctccgagaggatgacagtgccgaccggaaggcagagaggaccagtccatcttcccctgcaccactgccccaccaggaggcgactcctcgggcctccaaaggggcccaggctggaacccaggtggaggaggcggaggcggaggcggtggcggtggccagtggcacagcagggggtgacgacgggggtgcctcggggcgccccctgcccaaagcccagcctgggcaccgcagctacaaccttcaggagaggaggcgcatcgggagcatgactggggctagacaggcactgctgccccgggtccccacggatgagattgaggcccagacgctggccacggccgacctagacctcatgaagagtcaccggtttgaggacgttcctggggtgcggcggcacttggtgcggaagaatgccaaaggttccacacagagtggccgagaagggcgggagcctggccccacacctcgggctcgaccccgggccccccacaagccccatgaggtgtttgtggagctgaatgagttgctcctggacaaaaaccaggagccccagtggcgggagacagctcgctggatcaaatttgaagaggacgtggaggaggagaccgagcgctgggggaagccccacgtggcctccctctccttccgcagtctcctggagctccgcaggaccctggcccatggggctgtgctcttggatctggaccagcagaccctgcccggagtggcccaccaggtggtggagcagatggtcatctctgaccagatcaaggccgaggatagggccaacgtgctgcgggctctgctgttgaaacacagccacccaagtgatgagaaggacttctccttcccccgcaacatctcagctggctccgtgggctccctgctggggcatcaccatggtcagggggctgagagtgacccccacgtcaccgagcctctcatgggaggtgttcctgagacccggctggaggtggagcgagagcgtgacgtgccgcctccagcaccaccagctggcatcacccgctccaagtccaagcacgagctgaaactgctggagaagattcctgagaatgccgaggccacggtggtccttgtgggctgcgtggagttcctctcccgccccaccatggcctttgtgcggctccgggaggctgtggagttggacgcagtgttggaggtgccggtgcctgtgcgtttcctcttcctgctgctgggcccgagtagtgccaacatggactaccacgagatcggccgctccatctccaccctcatgtcagacaagcaattccacgaggcagcctacctggctgacgagcgggaggacctgctgacggccatcaacgccttcctggactgcagcgtggtgctgccgccttcagaagtgcagggcgaggagctgctgcgctctgtggcccacttccagcgccagatgctcaagaagcgagaggagcagggccggctgctacctacaggggctgggctggagcccaaatctgcccaagataaggcgctcctgcagatggtagagcggcaggggcagctgaagatgatcccttcggcggacggggcggcctttggggggctgatccgagatgtgcggcgccgctatccccactacctgagtgacttccgagatgcacttgaccctcagtgcctggccgcagtcatcttcatctactttgccgccctgtctcctgccatcacctttggggggctgctgggagagaagacacaggacctgataggggtgtcggagctgattatgtccacagcgctccagggcgtggtcttctgcctgctgggtgcccagcccctgttggtgatcggcttctcagggcccctgctggtctttgaggaggccttcttctcgttctgtagcagcaaccacctggagtacctggtgggccgtgtgtggatcggcttctggctggtgttcctggccctgctcatggtggccctggaggggagcttcctggtccgcttcgtctcccgcttcacccgagagatcttcgccttcttgatctcactcatcttcatctatgagaccttctacaagctggtgaagatcttccaggagcaccccctgcatggctgctcagcctccaacagctcagaggtggacggcggtgagaacatgacatgggccggggcaagacccacgctggggccgggcaacaggagcttggctgggcagtctgggcaggggaagccccggggccagcccaacacggcccctctgtcgctggtgctcatggccggcaccttcttcatcgccttcttcctgcgcaaattcaagaacagccggttctttcctggccggatccggcgggtgattggggactttggggtgcccatcgccatcctcatcatggtgcttgtggattacagtattgaggacacctatacccagaagctgagcgttcccagtggattctcggtgactgccccagaaaagaggggctgggtcatcaaccccctgggagagaagagccccttccctgtgtggatgatggttgccagcctgctgcccgccatcctggtcttcattctcatcttcatggagacacagatcaccacgctcatcatctccaagaaggagcgcatgctgcagaagggctccggcttccacctggacctgctgctcatcgtggccatgggcggcatctgtgccctctttggcctgccctggttggctgctgccactgtccgctctgtcactcacgccaacgcgctcactgtcatgagcaaggctgtggcacctggggacaagcccaagattcaggaagtcaaggagcagcgggtgacggggctgctggttgccctgcttgtgggcctctccatagttatcggggatctgctccggcagatccccctggccgtgctctttggaattttcctgtacatgggagtcacctcccttaacgggatccagttctatgagcggctgcatctgctgctcatgccgcccaaacaccacccagatgtcacttacgtcaagaaggtccggaccctccgtatgcacctgttcacggccctgcagctgctctgcctggccctgctctgggccgtcatgtccacagctgcctccctggccttccccttcatcctcatcctcacagtgccgctccgcatggtggtgctcacccgtatcttcaccgaccgagagatgaaatgtctggatgctaacgaggcagagccggtgtttgatgagcgggagggtgtggacgagtacaatgagatgcccatgcctgtgtagccgccaccgagggacagccgagggaccgatggacgaggggacaggctggtgggatgggttccccctcccatgcccctccctcctttttatttaagtgaataatttaaagtattctcctcccccactgcccctgcagtaaagtgctttggcccccaaaaaaaa

SEQ ID NO: 57 - DNA, 4914 bp, Homo sapiens
  misc_feature (1323)..(1323): n is a, c, g, or t
aactgggctggccatagggggaagcggcgcgttaaatccgccccctctccataccctactttcaaggggtgggcggtagaggggaggttggggttggagcagcggcgagggggcccctcccttgcacctccccccaccggacttggtcgcgcccgaagtgtaacactttctctttgtcggaggagctcctctgtttcctgtgcagtagctcccgttgcggcggcacccgtggcagccctggcggacgcaggagcgatggcagcgaccgatatagctcgccaggtgggtgaaggttgccgaactgtccccctggctggacatgtggggtttgacagcttgcctgaccagctggtgaataagtccgtcagccagggcttctgcttcaacatcctgtgcgtgggagagacaggtttgggcaagtccaccctcatggacaccctgttcaacaccaaattcgaaggggagccagccacccacacacagccgggtgtccagctccagtctaatacctatgacctccaagagagcaacgtgaggctaaagctcacgatcgttagcacagttggctttggggaccagatcaacaaagaggacagctacaagcctatcgtggaattcatcgatgcacaattcgaggcctacctgcaggaagagctaaagatccgaagagtgctacacacctaccatgactcccgaatccatgtctgcttgtatttcattgcccccacgggtcattccctgaagtctctggacctagtgactatgaagaagctggacagtaaggtgaacatcatccccatcattgccaaagcagatgccatttcgaagagtgagctaacaaagttcaaaatcaaaatcaccagcgagcttgtcagcaacggagtccagatctatcagtttcctacagatgatgagtcggtggcagagatcaatggaaccatgaacgcccacctgccgtttgctgtcattggcagcacagaagaactgaagataggcaacaagatgatgagggcgcggcagtatccttggggcactgtgcaggttgaaaacgaggcccactgcgactttgtgaagctgcgggagatgctgattcgggtcaacatggaggatctgcgggagcagacccacacccggcactatgagctgtatcgccgctgtaagctggaggagatgggcttcaaggacaccgaccctgacagcaaacccttcagtttacaggagacatatgaggccaaaaggaacgagttcctaggggaactccagaaaaaagaagaggagatgagacagatgttcgtccagcgagtcaaagagaaagaagcgganctcaaagaggcagagaaagagctgcacgagaagtttgaccgtctgaagaaactgcaccaggacgagaagaagaaactggaggataagaagaaatccctggatgatgaagtgaatgctttcaagcaaagaaagacggcggctgagctgctccagtcccagggctcccaggctggaggctcacagactctgaagagagacaaagagaagaaaaattaactctgctgtttgctgcatgctgcatgagacccagggtcctgtttgggcttcctgtagacacccttttcctgcgcaacagagctgggcctccctttctctaatttcccccttaacatgcctggggggcatacaatccaacccgcgccctctcctctcttcctgccanggtttatagaaacctgagaatctgagggtgatgtctggccgctggtcaagaagccaacagtcatgtggctcgcagatgcatcctgcatcccagtccccctcccagcacccccagccatcccccctgtcttcccccacatctttgccagaggtgtgacatggtcagggggcccatctgctactctttcccaccagctcccctgttccagttctggttgctgttagtttccctgaggtatttgcaaccaccatggctgggtaaccaccgatcagcacagctgtccccttggtctcctgtatcccagtcactagtcctccctggtccaccccaccctcatcctcaggagccacagccatttcttagagggtttcaaaaggacagcctttggcgccttttccttctaacctttgagtccagccctttccagttttcattcactcgaagtaactgcactcaagctgtgctcaaaatcggcaacgcatttatttacaccaagcccttcccataaaacacaactgctgaagaaaatagcagacgtttcccctctctctaactctgggtatcccacagatgcaaaagggagaataaacctgaatattattaccagccnanannnnnnaannanannnnnancgaattcttcttgtgaggtatttcagcatctcggggggtaatttccggaagggctccatactgtcccaataaggtgaggccagtagcaggaataataaatcccactttgtaggctggaaaactgagctgtcaaaagaatcaagtgtttgggggtttgctctgatgagtcttctagttcatttggtgaatgtcatgatgatttttaacatgcattttgcatgcatcccccaataagaagagatgagactcggcngnngagaagannaggcccttaactttctttccaanntnnggagttgagagtttaaaaatattccagccctaangtttttatcatgggtcccatctgatagtggctttgggaacctctgtgaagtagagagccctcccttgtcagggttatgaggcacagtggcctttggtgtttggccagtgacagtgtgaganatggagttgacctggcaatgatctgtggctaacatgccgtctctctgcccttcctttgcagtaatccatggctgtgtactgaatagtattccccgctacagctggactggactccatttagccttttaagccgaggttcctattttaactgacagctttcctttggggtgccaggcagcgaggccccccacccctatcctgccatgtacttcaagctcacttcttctttttgagttccgcaacttgctcctgcctcccagccccactggcactgaccatgaccacctacttctattttttttttagagtttctttttttgatcacttactttcaaagcacacagtcaaacaaggttatgccaaatttccaggcctttttgaagtattgagaaggggaaggggatttctcacttcaattatagatcataataggaagcaaaaagaaaaaaatgaaaagcaaacatatgcacgcacttttcttgttgacaaagcaagaatntaggtttgctgtgtaggtttggtgctctattgattggtgagtgaccagagcaagtatgaaggtgatgctgccaaagcacaagcctttttgaagtattgagaaggggaaggggatttctcacttcaattatagatcataataggaagcaaaaagaaaaaaatgaaaagcaaacatatgcacgcacttttcttgttgacaaagcaagaatataggtttgctgtgtaggtttggtgctctattgattggtgagtgaccagagcaagtatgaaggtgatgctgccaaagcacaagccagtttcttgggaaaattcaagttacagtggagtatttttttnaanaccatatncntggnggtagaaacnnncnnncnncnnnaaaaaaaaaaaaaaaaaanntctgctcagatactcagccagtagctcagagagatgctgagttaggcctgtcaggtctccttgggaaaggcttcatatttgcaactttgatgattctatgtccagcttcagagctgctttcccagaaattcacgcttaaacaaccaaccggtaaccaccacttccccacaccgccgcctggtaattatttgcattacaaaccggagncgccctcatttgcatttgtgtacagattaactagttaaggcttgagaagctctgaataattcaaaagtattagacccacacagccttggagagaccttcagaaactaaggaggagttttatattaagggagacattttagtcagtaagacgatataacctacttactccgtaaggggaaatgaaggcccggagaagggaagggacttgaccgaggtcccacttctgtttcgaggcagaagccagactaattttcatgcctcctgactcccaatcagtttcacaaagggattcaatctgtttatatacgttacattcctggatacgaggtcttttgatgttcagagtaactgactagttagtattagaagaccctcgaggtttttttccacagaaaaacatctgaagatggattgggtgagggctggcaaaacgaaggcatgccgggccagctccttaacccaatgacccagtgatgctgcaaggctggaacggggtccaggagactgtgtgtaacaggtgccctaggtgacccttataatcagggaagtttggtgaacaaaaatcgaacccatgagtgaacataaattaaaaagttgatcaacctattaaaatgtgtatttcattgggtagcttttctcactgtagacagattttttccttcttcaatgaaaaggcttttaaattagtacaactgttactatttaaaaaaaaaataccctaagtactctgtttacttctggtgaaacaaaaccagtcattagaaatggtctgtgcttttattttcccagactggagtggcttttctgaaacacacacacacacacacacacacacacacacacacacacnngtacacacatccctcacttctcttaagccaagaagtttgctttccctagctgcagtgtagatggctcttgtttttgtttttttgttttaatcatttggcattcacatgtggctgttaatatgtgcttgtttttaattaaaacaagaagctttaaaaaaaaaaaaaaaaaaaaaaaaa

SEQ ID NO: 58 - DNA, 2809 bp, Homo sapiens
ccggctcgcggagagcgtagcgcggccttggtggcggaatggcgttgagtgacggcccggccccgccatctggttaaagggactcgttcaacacggaagtgtcccggggctgcattgtgctacagctagaatgaagcacatcaacctatcatttgcagcgtgtggatttctgggcatttaccacttgggggcagcatctgcactttgcagacatggcaaaaaacttgtgaaggatgtcaaagccttcgctggggcgtctgcgggatcgttggttgcttctgttctgctaacagcaccagaaaaaatagaggaatgtaaccaatttacctacaagtttgccgaagaaatcagaaggcagtctttcggggcagtaacgcccggttatgacttcatggcccgactaagaagtgggatggagtcgattcttcctcccagcgctcacgagctggcccagaaccgactgcacgtatccatcaccaacgccaaaaccagagaaaatcacttagtctccactttttcctccagggaggacctcattaaggtcctcctagccagcagttttgtgcccatttatgcaggactgaagctagtggaatacaaagggcagaagtgggtggacggaggcctcaccaacgctcttcccatcctgcccgtcggccggacagtaaccatctcccccttcagtggacgactggacatctccccgcaggacaaagggcagctagatctgtatgttaatatcgccaagcaggatatcatgttgtccctggcaaacctggtgagactcaaccaagccctttttcccccaagcaagaggaaaatggaatctttgtatcagtgtggttttgatgacactgttaagtttttacttaaagaaaattggtttgaataaaatgcataaaagtttataatgcaaaacacgttttagatagtttttgatggaagtttctaatcaaatcctttttagaaaatctatcatgactcaattgtattactccttgtaatatttgtgtttatttttaatatttgaattattacttagcacatggatataagaggcactttattagaatttgacaacgtcattaagaaggatacacttaggtgataaggaatcatttttggtgaactgttgtgtcctttaaaaaatggaggaggataggttctttgggtaaattgaggaacatggaaaatggagggccaccacttaggttccatggagaatatgtggcatgatcttgacgtgaagcatgggtgtttccgtgacttagctgtgtccctgtagctctggaactgaaggaactaggtaggagaggaaggatttgagaaataggcaactcatggttaaagacctttgatcaggactggaggaacaaaggtttgtgtattagtccattttcacactgctgagaaagacatacccaagactgggcaatttacaaaagaaaggtttaatgggctcacagttccacatgactgaggaggcctcacaattgtggtggaaggtgaaaggcatgtctcacatggcagcagacaagagaagaaaacttgtgcagggaaactcccctttataaaaccatcggatctcttgagacgcgttcactatcatgagaatagcatgggaaagacctgcctccatgattcagttacctcccactgggtccctcccacaacgtgtggcaattgtgggagctacatacacttcaagatgagatttgggtggggacacagccaaaccataacagtttgctttccacaggtgtgtgcagttgctcccagtgcagttaggggcagatatccagaagcctaggagattttgaagaggcttatcccctctcatcttcctcccctaaaccccaccccagtttaggagattatcaagctggttgtcattcattctctctctctctctctctctctctctctctctctctctctctctctcgccatacctctctgagtatttagctattgttctatatactggatacatctattatgggaacttaaatagataaaatactactaaacacaaagaaccaagtagcctagtaaataagaattaaatccagaataatcattcacttctcatttttaaatcacgttgtactgatcccaggttttttactttctgagtagttgtcgatcactttgaggacttgtgtatgggcttgtaccaatttactgacactgtcttaaatcctaaatctgtttttatgaggttttgccaagcaagaactcttgattactaagaggcagttagagacaaaccaatttaattccatgtcttcaattgtcacgcatttcttcttttactctttgaaactattctttgagtcaattttatggttctcagaagccaaaatacacaacttttagcacataaacaccaacgatggcctctttttgaggagttatgcatagacccactctagagtaatgatggtccctgtggtatatactttctcctactctagcaaacatgtagtttaatcttaatgtgttgtttccataagtgacatgaagtggatagattctcaattgttatgtccacttattcactaggtaaattttcagttttaatacttttctccttaccccttcctttgatcatttcatgtgaatattctatttgtatgatacactgtatttcaataaattccttgttgatgtacccttaaattgaagaaatttaagctgcaaaaccaaatctcattgtataagacttttttgaagtatcttgtatcgactacatatgtatttgaccctgtgggaggatggtacttttctttttaacttttattttaag